SEQUENCE LISTING (501 sequences)

SEQ ID NO: 1 - protein, 12 aa, Artificial Sequence
  Description of Artificial Sequence: Synthetic peptide
LLADTTHHRPWT

SEQ ID NO: 2 - protein, 12 aa, Artificial Sequence
  Description of Artificial Sequence: Synthetic peptide
VIGESTHHRPWS

SEQ ID NO: 3 - protein, 12 aa, Artificial Sequence
  Description of Artificial Sequence: Synthetic peptide
LIADSTHHSPWT

SEQ ID NO: 4 - protein, 12 aa, Artificial Sequence
  Description of Artificial Sequence: Synthetic peptide
ILAESTHHKPWT

SEQ ID NO: 5 - protein, 12 aa, Artificial Sequence
  Description of Artificial Sequence: Synthetic peptide
ILAETTHHRPWS

SEQ ID NO: 6 - protein, 12 aa, Artificial Sequence
  Description of Artificial Sequence: Synthetic peptide
IIGESSHHKPFT

SEQ ID NO: 7 - protein, 12 aa, Artificial Sequence
  Description of Artificial Sequence: Synthetic peptide
GLGDTTHHRPWG

SEQ ID NO: 8 - protein, 12 aa, Artificial Sequence
  Description of Artificial Sequence: Synthetic peptide
VLGDTTHHKPWT

SEQ ID NO: 9 - protein, 12 aa, Artificial Sequence
  Description of Artificial Sequence: Synthetic peptide
IVADSTHHRPWT

SEQ ID NO: 10 - protein, 11 aa, Artificial Sequence
  Description of Artificial Sequence: Synthetic peptide
STADTSHHRPS

SEQ ID NO: 11 - protein, 12 aa, Artificial Sequence
  Description of Artificial Sequence: Synthetic peptide
TSGGESTHHRPS

SEQ ID NO: 12 - protein, 12 aa, Artificial Sequence
  Description of Artificial Sequence: Synthetic peptide
TSGGESSHHKPS

SEQ ID NO: 13 - protein, 12 aa, Artificial Sequence
  Description of Artificial Sequence: Synthetic peptide
TGSGDSSHHRPS

SEQ ID NO: 14 - protein, 13 aa, Artificial Sequence
  Description of Artificial Sequence: Synthetic peptide
GSSGESTHHKPST

SEQ ID NO: 15 - protein, 12 aa, Artificial Sequence
  Description of Artificial Sequence: Synthetic peptide
VGADSTHHRPVT

SEQ ID NO: 16 - protein, 12 aa, Artificial Sequence
  Description of Artificial Sequence: Synthetic peptide
GAADTTHHRPVT

SEQ ID NO: 17 - protein, 12 aa, Artificial Sequence
  Description of Artificial Sequence: Synthetic peptide
AGADTTHHRPVT

SEQ ID NO: 18 - protein, 12 aa, Artificial Sequence
  Description of Artificial Sequence: Synthetic peptide
GGADTTHHRPAT

SEQ ID NO: 19 - protein, 12 aa, Artificial Sequence
  Description of Artificial Sequence: Synthetic peptide
GGADTTHHRPGT

SEQ ID NO: 20 - protein, 24 aa, Artificial Sequence
  Description of Artificial Sequence: Synthetic peptide
LLADTTHHRPWTVIGESTHHRPWS

SEQ ID NO: 21 - protein, 36 aa, Artificial Sequence
  Description of Artificial Sequence: Synthetic polypeptide
LLADTTHHRPWTVIGESTHHRPWSIIGESSHHKPFT

SEQ ID NO: 22 - protein, 60 aa, Artificial Sequence
  Description of Artificial Sequence: Synthetic polypeptide
LLADTTHHRPWTVIGESTHHRPWSIIGESSHHKPFTGLGDTTHHRPWGILAESTHHKPWT

SEQ ID NO: 23 - protein, 24 aa, Artificial Sequence
  Description of Artificial Sequence: Synthetic peptide
LLADTTHHRPWTILAESTHHKPWT

SEQ ID NO: 24 - protein, 60 aa, Artificial Sequence
  Description of Artificial Sequence: Synthetic polypeptide
LLADTTHHRPWTILAESTHHKPWTLLADTTHHRPWTILAESTHHKPWTLLADTTHHRPWT

SEQ ID NO: 25 - protein, 24 aa, Artificial Sequence
  Description of Artificial Sequence: Synthetic peptide
LLADTTHHRPWTGLGDTTHHRPWG

SEQ ID NO: 26 - protein, 36 aa, Artificial Sequence
  Description of Artificial Sequence: Synthetic polypeptide
LLADTTHHRPWTGLGDTTHHRPWGLLADTTHHRPWT

SEQ ID NO: 27 - protein, 60 aa, Artificial Sequence
  Description of Artificial Sequence: Synthetic polypeptide
LLADTTHHRPWTGLGDTTHHRPWGLLADTTHHRPWTGLGDTTHHRPWGLLADTTHHRPWT

SEQ ID NO: 28 - protein, 84 aa, Artificial Sequence
  Description of Artificial Sequence: Synthetic polypeptide
LLADTTHHRPWTGLGDTTHHRPWGLLADTTHHRPWTGLGDTTHHRPWGLLADTTHHRPWTGLGDTTHHRPWGLLADTTHHRPWT

SEQ ID NO: 29 - protein, 60 aa, Artificial Sequence
  Description of Artificial Sequence: Synthetic polypeptide
STADTSHHRPSTSGGESTHHRPSTSGGESSHHKPSTGSGDSSHHRPSGSSGESTHHKPST

SEQ ID NO: 30 - protein, 60 aa, Artificial Sequence
  Description of Artificial Sequence: Synthetic polypeptide
VGADSTHHRPVTGAADTTHHRPVTAGADTTHHRPVTGGADTTHHRPATGGADTTHHRPGT

SEQ ID NO: 31 - protein, 108 aa, Artificial Sequence
  Description of Artificial Sequence: Synthetic polypeptide
STADTSHHRPSLLADTTHHRPWTTSGGESTHHRPSVGADSTHHRPVTTSGGESSHHKPSGAADTTHHRPVTTGSGDSSHHRPSGSSGESTHHKPSTGGADTTHHRPAT

SEQ ID NO: 32 - protein, 12 aa, Artificial Sequence
  Description of Artificial Sequence: Synthetic peptide
AAADTTHHRPWT

SEQ ID NO: 33 - protein, 60 aa, Artificial Sequence
  Description of Artificial Sequence: Synthetic polypeptide
AAADTTHHRPWTAAADTTHHRPWTAAADTTHHRPWTAAADTTHHRPWTAAADTTHHRPWT

SEQ ID NO: 34 - protein, 60 aa, Artificial Sequence
  Description of Artificial Sequence: Synthetic polypeptide
LLADAAHHRPWTLLADAAHHRPWTLLADAAHHRPWTLLADAAHHRPWTLLADAAHHRPWT

SEQ ID NO: 35 - protein, 60 aa, Artificial Sequence
  Description of Artificial Sequence: Synthetic polypeptide
LLADTTAARPWTLLADTTAARPWTLLADTTAARPWTLLADTTAARPWTLLADTTAARPWT

SEQ ID NO: 36 - protein, 24 aa, Artificial Sequence
  Description of Artificial Sequence: Synthetic peptide
LLADTTHHRPWTLLADTTHHRPWT

SEQ ID NO: 37 - protein, 36 aa, Artificial Sequence
  Description of Artificial Sequence: Synthetic polypeptide
LLADTTHHRPWTLLADTTHHRPWTLLADTTHHRPWT

SEQ ID NO: 38 - protein, 60 aa, Artificial Sequence
  Description of Artificial Sequence: Synthetic polypeptide
LLADTTHHRPWTLLADTTHHRPWTLLADTTHHRPWTLLADTTHHRPWTLLADTTHHRPWT

SEQ ID NO: 39 - protein, 120 aa, Artificial Sequence
  Description of Artificial Sequence: Synthetic polypeptide
STSGSTVIGESTHHRPWSLIADSTHHSPWTILAESTHHKPWTILAETTHHRPWSIIGESSHHKPFTGLGDTTHHRPWGVLGDTTHHKPWTIVADSTHHRPWTGQVLPTTTPSSPSTTSGS

SEQ ID NO: 40 - protein, 48 aa, Artificial Sequence
  Description of Artificial Sequence: Synthetic polypeptide
LLADTTHHRPWTVIGESTHHRPWSIIGESSHHKPFTGLGDTTHHRPWG

SEQ ID NO: 41 - protein, 48 aa, Artificial Sequence
  Description of Artificial Sequence: Synthetic polypeptide
VIGESTHHRPWSIIGESSHHKPFTGLGDTTHHRPWGILAESTHHKPWT

SEQ ID NO: 42 - protein, 132 aa, Artificial Sequence
  Description of Artificial Sequence: Synthetic polypeptide
  MOD_RES (13)..(132): This region may encompass one of the following sequences: “LLADTTHHRPWT” or “VIGESTHHRPWS” or “LIADSTHHSPWT” or “ILAESTHHKPWT” or “ILAETTHHRPWS” or “IIGESSHHKPFT” or “GLGDTTHHRPWG” or “VLGDTTHHKPWT” or “IVADSTHHRPWT” or
  MOD_RES (13)..(132): CONT. FROM ABOVE: “STADTSHHRPS” or “TSGGESTHHRPS” or “TSGGESSHHKPS” or “TGSGDSSHHRPS” or “GSSGESTHHKPST” or “VGADSTHHRPVT” or “GAADTTHHRPVT” or “AGADTTHHRPVT” or “GGADTTHHRPAT” or “GGADTTHHRPGT” or
  MOD_RES (13)..(132): CONT. FROM ABOVE: “LLADTTHHRPWTVIGESTHHRPWS” or “LLADTTHHRPWTVIGESTHHRPWSIIGESSHHKPFT” or “LLADTTHHRPWTVIGESTHHRPWSIIGESSHHKPFTGLGDTTHHRPWGILAESTHHKPWT” or “LLADTTHHRPWTILAESTHHKPWT” or “LLADTTHHRPWTILAESTHHK
  MOD_RES (13)..(132): CONT. FROM ABOVE: PWTLLADTTHHRPWTILAESTHHKPWTLLADTTHHRPWT” or “LLADTTHHRPWTGLGDTTHHRPWG” or “LLADTTHHRPWTGLGDTTHHRPWGLLADTTHHRPWT” or “LLADTTHHRPWTGLGDTTHHRPWGLLADTTHHRPWTGLGDTTHHRPWGLLADTTHHRPWT” or “LLADTTHHRPWTGLGDTTHHRPWGLLADTTHHRPWTGLGD
  MOD_RES (13)..(132): CONT. FROM ABOVE: TTHHRPWGLLADTTHHRPWTGLGDTTHHRPWGLLADTTHHRPWT” or “STADTSHHRPSTSGGESTHHRPSTSGGESSHHKPSTGSGDSSHHRPSGSSGESTHHKPST” or “VGADSTHHRPVTGAADTTHHRPVTAGADTTHHRPVTGGADTTHHRPATGGADTTHHRPGT” or “STADTSHHRPSLLADTTHHRPWTTSGGESTHHRPSVGADSTHHRPVTTSGGESSHHKPSG
  MOD_RES (13)..(132): CONT. FROM ABOVE: AADTTHHRPVTTGSGDSSHHRPSGSSGESTHHKPSTGGADTTHHRPAT” or “AAADTTHHRPWT” or “AAADTTHHRPWTAAADTTHHRPWTAAADTTHHRPWTAAADTTHHRPWTAAADTTHHRPWT” or “LLADAAHHRPWTLLADAAHHRPWTLLADAAHHRPWTLLADAAHHRPWTLLADAAHHRPWT” or “LLADTTAARPWTLLADTTAARPWTLLADTT
  MOD_RES (13)..(132): CONT. FROM ABOVE: AARPWTLLADTTAARPWTLLADTTAARPWT” or “LLADTTHHRPWTLLADTTHHRPWT” or “LLADTTHHRPWTLLADTTHHRPWTLLADTTHHRPWT” or “LLADTTHHRPWTLLADTTHHRPWTLLADTTHHRPWTLLADTTHHRPWTLLADTTHHRPWT” or “STSGSTVIGESTHHRPWSLIADSTHHSPWTILAESTHHKPWT
  MOD_RES (13)..(132): CONT. FROM ABOVE: ILAETTHHRPWSIIGESSHHKPFTGLGDTTHHRPWGVLGDTTHHKPWTIVADSTHHRPWTGQVLPT TTPSSPSTTSGS” or “LLADTTHHRPWTVIGESTHHRPWSIIGESSHHKPFTGLGDTTHHRPWG” or “VIGESTHHRPWSIIGESSHHKPFTGLGDTTHHRPWGILAESTHHKPWT”
  See specification as filed for detailed description of substitutions and preferred embodiments
LLADTTHHRPWTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 43 - protein, 132 aa, Artificial Sequence
  Description of Artificial Sequence: Synthetic polypeptide
  MOD_RES (13)..(132): This region may encompass one of the following sequences: “LLADTTHHRPWT” or “VIGESTHHRPWS” or “LIADSTHHSPWT” or “ILAESTHHKPWT” or “ILAETTHHRPWS” or “IIGESSHHKPFT” or “GLGDTTHHRPWG” or “VLGDTTHHKPWT” or “IVADSTHHRPWT” or
  MOD_RES (13)..(132): CONT. FROM ABOVE: “STADTSHHRPS” or “TSGGESTHHRPS” or “TSGGESSHHKPS” or “TGSGDSSHHRPS” or “GSSGESTHHKPST” or “VGADSTHHRPVT” or “GAADTTHHRPVT” or “AGADTTHHRPVT” or “GGADTTHHRPAT” or “GGADTTHHRPGT” or
  MOD_RES (13)..(132): CONT. FROM ABOVE: “LLADTTHHRPWTVIGESTHHRPWS” or “LLADTTHHRPWTVIGESTHHRPWSIIGESSHHKPFT” or “LLADTTHHRPWTVIGESTHHRPWSIIGESSHHKPFTGLGDTTHHRPWGILAESTHHKPWT” or “LLADTTHHRPWTILAESTHHKPWT” or “LLADTTHHRPWTILAESTHHK
  MOD_RES (13)..(132): CONT. FROM ABOVE: PWTLLADTTHHRPWTILAESTHHKPWTLLADTTHHRPWT” or “LLADTTHHRPWTGLGDTTHHRPWG” or “LLADTTHHRPWTGLGDTTHHRPWGLLADTTHHRPWT” or “LLADTTHHRPWTGLGDTTHHRPWGLLADTTHHRPWTGLGDTTHHRPWGLLADTTHHRPWT” or “LLADTTHHRPWTGLGDTTHHRPWGLLADTTHHRPWTGLGD
  MOD_RES (13)..(132): CONT. FROM ABOVE: TTHHRPWGLLADTTHHRPWTGLGDTTHHRPWGLLADTTHHRPWT” or “STADTSHHRPSTSGGESTHHRPSTSGGESSHHKPSTGSGDSSHHRPSGSSGESTHHKPST” or “VGADSTHHRPVTGAADTTHHRPVTAGADTTHHRPVTGGADTTHHRPATGGADTTHHRPGT” or “STADTSHHRPSLLADTTHHRPWTTSGGESTHHRPSVGADSTHHRPVTTSGGESSHHKPSG
  MOD_RES (13)..(132): CONT. FROM ABOVE: AADTTHHRPVTTGSGDSSHHRPSGSSGESTHHKPSTGGADTTHHRPAT” or “AAADTTHHRPWT” or “AAADTTHHRPWTAAADTTHHRPWTAAADTTHHRPWTAAADTTHHRPWTAAADTTHHRPWT” or “LLADAAHHRPWTLLADAAHHRPWTLLADAAHHRPWTLLADAAHHRPWTLLADAAHHRPWT” or “LLADTTAARPWTLLADTTAARPWTLLADTT
  MOD_RES (13)..(132): CONT. FROM ABOVE: AARPWTLLADTTAARPWTLLADTTAARPWT” or “LLADTTHHRPWTLLADTTHHRPWT” or “LLADTTHHRPWTLLADTTHHRPWTLLADTTHHRPWT” or “LLADTTHHRPWTLLADTTHHRPWTLLADTTHHRPWTLLADTTHHRPWTLLADTTHHRPWT” or “STSGSTVIGESTHHRPWSLIADSTHHSPWTILAESTHHKPWT
  MOD_RES (13)..(132): CONT. FROM ABOVE: ILAETTHHRPWSIIGESSHHKPFTGLGDTTHHRPWGVLGDTTHHKPWTIVADSTHHRPWTGQVLP TTTPSSPSTTSGS” or “LLADTTHHRPWTVIGESTHHRPWSIIGESSHHKPFTGLGDTTHHRPWG” or “VIGESTHHRPWSIIGESSHHKPFTGLGDTTHHRPWGILAESTHHKPWT”
  See specification as filed for detailed description of substitutions and preferred embodiments
VIGESTHHRPWSXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 44 - protein, 132 aa, Artificial Sequence
  Description of Artificial Sequence: Synthetic polypeptide
  MOD_RES (13)..(132): This region may encompass one of the following sequences: “LLADTTHHRPWT” or “VIGESTHHRPWS” or “LIADSTHHSPWT” or “ILAESTHHKPWT” or “ILAETTHHRPWS” or “IIGESSHHKPFT” or “GLGDTTHHRPWG” or “VLGDTTHHKPWT” or “IVADSTHHRPWT” or
  MOD_RES (13)..(132): CONT. FROM ABOVE: “STADTSHHRPS” or “TSGGESTHHRPS” or “TSGGESSHHKPS” or “TGSGDSSHHRPS” or “GSSGESTHHKPST” or “VGADSTHHRPVT” or “GAADTTHHRPVT” or “AGADTTHHRPVT” or “GGADTTHHRPAT” or “GGADTTHHRPGT” or
  MOD_RES (13)..(132): CONT. FROM ABOVE: “LLADTTHHRPWTVIGESTHHRPWS” or “LLADTTHHRPWTVIGESTHHRPWSIIGESSHHKPFT” or “LLADTTHHRPWTVIGESTHHRPWSIIGESSHHKPFTGLGDTTHHRPWGILAESTHHKPWT” or “LLADTTHHRPWTILAESTHHKPWT” or “LLADTTHHRPWTILAESTHHK
  MOD_RES (13)..(132): CONT. FROM ABOVE: PWTLLADTTHHRPWTILAESTHHKPWTLLADTTHHRPWT” or “LLADTTHHRPWTGLGDTTHHRPWG” or “LLADTTHHRPWTGLGDTTHHRPWGLLADTTHHRPWT” or “LLADTTHHRPWTGLGDTTHHRPWGLLADTTHHRPWTGLGDTTHHRPWGLLADTTHHRPWT” or “LLADTTHHRPWTGLGDTTHHRPWGLLADTTHHRPWTGLGD
  MOD_RES (13)..(132): CONT. FROM ABOVE: TTHHRPWGLLADTTHHRPWTGLGDTTHHRPWGLLADTTHHRPWT” or “STADTSHHRPSTSGGESTHHRPSTSGGESSHHKPSTGSGDSSHHRPSGSSGESTHHKPST” or “VGADSTHHRPVTGAADTTHHRPVTAGADTTHHRPVTGGADTTHHRPATGGADTTHHRPGT” or “STADTSHHRPSLLADTTHHRPWTTSGGESTHHRPSVGADSTHHRPVTTSGGESSHHKPSG
  MOD_RES (13)..(132): CONT. FROM ABOVE: AADTTHHRPVTTGSGDSSHHRPSGSSGESTHHKPSTGGADTTHHRPAT” or “AAADTTHHRPWT” or “AAADTTHHRPWTAAADTTHHRPWTAAADTTHHRPWTAAADTTHHRPWTAAADTTHHRPWT” or “LLADAAHHRPWTLLADAAHHRPWTLLADAAHHRPWTLLADAAHHRPWTLLADAAHHRPWT” or “LLADTTAARPWTLLADTTAARPWTLLADTT
  MOD_RES (13)..(132): CONT. FROM ABOVE: AARPWTLLADTTAARPWTLLADTTAARPWT” or “LLADTTHHRPWTLLADTTHHRPWT” or “LLADTTHHRPWTLLADTTHHRPWTLLADTTHHRPWT” or “LLADTTHHRPWTLLADTTHHRPWTLLADTTHHRPWTLLADTTHHRPWTLLADTTHHRPWT” or “STSGSTVIGESTHHRPWSLIADSTHHSPWTILAESTHHKPWT
  MOD_RES (13)..(132): CONT. FROM ABOVE: ILAETTHHRPWSIIGESSHHKPFTGLGDTTHHRPWGVLGDTTHHKPWTIVADSTHHRPWTGQVLP TTTPSSPSTTSGS” or “LLADTTHHRPWTVIGESTHHRPWSIIGESSHHKPFTGLGDTTHHRPWG” or “VIGESTHHRPWSIIGESSHHKPFTGLGDTTHHRPWGILAESTHHKPWT”
  See specification as filed for detailed description of substitutions and preferred embodiments
ILAESTHHKPWTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 45 - protein, 132 aa, Artificial Sequence
  Description of Artificial Sequence: Synthetic polypeptide
  MOD_RES (13)..(132): This region may encompass one of the following sequences: “LLADTTHHRPWT” or “VIGESTHHRPWS” or “LIADSTHHSPWT” or “ILAESTHHKPWT” or “ILAETTHHRPWS” or “IIGESSHHKPFT” or “GLGDTTHHRPWG” or “VLGDTTHHKPWT” or “IVADSTHHRPWT” or
  MOD_RES (13)..(132): CONT. FROM ABOVE: “STADTSHHRPS” or “TSGGESTHHRPS” or “TSGGESSHHKPS” or “TGSGDSSHHRPS” or “GSSGESTHHKPST” or “VGADSTHHRPVT” or “GAADTTHHRPVT” or “AGADTTHHRPVT” or “GGADTTHHRPAT” or “GGADTTHHRPGT” or
  MOD_RES (13)..(132): CONT. FROM ABOVE: “LLADTTHHRPWTVIGESTHHRPWS” or “LLADTTHHRPWTVIGESTHHRPWSIIGESSHHKPFT” or “LLADTTHHRPWTVIGESTHHRPWSIIGESSHHKPFTGLGDTTHHRPWGILAESTHHKPWT” or “LLADTTHHRPWTILAESTHHKPWT” or “LLADTTHHRPWTILAESTHHK
  MOD_RES (13)..(132): CONT. FROM ABOVE: PWTLLADTTHHRPWTILAESTHHKPWTLLADTTHHRPWT” or “LLADTTHHRPWTGLGDTTHHRPWG” or “LLADTTHHRPWTGLGDTTHHRPWGLLADTTHHRP WT” or “LLADTTHHRPWTGLGDTTHHRPWGLLADTTHHRPWTGLGDTTHHRPWGLLADTTHHR PWT” or “LLADTTHHRPWTGLGDTTHHRPWGLLADTTHHRPWTGLGD
  MOD_RES (13)..(132): CONT. FROM ABOVE: TTHHRPWGLLADTTHHRPWTGLGDTTHHRPWGLLADTTHHRPWT” or “STADTSHHRPSTSGGESTHHRPSTSGGESSHHKPSTGSGDSSHHRPSGSSGESTHHKPST” or “VGADSTHHRPVTGAADTTHHRPVTAGADTTHHRPVTGGADTTHHRPATGGADTTHHRPGT” or “STADTSHHRPSLLADTTHHRPWTTSGGESTHHRPSVGADSTHHRPVTTSGGESSHHKPSG
  MOD_RES (13)..(132): CONT. FROM ABOVE: AADTTHHRPVTTGSGDSSHHRPSGSSGESTHHKPSTGGADTTHHRPAT” or “AAADTTHHRPWT” or “AAADTTHHRPWTAAADTTHHRPWTAAADTTHHRPWTAAADTTHHRPWTAAADTTHHRPWT” or “LLADAAHHRPWTLLADAAHHRPWTLLADAAHHRPWTLLADAAHHRPWTLLADAAHHRPWT” or “LLADTTAARPWTLLADTTAARPWTLLADTT
  MOD_RES (13)..(132): CONT. FROM ABOVE: AARPWTLLADTTAARPWTLLADTTAARPWT” or “LLADTTHHRPWTLLADTTHHRPWT” or “LLADTTHHRPWTLLADTTHHRPWTLLADTTHHRPWT” or “LLADTTHHRPWTLLADTTHHRPWTLLADTTHHRPWTLLADTTHHRPWTLLADTTHHRPWT” or “STSGSTVIGESTHHRPWSLIADSTHHSPWTILAESTHHKPWT
  MOD_RES (13)..(132): CONT. FROM ABOVE: ILAETTHHRPWSIIGESSHHKPFTGLGDTTHHRPWGVLGDTTHHKPWTIVADSTHHRPWTGQVLP TTTPSSPSTTSGS” or “LLADTTHHRPWTVIGESTHHRPWSIIGESSHHKPFTGLGDTTHHRPWG” or “VIGESTHHRPWSIIGESSHHKPFTGLGDTTHHRPWGILAESTHHKPWT”
  See specification as filed for detailed description of substitutions and preferred embodiments
IIGESSHHKPFTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 46 - protein, 132 aa, Artificial Sequence
  Description of Artificial Sequence: Synthetic polypeptide
  MOD_RES (13)..(132): This region may encompass one of the following sequences: “LLADTTHHRPWT” or “VIGESTHHRPWS” or “LIADSTHHSPWT” or “ILAESTHHKPWT” or “ILAETTHHRPWS” or “IIGESSHHKPFT” or “GLGDTTHHRPWG” or “VLGDTTHHKPWT” or “IVADSTHHRPWT” or
  MOD_RES (13)..(132): CONT. FROM ABOVE: “STADTSHHRPS” or “TSGGESTHHRPS” or “TSGGESSHHKPS” or “TGSGDSSHHRPS” or “GSSGESTHHKPST” or “VGADSTHHRPVT” or “GAADTTHHRPVT” or “AGADTTHHRPVT” or “GGADTTHHRPAT” or “GGADTTHHRPGT” or
  MOD_RES (13)..(132): CONT. FROM ABOVE: “LLADTTHHRPWTVIGESTHHRPWS” or “LLADTTHHRPWTVIGESTHHRPWSIIGESSHHKPFT” or “LLADTTHHRPWTVIGESTHHRPWSIIGESSHHKPFTGLGDTTHHRPWGILAESTHHKPWT” or “LLADTTHHRPWTILAESTHHKPWT” or “LLADTTHHRPWTILAESTHHK
  MOD_RES (13)..(132): CONT. FROM ABOVE: PWTLLADTTHHRPWTILAESTHHKPWTLLADTTHHRPWT” or “LLADTTHHRPWTGLGDTTHHRPWG” or “LLADTTHHRPWTGLGDTTHHRPWGLLADTTHHRPWT” or “LLADTTHHRPWTGLGDTTHHRPWGLLADTTHHRPWTGLGDTTHHRPWGLLADTTHHRPWT” or “LLADTTHHRPWTGLGDTTHHRPWGLLADTTHHRPWTGLGD
  MOD_RES (13)..(132): CONT. FROM ABOVE: TTHHRPWGLLADTTHHRPWTGLGDTTHHRPWGLLADTTHHRPWT” or “STADTSHHRPSTSGGESTHHRPSTSGGESSHHKPSTGSGDSSHHRPSGSSGESTHHKPST” or “VGADSTHHRPVTGAADTTHHRPVTAGADTTHHRPVTGGADTTHHRPATGGADTTHHRPGT” or “STADTSHHRPSLLADTTHHRPWTTSGGESTHHRPSVGADSTHHRPVTTSGGESSHHKPSG
  MOD_RES (13)..(132): CONT. FROM ABOVE: AADTTHHRPVTTGSGDSSHHRPSGSSGESTHHKPSTGGADTTHHRPAT” or “AAADTTHHRPWT” or “AAADTTHHRPWTAAADTTHHRPWTAAADTTHHRPWTAAADTTHHRPWTAAADTTHHRPWT” or “LLADAAHHRPWTLLADAAHHRPWTLLADAAHHRPWTLLADAAHHRPWTLLADAAHHRPWT” or “LLADTTAARPWTLLADTTAARPWTLLADTT
  MOD_RES (13)..(132): CONT. FROM ABOVE: AARPWTLLADTTAARPWTLLADTTAARPWT” or “LLADTTHHRPWTLLADTTHHRPWT” or “LLADTTHHRPWTLLADTTHHRPWTLLADTTHHRPWT” or “LLADTTHHRPWTLLADTTHHRPWTLLADTTHHRPWTLLADTTHHRPWTLLADTTHHRPWT” or “STSGSTVIGESTHHRPWSLIADSTHHSPWTILAESTHHKPWT
  MOD_RES (13)..(132): CONT. FROM ABOVE: ILAETTHHRPWSIIGESSHHKPFTGLGDTTHHRPWGVLGDTTHHKPWTIVADSTHHRPWTGQVLP TTTPSSPSTTSGS” or “LLADTTHHRPWTVIGESTHHRPWSIIGESSHHKPFTGLGDTTHHRPWG” or “VIGESTHHRPWSIIGESSHHKPFTGLGDTTHHRPWGILAESTHHKPWT”
  See specification as filed for detailed description of substitutions and preferred embodiments
GLGDTTHHRPWGXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 47 - protein, 24 aa, Artificial Sequence
  Description of Artificial Sequence: Synthetic peptide
  MOD_RES (13)..(24): This region may encompass one of the following sequences: “VIGESTHHRPWS” or “ILAESTHHKPWT” or “IIGESSHHKPFT” or “GLGDTTHHRPWG”
  See specification as filed for detailed description of substitutions and preferred embodiments
LLADTTHHRPWTXXXXXXXXXXXX

SEQ ID NO: 48 - protein, 24 aa, Artificial Sequence
  Description of Artificial Sequence: Synthetic peptide
  MOD_RES (13)..(24): This region may encompass one of the following sequences: “LLADTTHHRPWT” or “ILAESTHHKPWT” or “IIGESSHHKPFT” or “GLGDTTHHRPWG”
  See specification as filed for detailed description of substitutions and preferred embodiments
VIGESTHHRPWSXXXXXXXXXXXX

SEQ ID NO: 49 - protein, 24 aa, Artificial Sequence
  Description of Artificial Sequence: Synthetic peptide
  MOD_RES (13)..(24): This region may encompass one of the following sequences: “LLADTTHHRPWT” or “VIGESTHHRPWS” or “IIGESSHHKPFT” or “GLGDTTHHRPWG”
  See specification as filed for detailed description of substitutions and preferred embodiments
ILAESTHHKPWTXXXXXXXXXXXX

SEQ ID NO: 50 - protein, 24 aa, Artificial Sequence
  Description of Artificial Sequence: Synthetic peptide
  MOD_RES (13)..(24): This region may encompass one of the following sequences: “LLADTTHHRPWT” or “ILAESTHHKPWT” or “VIGESTHHRPWS” or “GLGDTTHHRPWG”
  See specification as filed for detailed description of substitutions and preferred embodiments
IIGESSHHKPFTXXXXXXXXXXXX

SEQ ID NO: 51 - protein, 24 aa, Artificial Sequence
  Description of Artificial Sequence: Synthetic peptide
  MOD_RES (13)..(24): This region may encompass one of the following sequences: “LLADTTHHRPWT” or “ILAESTHHKPWT” or “IIGESSHHKPFT” or “VIGESTHHRPWS”
  See specification as filed for detailed description of substitutions and preferred embodiments
GLGDTTHHRPWGXXXXXXXXXXXX

SEQ ID NO: 52 - protein, 36 aa, Artificial Sequence
  Description of Artificial Sequence: Synthetic polypeptide
  MOD_RES (13)..(36): This region may encompass two of the following sequences: “VIGESTHHRPWS” or “ILAESTHHKPWT” or “IIGESSHHKPFT” or “GLGDTTHHRPWG”
  See specification as filed for detailed description of substitutions and preferred embodiments
LLADTTHHRPWTXXXXXXXXXXXXXXXXXXXXXXXX

SEQ ID NO: 53 - protein, 36 aa, Artificial Sequence
  Description of Artificial Sequence: Synthetic polypeptide
  MOD_RES (13)..(36): This region may encompass two of the following sequences: “LLADTTHHRPWT” or “ILAESTHHKPWT” or “IIGESSHHKPFT” or “GLGDTTHHRPWG”
  See specification as filed for detailed description of substitutions and preferred embodiments
VIGESTHHRPWSXXXXXXXXXXXXXXXXXXXXXXXX

SEQ ID NO: 54 - protein, 36 aa, Artificial Sequence
  Description of Artificial Sequence: Synthetic polypeptide
  MOD_RES (13)..(36): This region may encompass two of the following sequences: “LLADTTHHRPWT” or “VIGESTHHRPWS” or “IIGESSHHKPFT” or “GLGDTTHHRPWG”
  See specification as filed for detailed description of substitutions and preferred embodiments
ILAESTHHKPWTXXXXXXXXXXXXXXXXXXXXXXXX

SEQ ID NO: 55 - protein, 36 aa, Artificial Sequence
  Description of Artificial Sequence: Synthetic polypeptide
  MOD_RES (13)..(36): This region may encompass two of the following sequences: “LLADTTHHRPWT” or “ILAESTHHKPWT” or “VIGESTHHRPWS” or “GLGDTTHHRPWG”
  See specification as filed for detailed description of substitutions and preferred embodiments
IIGESSHHKPFTXXXXXXXXXXXXXXXXXXXXXXXX

SEQ ID NO: 56 - protein, 36 aa, Artificial Sequence
  Description of Artificial Sequence: Synthetic polypeptide
  MOD_RES (13)..(36): This region may encompass two of the following sequences: “LLADTTHHRPWT” or “ILAESTHHKPWT” or “IIGESSHHKPFT” or “VIGESTHHRPWS”
  See specification as filed for detailed description of substitutions and preferred embodiments
GLGDTTHHRPWGXXXXXXXXXXXXXXXXXXXXXXXX

SEQ ID NO: 57 - protein, 48 aa, Artificial Sequence
  Description of Artificial Sequence: Synthetic polypeptide
  MOD_RES (13)..(48): This region may encompass three of the following sequences: “VIGESTHHRPWS” or “ILAESTHHKPWT” or “IIGESSHHKPFT” or “GLGDTTHHRPWG”
  See specification as filed for detailed description of substitutions and preferred embodiments
LLADTTHHRPWTXXXXXXXXXXXXXXXXXXXXXXXXXXXXXXXXXXXX

SEQ ID NO: 58 - protein, 48 aa, Artificial Sequence
  Description of Artificial Sequence: Synthetic polypeptide
  MOD_RES (13)..(48): This region may encompass three of the following sequences: “LLADTTHHRPWT” or “ILAESTHHKPWT” or “IIGESSHHKPFT” or “GLGDTTHHRPWG”
  See specification as filed for detailed description of substitutions and preferred embodiments
VIGESTHHRPWSXXXXXXXXXXXXXXXXXXXXXXXXXXXXXXXXXXXX

SEQ ID NO: 59 - protein, 48 aa, Artificial Sequence
  Description of Artificial Sequence: Synthetic polypeptide
  MOD_RES (13)..(48): This region may encompass three of the following sequences: “LLADTTHHRPWT” or “VIGESTHHRPWS” or “IIGESSHHKPFT” or “GLGDTTHHRPWG”
  See specification as filed for detailed description of substitutions and preferred embodiments
ILAESTHHKPWTXXXXXXXXXXXXXXXXXXXXXXXXXXXXXXXXXXXX

SEQ ID NO: 60 - protein, 48 aa, Artificial Sequence
  Description of Artificial Sequence: Synthetic polypeptide
  MOD_RES (13)..(48): This region may encompass three of the following sequences: “LLADTTHHRPWT” or “ILAESTHHKPWT” or “VIGESTHHRPWS” or “GLGDTTHHRPWG”
  See specification as filed for detailed description of substitutions and preferred embodiments
IIGESSHHKPFTXXXXXXXXXXXXXXXXXXXXXXXXXXXXXXXXXXXX

SEQ ID NO: 61 - protein, 48 aa, Artificial Sequence
  Description of Artificial Sequence: Synthetic polypeptide
  MOD_RES (13)..(48): This region may encompass three of the following sequences: “LLADTTHHRPWT” or “ILAESTHHKPWT” or “IIGESSHHKPFT” or “VIGESTHHRPWS”
  See specification as filed for detailed description of substitutions and preferred embodiments
GLGDTTHHRPWGXXXXXXXXXXXXXXXXXXXXXXXXXXXXXXXXXXXX

SEQ ID NO: 62 - protein, 10 aa, Artificial Sequence
  Description of Artificial Sequence: Synthetic peptide
ACAPLMFSQC

SEQ ID NO: 63 - protein, 10 aa, Artificial Sequence
  Description of Artificial Sequence: Synthetic peptide
ACHASLKHRC

SEQ ID NO: 64 - protein, 10 aa, Artificial Sequence
  Description of Artificial Sequence: Synthetic peptide
ACLSTKTNIC

SEQ ID NO: 65 - protein, 10 aa, Artificial Sequence
  Description of Artificial Sequence: Synthetic peptide
ACTTPSKHQC

SEQ ID NO: 66 - protein, 11 aa, Artificial Sequence
  Description of Artificial Sequence: Synthetic peptide
AHFSPNLLLGG

SEQ ID NO: 67 - protein, 12 aa, Artificial Sequence
  Description of Artificial Sequence: Synthetic peptide
AHSLKSITNHGL

SEQ ID NO: 68 - protein, 7 aa, Artificial Sequence
  Description of Artificial Sequence: Synthetic peptide
AKQTVPV

SEQ ID NO: 69 - protein, 12 aa, Artificial Sequence
  Description of Artificial Sequence: Synthetic peptide
AKTLMPSPFPRT

SEQ ID NO: 70 - protein, 12 aa, Artificial Sequence
  Description of Artificial Sequence: Synthetic peptide
AMSQTMTAAIEK

SEQ ID NO: 71 - protein, 7 aa, Artificial Sequence
  Description of Artificial Sequence: Synthetic peptide
ANPPLSL

SEQ ID NO: 72 - protein, 7 aa, Artificial Sequence
  Description of Artificial Sequence: Synthetic peptide
ANPYHRH

SEQ ID NO: 73 - protein, 7 aa, Artificial Sequence
  Description of Artificial Sequence: Synthetic peptide
APLSLSL

SEQ ID NO: 74 - protein, 16 aa, Artificial Sequence
  Description of Artificial Sequence: Synthetic peptide
APYHPTIPASVHGGGK

SEQ ID NO: 75 - protein, 12 aa, Artificial Sequence
  Description of Artificial Sequence: Synthetic peptide
  MOD_RES (12)..(12): Any amino acid
ASAVGSLSIRWX

SEQ ID NO: 76 - protein, 7 aa, Artificial Sequence
  Description of Artificial Sequence: Synthetic peptide
ASGPTNV

SEQ ID NO: 77 - protein, 7 aa, Artificial Sequence
  Description of Artificial Sequence: Synthetic peptide
ASHNPKL

SEQ ID NO: 78 - protein, 12 aa, Artificial Sequence
  Description of Artificial Sequence: Synthetic peptide
ASWVDSRQPSAA

SEQ ID NO: 79 - protein, 7 aa, Artificial Sequence
  Description of Artificial Sequence: Synthetic peptide
ATFSPPL

SEQ ID NO: 80 - protein, 12 aa, Artificial Sequence
  Description of Artificial Sequence: Synthetic peptide
ATWSHHLSSAGL

SEQ ID NO: 81 - protein, 16 aa, Artificial Sequence
  Description of Artificial Sequence: Synthetic peptide
ATWSHHLSSAGLGGGS

SEQ ID NO: 82 - protein, 9 aa, Artificial Sequence
  Description of Artificial Sequence: Synthetic peptide
CAHLSPHKC

SEQ ID NO: 83 - protein, 9 aa, Artificial Sequence
  Description of Artificial Sequence: Synthetic peptide
CDIPWRNEC

SEQ ID NO: 84 - protein, 9 aa, Artificial Sequence
  Description of Artificial Sequence: Synthetic peptide
CDPLRQHSC

SEQ ID NO: 85 - protein, 9 aa, Artificial Sequence
  Description of Artificial Sequence: Synthetic peptide
CDSLGHWLC

SEQ ID NO: 86 - protein, 9 aa, Artificial Sequence
  Description of Artificial Sequence: Synthetic peptide
CDYTTRHSC

SEQ ID NO: 87 - protein, 9 aa, Artificial Sequence
  Description of Artificial Sequence: Synthetic peptide
CHGTLNPEC

SEQ ID NO: 88 - protein, 9 aa, Artificial Sequence
  Description of Artificial Sequence: Synthetic peptide
CHHNLSWEC

SEQ ID NO: 89 - protein, 9 aa, Artificial Sequence
  Description of Artificial Sequence: Synthetic peptide
CHIWTLASC

SEQ ID NO: 90 - protein, 9 aa, Artificial Sequence
  Description of Artificial Sequence: Synthetic peptide
CHNTFSPRC

SEQ ID NO: 91 - protein, 9 aa, Artificial Sequence
  Description of Artificial Sequence: Synthetic peptide
CIPLHASLC

SEQ ID NO: 92 - protein, 9 aa, Artificial Sequence
  Description of Artificial Sequence: Synthetic peptide
CITTTSLSC

SEQ ID NO: 93 - protein, 9 aa, Artificial Sequence
  Description of Artificial Sequence: Synthetic peptide
CKLTTCKDC

SEQ ID NO: 94 - protein, 9 aa, Artificial Sequence
  Description of Artificial Sequence: Synthetic peptide
CKNHTTFWC

SEQ ID NO: 95 - protein, 9 aa, Artificial Sequence
  Description of Artificial Sequence: Synthetic peptide
CLKLLSRSC

SEQ ID NO: 96 - protein, 9 aa, Artificial Sequence
  Description of Artificial Sequence: Synthetic peptide
CLLKAHPSC

SEQ ID NO: 97 - protein, 9 aa, Artificial Sequence
  Description of Artificial Sequence: Synthetic peptide
CLNQLKQAC

SEQ ID NO: 98 - protein, 9 aa, Artificial Sequence
  Description of Artificial Sequence: Synthetic peptide
CLSTKTNIC

SEQ ID NO: 99 - protein, 9 aa, Artificial Sequence
  Description of Artificial Sequence: Synthetic peptide
CMNFPSPHC

SEQ ID NO: 100 - protein, 9 aa, Artificial Sequence
  Description of Artificial Sequence: Synthetic peptide
CNYPTLKSC

SEQ ID NO: 101 - protein, 11 aa, Artificial Sequence
  Description of Artificial Sequence: Synthetic peptide
CPQLTVGQHRT

SEQ ID NO: 102 - protein, 9 aa, Artificial Sequence
  Description of Artificial Sequence: Synthetic peptide
CPQSPTYTC

SEQ ID NO: 103 - protein, 9 aa, Artificial Sequence
  Description of Artificial Sequence: Synthetic peptide
CPSSAIHTC

SEQ ID NO: 104 - protein, 9 aa, Artificial Sequence
  Description of Artificial Sequence: Synthetic peptide
CPTSTARIC

SEQ ID NO: 105 - protein, 9 aa, Artificial Sequence
  Description of Artificial Sequence: Synthetic peptide
CQASSFPSC

SEQ ID NO: 106 - protein, 9 aa, Artificial Sequence
  Description of Artificial Sequence: Synthetic peptide
CQPYFWYRC

SEQ ID NO: 107 - protein, 9 aa, Artificial Sequence
  Description of Artificial Sequence: Synthetic peptide
CQTLTPSIC

SEQ ID NO: 108 - protein, 9 aa, Artificial Sequence
  Description of Artificial Sequence: Synthetic peptide
CSKLGHLWC

SEQ ID NO: 109 - protein, 9 aa, Artificial Sequence
  Description of Artificial Sequence: Synthetic peptide
  MOD_RES (9)..(9): Any amino acid
CSKTPERIX

SEQ ID NO: 110 - protein, 9 aa, Artificial Sequence
  Description of Artificial Sequence: Synthetic peptide
CSNNNRMTC

SEQ ID NO: 111 - protein, 9 aa, Artificial Sequence
  Description of Artificial Sequence: Synthetic peptide
CSPILSLSC

SEQ ID NO: 112 - protein, 9 aa, Artificial Sequence
  Description of Artificial Sequence: Synthetic peptide
CSPTNFTRC

SEQ ID NO: 113 - protein, 9 aa, Artificial Sequence
  Description of Artificial Sequence: Synthetic peptide
CSRPAMNVC

SEQ ID NO: 114 - protein, 9 aa, Artificial Sequence
  Description of Artificial Sequence: Synthetic peptide
CSTKAYPNC

SEQ ID NO: 115 - protein, 9 aa, Artificial Sequence
  Description of Artificial Sequence: Synthetic peptide
CSTSSCGSC

SEQ ID NO: 116 - protein, 9 aa, Artificial Sequence
  Description of Artificial Sequence: Synthetic peptide
CSYWGHRDC

SEQ ID NO: 117 - protein, 9 aa, Artificial Sequence
  Description of Artificial Sequence: Synthetic peptide
CTAHDANAC

SEQ ID NO: 118 - protein, 9 aa, Artificial Sequence
  Description of Artificial Sequence: Synthetic peptide
CTANSEKTC

SEQ ID NO: 119 - protein, 9 aa, Artificial Sequence
  Description of Artificial Sequence: Synthetic peptide
CTHPKASMC

SEQ ID NO: 120 - protein, 9 aa, Artificial Sequence
  Description of Artificial Sequence: Synthetic peptide
CTKTINGKC

SEQ ID NO: 121 - protein, 9 aa, Artificial Sequence
  Description of Artificial Sequence: Synthetic peptide
CTNMQSPLC

SEQ ID NO: 122 - protein, 9 aa, Artificial Sequence
  Description of Artificial Sequence: Synthetic peptide
CTPFTKLPC

SEQ ID NO: 123 - protein, 9 aa, Artificial Sequence
  Description of Artificial Sequence: Synthetic peptide
CTPTTDSIC

SEQ ID NO: 124 - protein, 9 aa, Artificial Sequence
  Description of Artificial Sequence: Synthetic peptide
CTQQNGHPC

SEQ ID NO: 125 - protein, 9 aa, Artificial Sequence
  Description of Artificial Sequence: Synthetic peptide
CTTPSKHQC

SEQ ID NO: 126 - protein, 9 aa, Artificial Sequence
  Description of Artificial Sequence: Synthetic peptide
CTYNVAKPC

SEQ ID NO: 127 - protein, 7 aa, Artificial Sequence
  Description of Artificial Sequence: Synthetic peptide
DKLHRLA

SEQ ID NO: 128 - protein, 12 aa, Artificial Sequence
  Description of Artificial Sequence: Synthetic peptide
DLNYFTLSSKRE

SEQ ID NO: 129 - protein, 12 aa, Artificial Sequence
  Description of Artificial Sequence: Synthetic peptide
DLPPTLHTTGSP

SEQ ID NO: 130 - protein, 7 aa, Artificial Sequence
  Description of Artificial Sequence: Synthetic peptide
DMRQQRS

SEQ ID NO: 131 - protein, 7 aa, Artificial Sequence
  Description of Artificial Sequence: Synthetic peptide
DQYWGLR

SEQ ID NO: 132 - protein, 12 aa, Artificial Sequence
  Description of Artificial Sequence: Synthetic peptide
DSSNPIFWRPSS

SEQ ID NO: 133 - protein, 16 aa, Artificial Sequence
  Description of Artificial Sequence: Synthetic peptide
DSSNPIFWRPSSGGGS

SEQ ID NO: 134 - protein, 12 aa, Artificial Sequence
  Description of Artificial Sequence: Synthetic peptide
EFLGVPASLVNP

SEQ ID NO: 135 - protein, 7 aa, Artificial Sequence
  Description of Artificial Sequence: Synthetic peptide
EPNHTRF

SEQ ID NO: 136 - protein, 9 aa, Artificial Sequence
  Description of Artificial Sequence: Synthetic peptide
EPRRAVAAL

SEQ ID NO: 137 - protein, 9 aa, Artificial Sequence
  Description of Artificial Sequence: Synthetic peptide
EPRRAVAEL

SEQ ID NO: 138 - protein, 9 aa, Artificial Sequence
  Description of Artificial Sequence: Synthetic peptide
EPRREVAEL

SEQ ID NO: 139 - protein, 9 aa, Artificial Sequence
  Description of Artificial Sequence: Synthetic peptide
EPRREVCEL

SEQ ID NO: 140 - protein, 12 aa, Artificial Sequence
  Description of Artificial Sequence: Synthetic peptide
ESDLTHALHWLG

SEQ ID NO: 141 - protein, 7 aa, Artificial Sequence
  Description of Artificial Sequence: Synthetic peptide
ESLKSIS

SEQ ID NO: 142 - protein, 7 aa, Artificial Sequence
  Description of Artificial Sequence: Synthetic peptide
ETRTQLL

SEQ ID NO: 143 - protein, 7 aa, Artificial Sequence
  Description of Artificial Sequence: Synthetic peptide
ETVCASS

SEQ ID NO: 144 - protein, 7 aa, Artificial Sequence
  Description of Artificial Sequence: Synthetic peptide
ETYARPL

SEQ ID NO: 145 - protein, 7 aa, Artificial Sequence
  Description of Artificial Sequence: Synthetic peptide
ETYQQPL

SEQ ID NO: 146 - protein, 12 aa, Artificial Sequence
  Description of Artificial Sequence: Synthetic peptide
EVHSTDRYRSIP

SEQ ID NO: 147 - protein, 12 aa, Artificial Sequence
  Description of Artificial Sequence: Synthetic peptide
FGLQPTGDIARR

SEQ ID NO: 148 - protein, 12 aa, Artificial Sequence
  Description of Artificial Sequence: Synthetic peptide
FSMDDPERVRSP

SEQ ID NO: 149 - protein, 12 aa, Artificial Sequence
  Description of Artificial Sequence: Synthetic peptide
FSPLHTSTYRPS

SEQ ID NO: 150 - protein, 7 aa, Artificial Sequence
  Description of Artificial Sequence: Synthetic peptide
FTLPTIR

SEQ ID NO: 151 - protein, 7 aa, Artificial Sequence
  Description of Artificial Sequence: Synthetic peptide
FVNLLGQ

SEQ ID NO: 152 - protein, 12 aa, Artificial Sequence
  Description of Artificial Sequence: Synthetic peptide
GDFNSGHHTTTR

SEQ ID NO: 153 - protein, 7 aa, Artificial Sequence
  Description of Artificial Sequence: Synthetic peptide
GGGAAAA

SEQ ID NO: 154 - protein, 12 aa, Artificial Sequence
  Description of Artificial Sequence: Synthetic peptide
GIHVPWMPPVAF

SEQ ID NO: 155 - protein, 16 aa, Artificial Sequence
  Description of Artificial Sequence: Synthetic peptide
GIHVPWMPPVAFGGGS

SEQ ID NO: 156 - protein, 12 aa, Artificial Sequence
  Description of Artificial Sequence: Synthetic peptide
GPSNNLPWSNTP

SEQ ID NO: 157 - protein, 12 aa, Artificial Sequence
  Description of Artificial Sequence: Synthetic peptide
GSAGLKYPLYKS

SEQ ID NO: 158 - protein, 7 aa, Artificial Sequence
  Description of Artificial Sequence: Synthetic peptide
GSCPPKK

SEQ ID NO: 159 - protein, 7 aa, Artificial Sequence
  Description of Artificial Sequence: Synthetic peptide
GSLFKAL

SEQ ID NO: 160 - protein, 7 aa, Artificial Sequence
  Description of Artificial Sequence: Synthetic peptide
GTQTPQP

SEQ ID NO: 161 - protein, 7 aa, Artificial Sequence
  Description of Artificial Sequence: Synthetic peptide
GTSRLFS

SEQ ID NO: 162 - protein, 12 aa, Artificial Sequence
  Description of Artificial Sequence: Synthetic peptide
GVHKHFYSRWLG

SEQ ID NO: 163 - protein, 12 aa, Artificial Sequence
  Description of Artificial Sequence: Synthetic peptide
HAPLTRSPAPNL

SEQ ID NO: 164 - protein, 7 aa, Artificial Sequence
  Description of Artificial Sequence: Synthetic peptide
HAPVQPN

SEQ ID NO: 165 - protein, 12 aa, Artificial Sequence
  Description of Artificial Sequence: Synthetic peptide
  MOD_RES (8)..(8): Any amino acid
HGSLTTLXRYEP

SEQ ID NO: 166 - protein, 12 aa, Artificial Sequence
  Description of Artificial Sequence: Synthetic peptide
HHFHLPKLRPPV

SEQ ID NO: 167 - protein, 7 aa, Artificial Sequence
  Description of Artificial Sequence: Synthetic peptide
HHQRSPA

SEQ ID NO: 168 - protein, 12 aa, Artificial Sequence
  Description of Artificial Sequence: Synthetic peptide
HHTWDTRIWQAF

SEQ ID NO: 169 - protein, 7 aa, Artificial Sequence
  Description of Artificial Sequence: Synthetic peptide
HMLAQTF

SEQ ID NO: 170 - protein, 12 aa, Artificial Sequence
  Description of Artificial Sequence: Synthetic peptide
HNVTTRTQRLMP

SEQ ID NO: 171 - protein, 11 aa, Artificial Sequence
  Description of Artificial Sequence: Synthetic peptide
HPTTPIHMPNF

SEQ ID NO: 172 - protein, 12 aa, Artificial Sequence
  Description of Artificial Sequence: Synthetic peptide
HQFISPEPFLIS

SEQ ID NO: 173 - protein, 12 aa, Artificial Sequence
  Description of Artificial Sequence: Synthetic peptide
  MOD_RES (5)..(5): Any amino acid
HQFPXSNLVWKP

SEQ ID NO: 174 - protein, 7 aa, Artificial Sequence
  Description of Artificial Sequence: Synthetic peptide
HQWDHKY

SEQ ID NO: 175 - protein, 13 aa, Artificial Sequence
  Description of Artificial Sequence: Synthetic peptide
  MOD_RES (5)..(5): Any amino acid
  MOD_RES (7)..(7): Any amino acid
  MOD_RES (11)..(11): Any amino acid
HRDPXSXPSAXRP

SEQ ID NO: 176 - protein, 7 aa, Artificial Sequence
  Description of Artificial Sequence: Synthetic peptide
HRLGHMS

SEQ ID NO: 177 - protein, 12 aa, Artificial Sequence
  Description of Artificial Sequence: Synthetic peptide
HSACHASLKHRC

SEQ ID NO: 178 - protein, 12 aa, Artificial Sequence
  Description of Artificial Sequence: Synthetic peptide
HSACKLTTCKDG

SEQ ID NO: 179 - protein, 12 aa, Artificial Sequence
  Description of Artificial Sequence: Synthetic peptide
HSACLSTKTNIC

SEQ ID NO: 180 - protein, 12 aa, Artificial Sequence
  Description of Artificial Sequence: Synthetic peptide
HSMPHMGTYLLT

SEQ ID NO: 181 - protein, 7 aa, Artificial Sequence
  Description of Artificial Sequence: Synthetic peptide
HSTGPTR

SEQ ID NO: 182 - protein, 7 aa, Artificial Sequence
  Description of Artificial Sequence: Synthetic peptide
HTLLSTT

SEQ ID NO: 183 - protein, 7 aa, Artificial Sequence
  Description of Artificial Sequence: Synthetic peptide
HYPTVNF

SEQ ID NO: 184 - protein, 12 aa, Artificial Sequence
  Description of Artificial Sequence: Synthetic peptide
IAHVPETRLAQM

SEQ ID NO: 185 - protein, 12 aa, Artificial Sequence
  Description of Artificial Sequence: Synthetic peptide
IFSMGTALARPL

SEQ ID NO: 186 - protein, 7 aa, Artificial Sequence
  Description of Artificial Sequence: Synthetic peptide
IGYPVLP

SEQ ID NO: 187 - protein, 12 aa, Artificial Sequence
  Description of Artificial Sequence: Synthetic peptide
INFQFLKPSTTR

SEQ ID NO: 188 - protein, 12 aa, Artificial Sequence
  Description of Artificial Sequence: Synthetic peptide
INKHPQQVSTLL

SEQ ID NO: 189 - protein, 7 aa, Artificial Sequence
  Description of Artificial Sequence: Synthetic peptide
IQHQAKT

SEQ ID NO: 190 - protein, 7 aa, Artificial Sequence
  Description of Artificial Sequence: Synthetic peptide
  MOD_RES (3)..(3): Any amino acid
  MOD_RES (5)..(5): Any amino acid
IRXLXIS

SEQ ID NO: 191 - protein, 12 aa, Artificial Sequence
  Description of Artificial Sequence: Synthetic peptide
ISPSHSQAQADL

SEQ ID NO: 192 - protein, 7 aa, Artificial Sequence
  Description of Artificial Sequence: Synthetic peptide
KAFDKHG

SEQ ID NO: 193 - protein, 7 aa, Artificial Sequence
  Description of Artificial Sequence: Synthetic peptide
KATITGM

SEQ ID NO: 194 - protein, 12 aa, Artificial Sequence
  Description of Artificial Sequence: Synthetic peptide
KEIPPIPLLAPS

SEQ ID NO: 195 - protein, 16 aa, Artificial Sequence
  Description of Artificial Sequence: Synthetic peptide
KEIPPIPLLAPSGGGS

SEQ ID NO: 196 - protein, 20 aa, Artificial Sequence
  Description of Artificial Sequence: Synthetic peptide
KIPKACCVPTELSAISMLYL

SEQ ID NO: 197 - protein, 33 aa, Artificial Sequence
  Description of Artificial Sequence: Synthetic polypeptide
KIPKASSVPTELSAIATLYLAAAAEPRRAVAAL

SEQ ID NO: 198 - protein, 20 aa, Artificial Sequence
  Description of Artificial Sequence: Synthetic peptide
KIPKASSVPTELSAISTLYL

SEQ ID NO: 199 - protein, 33 aa, Artificial Sequence
  Description of Artificial Sequence: Synthetic polypeptide
KIPKASSVPTELSAISTLYLAAAAEPRRAVAAL

SEQ ID NO: 200 - protein, 33 aa, Artificial Sequence
  Description of Artificial Sequence: Synthetic polypeptide
KIPKASSVPTELSAISTLYLAAAAEPRRAVAEL

SEQ ID NO: 201 - protein, 33 aa, Artificial Sequence
  Description of Artificial Sequence: Synthetic polypeptide
KIPKASSVPTELSAISTLYLAAAAEPRREVAEL

SEQ ID NO: 202 - protein, 33 aa, Artificial Sequence
  Description of Artificial Sequence: Synthetic polypeptide
  MOD_RES (25)..(25): Any amino acid
  MOD_RES (29)..(29): Any amino acid
  MOD_RES (32)..(32): Any amino acid
KIPKASSVPTELSAISTLYLAAAAXPRRXVAXL

SEQ ID NO: 203 - protein, 29 aa, Artificial Sequence
  Description of Artificial Sequence: Synthetic peptide
  MOD_RES (21)..(21): Any amino acid
  MOD_RES (25)..(25): Any amino acid
  MOD_RES (28)..(28): Any amino acid
KIPKASSVPTELSAISTLYLXPRRXVAXL

SEQ ID NO: 204 - protein, 7 aa, Artificial Sequence
  Description of Artificial Sequence: Synthetic peptide
KLHASLA

SEQ ID NO: 205 - protein, 7 aa, Artificial Sequence
  Description of Artificial Sequence: Synthetic peptide
KLSAWSF

SEQ ID NO: 206 - protein, 15 aa, Artificial Sequence
  Description of Artificial Sequence: Synthetic peptide
KLTWQELYQLKYKGI

SEQ ID NO: 207 - protein, 31 aa, Artificial Sequence
  Description of Artificial Sequence: Synthetic polypeptide
KLTWQELYQLKYKGIGGGAAAAEPRREVAEL

SEQ ID NO: 208 - protein, 7 aa, Artificial Sequence
  Description of Artificial Sequence: Synthetic peptide
KMNHMPN

SEQ ID NO: 209 - protein, 7 aa, Artificial Sequence
  Description of Artificial Sequence: Synthetic peptide
KPMQFVH

SEQ ID NO: 210 - protein, 7 aa, Artificial Sequence
  Description of Artificial Sequence: Synthetic peptide
KTSSWAN

SEQ ID NO: 211 - protein, 7 aa, Artificial Sequence
  Description of Artificial Sequence: Synthetic peptide
LASTTHV

SEQ ID NO: 212 - protein, 12 aa, Artificial Sequence
  Description of Artificial Sequence: Synthetic peptide
LDYPIPQTVLHH

SEQ ID NO: 213 - protein, 12 aa, Artificial Sequence
  Description of Artificial Sequence: Synthetic peptide
LFAAVPSTQFFR

SEQ ID NO: 214 - protein, 12 aa, Artificial Sequence
  Description of Artificial Sequence: Synthetic peptide
LGFDPTSTRFYT

SEQ ID NO: 215 - protein, 7 aa, Artificial Sequence
  Description of Artificial Sequence: Synthetic peptide
LGPGKAF

SEQ ID NO: 216 - protein, 7 aa, Artificial Sequence
  Description of Artificial Sequence: Synthetic peptide
LKPFSGA

SEQ ID NO: 217 - protein, 12 aa, Artificial Sequence
  Description of Artificial Sequence: Synthetic peptide
LLADTTHHRPWP

SEQ ID NO: 218 - protein, 12 aa, Artificial Sequence
  Description of Artificial Sequence: Synthetic peptide
LLADTTHHRPWT

SEQ ID NO: 219 - protein, 16 aa, Artificial Sequence
  Description of Artificial Sequence: Synthetic peptide
LLADTTHHRPWTGGGS

SEQ ID NO: 220 - protein, 7 aa, Artificial Sequence
  Description of Artificial Sequence: Synthetic peptide
LLPLKFK

SEQ ID NO: 221 - protein, 7 aa, Artificial Sequence
  Description of Artificial Sequence: Synthetic peptide
LPFQPPI

SEQ ID NO: 222 - protein, 7 aa, Artificial Sequence
  Description of Artificial Sequence: Synthetic peptide
LPLTPLP

SEQ ID NO: 223 - protein, 12 aa, Artificial Sequence
  Description of Artificial Sequence: Synthetic peptide
LPRDLHATPQQI

SEQ ID NO: 224 - protein, 7 aa, Artificial Sequence
  Description of Artificial Sequence: Synthetic peptide
LPSIHNL

SEQ ID NO: 225 - protein, 12 aa, Artificial Sequence
  Description of Artificial Sequence: Synthetic peptide
LPWAPNLPDSTA

SEQ ID NO: 226 - protein, 12 aa, Artificial Sequence
  Description of Artificial Sequence: Synthetic peptide
LPWTEPSFWRTP

SEQ ID NO: 227 - protein, 16 aa, Artificial Sequence
  Description of Artificial Sequence: Synthetic peptide
LPWTEPSFWRTPGGGS

SEQ ID NO: 228 - protein, 7 aa, Artificial Sequence
  Description of Artificial Sequence: Synthetic peptide
LQKSPSL

SEQ ID NO: 229 - protein, 12 aa, Artificial Sequence
  Description of Artificial Sequence: Synthetic peptide
LQPSQPQRFAPT

SEQ ID NO: 230 - protein, 12 aa, Artificial Sequence
  Description of Artificial Sequence: Synthetic peptide
LRAFPSLPHTVT

SEQ ID NO: 231 - protein, 7 aa, Artificial Sequence
  Description of Artificial Sequence: Synthetic peptide
LSAPMEY

SEQ ID NO: 232 - protein, 7 aa, Artificial Sequence
  Description of Artificial Sequence: Synthetic peptide
LSKNPLL

SEQ ID NO: 233 - protein, 12 aa, Artificial Sequence
  Description of Artificial Sequence: Synthetic peptide
LSLRASAATDFQ

SEQ ID NO: 234 - protein, 12 aa, Artificial Sequence
  Description of Artificial Sequence: Synthetic peptide
LSPPMQLQPTYS

SEQ ID NO: 235 - protein, 12 aa, Artificial Sequence
  Description of Artificial Sequence: Synthetic peptide
LTPTMFNMHGVL

SEQ ID NO: 236 - protein, 7 aa, Artificial Sequence
  Description of Artificial Sequence: Synthetic peptide
LTQTLQY

SEQ ID NO: 237 - protein, 12 aa, Artificial Sequence
  Description of Artificial Sequence: Synthetic peptide
MHNVSDSNDSAI

SEQ ID NO: 238 - protein, 7 aa, Artificial Sequence
  Description of Artificial Sequence: Synthetic peptide
MKVHERS

SEQ ID NO: 239 - protein, 12 aa, Artificial Sequence
  Description of Artificial Sequence: Synthetic peptide
MPQTLVLPRSLL

SEQ ID NO: 240 - protein, 12 aa, Artificial Sequence
  Description of Artificial Sequence: Synthetic peptide
MQFTPAPSPSDH

SEQ ID NO: 241 - protein, 7 aa, Artificial Sequence
  Description of Artificial Sequence: Synthetic peptide
MTSQTLR

SEQ ID NO: 242 - protein, 7 aa, Artificial Sequence
  Description of Artificial Sequence: Synthetic peptide
MYPLPAP

SEQ ID NO: 243 - protein, 7 aa, Artificial Sequence
  Description of Artificial Sequence: Synthetic peptide
NERQMEL

SEQ ID NO: 244 - protein, 7 aa, Artificial Sequence
  Description of Artificial Sequence: Synthetic peptide
NFAMNLR

SEQ ID NO: 245 - protein, 7 aa, Artificial Sequence
  Description of Artificial Sequence: Synthetic peptide
NITQLGS

SEQ ID NO: 246 - protein, 7 aa, Artificial Sequence
  Description of Artificial Sequence: Synthetic peptide
NKPLSTL

SEQ ID NO: 247 - protein, 12 aa, Artificial Sequence
  Description of Artificial Sequence: Synthetic peptide
NNVSQKWQQRLI

SEQ ID NO: 248 - protein, 16 aa, Artificial Sequence
  Description of Artificial Sequence: Synthetic peptide
NNVSQKWQQRLIGGGS

SEQ ID NO: 249 - protein, 16 aa, Artificial Sequence
  Description of Artificial Sequence: Synthetic peptide
NPDHPDIPQDVHGGGK

SEQ ID NO: 250 - protein, 7 aa, Artificial Sequence
  Description of Artificial Sequence: Synthetic peptide
NPMIMNQ

SEQ ID NO: 251 - protein, 7 aa, Artificial Sequence
  Description of Artificial Sequence: Synthetic peptide
NPQMQRS

SEQ ID NO: 252 - protein, 7 aa, Artificial Sequence
  Description of Artificial Sequence: Synthetic peptide
NPRSQAT

SEQ ID NO: 253 - protein, 16 aa, Artificial Sequence
  Description of Artificial Sequence: Synthetic peptide
NPYAPTIPQSVAGGGK

SEQ ID NO: 254 - protein, 12 aa, Artificial Sequence
  Description of Artificial Sequence: Synthetic peptide
NPYHPTIPQSVH

SEQ ID NO: 255 - protein, 16 aa, Artificial Sequence
  Description of Artificial Sequence: Synthetic peptide
NPYHPTIPQSVHGGGK

SEQ ID NO: 256 - protein, 12 aa, Artificial Sequence
  Description of Artificial Sequence: Synthetic peptide
NSMIAHNKTRMH

SEQ ID NO: 257 - protein, 16 aa, Artificial Sequence
  Description of Artificial Sequence: Synthetic peptide
NSMIAHNKTRMHGGGS

SEQ ID NO: 258 - protein, 12 aa, Artificial Sequence
  Description of Artificial Sequence: Synthetic peptide
NSSMLGMLPSSF

SEQ ID NO: 259 - protein, 12 aa, Artificial Sequence
  Description of Artificial Sequence: Synthetic peptide
NTSSSQGTQRLG

SEQ ID NO: 260 - protein, 12 aa, Artificial Sequence
  Description of Artificial Sequence: Synthetic peptide
NTTTDIPSPSQF

SEQ ID NO: 261 - protein, 7 aa, Artificial Sequence
  Description of Artificial Sequence: Synthetic peptide
NYPTLKS

SEQ ID NO: 262 - protein, 12 aa, Artificial Sequence
  Description of Artificial Sequence: Synthetic peptide
NYSHLRVKLPTP

SEQ ID NO: 263 - protein, 16 aa, Artificial Sequence
  Description of Artificial Sequence: Synthetic peptide
NYSHLRVKLPTPGGGS

SEQ ID NO: 264 - protein, 7 aa, Artificial Sequence
  Description of Artificial Sequence: Synthetic peptide
PAKQKAH

SEQ ID NO: 265 - protein, 7 aa, Artificial Sequence
  Description of Artificial Sequence: Synthetic peptide
PDIPLSR

SEQ ID NO: 266 - protein, 12 aa, Artificial Sequence
  Description of Artificial Sequence: Synthetic peptide
PGQWPSSLTLYK

SEQ ID NO: 267 - protein, 7 aa, Artificial Sequence
  Description of Artificial Sequence: Synthetic peptide
PHNPGKL

SEQ ID NO: 268 - protein, 7 aa, Artificial Sequence
  Description of Artificial Sequence: Synthetic peptide
PIDAFFD

SEQ ID NO: 269 - protein, 7 aa, Artificial Sequence
  Description of Artificial Sequence: Synthetic peptide
PLTQPSH

SEQ ID NO: 270 - protein, 7 aa, Artificial Sequence
  Description of Artificial Sequence: Synthetic peptide
PPKDSRG

SEQ ID NO: 271 - protein, 7 aa, Artificial Sequence
  Description of Artificial Sequence: Synthetic peptide
PPNMARA

SEQ ID NO: 272 - protein, 7 aa, Artificial Sequence
  Description of Artificial Sequence: Synthetic peptide
PSMKHWR

SEQ ID NO: 273 - protein, 7 aa, Artificial Sequence
  Description of Artificial Sequence: Synthetic peptide
PTNKPHT

SEQ ID NO: 274 - protein, 7 aa, Artificial Sequence
  Description of Artificial Sequence: Synthetic peptide
PTTMTRW

SEQ ID NO: 275 - protein, 7 aa, Artificial Sequence
  Description of Artificial Sequence: Synthetic peptide
PTTWGHL

SEQ ID NO: 276 - protein, 36 aa, Artificial Sequence
  Description of Artificial Sequence: Synthetic polypeptide
  MOD_RES (2)..(2): Any amino acid
  MOD_RES (5)..(5): Any amino acid
  MOD_RES (8)..(8): Any amino acid
  MOD_RES (11)..(11): Any amino acid
  MOD_RES (14)..(14): Any amino acid
  MOD_RES (17)..(17): Any amino acid
  MOD_RES (20)..(20): Any amino acid
  MOD_RES (23)..(23): Any amino acid
  MOD_RES (26)..(26): Any amino acid
  MOD_RES (29)..(29): Any amino acid
  MOD_RES (32)..(32): Any amino acid
  MOD_RES (35)..(35): Any amino acid
PXGPXGPXGPXGPXGPXAPXGPXGPXGPXGPXGPXG

SEQ ID NO: 277 - protein, 12 aa, Artificial Sequence
  Description of Artificial Sequence: Synthetic peptide
QHNFRGASSSAP

SEQ ID NO: 278 - protein, 12 aa, Artificial Sequence
  Description of Artificial Sequence: Synthetic peptide
QIPQMRILHPYG

SEQ ID NO: 279 - protein, 12 aa, Artificial Sequence
  Description of Artificial Sequence: Synthetic peptide
QIQKPPRTPPSL

SEQ ID NO: 280 - protein, 12 aa, Artificial Sequence
  Description of Artificial Sequence: Synthetic peptide
QLTQTMWKDTTL

SEQ ID NO: 281 - protein, 12 aa, Artificial Sequence
  Description of Artificial Sequence: Synthetic peptide
QNLPPERYSEAT

SEQ ID NO: 282 - protein, 7 aa, Artificial Sequence
  Description of Artificial Sequence: Synthetic peptide
QNPRQIY

SEQ ID NO: 283 - protein, 7 aa, Artificial Sequence
  Description of Artificial Sequence: Synthetic peptide
QNYLLPK

SEQ ID NO: 284 - protein, 7 aa, Artificial Sequence
  Description of Artificial Sequence: Synthetic peptide
QPGLWPS

SEQ ID NO: 285 - protein, 12 aa, Artificial Sequence
  Description of Artificial Sequence: Synthetic peptide
QRSWTLDSALSM

SEQ ID NO: 286 - protein, 16 aa, Artificial Sequence
  Description of Artificial Sequence: Synthetic peptide
QRSWTLDSALSMGGGS

SEQ ID NO: 287 - protein, 12 aa, Artificial Sequence
  Description of Artificial Sequence: Synthetic peptide
QSLSFAGPPAWQ

SEQ ID NO: 288 - protein, 7 aa, Artificial Sequence
  Description of Artificial Sequence: Synthetic peptide
QSSYNPI

SEQ ID NO: 289 - protein, 7 aa, Artificial Sequence
  Description of Artificial Sequence: Synthetic peptide
QTHARHQ

SEQ ID NO: 290 - protein, 7 aa, Artificial Sequence
  Description of Artificial Sequence: Synthetic peptide
QTHSSLW

SEQ ID NO: 291 - protein, 12 aa, Artificial Sequence
  Description of Artificial Sequence: Synthetic peptide
QTTMTPLWPSFS

SEQ ID NO: 292 - protein, 12 aa, Artificial Sequence
  Description of Artificial Sequence: Synthetic peptide
RCMSEVISFNCP

SEQ ID NO: 293 - protein, 7 aa, Artificial Sequence
  Description of Artificial Sequence: Synthetic peptide
RHTLPLH

SEQ ID NO: 294 - protein, 7 aa, Artificial Sequence
  Description of Artificial Sequence: Synthetic peptide
RPHTITN

SEQ ID NO: 295 - protein, 12 aa, Artificial Sequence
  Description of Artificial Sequence: Synthetic peptide
RSPYYNKWSSKF

SEQ ID NO: 296 - protein, 12 aa, Artificial Sequence
  Description of Artificial Sequence: Synthetic peptide
RTPLQPLEDFRP

SEQ ID NO: 297 - protein, 12 aa, Artificial Sequence
  Description of Artificial Sequence: Synthetic peptide
SAGHIHEAHRPL

SEQ ID NO: 298 - protein, 12 aa, Artificial Sequence
  Description of Artificial Sequence: Synthetic peptide
SAISDHRAHRSH

SEQ ID NO: 299 - protein, 7 aa, Artificial Sequence
  Description of Artificial Sequence: Synthetic peptide
SAKGRAD

SEQ ID NO: 300 - protein, 7 aa, Artificial Sequence
  Description of Artificial Sequence: Synthetic peptide
SAKKVFS

SEQ ID NO: 301 - protein, 7 aa, Artificial Sequence
  Description of Artificial Sequence: Synthetic peptide
SASGTPS

SEQ ID NO: 302 - protein, 12 aa, Artificial Sequence
  Description of Artificial Sequence: Synthetic peptide
SEPTYWRPNMSG

SEQ ID NO: 303 - protein, 12 aa, Artificial Sequence
  Description of Artificial Sequence: Synthetic peptide
SFAPDIKYPVPS

SEQ ID NO: 304 - protein, 12 aa, Artificial Sequence
  Description of Artificial Sequence: Synthetic peptide
SFQSMSLMTLVV

SEQ ID NO: 305 - protein, 12 aa, Artificial Sequence
  Description of Artificial Sequence: Synthetic peptide
SFWHHHSPRSPL

SEQ ID NO: 306 - protein, 12 aa, Artificial Sequence
  Description of Artificial Sequence: Synthetic peptide
SGHQLLLNKMPN

SEQ ID NO: 307 - protein, 16 aa, Artificial Sequence
  Description of Artificial Sequence: Synthetic peptide
SGHQLLLNKMPNGGGS

SEQ ID NO: 308 - protein, 12 aa, Artificial Sequence
  Description of Artificial Sequence: Synthetic peptide
SIFAHQTPTHKN

SEQ ID NO: 309 - protein, 12 aa, Artificial Sequence
  Description of Artificial Sequence: Synthetic peptide
SIPKMIPTESLL

SEQ ID NO: 310 - protein, 12 aa, Artificial Sequence
  Description of Artificial Sequence: Synthetic peptide
SIPSHSIHSAKA

SEQ ID NO: 311 - protein, 12 aa, Artificial Sequence
  Description of Artificial Sequence: Synthetic peptide
SIRTSMNPPNLL

SEQ ID NO: 312 - protein, 7 aa, Artificial Sequence
  Description of Artificial Sequence: Synthetic peptide
SKTSSTS

SEQ ID NO: 313 - protein, 7 aa, Artificial Sequence
  Description of Artificial Sequence: Synthetic peptide
SLLTPWL

SEQ ID NO: 314 - protein, 12 aa, Artificial Sequence
  Description of Artificial Sequence: Synthetic peptide
SLPHYIDNPFRQ

SEQ ID NO: 315 - protein, 12 aa, Artificial Sequence
  Description of Artificial Sequence: Synthetic peptide
SLSKANILHLYG

SEQ ID NO: 316 - protein, 12 aa, Artificial Sequence
  Description of Artificial Sequence: Synthetic peptide
SLVTADASFTPS

SEQ ID NO: 317 - protein, 7 aa, Artificial Sequence
  Description of Artificial Sequence: Synthetic peptide
SMAAKSS

SEQ ID NO: 318 - protein, 12 aa, Artificial Sequence
  Description of Artificial Sequence: Synthetic peptide
SMVYGNRLPSAL

SEQ ID NO: 319 - protein, 7 aa, Artificial Sequence
  Description of Artificial Sequence: Synthetic peptide
SMYDTHS

SEQ ID NO: 320 - protein, 7 aa, Artificial Sequence
  Description of Artificial Sequence: Synthetic peptide
SPEMKPR

SEQ ID NO: 321 - protein, 12 aa, Artificial Sequence
  Description of Artificial Sequence: Synthetic peptide
SPNFSWLPLGTT

SEQ ID NO: 322 - protein, 12 aa, Artificial Sequence
  Description of Artificial Sequence: Synthetic peptide
SPNLPWSKLSAY

SEQ ID NO: 323 - protein, 7 aa, Artificial Sequence
  Description of Artificial Sequence: Synthetic peptide
SPNNPRE

SEQ ID NO: 324 - protein, 7 aa, Artificial Sequence
  Description of Artificial Sequence: Synthetic peptide
SPNNTRE

SEQ ID NO: 325 - protein, 12 aa, Artificial Sequence
  Description of Artificial Sequence: Synthetic peptide
SPSLMARSSPYW

SEQ ID NO: 326 - protein, 7 aa, Artificial Sequence
  Description of Artificial Sequence: Synthetic peptide
SQHSTQD

SEQ ID NO: 327 - protein, 12 aa, Artificial Sequence
  Description of Artificial Sequence: Synthetic peptide
SQTLPYSNAPSP

SEQ ID NO: 328 - protein, 7 aa, Artificial Sequence
  Description of Artificial Sequence: Synthetic peptide
SRTGAHH

SEQ ID NO: 329 - protein, 7 aa, Artificial Sequence
  Description of Artificial Sequence: Synthetic peptide
SSHHHRH

SEQ ID NO: 330 - protein, 7 aa, Artificial Sequence
  Description of Artificial Sequence: Synthetic peptide
SSPPRVY

SEQ ID NO: 331 - protein, 7 aa, Artificial Sequence
  Description of Artificial Sequence: Synthetic peptide
SSSMAKM

SEQ ID NO: 332 - protein, 12 aa, Artificial Sequence
  Description of Artificial Sequence: Synthetic peptide
SSTLKTFFGFPD

SEQ ID NO: 333 - protein, 16 aa, Artificial Sequence
  Description of Artificial Sequence: Synthetic peptide
SSTLKTFFGFPDGGGS

SEQ ID NO: 334 - protein, 12 aa, Artificial Sequence
  Description of Artificial Sequence: Synthetic peptide
SSTQAHPFAPQL

SEQ ID NO: 335 - protein, 12 aa, Artificial Sequence
  Description of Artificial Sequence: Synthetic peptide
SSTQVQHTLLQT

SEQ ID NO: 336 - protein, 7 aa, Artificial Sequence
  Description of Artificial Sequence: Synthetic peptide
SSVPGRP

SEQ ID NO: 337 - protein, 7 aa, Artificial Sequence
  Description of Artificial Sequence: Synthetic peptide
SSYEYHA

SEQ ID NO: 338 - protein, 7 aa, Artificial Sequence
  Description of Artificial Sequence: Synthetic peptide
STLASMR

SEQ ID NO: 339 - protein, 12 aa, Artificial Sequence
  Description of Artificial Sequence: Synthetic peptide
STPNSYSLPQAR

SEQ ID NO: 340 - protein, 7 aa, Artificial Sequence
  Description of Artificial Sequence: Synthetic peptide
STQAHPW

SEQ ID NO: 341 - protein, 7 aa, Artificial Sequence
  Description of Artificial Sequence: Synthetic peptide
STSAKHW

SEQ ID NO: 342 - protein, 12 aa, Artificial Sequence
  Description of Artificial Sequence: Synthetic peptide
STVVMQPPPRPA

SEQ ID NO: 343 - protein, 12 aa, Artificial Sequence
  Description of Artificial Sequence: Synthetic peptide
SVFLPTRHSPDL

SEQ ID NO: 344 - protein, 12 aa, Artificial Sequence
  Description of Artificial Sequence: Synthetic peptide
SVQTRPLFHSHF

SEQ ID NO: 345 - protein, 12 aa, Artificial Sequence
  Description of Artificial Sequence: Synthetic peptide
SVSVGMKPSPRP

SEQ ID NO: 346 - protein, 12 aa, Artificial Sequence
  Description of Artificial Sequence: Synthetic peptide
  MOD_RES (11)..(11): Any amino acid
SVSVGMNAESXA

SEQ ID NO: 347 - protein, 12 aa, Artificial Sequence
  Description of Artificial Sequence: Synthetic peptide
SVSVGMNAESYG

SEQ ID NO: 348 - protein, 12 aa, Artificial Sequence
  Description of Artificial Sequence: Synthetic peptide
  MOD_RES (11)..(11): Any amino acid
SVSVGTEAESXA

SEQ ID NO: 349 - protein, 12 aa, Artificial Sequence
  Description of Artificial Sequence: Synthetic peptide
SWPLYSRDSGLG

SEQ ID NO: 350 - protein, 12 aa, Artificial Sequence
  Description of Artificial Sequence: Synthetic peptide
SYIDSMVPSTQT

SEQ ID NO: 351 - protein, 12 aa, Artificial Sequence
  Description of Artificial Sequence: Synthetic peptide
SYKTTDSDTSPL

SEQ ID NO: 352 - protein, 16 aa, Artificial Sequence
  Description of Artificial Sequence: Synthetic peptide
SYSQMDPPRSLPGGGS

SEQ ID NO: 353 - protein, 12 aa, Artificial Sequence
  Description of Artificial Sequence: Synthetic peptide
TAAASNLRAVPP

SEQ ID NO: 354 - protein, 12 aa, Artificial Sequence
  Description of Artificial Sequence: Synthetic peptide
TAPLSHPPRPGA

SEQ ID NO: 355 - protein, 7 aa, Artificial Sequence
  Description of Artificial Sequence: Synthetic peptide
TDHPPKA

SEQ ID NO: 356 - protein, 7 aa, Artificial Sequence
  Description of Artificial Sequence: Synthetic peptide
TGLAKTA

SEQ ID NO: 357 - protein, 12 aa, Artificial Sequence
  Description of Artificial Sequence: Synthetic peptide
TGLLPNSSGAGI

SEQ ID NO: 358 - protein, 12 aa, Artificial Sequence
  Description of Artificial Sequence: Synthetic peptide
TGPPSRQPAPLH

SEQ ID NO: 359 - protein, 7 aa, Artificial Sequence
  Description of Artificial Sequence: Synthetic peptide
TGPTSLS

SEQ ID NO: 360 - protein, 12 aa, Artificial Sequence
  Description of Artificial Sequence: Synthetic peptide
THPVVFEDERLF

SEQ ID NO: 361 - protein, 7 aa, Artificial Sequence
  Description of Artificial Sequence: Synthetic peptide
TIHSKPA

SEQ ID NO: 362 - protein, 7 aa, Artificial Sequence
  Description of Artificial Sequence: Synthetic peptide
TKDWLPS

SEQ ID NO: 363 - protein, 7 aa, Artificial Sequence
  Description of Artificial Sequence: Synthetic peptide
TLAFQTA

SEQ ID NO: 364 - protein, 7 aa, Artificial Sequence
  Description of Artificial Sequence: Synthetic peptide
TLAPTFR

SEQ ID NO: 365 - protein, 12 aa, Artificial Sequence
  Description of Artificial Sequence: Synthetic peptide
TLDKYTRLLSRY

SEQ ID NO: 366 - protein, 7 aa, Artificial Sequence
  Description of Artificial Sequence: Synthetic peptide
TLGLPML

SEQ ID NO: 367 - protein, 7 aa, Artificial Sequence
  Description of Artificial Sequence: Synthetic peptide
TLLRTQV

SEQ ID NO: 368 - protein, 7 aa, Artificial Sequence
  Description of Artificial Sequence: Synthetic peptide
TLMTTPP

SEQ ID NO: 369 - protein, 12 aa, Artificial Sequence
  Description of Artificial Sequence: Synthetic peptide
TLPSPLALLTVH

SEQ ID NO: 370 - protein, 7 aa, Artificial Sequence
  Description of Artificial Sequence: Synthetic peptide
TLQRMGQ

SEQ ID NO: 371 - protein, 12 aa, Artificial Sequence
  Description of Artificial Sequence: Synthetic peptide
TLSNGHRYLELL

SEQ ID NO: 372 - protein, 12 aa, Artificial Sequence
  Description of Artificial Sequence: Synthetic peptide
TMGFTAPRFPHY

SEQ ID NO: 373 - protein, 12 aa, Artificial Sequence
  Description of Artificial Sequence: Synthetic peptide
TMRNPITSLISV

SEQ ID NO: 374 - protein, 16 aa, Artificial Sequence
  Description of Artificial Sequence: Synthetic peptide
TMRNPITSLISVGGGS

SEQ ID NO: 375 - protein, 7 aa, Artificial Sequence
  Description of Artificial Sequence: Synthetic peptide
TMTNMAK

SEQ ID NO: 376 - protein, 12 aa, Artificial Sequence
  Description of Artificial Sequence: Synthetic peptide
TPLSYLKGLVTV

SEQ ID NO: 377 - protein, 12 aa, Artificial Sequence
  Description of Artificial Sequence: Synthetic peptide
TPLTSPSLVRPQ

SEQ ID NO: 378 - protein, 12 aa, Artificial Sequence
  Description of Artificial Sequence: Synthetic peptide
TPSPKLLQVFQA

SEQ ID NO: 379 - protein, 12 aa, Artificial Sequence
  Description of Artificial Sequence: Synthetic peptide
TPSTGLGMSPAV

SEQ ID NO: 380 - protein, 12 aa, Artificial Sequence
  Description of Artificial Sequence: Synthetic peptide
TPVYSLKLGPWP

SEQ ID NO: 381 - protein, 12 aa, Artificial Sequence
  Description of Artificial Sequence: Synthetic peptide
TQTWPQSSSHGL

SEQ ID NO: 382 - protein, 7 aa, Artificial Sequence
  Description of Artificial Sequence: Synthetic peptide
TRFYDSL

SEQ ID NO: 383 - protein, 12 aa, Artificial Sequence
  Description of Artificial Sequence: Synthetic peptide
TRLVPSRYYHHP

SEQ ID NO: 384 - protein, 12 aa, Artificial Sequence
  Description of Artificial Sequence: Synthetic peptide
TSPIPQMRTVPP

SEQ ID NO: 385 - protein, 7 aa, Artificial Sequence
  Description of Artificial Sequence: Synthetic peptide
TTKNFNK

SEQ ID NO: 386 - protein, 7 aa, Artificial Sequence
  Description of Artificial Sequence: Synthetic peptide
TTLSPRT

SEQ ID NO: 387 - protein, 12 aa, Artificial Sequence
  Description of Artificial Sequence: Synthetic peptide
TTNSSMTMQLQR

SEQ ID NO: 388 - protein, 12 aa, Artificial Sequence
  Description of Artificial Sequence: Synthetic peptide
TTTLPVQPTLRN

SEQ ID NO: 389 - protein, 12 aa, Artificial Sequence
  Description of Artificial Sequence: Synthetic peptide
TTTWTTTARWPL

SEQ ID NO: 390 - protein, 7 aa, Artificial Sequence
  Description of Artificial Sequence: Synthetic peptide
TTYNSPP

SEQ ID NO: 391 - protein, 12 aa, Artificial Sequence
  Description of Artificial Sequence: Synthetic peptide
TVAQMPPHWQLT

SEQ ID NO: 392 - protein, 7 aa, Artificial Sequence
  Description of Artificial Sequence: Synthetic peptide
TVLGTFP

SEQ ID NO: 393 - protein, 12 aa, Artificial Sequence
  Description of Artificial Sequence: Synthetic peptide
TWNSNSTQYGNR

SEQ ID NO: 394 - protein, 12 aa, Artificial Sequence
  Description of Artificial Sequence: Synthetic peptide
TWTLPAMHPRPA

SEQ ID NO: 395 - protein, 7 aa, Artificial Sequence
  Description of Artificial Sequence: Synthetic peptide
VHLTHGQ

SEQ ID NO: 396 - protein, 7 aa, Artificial Sequence
  Description of Artificial Sequence: Synthetic peptide
VHPRPSL

SEQ ID NO: 397 - protein, 12 aa, Artificial Sequence
  Description of Artificial Sequence: Synthetic peptide
VHTSLLQKHPLP

SEQ ID NO: 398 - protein, 11 aa, Artificial Sequence
  Description of Artificial Sequence: Synthetic peptide
VLPNIYMTLSA

SEQ ID NO: 399 - protein, 12 aa, Artificial Sequence
  Description of Artificial Sequence: Synthetic peptide
VMDFASPAHVLP

SEQ ID NO: 400 - protein, 12 aa, Artificial Sequence
  Description of Artificial Sequence: Synthetic peptide
VNQEYWFFPRRP

SEQ ID NO: 401 - protein, 15 aa, Artificial Sequence
  Description of Artificial Sequence: Synthetic peptide
  MOD_RES (6)..(6): Any amino acid
  MOD_RES (11)..(11): Any amino acid
  MOD_RES (14)..(14): Any amino acid
VPPISXTFLFXSTXS

SEQ ID NO: 402 - protein, 12 aa, Artificial Sequence
  Description of Artificial Sequence: Synthetic peptide
  MOD_RES (11)..(11): Any amino acid
VPPLHPALSRXN

SEQ ID NO: 403 - protein, 12 aa, Artificial Sequence
  Description of Artificial Sequence: Synthetic peptide
VSPFLSPTPLLF

SEQ ID NO: 404 - protein, 12 aa, Artificial Sequence
  Description of Artificial Sequence: Synthetic peptide
VSRLGTPSMHPS

SEQ ID NO: 405 - protein, 7 aa, Artificial Sequence
  Description of Artificial Sequence: Synthetic peptide
VVKSNGE

SEQ ID NO: 406 - protein, 11 aa, Artificial Sequence
  Description of Artificial Sequence: Synthetic peptide
VYSSPLSQLPR

SEQ ID NO: 407 - protein, 7 aa, Artificial Sequence
  Description of Artificial Sequence: Synthetic peptide
WLPPRTQ

SEQ ID NO: 408 - protein, 12 aa, Artificial Sequence
  Description of Artificial Sequence: Synthetic peptide
WPANKLSTKSMY

SEQ ID NO: 409 - protein, 12 aa, Artificial Sequence
  Description of Artificial Sequence: Synthetic peptide
WPFNHFPWWNVP

SEQ ID NO: 410 - protein, 12 aa, Artificial Sequence
  Description of Artificial Sequence: Synthetic peptide
WPTYLNPSSLKA

SEQ ID NO: 411 - protein, 12 aa, Artificial Sequence
  Description of Artificial Sequence: Synthetic peptide
WSAHIVPYSHKP

SEQ ID NO: 412 - protein, 12 aa, Artificial Sequence
  Description of Artificial Sequence: Synthetic peptide
WWPNSLNWVPRP

SEQ ID NO: 413 - protein, 7 aa, Artificial Sequence
  Description of Artificial Sequence: Synthetic peptide
WYPNHLA

SEQ ID NO: 414 - protein, 7 aa, Artificial Sequence
  Description of Artificial Sequence: Synthetic peptide
  MOD_RES (1)..(1): Any amino acid
  MOD_RES (4)..(4): Any amino acid
XITXGAY

SEQ ID NO: 415 - protein, 9 aa, Artificial Sequence
  Description of Artificial Sequence: Synthetic peptide
  MOD_RES (1)..(1): Any amino acid
XPRRAVAAL

SEQ ID NO: 416 - protein, 9 aa, Artificial Sequence
  Description of Artificial Sequence: Synthetic peptide
  MOD_RES (1)..(1): Any amino acid
  MOD_RES (8)..(8): Any amino acid
XPRRAVAXL

SEQ ID NO: 417 - protein, 9 aa, Artificial Sequence
  Description of Artificial Sequence: Synthetic peptide
  MOD_RES (1)..(1): Any amino acid
  MOD_RES (5)..(5): Any amino acid
  MOD_RES (8)..(8): Any amino acid
XPRRXVAXL

SEQ ID NO: 418 - protein, 7 aa, Artificial Sequence
  Description of Artificial Sequence: Synthetic peptide
  MOD_RES (1)..(2): Any amino acid
  MOD_RES (6)..(6): Any amino acid
XXFPLXG

SEQ ID NO: 419 - protein, 12 aa, Artificial Sequence
  Description of Artificial Sequence: Synthetic peptide
YATQHNWRLKHE

SEQ ID NO: 420 - protein, 10 aa, Artificial Sequence
  Description of Artificial Sequence: Synthetic peptide
YCPMRLCTDC

SEQ ID NO: 421 - protein, 12 aa, Artificial Sequence
  Description of Artificial Sequence: Synthetic peptide
YELQMPLTLPLN

SEQ ID NO: 422 - protein, 7 aa, Artificial Sequence
  Description of Artificial Sequence: Synthetic peptide
YEPAAAE

SEQ ID NO: 423 - protein, 12 aa, Artificial Sequence
  Description of Artificial Sequence: Synthetic peptide
YGKGFSPYFHVT

SEQ ID NO: 424 - protein, 12 aa, Artificial Sequence
  Description of Artificial Sequence: Synthetic peptide
YPHYSLPGSSTL

SEQ ID NO: 425 - protein, 12 aa, Artificial Sequence
  Description of Artificial Sequence: Synthetic peptide
YPIMSHTCCHGV

SEQ ID NO: 426 - protein, 7 aa, Artificial Sequence
  Description of Artificial Sequence: Synthetic peptide
YPKALRN

SEQ ID NO: 427 - protein, 12 aa, Artificial Sequence
  Description of Artificial Sequence: Synthetic peptide
YPSLLKMQPQFS

SEQ ID NO: 428 - protein, 12 aa, Artificial Sequence
  Description of Artificial Sequence: Synthetic peptide
YQPRPFVTTSPM

SEQ ID NO: 429 - protein, 12 aa, Artificial Sequence
  Description of Artificial Sequence: Synthetic peptide
YSAPLARSNVVM

SEQ ID NO: 430 - protein, 12 aa, Artificial Sequence
  Description of Artificial Sequence: Synthetic peptide
YTRLSHNPYTLS

SEQ ID NO: 431 - protein, 12 aa, Artificial Sequence
  Description of Artificial Sequence: Synthetic peptide
YTTHVLPFAPSS

SEQ ID NO: 432 - protein, 12 aa, Artificial Sequence
  Description of Artificial Sequence: Synthetic peptide
YTWQTIREQYEM

SEQ ID NO: 433 - protein, 152 aa, Artificial Sequence
  Description of Artificial Sequence: Synthetic polypeptide
ASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 434 - protein, 217 aa, Artificial Sequence
  Description of Artificial Sequence: Synthetic polypeptide
MPIGSLLADTTHHRPWTVIGESTHHRPWSIIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 435 - protein, 212 aa, Artificial Sequence
  Description of Artificial Sequence: Synthetic polypeptide
LLADTTHHRPWTVIGESTHHRPWSIIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 436 - protein, 200 aa, Artificial Sequence
  Description of Artificial Sequence: Synthetic polypeptide
VIGESTHHRPWSIIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 437 - protein, 188 aa, Artificial Sequence
  Description of Artificial Sequence: Synthetic polypeptide
IIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 438 - protein, 176 aa, Artificial Sequence
  Description of Artificial Sequence: Synthetic polypeptide
GLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 439 - protein, 164 aa, Artificial Sequence
  Description of Artificial Sequence: Synthetic polypeptide
ILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 440 - protein, 115 aa, Artificial Sequence
  Description of Artificial Sequence: Synthetic polypeptide
  MOD_RES (1)..(1): Any amino acid
  See specification as filed for detailed description of substitutions and preferred embodiments
XQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 441 - protein, 153 aa, Artificial Sequence
  Description of Artificial Sequence: Synthetic polypeptide
  MOD_RES (1)..(1): Any amino acid
  See specification as filed for detailed description of substitutions and preferred embodiments
XASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 442 - protein, 53 aa, Artificial Sequence
  Description of Artificial Sequence: Synthetic polypeptide
NSDSECPLSHDGYCLHDGVCMYIEALDKYACNCVVGYIGERCQYRDLKWWELR

SEQ ID NO: 443 - protein, 66 aa, Artificial Sequence
  Description of Artificial Sequence: Synthetic polypeptide
EEAEIPREVIERLARSQIHSIRDLQRLLEIDSVGSEDSLDTSLRAHGVHATKHVPEKRPLPIRRKR

SEQ ID NO: 444 - protein, 70 aa, Artificial Sequence
  Description of Artificial Sequence: Synthetic polypeptide
GPETLCGAELVDALQFVCGDRGFYFNKPTGYGSSSRRAPQTGIVDECCFRSCDLRRLEMYCAPLKPAKSA

SEQ ID NO: 445 - protein, 140 aa, Artificial Sequence
  Description of Artificial Sequence: Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 446 - protein, 146 aa, Artificial Sequence
  Description of Artificial Sequence: Synthetic polypeptide
PALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 447 - protein, 180 aa, Artificial Sequence
  Description of Artificial Sequence: Synthetic polypeptide
EENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 448 - protein, 137 aa, Artificial Sequence
  Description of Artificial Sequence: Synthetic polypeptide
ENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 449 - protein, 50 aa, Artificial Sequence
  Description of Artificial Sequence: Synthetic polypeptide
VVSHFNDCPDSHTQFCFHGTCRFLVQEDKPACVCHSGYVGARCEHADLLA

SEQ ID NO: 450 - protein, 112 aa, Artificial Sequence
  Description of Artificial Sequence: Synthetic polypeptide
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 451 - protein, 112 aa, Artificial Sequence [sequence omitted]

SEQ ID NO: 452 - protein, 139 aa, Artificial Sequence
  Description of Artificial Sequence: Synthetic polypeptide
AVRPLRRRQPKKSNELPQANRLPGIFDDVHGSHGRQVCRRHELYVSFQDLGWLDWVIAPQGYSAYYCEGECSFPLDSCMNATNHAILQSLVHLMMPDAVPKACCAPTKLSATSVLYYDSSNNVILRKHRNMVVKACGCH

SEQ ID NO: 453 - protein, 139 aa, Artificial Sequence
  Description of Artificial Sequence: Synthetic polypeptide
AVRPLRRRQPKKSNELPQANRLPGIFDDVRGSHGRQVCRRHELYVSFQDLGWLDWVIAPQGYSAYYCEGECSFPLDSCMNATNHAILQSLVHLMKPNAVPKACCAPTKLSATSVLYYDSSNNVILRKHRNMVVKACGCH

SEQ ID NO: 454 - protein, 114 aa, Artificial Sequence
  Description of Artificial Sequence: Synthetic polypeptide
QAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 455 - protein, 110 aa, Artificial Sequence
  Description of Artificial Sequence: Synthetic polypeptide
QWIEPRNCARRYLKVDFADIGWSEWIISPKSFDAYYCSGACQFPMPKSLKPSNHATIQSIVRAVGVVPGIPEPCCVPEKMSSLSILFFDENKNVVLKVYPNMTVESCACR

SEQ ID NO: 456 - protein, 116 aa, Artificial Sequence
  Description of Artificial Sequence: Synthetic polypeptide
SPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 457 - protein, 138 aa, Artificial Sequence
  Description of Artificial Sequence: Synthetic polypeptide
AANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 458 - protein, 139 aa, Artificial Sequence
  Description of Artificial Sequence: Synthetic polypeptide
SASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 459 - protein, 139 aa, Artificial Sequence
  Description of Artificial Sequence: Synthetic polypeptide
STGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 460 - protein, 110 aa, Artificial Sequence
  Description of Artificial Sequence: Synthetic polypeptide
SAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 461 - protein, 108 aa, Artificial Sequence
  Description of Artificial Sequence: Synthetic polypeptide
NAKGNYCKRTPLYIDFKEIGWDSWIIAPPGYEAYECRGVCNYPLAEHLTPTKHAIIQALVHLKNSQKASKACCVPTKLEPISILYLDKGVVTYKFKYEGMAVSECGCR

SEQ ID NO: 462 - protein, 109 aa, Artificial Sequence
  Description of Artificial Sequence: Synthetic polypeptide
NLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 463 - protein, 129 aa, Artificial Sequence
  Description of Artificial Sequence: Synthetic polypeptide
TALAGTRTAQGSGGGAGRGHGRRGRSRCSRKPLHVDFKELGWDDWIIAPLDYEAYHCEGLCDFPLRSHLEPTNHAIIQTLLNSMAPDAAPASCCVPARLSPISILYIDAANNVVYKQYEDMVVEACGCR

SEQ ID NO: 464 - protein, 120 aa, Artificial Sequence
  Description of Artificial Sequence: Synthetic polypeptide
TAFASRHGKRHGKKSRLRCSKKPLHVNFKELGWDDWIIAPLEYEAYHCEGVCDFPLRSHLEPTNHAIIQTLMNSMDPGSTPPSCCVPTKLTPISILYIDAGNNVVYKQYEDMVVESCGCR

SEQ ID NO: 465 - protein, 125 aa, Artificial Sequence
  Description of Artificial Sequence: Synthetic polypeptide
QADGISAEVTASSSKHSGPENNQCSLHPFQISFRQLGWDHWIIAPPFYTPNYCKGTCLRVLRDGLNSPNHAIIQNLINQLVDQSVPRPSCVPYKYVPISVLMIEANGSILYKEYEGMIAESCTCR

SEQ ID NO: 466 - protein, 447 aa, Artificial Sequence
  Description of Artificial Sequence: Synthetic polypeptide
IPVPQSKPLERHVEKSMNLHLLARSNVSVQDELNASGTIKESGVLVHEGDRGRQENTQDGHKGEGNGSKWAEVGGKSFSTYSTLANEEGNIEGWNGDTGKAETYGHDGIHGKEENITANGIQGQVSIIDNAGATNRSNTNGNTDKNTQNGDVGDAGHNEDVAVVQEDGPQVAGSNNSTDNEDEIIENSCRNEGNTSEITPQINSKRNGTKEAEVTPGTGEDAGLDNSDGSPSGNGADEDEDEGSGDDEDEEAGNGKDSSNNSKGQEGQDHGKEDDHDSSIGQNSDSKEYYDPEGKEDPHNEVDGDKTSKSEENSAGIPEDNGSQRIEDTQKLNHRESKRVENRITKESETHAVGKSQDKGIEIKGPSSGNRNITKEVGKGNEGKEDKGQHGMILGKGNVKTQGEVVNIEGPGQKSEPGNKVGHSNTGSDSNSDGYDSYDFDDKSMQG

SEQ ID NO: 467 - protein, 1286 aa, Artificial Sequence
  Description of Artificial Sequence: Synthetic polypeptide
IPVPQSKPLERHVEKSMNLHLLARSNVSVQDELNASGTIKESGVLVHEGDRGRQENTQDGHKGEGNGSKWAEVGGKSFSTYSTLANEEGNIEGWNGDTGKAETYGHDGIHGKEENITANGIQGQVSIIDNAGATNRSNTNGNTDKNTQNGDVGDAGHNEDVAVVQEDGPQVAGSNNSTDNEDEIIENSCRNEGNTSEITPQINSKRNGTKEAEVTPGTGEDAGLDNSDGSPSGNGADEDEDEGSGDDEDEEAGNGKDSSNNSKGQEGQDHGKEDDHDSSIGQNSDSKEYYDPEGKEDPHNEVDGDKTSKSEENSAGIPEDNGSQRIEDTQKLNHRESKRVENRITKESETHAVGKSQDKGIEIKGPSSGNRNITKEVGKGNEGKEDKGQHGMILGKGNVKTQGEVVNIEGPGQKSEPGNKVGHSNTGSDSNSDGYDSYDFDDKSMQGDDPNSSDESNGNDDANSESDNNSSSRGDASYNSDESKDNGNGSDSKGAEDDDSDSTSDTNNSDSNGNGNNGNDDNDKSDSGKGKSDSSDSDSSDSSNSSDSSDSSDSDSSDSNSSSDSDSSDSDSSDSSDSDSSDSSNSSDSSDSSDSSDSSDSSDSSDSKSDSSKSESDSSDSDSKSDSSDSNSSDSSDNSDSSDSSNSSNSSDSSDSSDSSDSSSSSDSSNSSDSSDSSDSSNSSESSDSSDSSDSDSSDSSDSSNSNSSDSDSSNSSDSSDSSNSSDSSDSSDSSNSSDSSDSSDSSNSSDSSDSSDSSDSSDSSNSSDSNDSSNSSDSSDSSNSSDSSNSSDSSDSSDSSDSDSSNSSDSSNSSDSSDSSNSSDSSDSSDSSDGSDSDSSNRSDSSNSSDSSDSSDSSNSSDSSDSSDSNESSNSSDSSDSSNSSDSDSSDSSNSSDSSDSSNSSDSSESSNSSDNSNSSDSSNSSDSSDSSDSSNSSDSSNSSDSSNSSDSSDSNSSDSSDSSNSSDSSDSSDSSDSSDSSDSSNSSDSSDSSDSSDSSNSSDSSNSSDSSNSSDSSDSSDSSDSSDSSDSSDSSDSSNSSDSSDSSDSSDSSDSSDSSDSSDSSESSDSSDSSNSSDSSDSSDSSDSSDSSDSSDSSDSSDSSNSSDSSDSSDSSDSSDSSNSSDSSDSSESSDSSDSSDSSDSSDSSDSSDSSDSSDSSNSSDSSDSSDSSDSSDSSDSSDSSDSSDSSDSSDSSDSSDSSDSSDSSDSSDSNESSDSSDSSDSSDSSNSSDSSDSSDSSDSTSDSNDESDSQSKSGNGNNNGSDSDSDSEGSDSNHSTSDD

SEQ ID NO: 468 - protein, 839 aa, Artificial Sequence
  Description of Artificial Sequence: Synthetic polypeptide
DDPNSSDESNGNDDANSESDNNSSSRGDASYNSDESKDNGNGSDSKGAEDDDSDSTSDTNNSDSNGNGNNGNDDNDKSDSGKGKSDSSDSDSSDSSNSSDSSDSSDSDSSDSNSSSDSDSSDSDSSDSSDSDSSDSSNSSDSSDSSDSSDSSDSSDSSDSKSDSSKSESDSSDSDSKSDSSDSNSSDSSDNSDSSDSSNSSNSSDSSDSSDSSDSSSSSDSSNSSDSSDSSDSSNSSESSDSSDSSDSDSSDSSDSSNSNSSDSDSSNSSDSSDSSNSSDSSDSSDSSNSSDSSDSSDSSNSSDSSDSSDSSDSSDSSNSSDSNDSSNSSDSSDSSNSSDSSNSSDSSDSSDSSDSDSSNSSDSSNSSDSSDSSNSSDSSDSSDSSDGSDSDSSNRSDSSNSSDSSDSSDSSNSSDSSDSSDSNESSNSSDSSDSSNSSDSDSSDSSNSSDSSDSSNSSDSSESSNSSDNSNSSDSSNSSDSSDSSDSSNSSDSSNSSDSSNSSDSSDSNSSDSSDSSNSSDSSDSSDSSDSSDSSDSSNSSDSSDSSDSSDSSNSSDSSNSSDSSNSSDSSDSSDSSDSSDSSDSSDSSDSSNSSDSSDSSDSSDSSDSSDSSDSSDSSESSDSSDSSNSSDSSDSSDSSDSSDSSDSSDSSDSSDSSNSSDSSDSSDSSDSSDSSNSSDSSDSSESSDSSDSSDSSDSSDSSDSSDSSDSSDSSNSSDSSDSSDSSDSSDSSDSSDSSDSSDSSDSSDSSDSSDSSDSSDSSDSSDSNESSDSSDSSDSSDSSNSSDSSDSSDSSDSTSDSNDESDSQSKSGNGNNNGSDSDSDSEGSDSNHSTSDD

SEQ ID NO: 469 - protein, 119 aa, Artificial Sequence
  Description of Artificial Sequence: Synthetic polypeptide
DAEPVLGGGPGGACRARRLYVSFREVGWHRWVIAPRGFLANYCQGQCALPVALSGSGGPPALNHAVLRALMHAAAPGAADLPCCVPARLSPISVLFFDNSDNVVLRQYEDMVVDECGCR

SEQ ID NO: 470 - protein, 114 aa, Artificial Sequence
  Description of Artificial Sequence: Synthetic polypeptide
AAIPVPKLSCKNLCHRHQLFINFRDLGWHKWIIAPKGFMANYCHGECPFSLTISLNSSNYAFMQALMHAVDPEIPQAVCIPTKLSPISMLYQDNNDNVILRHYEDMVVDECGCG

SEQ ID NO: 471 - protein, 120 aa, Artificial Sequence
  Description of Artificial Sequence: Synthetic polypeptide
APLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 472 - protein, 109 aa, Artificial Sequence
  Description of Artificial Sequence: Synthetic polypeptide
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 473 - protein, 114 aa, Artificial Sequence
  Description of Artificial Sequence: Synthetic polypeptide
ARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 474 - protein, 206 aa, Artificial Sequence
  Description of Artificial Sequence: Synthetic polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 475 - protein, 290 aa, Artificial Sequence
  Description of Artificial Sequence: Synthetic polypeptide
FSLMSLLESLDPDWTPDQYDYSYEDYNQEENTSSTLTHAENPDWYYTEDQADPCQPNPCEHGGDCLVHGSTFTCSCLAPFSGNKCQKVQNTCKDNPCGRGQCLITQSPPYYRCVCKHPYTGPSCSQVVPVCRPNPCQNGATCSRHKRRSKFTCACPDQFKGKFCEIGSDDCYVGDGYSYRGKMNRTVNQHACLYWNSHLLLQENYNMFMEDAETHGIGEHNFCRNPDADEKPWCFIKVTNDKVKWEYCDVSACSAQDVAYPEESPTEPSTKLPGFDSCGKTEIAERKIKR

SEQ ID NO: 476 - skipped

SEQ ID NO: 477 - protein, 247 aa, Artificial Sequence
  Description of Artificial Sequence: Synthetic polypeptide
IYGGFKSTAGKHPWQASLQSSLPLTISMPQGHFCGGALIHPCWVLTAAHCTDIKTRHLKVVLGDQDLKKEEFHEQSFRVEKIFKYSHYNERDEIPHNDIALLKLKPVDGHCALESKYVKTVCLPDGSFPSGSECHISGWGVTETGKGSRQLLDAKVKLIANTLCNSRQLYDHMIDDSMICAGNLQKPGQDTCQGDSGGPLTCEKDGTYYVYGIVSWGLECGKRPGVYTQVTKFLNWIKATIKSESGF

SEQ ID NO: 478 - skipped

SEQ ID NO: 479 - protein, 400 aa, Artificial Sequence
  Description of Artificial Sequence: Synthetic polypeptide
AEVKKPAAAAAPGTAEKLSPKAATLAERSAGLAFSLYQAMAKDQAVENILVSPVVVASSLGLVSLGGKATTASQAKAVLSAEQLRDEEVHAGLGELLRSLSNSTARNVTWKLGSRLYGPSSVSFADDFVRSSKQHYNCEHSKINFRDKRSALQSINEWAAQTTDGKLPEVTKDVERTDGALLVNAMFFKPHWDEKFHHKMVDNRGFMVTRSYTVGVMMMHRTGLYNYYDDEKEKLQIVEMPLAHKLSSLIILMPHHVEPLERLEKLLTKEQLKIWMGKMQKKAVAISLPKGVVEVTHDLQKHLAGLGLTEAIDKNKADLSRMSGKKDLYLASVFHATAFELDTDGNPFDQDIYGREELRSPKLFYADHPFIFLVRDTQSGSLLFIGRLVRPKGDKMRDEL

SEQ ID NO: 480 - protein, 163 aa, Artificial Sequence
  Description of Artificial Sequence: Synthetic polypeptide
CNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 481 - protein, 177 aa, Artificial Sequence
  Description of Artificial Sequence: Synthetic polypeptide
GPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 482 - protein, 243 aa, Artificial Sequence
  Description of Artificial Sequence: Synthetic polypeptide
TNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 483 - protein, 343 aa, Artificial Sequence
  Description of Artificial Sequence: Synthetic polypeptide
ANSSGRWWGIVNVASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 484 - protein, 335 aa, Artificial Sequence
  Description of Artificial Sequence: Synthetic polypeptide
SWWYMRATGGSSRVMCDNVPGLVSSQRQLCHRHPDVMRAISQGVAEWTAECQHQFRQHRWNCNTLDRDHSLFGRVLLRSSRESAFVYAISSAGVVFAITRACSQGEVKSCSCDPKKMGSAKDSKGIFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQDGTGFTVANERFKKPTKNDLVYFENSPDYCIRDREAGSLGTAGRVCNLTSRGMDSCEVMCCGRGYDTSHVTRMTKCGCKFHWCCAVRCQDCLEALDVHTCKAPKNADWTTAT

SEQ ID NO: 485 - protein, 333 aa, Artificial Sequence
  Description of Artificial Sequence: Synthetic polypeptide
SWWYIGALGARVICDNIPGLVSRQRQLCQRYPDIMRSVGEGAREWIRECQHQFRHHRWNCTTLDRDHTVFGRVMLRSSREAAFVYAISSAGVVHAITRACSQGELSVCSCDPYTRGRHHDQRGDFDWGGCSDNIHYGVRFAKAFVDAKEKRLKDARALMNLHNNRCGRTAVRRFLKLECKCHGVSGSCTLRTCWRALSDFRRTGDYLRRRYDGAVQVMATQDGANFTAARQGYRRATRTDLVYFDNSPDYCVLDKAAGSLGTAGRVCSKTSKGTDGCEIMCCGRGYDTTRVTRVTQCECKFHWCCAVRCKECRNTVDVHTCKAPKKAEWLDQT

SEQ ID NO: 486 - protein, 334 aa, Artificial Sequence
  Description of Artificial Sequence: Synthetic polypeptide
GYPIWWSLALGQQYTSLGSQPLLCGSIPGLVPKQLRFCRNYIEIMPSVAEGVKLGIQECQHQFRGRRWNCTTIDDSLAIFGPVLDKATRESAFVHAIASAGVAFAVTRSCAEGTSTICGCDSHHKGPPGEGWKWGGCSEDADFGVLVSREFADARENRPDARSAMNKHNNEAGRTTILDHMHLKCKCHGLSGSCEVKTCWWAQPDFRAIGDFLKDKYDSASEMVVEKHRESRGWVETLRAKYSLFKPPTERDLVYYENSPNFCEPNPETGSFGTRDRTCNVTSHGIDGCDLLCCGRGHNTRTEKRKEKCHCIFHWCCYVSCQECIRIYDVHTCK

SEQ ID NO: 487 - protein, 334 aa, Artificial Sequence
  Description of Artificial Sequence: Synthetic polypeptide
SYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 488 - protein, 329 aa, Artificial Sequence
  Description of Artificial Sequence: Synthetic polypeptide
SNWLYLAKLSSVGSISEEETCEKLKGLIQRQVQMCKRNLEVMDSVRRGAQLAIEECQYQFRNRRWNCSTLDSLPVFGKVVTQGTREAAFVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDGATEVEPRRVGSSRALVPRNAQFKPHTDEDLVYLEPSPDFCEQDMRSGVLGTRGRTCNKTSKAIDGCELLCCGRGFHTAQVELAERCSCKFHWCCFVKCRQCQRLVELHTCR

SEQ ID NO: 489 - protein, 319 aa, Artificial Sequence
  Description of Artificial Sequence: Synthetic polypeptide
IIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 490 - protein, 342 aa, Artificial Sequence
  Description of Artificial Sequence: Synthetic polypeptide
QLLTDANSWWSLALNPVQRPEMFIIGAQPVCSQLPGLSPGQRKLCQLYQEHMAYIGEGAKTGIKECQHQFRQRRWNCSTADNASVFGRVMQIGSRETAFTHAVSAAGVVNAISRACREGELSTCGCSRTARPKDLPRDWLWGGCGDNVEYGYRFAKEFVDAREREKNFAKGSEEQGRVLMNLQNNEAGRRAVYKMADVACKCHGVSGSCSLKTCWLQLAEFRKVGDRLKEKYDSAAAMRVTRKGRLELVNSRFTQPTPEDLVYVDPSPDYCLRNESTGSLGTQGRLCNKTSEGMDGCELMCCGRGYNQFKSVQVERCHCKFHWCCFVRCKKCTEIVDQYICK

SEQ ID NO: 491 - protein, 341 aa, Artificial Sequence
  Description of Artificial Sequence: Synthetic polypeptide
LWWAVGSPLVMDPTSICRKARRLAGRQAELCQAEPEVVAELARGARLGVRECQFQFRFRRWNCSSHSKAFGRILQQDIRETAFVFAITAAGASHAVTQACSMGELLQCGCQAPRGRAPPRPSGLPGTPGPPGPAGSPEGSAAWEWGGCGDDVDFGDEKSRLFMDARHKRGRGDIRALVQLHNNEAGRLAVRSHTRTECKCHGLSGSCALRTCWQKLPPFREVGARLLERFHGASRVMGTNDGKALLPAVRTLKPPGRADLLYAADSPDFCAPNRRTGSPGTRGRACNSSAPDLSGCDLLCCGRGHRQESVQLEENCLCRFHWCCVVQCHRCRVRKELSLCL

SEQ ID NO: 492 - protein, 318 aa, Artificial Sequence
  Description of Artificial Sequence: Synthetic polypeptide
LGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 493 - protein, 325 aa, Artificial Sequence
  Description of Artificial Sequence: Synthetic polypeptide
ALSSVVALGANIICNKIPGLAPRQRAICQSRPDAIIVIGEGAQMGINECQYQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMQLECKCHGVSGSCTTKTCWTTLPKFREVGHLLKEKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 494 - protein, 327 aa, Artificial Sequence
  Description of Artificial Sequence: Synthetic polypeptide
VNNFLITGPKAYLTYTTSVALGAQSGIEECKFQFAWERWNCPENALQLSTHNRLRSATRETSFIHAISSAGVMYIITKNCSMGDFENCGCDGSNNGKTGGHGWIWGGCSDNVEFGERISKLFVDSLEKGKDARALMNLHNNRAGRLAVRATMKRTCKCHGISGSCSIQTCWLQLAEFREMGDYLKAKYDQALKIEMDKRQLRAGNSAEGHWVPAEAFLPSAEAELIFLEESPDYCTCNSSLGIYGTEGRECLQNSHNTSRWERRSCGRLCTECGLQVEERKTEVISSCNCKFQWCCTVKCDQCRHVVSKYYCARSPGSAQSLGKGSA

SEQ ID NO: 495 - protein, 329 aa, Artificial Sequence
  Description of Artificial Sequence: Synthetic polypeptide
WSVNNFLMTGPKAYLIYSSSVAAGAQSGIEECKYQFAWDRWNCPERALQLSSHGGLRSANRETAFVHAISSAGVMYTLTRNCSLGDFDNCGCDDSRNGQLGGQGWLWGGCSDNVGFGEAISKQFVDALETGQDARAAMNLHNNEAGRKAVKGTMKRTCKCHGVSGSCTTQTCWLQLPEFREVGAHLKEKYHAALKVDLLQGAGNSAAGRGAIADTFRSISTRELVHLEDSPDYCLENKTLGLLGTEGRECLRRGRALGRWERRSCRRLCGDCGLAVEERRAETVSSCNCKFHWCCAVRCEQCRRRVTKYFCSRAERPRGGAAHKPGRKP

SEQ ID NO: 496 - protein, 336 aa, Artificial Sequence
  Description of Artificial Sequence: Synthetic polypeptide
YFGLTGSEPLTILPLTLEPEAAAQAHYKACDRLKLERKQRRMCRRDPGVAETLVEAVSMSALECQFQFRFERWNCTLEGRYRASLLKRGFKETAFLYAISSAGLTHALAKACSAGRMERCTCDEAPDLENREAWQWGGCGDNLKYSSKFVKEFLGRRSSKDLRARVDFHNNLVGVKVIKAGVETTCKCHGVSGSCTVRTCWRQLAPFHEVGKHLKHKYETALKVGSTTNEAAGEAGAISPPRGRASGAGGSDPLPRTPELVHLDDSPSFCLAGRFSPGTAGRRCHREKNCESICCGRGHNTQSRVVTRPCQCQVRWCCYVECRQCTQREEVYTCKG

SEQ ID NO: 497 - protein, 335 aa, Artificial Sequence
  Description of Artificial Sequence: Synthetic polypeptide
SYFGLTGREVLTPFPGLGTAAAPAQGGAHLKQCDLLKLSRRQKQLCRREPGLAETLRDAAHLGLLECQFQFRHERWNCSLEGRMGLLKRGFKETAFLYAVSSAALTHTLARACSAGRMERCTCDDSPGLESRQAWQWGVCGDNLKYSTKFLSNFLGSKRGNKDLRARADAHNTHVGIKAVKSGLRTTCKCHGVSGSCAVRTCWKQLSPFRETGQVLKLRYDSAVKVSSATNEALGRLELWAPARQGSLTKGLAPRSGDLVYMEDSPSFCRPSKYSPGTAGRVCSREASCSSLCCGRGYDTQSRLVAFSCHCQVQWCCYVECQQCVQEELVYTCKH

SEQ ID NO: 498 - protein, 382 aa, Artificial Sequence
  Description of Artificial Sequence: Synthetic polypeptide
MPRSAPNDILDLRLPPEPVLNANTVCLTLPGLSRRQMEVCVRHPDVAASAIQGIQIAIHECQHQFRDQRWNCSSLETRNKIPYESPIFSRGFRESAFAYAIAAAGVVHAVSNACALGKLKACGCDASRRGDEEAFRRKLHRLQLDALQRGKGLSHGVPEHPALPTASPGLQDSWEWGGCSPDMGFGERFSKDFLDSREPHRDIHARMRLHNNRVGRQAVMENMRRKCKCHGTSGSCQLKTCWQVTPEFRTVGALLRSRFHRATLIRPHNRNGGQLEPGPAGAPSPAPGAPGPRRRASPADLVYFEKSPDFCEREPRLDSAGTVGRLCNKSSAGSDGCGSMCCGRGHNILRQTRSERCHCRFHWCCFVVCEECRITEWVSVCK

SEQ ID NO: 499 - protein, 361 aa, Artificial Sequence
  Description of Artificial Sequence: Synthetic polypeptide
NEILGLKLPGEPPLTANTVCLTLSGLSKRQLGLCLRNPDVTASALQGLHIAVHECQHQLRDQRWNCSALEGGGRLPHHSAILKRGFRESAFSFSMLAAGVMHAVATACSLGKLVSCGCGWKGSGEQDRLRAKLLQLQALSRGKSFPHSLPSPGPGSSPSPGPQDTWEWGGCNHDMDFGEKFSRDFLDSREAPRDIQARMRIHNNRVGRQVVTENLKRKCKCHGTSGSCQFKTCWRAAPEFRAVGAALRERLGRAIFIDTHNRNSGAFQPRLRPRRLSGELVYFEKSPDFCERDPTMGSPGTRGRACNKTSRLLDGCGSLCCGRGHNVLRQTRVERCHCRFHWCCYVLCDECKVTEWVNVCK

SEQ ID NO: 500 - protein, 330 aa, Artificial Sequence
  Description of Artificial Sequence: Synthetic polypeptide
IKWLALSKTPSALALNQTQHCKQLEGLVSAQVQLCRSNLELMHTVVHAAREVMKACRRAFADMRWNCSSIELAPNYLLDLERGTRESAFVYALSAAAISHAIARACTSGDLPGCSCGPVPGEPPGPGNRWGGCADNLSYGLLMGAKFSDAPMKVKKTGSQANKLMRLHNSEVGRQALRASLEMKCKCHGVSGSCSIRTCWKGLQELQDVAADLKTRYLSATKVVHRPMGTRKHLVPKDLDIRPVKDSELVYLQSSPDFCMKNEKVGSHGTQDRQCNKTSNGSDSCDLMCCGRGYNPYTDRVVERCHCKYHWCCYVTCRRCERTVERYVCK

SEQ ID NO: 501 - protein, 336 aa, Artificial Sequence
  Description of Artificial Sequence: Synthetic polypeptide
NWMWLGIASFGVPEKLGCANLPLNSRQKELCKRKPYLLPSIREGARLGIQECGSQFRHERWNCMITAAATTAPMGASPLFGYELSSGTKETAFIYAVMAAGLVHSVTRSCSAGNMTECSCDTTLQNGGSASEGWHWGGCSDDVQYGMWFSRKFLDFPIGNTTGKENKVLLAMNLHNNEAGRQAVAKLMSVDCRCHGVSGSCAVKTCWKTMSSFEKIGHLLKDKYENSIQISDKTKRKMRRREKDQRKIPIHKDDLLYVNKSPNYCVEDKKLGIPGTQGRECNRTSEGADGCNLLCCGRGYNTHVVRHVERCECKFIWCCYVRCRRCESMTDVHTCK

(19) United States
(12) Patent Application Publication
(10) Pub. No.: US 2024/0226383 A9
(43) Pub. Date: Jul. 11, 2024

(54) CERAMIC COMPOSITIONS AND METHODS OF USE

(71) Applicant: Theradaptive, Inc., Frederick, MD (US)

(72) Inventors: Luis ALVAREZ, Lexington, MD (US); Todd HEIL, Mt. Airy, MD (US)

(21) Appl. No.: 18/547,668

(22) PCT Filed: Feb. 23, 2022
(86) PCT No.: PCT/US2022/017508

§ 371 Date: Aug. 23, 2023

Related U.S. Application Data
(60) Provisional application No. 63/153,279, filed on Feb. 24, 2021.

(51) Int. Cl. A61L 27/56; A61K 47/52; A61K 47/59; A61L 27/12; A61L 27/26; A61L 27/54; B33Y 70/10; B33Y 80/00

(52) U.S. Cl. CPC A61L 27/56; A61K 47/52; A61K 47/593; A61L 27/12; A61L 27/26; A61L 27/54; B33Y 70/10; B33Y 80/00; A61L 2300/414; A61L 2430/02; A61L 2430/38

(57) ABSTRACT

The present disclosure provides devices comprising a therapeutic agent bound to a printed three-dimensional structure. Ink formulations for three-dimensional printing are also disclosed. Additionally, provided herein are methods for manufacturing devices and uses there e.g., in treating a condition in a subject in need thereof. The devices may be coated with therapeutic agents, such as those that promo bone growth, and/or seeded with cells to generate devices for use in tissue replacement and grafting.

### CROSS REFERENCE TO RELATED APPLICATIONS

This application claims the benefit of U.S. Application No. 63/153,279, filed Feb. 24, 2021, the entirety of which is incorporated by reference herein.

### GOVERNMENT RIGHTS

This invention was made with government support under W81XWH-18-C-0182 awarded by the U.S. Army Medical Material Command and W81XWH-20-C-0069 awarded by U.S. Army Medical Research Acquisition Activity. The government has certain rights in the invention.

### SEQUENCE LISTING

The application contains a Sequence Listing which has been submitted in ASCII format via EFS-Web and is hereby incorporated by reference in its entirety. The ASCII copy, created on Dec. 3, 2020, is named 50222-709_601_SL.txt and is 285,147 bytes in size.

### BACKGROUND

Three-dimensional (3D) printing is a manufacturing process of making three dimensional solid objects from a digital file. In the additive process of 3D printing an object is created by laying down successive layers of material until the desired object is created, with up to micrometer accuracy. Paired with computer-aided design (CAD) software, 3D printing enables production of complex functional shapes that can be easily customized compared with traditional manufacturing methods.

Surgically implanting scaffolds and/or other forms of graft materials to promote tissue regeneration is a useful technique if the implant can match the mechanical properties of the native tissue. Various materials can be used as ink in the fabrication of porous 3D-printed structures for implantation, including materials that mimic tissue and enable tissue regrowth. Inks with effective bioactive and mechanical properties are required for regeneration of native tissue, and if used in 3D printing can be customized and adopted for large tissue defect repair.

### SUMMARY

The present disclosure provides ink formulations and methods for 3D printing scaffolds. Further provided are scaffolds that may be generated using such ink formulations and methods. The scaffolds may be coated with therapeutic agents, such as those that promote bone growth, and/or seeded with cells to generate devices for use in tissue replacement and grafting. For some devices, therapeutic agents may be tethered to the scaffold via a targeting moiety that interacts with a scaffold component, such as a ceramic material. Advantages of the materials and methods described herein include providing a 3D-printed, customizable implant, as well as more universal objects, such as strip, block, or cylindrical objects. As the implants are 3D-printed, precise control of the implant geometry is possible. Implants printed with these inks are thus suitable for many different therapies including long bone repair, spinal fusion, maxio-facial structures, etc. The different formulations of the inks produce materials that result in implantable devices with differing properties, including differing porosity and flexibility.

Various inks and scaffolds of the present disclosure are designed to enhance the accessibility of therapeutic agents and/or targeting moieties to scaffold components, such as ceramic materials like beta-tricalcium phosphate. For instance, ink formulations described in Examples 1-2.

Various inks and methods of the present disclosure are designed to improve the manufacturability of 3D printed scaffolds by converting the ink to 1.75 mm diameter filaments. In filament form (rather than syringe-based bioprinting ink), a larger quantity of material can be prepared, stored, and used continuously (e.g., 1 kg of filament wound around a spool) before needing to recharge material in the 3D printer. Additionally, fused filament fabrication (FFF) 3D printers generally have a larger available build volume than syringe-based bioprinters and can be printed at a faster nozzle speed, both of which improve manufacturing throughput. Also, there is a very wide variety of FFF printers on the market compared to a relatively small assortment of syringe-based bioprinters. For instance, ink formulations #5 and #6 were both fabricated into 1.75 mm diameter filaments material and demonstrated in a FFF 3D printer, as described further in Examples 1-2.

Various inks and scaffolds of the present disclosure are designed to optimize bioresorption characteristics of the scaffolds. For instance, the copolymers used in ink formulation #2 (caprolactone/glycolide copolymer (95:5)), ink formulation #3 (caprolactone/glycolide copolymer (90:10)), and ink formulation #4 (poly(D,L-lactide-co-glycolide) copolymer (50:50)) have faster resorption rates than polycaprolactone. The resorption rates vary from slowest to fastest as: polycaprolactone, polycaprolactone/polyglycolide copolymer (95:5), polycaprolactone/glycolide copolymer (90:10), polydioxanone/L-lactide copolymer (90:10), poly(D,L-lactide-co-glycolide) copolymer (50:50).

In one aspect, provided herein is a three-dimensional structure comprising about 50% to about 90% by weight ceramic and about 10% to about 50% by weight polymer, wherein the structure has a plurality of micropores. In some embodiments, the micropores have an average pore size of about 1 micron to about 500 microns, or about 50 microns to about 250 microns, or about 150 microns in diameter. In some embodiments, the micropores provide additional surface area to the structure for contact with a therapeutic agent. In some embodiments, micropores are formed after removal of one or more pore formers from the structure. In some embodiments, the pore former comprises a second polymer, a sugar, or a salt, or a combination thereof. In some embodiments, micropores are formed by release of carbon dioxide from a blowing agent present during manufacture of the structure. In some embodiments, the blowing agent comprises sodium bicarbonate.

In another aspect, provided herein is a three-dimensional structure comprising about 50% to about 90% by weight ceramic and about 10% to about 50% by weight polymer, wherein the structure has an open porosity of about 15% to about 50%.

In another aspect, provided herein is a three-dimensional structure comprising about 50% to about 90% by weight ceramic and about 10% to about 50% by weight polymer, wherein the structure has a density of about 1 g/cm3 to about 2 g/cm3.

In another aspect, provided herein is a three-dimensional structure comprising about 50% to about 90% by weight ceramic and about 10% to about 50% by weight polymer, wherein the structure has a strut diameter of about 300 micrometers to about 600 micrometers.

In some embodiments of a structure provided herein, the ceramic comprises calcium phosphate, hydroxyapatite, fluorapatite, bone, silicate, orvanadate, or a combination thereof. In some embodiments of a structure provided herein, the polymer comprises polycaprolactone, caprolactone/glycolide copolymer, poly(D,L-lactide-co-glycolide) copolymer, or dioxanone/L-lactide copolymer, or a combination thereof. In some embodiments of a structure provided herein, the ceramic comprises calcium phosphate, hydroxyapatite, fluorapatite, bone, silicate, orvanadate, or a combination thereof, and the polymer comprises polycaprolactone, caprolactone/glycolide copolymer, poly(D,L-lactide-co-glycolide) copolymer, or dioxanone/L-lactide copolymer, or a combination thereof.

In some embodiments of a structure provided herein, the polymer comprises polycaprolactone and the ceramic comprises calcium phosphate. In some embodiments, the structure comprises about 65% to about 85% by weight calcium phosphate and about 15% to about 35% by weight polycaprolactone. In some embodiments, provided is a structure comprising about 65% to about 85% by weight calcium phosphate and about 15% to about 35% by weight polycaprolactone.

In some embodiments of a structure provided herein, the polymer comprises caprolactone/glycolide copolymer and the ceramic comprises calcium phosphate. In some embodiments, the structure comprises about 65% to about 85% by weight calcium phosphate and about 15% to about 35% by weight caprolactone/glycolide copolymer. In some embodiments, provided is a structure comprising about 65% to about 85% by weight calcium phosphate and about 15% to about 35% by weight caprolactone/glycolide copolymer.

In some embodiments of a structure provided herein, the polymer comprises poly(D,L-lactide-co-glycolide) copolymer or dioxanone/L-lactide copolymer, and the ceramic comprises calcium phosphate. In some embodiments, the structure comprises about 65% to about 85% by weight calcium phosphate and about 15% to about 35% by weight poly(D,L-lactide-co-glycolide) copolymer or dioxanone/L-lactide copolymer. In some embodiments, provided is a structure comprising about 65% to about 85% by weight calcium phosphate and about 15% to about 35% by weight poly(D,L-lactide-co-glycolide) copolymer or dioxanone/L-lactide copolymer.

In some embodiments of a structure provided herein, the structure is formed from an ink comprising the ceramic, the polymer, and a sacrificial pore former. In some embodiments of a structure provided herein, the structure is formed from a filament using an extrusion based three dimensional printing method.

Further provided is a device comprising a structure provided herein, and a therapeutic agent. In some embodiments, the therapeutic agent comprises a bone morphogenetic protein (BMP). In some embodiments, the therapeutic agent comprises a targeting moiety, and the targeting moiety is non-covalently bound to the three-dimensional structure. In some embodiments, the targeting moiety comprises a polypeptide at least about 70%, 75%, 80%, 85%, 90%, 95%, or 100% identical to any one of the sequences of Tables 2-3. In some embodiments, the therapeutic agent comprises a chimeric polypeptide comprising a sequence at least about 70%, 75%, 80%, 85%, 90%, 95%, or 100% identical to any one of SEQ ID NOS: 433-441.

In one aspect, provided herein is a device comprising: a therapeutic agent and a printed three-dimensional structure, the printed three-dimensional structure comprising about 50% to about 90% by weight ceramic and about 10% to about 50% by weight polymer. In some embodiments, the therapeutic agent is non-covalently bound to the printed three-dimensional structure. In some embodiments, the ceramic comprises calcium phosphate, hydroxyapatite, fluorapatite, bone, silicate, or vanadate, or a combination thereof. In some embodiments, the ceramic comprises beta-tricalcium phosphate (β-TCP). In some embodiments, the structure comprises about 70% to about 80% by weight ceramic. In some embodiments, the structure comprises about 75% by weight ceramic. In some embodiments, the polymer comprises a polyester. In some embodiments, the polymer comprises polycaprolactone (PCL), polyglycolide, lactide, or polydioxanone (PDS), or a combination thereof. In some embodiments, the polymer comprises polycaprolactone (PCL). In some embodiments, the polymer comprises polyglycolide. In some embodiments, the polymer comprises polydioxanone (PDS). In some embodiments, the polymer comprises a lactide. In some embodiments, the lactide comprises L-lactide. In some embodiments, the lactide comprises poly(D,L-lactide). In some embodiments, the polymer comprises a copolymer. In some embodiments, the copolymer comprises polycaprolactone (PCL) and polyglycolide. In some embodiments, the copolymer comprises about 90-95 percent by mole PCL and about 5-10 percent by mole polyglycolide. In some embodiments, the copolymer comprises poly(D,L-lactide-co-glycolide) copolymer. In some embodiments, the copolymer comprises about 40-60 percent by mole poly(D,L-lactide) and about 40-60 percent by mole polyglycolide. In some embodiments, the copolymer comprises PDS-glycolide copolymer. In some embodiments, the copolymer comprises about 90-95 percent by mole PDS and about 5-10 percent by mole glycolide. In some embodiments, the copolymer comprises PDS-L-lactide copolymer. In some embodiments, the copolymer comprises about 90-95 percent by mole PDS and about 5-10 percent by mole L-lactide. In some embodiments, the structure comprises about 15% to about 25% by weight polymer. In some embodiments, the structure comprises about 25% by weight polymer. In some embodiments, the printed three-dimensional structure is formed from an ink comprising about 30% to about 70% by weight the ceramic, about 10% to about 30% by weight the polymer, and optionally one or more additional agents. In some embodiments, the ink comprises about 50% to about 70% by weight the ceramic. In some embodiments, the ink comprises ab out 60% by weight the ceramic. In some embodiments, the ink comprises ab out 15% to about 25% by weight the ceramic. In some embodiments, the ink comprises about 20% by weight the ceramic. In some embodiments, the ink comprises the one or more additional agents. In some embodiments, the ink comprises about 1% to about 30% by weight the one or more additional agents. In some embodiments, the one or more additional agents comprises an additional polymer, particulate, or blowing agent, or a combination thereof. In some embodiments, the one or more additional agents comprises the additional polymer. In some embodiments, the additional polymer comprises polyethylene glycol. In some embodiments, the additional polymer is present in the ink at about 10% to about 30% by weight. In some embodiments, the one or more additional agents comprises the particulate. In some embodiments, the particulate comprises sodium chloride, calcium chloride, sucrose, trehalose (e.g., a, a trehalose dihydrate), or mannitol (e.g., D-mannitol), or a combination thereof. In some embodiments, the particulate is present in the ink at about 1% to about 10% by weight. In some embodiments, the one or more additional agents comprises the blowing agent. In some embodiments, the blowing agent comprises baking powder (e.g., monocalcium phosphate, sodium bicarbonate, corn starch) and/or azodicarbonamide. In some embodiments, the particulate is present in the ink at about 5% to about 20% by weight. In some embodiments, the therapeutic agent comprises a mammalian growth factor or a functional portion thereof. In some embodiments, the therapeutic agent comprises one or more polypeptides selected from Table 1, or a functional portion thereof. In some embodiments, the therapeutic agent comprises a bone morphogenetic protein (BMP). In some embodiments, the therapeutic agent comprises a targeting moiety, and the targeting moiety is non-covalently bound to the printed three-dimensional structure. In some embodiments, the targeting moiety is bound to the printed three-dimensional structure with an affinity of ab out 1 pM to about 100 μM. In some embodiments, the targeting moiety comprises a polypeptide at least about 70%, 75%, 80%, 85%, 90%, 95%, or 100% identical to any one of the sequences of Tables 2-3. In some embodiments, the targeting moiety comprises about 2, 3, 4, 5, 6, 7, 8, 9, or 10 sequences selected from a sequence from Tables 2-3. In some embodiments, the therapeutic agent comprises a chimeric polypeptide comprising a sequence at least about 70%, 75%, 80%, 85%, 90%, 95%, or 100% identical to any one of SEQ ID NOS: 433-441.

Further provided is a method of treating a condition in a subject in need thereof, the method comprising administering to the subject the device. In some embodiments, the condition comprises a bone defect, cartilage defect, soft tissue defect, tendon defect, fascia defect, ligament defect, organ defect, osteotendinous tissue defect, dermal defect, osteochondral defect, osteoporosis, avascular necrosis, or congenital skeletal malformation, or a combination thereof. In some embodiments, the method comprises spinal fusion. In some embodiments, the spinal fusion comprises posterior lumbar fusion (PLF) and/or interbody fusion. In some embodiments, the method comprises bone repair, dental repair, craniomaxillofacial repair, ankle fusion, kyphoplasty, osteoplasty, scaphoid fracture repair, tendeno-osseous repair, costal reconstruction, subchondral bone repair, cartilage repair, or surgical implantation of the three-dimensional structure or device, or a combination thereof.

Further provided is a formulation comprising about 30% to about 70% by weight ceramic, about 10% to about 30% by weight polymer, and optionally one or more additional agents. In some embodiments, the ceramic comprises calcium phosphate, hydroxyapatite, fluorapatite, bone, silicate, or vanadate, or a combination thereof. In some embodiments, the ceramic comprises beta-tricalcium phosphate (β-TCP). In some embodiments, the ink comprises about 50% to about 70% by weight the ceramic. In some embodiments, the ink comprises about 60% by weight the ceramic. In some embodiments, the polymer comprises a polyester. In some embodiments, the polymer comprises polycaprolactone (PCL), polyglycolide, lactide, or polydioxanone (PDS), or a combination thereof. In some embodiments, the polymer comprises polycaprolactone (PCL). In some embodiments, the polymer comprises polyglycolide. In some embodiments, the polymer comprises polydioxanone (PDS). In some embodiments, the polymer comprises a lactide. In some embodiments, the lactide comprises L-lactide. In some embodiments, the lactide comprises poly(D,L-lactide). In some embodiments, the polymer comprises a copolymer. In some embodiments, the copolymer comprises polycaprolactone (PCL) and polyglycolide. In some embodiments, the copolymer comprises about 90-95 percent by mole PCL and about 5-10 percent by mole polyglycolide. In some embodiments, the copolymer comprises poly(D,L-lactide-co-glycolide) copolymer. In some embodiments, the copolymer comprises about 40-60 percent by mole poly(D,L-lactide) and about 40-60 percent by mole polyglycolide. In some embodiments, the copolymer comprises PDS-glycolide copolymer. In some embodiments, the copolymer comprises about 90-95 percent by mole PDS and about 5-10 percent by mole glycolide. In some embodiments, the copolymer comprises PDS-L-lactide copolymer. In some embodiments, the copolymer comprises about 90-95 percent by mole PDS and about 5-10 percent by mole L-lactide. In some embodiments, the ink comprises the one or more additional agents. In some embodiments, the ink comprises about 1% to about 30% by weight the one or more additional agents. In some embodiments, the one or more additional agents comprises an additional polymer, particulate, or blowing agent, or a combination thereof. In some embodiments, the one or more additional agents comprises the additional polymer. In some embodiments, the additional polymer comprises polyethylene glycol. In some embodiments, the additional polymer is present in the ink at about 10% to about 30% by weight. In some embodiments, the one or more additional agents comprises the particulate. In some embodiments, the particulate comprises sodium chloride, calcium chloride, sucrose, trehalose (e.g., α,α trehalose dihydrate), or mannitol (e.g., D-mannitol), or a combination thereof. In some embodiments, the particulate is present in the ink at about 1% to about 10% by weight. In some embodiments, the one or more additional agents comprises the blowing agent. In some embodiments, the blowing agent comprises baking powder (e.g., monocalcium phosphate, sodium bicarbonate, corn starch) and/or azodicarbonamide. In some embodiments, the particulate is present in the ink at about 5% to about 20% by weight.

Further provided is a filament comprising the formulation. In some embodiments, the filament has a diameter of about 1.5 mm to about 2 mm.

Further provided is a method of preparing a three-dimensional structure, the method comprising using the formation in a three-dimensional printing method.

Further provided is a three-dimensional structure prepared using the ink formulation. In some embodiments, the structure comprises about 50% to about 90% by weight ceramic. In some embodiments, the structure comprises about 50% to about 90% by weight tricalcium phosphate. In some embodiments, the structure comprises about 10% to about 50% by weight polymer. In some embodiments, the polymer comprises a polyester. In some embodiments, the polymer comprises polycaprolactone (PCL), polyglycolide, lactide, or polydioxanone (PDS), or a combination thereof. In some embodiments, the polymer comprises polycaprolactone (PCL). In some embodiments, the polymer comprises polyglycolide. In some embodiments, the polymer comprises polydioxanone (PDS). In some embodiments, the polymer comprises a lactide. In some embodiments, the lactide comprises L-lactide. In some embodiments, the lactide comprises poly(D,L-lactide). In some embodiments, the polymer comprises a copolymer. In some embodiments, the copolymer comprises polycaprolactone (PCL) and polyglycolide. In some embodiments, the copolymer comprises about 90-95 percent by mole PCL and about 5-10 percent by mole polyglycolide. In some embodiments, the copolymer comprises poly(D,L-lactide-co-glycolide) copolymer. In some embodiments, the copolymer comprises about 40-60 percent by mole poly(D,L-lactide) and about 40-60 percent by mole polyglycolide. In some embodiments, the copolymer comprises PDS-glycolide copolymer. In some embodiments, the copolymer comprises about 90-95 percent by mole PDS and about 5-10 percent by mole glycolide. In some embodiments, the copolymer comprises PDS-L-lactide copolymer. In some embodiments, the copolymer comprises about 90-95 percent by mole PDS and about 5-10 percent by mole L-lactide. In some embodiments, the structure comprises about 15% to about 25% by weight polymer. In some embodiments, the structure comprises about 25% by weight polymer.

Further provided is a three-dimensional structure comprising about 50% to about 90% by weight ceramic, and about 10% to about 30% polymer. In some embodiments, the ceramic comprises calcium phosphate, hydroxyapatite, fluorapatite, bone, silicate, or vanadate, or a combination thereof. In some embodiments, the ceramic comprises beta-tricalcium phosphate (β-TCP). In some embodiments, the structure comprises ab out 65% to ab out 85% by weight ceramic. In some embodiments, the structure comprises about 70% to about 80% by weight ceramic. In some embodiments, the structure comprises about 75% by weight ceramic. In some embodiments, the polymer comprises a polyester. In some embodiments, the polymer comprises polycaprolactone (PCL), polyglycolide, lactide, or polydioxanone (PDS), or a combination thereof. In some embodiments, the polymer comprises polycaprolactone (PCL). In some embodiments, the polymer comprises polyglycolide. In some embodiments, the polymer comprises polydioxanone (PDS). In some embodiments, the polymer comprises a lactide. In some embodiments, the lactide comprises L-lactide. In some embodiments, the lactide comprises poly(D,L-lactide). In some embodiments, the polymer comprises a copolymer. In some embodiments, the copolymer comprises polycaprolactone (PCL) and polyglycolide. In some embodiments, the copolymer comprises about 90-95 percent by mole PCL and about 5-10 percent by mole polyglycolide. In some embodiments, the copolymer comprises poly(D,L-lactide-co-glycolide) copolymer. In some embodiments, the copolymer comprises about 40-60 percent by mole poly(D,L-lactide) and about 40-60 percent by mole polyglycolide. In some embodiments, the copolymer comprises PDS-glycolide copolymer. In some embodiments, the copolymer comprises about 90-95 percent by mole PDS and about 5-10 percent by mole glycolide. In some embodiments, the copolymer comprises PDS-L-lactide copolymer. In some embodiments, the copolymer comprises about 90-95 percent by mole PDS and about 5-10 percent by mole L-lactide. In some embodiments, the structure comprises about 15% to about 25% by weight polymer. In some embodiments, the structure comprises about 25% by weight polymer. In some embodiments, the structure is prepared by a three-dimensional printing method.

Further provided is a method of manufacturing the three-dimensional structure, the method comprising depositing an ink formulation in a three-dimensional form. In some embodiments, the ink formulation comprises the ink formulation herein.

Further provided is a method of treating a condition in a subject in need thereof, the method comprising delivering to an organ or tissue of the subject the structure.

Further provided is a method of treating a condition in a subject in need thereof, the method comprising delivering to an organ or tissue of the subject the structure manufactured by a method herein.

In some embodiments, the condition comprises a bone defect, cartilage defect, soft tissue defect, tendon defect, fascia defect, ligament defect, organ defect, osteotendinous tissue defect, dermal defect, osteochondral defect, osteoporosis, avascular necrosis, or congenital skeletal malformation, or a combination thereof. In some embodiments, the method comprises spinal fusion. In some embodiments, the spinal fusion comprises posterior lumbar fusion (PLF) and/or interbody fusion. In some embodiments, the method comprises bone repair, dental repair, craniomaxillofacial repair, ankle fusion, kyphoplasty, osteoplasty, scaphoid fracture repair, tendeno-osseous repair, costal reconstruction, sub chondral b one rep air, cartilage repair, or surgical implantation of the three-dimensional structure or device, or a combination thereof. In some embodiments, the method further comprises treating the subject with a therapeutic agent.

Further provided is a method of delivering a therapeutic agent to a subject in need thereof, the method comprising delivering to an organ or tissue of the subject a device comprising a therapeutic agent and the structure.

In some embodiments, the therapeutic agent comprises a mammalian growth factor or a functional portion thereof. In some embodiments, the therapeutic agent comprises one or more polypeptides selected from Table 1, or a functional portion thereof. In some embodiments, the therapeutic agent comprises a bone morphogenetic protein (BMP). In some embodiments, the therapeutic agent comprises a targeting moiety that non-covalently binds to the structure. In some embodiments, the targeting moiety binds to the printed three-dimensional structure with an affinity of about 1 pM to about 100 μm. In some embodiments, the targeting moiety comprises a polypeptide at least about 70%, 75%, 80%, 85%, 90%, 95%, or 100% identical to any one of the sequences of Tables 5-6. In some embodiments, the targeting moiety comprises about 2, 3, 4, 5, 6, 7, 8, 9, or 10 sequences selected from the sequences of Tables 5-6. In some embodiments, the therapeutic agent comprises or is part of a chimeric polypeptide comprising a sequence at least about 70%, 75%, 80%, 85%, 90%, 95%, or 100% identical to any one of SEQ ID NOS: 433-441.

In some embodiments of a device and/or structure herein, the structure has a density of about 1 g/cm3 to about 2 g/cm3. In some embodiments of a device and/or structure herein, the structure has an open porosity of about 15% to about 50%. In some embodiments of a device and/or structure herein, the structure of any one of claims 91-139, wherein the structure has a strut diameter of about 300 μm to about 600 μm.

The details of one or more embodiments of the disclosure are set forth in the accompanying drawings and the description below. Other features, objects, and advantages of the disclosure will be apparent from the description and drawings, and from the claims.

### BRIEF DESCRIPTION OF THE DRAWINGS

FIGS. 1A-1C are images from scanning electron microscopy (SEM images) of an example 3D printed object made with ink formulation #1 as outlined in Example 2. FIG. 1A and FIG. 1C are SEM images of the surface of the object at increasing magnifications. FIG. 1B is an SEM image of the side of the object.

FIGS. 2A-2C are images from scanning electron microscopy (SEM images) of an example 3D printed object made with ink formulation #2 as outlined in Example 2. FIG. 2A and FIG. 2C are SEM images of the surface of the object at increasing magnifications. FIG. 2B is an SEM image of the side of the object.

FIGS. 3A-3C are images from scanning electron microscopy (SEM images) of an example 3D printed object made with ink formulation #3 as outlined in Example 2. FIG. 3A and FIG. 3C are SEM images of the surface of the object at increasing magnifications. FIG. 3B is an SEM image of the side of the object.

FIGS. 4A-4C are images from scanning electron microscopy (SEM images) of an example 3D printed object made with ink formulation #4 as outlined in Example 2. FIG. 4A and FIG. 4C are SEM images of the surface of the object at increasing magnifications. FIG. 4B is an SEM image of the side of the object.

FIGS. 5A-5C are images from scanning electron microscopy (SEM images) of an example 3D printed object made with ink formulation #5 as outlined in Example 2. FIG. 5A and FIG. 5C are SEM images of the surface of the object at increasing magnifications. FIG. 5B is an SEM image of the side of the object.

FIGS. 6A-6C are images from scanning electron microscopy (SEM images) of an example 3D printed object made with ink formulation #6 as outlined in Example 2. FIG. 6A and FIG. 6C are SEM images of the surface of the object at increasing magnifications. FIG. 6B is an SEM image of the side of the object.

FIGS. 7A-7C are images from scanning electron microscopy (SEM images) of an example 3D printed object made with ink formulation #7 as outlined in Example 2. FIG. 7A and FIG. 7C are SEM images of the surface of the object at increasing magnifications. FIG. 7B is an SEM image of the side of the object.

FIGS. 8A-8C are images from scanning electron microscopy (SEM images) of an example 3D printed object made with ink formulation #8 as outlined in Example 2. FIG. 8A and FIG. 8C are SEM images of the surface of the object at increasing magnifications. FIG. 8B is an SEM image of the side of the object.

FIGS. 9A-9C are images from scanning electron microscopy (SEM images) of an example 3D printed object made with ink formulation #9 as outlined in Example 2. FIG. 9A and FIG. 9C are SEM images of the surface of the object at increasing magnifications. FIG. 9B is an SEM image of the side of the object.

FIGS. 10A-10C are images from scanning electron microscopy (SEM images) of an example 3D printed object made with ink formulation #10 as outlined in Example 2. FIG. 10A and FIG. 10C are SEM images of the surface of the object at increasing magnifications. FIG. 10B is an SEM image of the side of the object.

FIGS. 11A-11C are images from scanning electron microscopy (SEM images) of an example 3D printed object made with ink formulation #11 as outlined in Example 2. FIG. 11A and FIG. 11C are SEM images of the surface of the object at increasing magnifications. FIG. 11B is an SEM image of the side of the object.

FIGS. 12A-12C are images from scanning electron microscopy (SEM images) of an example 3D printed object made with ink formulation #12 as outlined in Example 2. FIG. 12A and FIG. 12C are SEM images of the surface of the object at increasing magnifications. FIG. 12B is an SEM image of the side of the object.

### DETAILED DESCRIPTION

Various formulations and structures are provided herein. The structures may be coated with a tetherable protein (for example, a growth factor) for a desired therapeutic effect, such as promotion of bone growth after implantation of the tethered structure.

### Formulations

In one aspect, provided herein are formulations for fabrication of 3D-printed structures. As a non-limiting example, the formulations include a ceramic material such as calcium phosphate (e.g., tricalcium phosphate, beta tricalcium phosphate, alpha tricalcium phosphate), hydroxyapatite, fluorapatite, bone (e.g., demineralized bone), glasses (bioglasses) such as silicates, vanadates, and related ceramic minerals, or chelated divalent metal ions, or a combination thereof. In some embodiments, the ceramic material comprises beta-tricalcium phosphate (β-TCP). In some embodiments, the formulation is about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70 percent ceramic by weight of the formulation. For instance, the formulation is about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% ceramic by weight. In some embodiments, the ceramic is β-TCP. In some embodiments, the β-TCP is introduced into the formulation as a powder. In some embodiments, the formulation comprises one or more additional components. Non-limiting examples of additional components include water, polymer (including copolymer), antifoaming agent, dispersing agent, solvent, particulate, blowing agent, and plasticizer.

In some embodiments, the formulation comprises one or more polymers, e.g., about 1, 2, 3, 4, or 5 polymers. Non-limiting examples of polymers include poly(ethylene oxide), poly(propylene oxide), polyethylene glycol (PEG), and polyester. In some embodiments, the formulation comprises a polymer that is about 5-30 percent by weight of the formulation. In some embodiments, the formulation is about 20-60 percent total polymer by weight. For instance, the total polymer includes two or more polymers in the formulation, where the total percentage of polymers in the formulation is about 20-60 percent of the weight of the formulation. In some embodiments, the formulation is about 30 to about 50 percent total polymer by weight. As non-limiting examples, the formulation is ab out 35-45 percent total polymer by weight. In an example, the polymer comprises a poloxamer. Poloxamers are block copolymers of poly(ethylene oxide) (PEO) and poly(propylene oxide) (PPO). A non-limiting example of a poloxamer is poloxamer 407, such as Pluronic® F-127. In some cases, the formulation comprises about 5-20, 5-15, 5-10, 10-20, 10-15, or 15-20 percent poloxamer 407 by weight. As another example, the polymer comprises polyethylene glycol (PEG). In some cases, the formulation comprises about 5-30, 5-25, 5-20, 5-15, 5-10, 10-30, 10-25, 10-20, 10-15, 15-30, 15-25, 15-20, 20-30, 20-25, or 25-30 percent by weight PEG. For instance, the formulation comprises about 5-30, or 5, 6, 7, 8, 9, 10, 11, 12, 13, 14, 15, 16, 17, 18, 19, 20, 21, 22, 23, 24, 25, 26, 27, 28, 29 or 30 percent by weight PEG. In some embodiments, there is a first PEG with a first molecular weight, and a second PEG with a second molecular weight. In some embodiments, PEG can have a molecular weight from 500 g/mol to 25,000 g/mol. In some cases, the molecular weight of PEG is about 500 g/mol, about 1,000 g/mol, about 1,500 g/mol, about 20.00 g/mol, about 2,500 g/mol, about 3,000 g/mol, about 3,500 g/mol, about 4,000 g/mol, about 4,500 g/mol, about 5,000 g/mol, about 5,500 g/mol, about 6,000 g/mol, about 6,500 g/mol, about 7,000 g/mol, about 7,500 g/mol, about 8,000 g/mol, about 8,500 g/mol, about 9,000 g/mol, about 9,500 g/mol, about 10,000 g/mol, about 10,500 g/mol, about 11,000 g/mol, about 11,500 g/mol, about 12,000 g/mol, about 12,500 g/mol, about 13,000 g/mol, about 13,500 g/mol, about 14,000 g/mol, about 14,500 g/mol, about 15,000 g/mol, about 15,500 g/mol, about 16,000 g/mol, about 16,500 g/mol, about 17,000 g/mol, about 17,500 g/mol, about 18,000 g/mol, about 18,500 g/mol, about 19,000 g/mol, about 19,500 g/mol, about 20,000 g/mol, about 20,500 g/mol, about 21,000 g/mol, about 21,500 g/mol, about 22,000 g/mol, about 22,500 g/mol, about 23,000 g/mol, about 23,500 g/mol, about 24,000 g/mol, about 24,500 g/mol, or about 25,000 g/mol. In a nonlimiting example embodiment, the formulation comprises PEG having a molecular weight of 1,500 g/mol. In a further nonlimiting example embodiment, the formulation comprises PEG having a molecular weight of 8,000 g/mol. In a further nonlimiting example embodiment, the formulation comprises PEG having a molecular weight of 20,000 g/mol. In a nonlimiting example embodiment, the formulation comprises a first PEG at about 15 percent by weight and a second PEG at about 15 percent by weight. As another example, the polymer comprises polydioxanone (PDS). In some cases, the formulation comprises about 10-30, 10-25, 10-20, 10-15, 15-30, 15-25, 15-20, 20-30, 20-25, or 25-30 percent by weight PDS. For instance, the formulation comprises about 15-25, or 15, 16, 17, 18, 19, 20, 21, 22, 23, 24, or 25 percent by weight PDS. As another example, the polymer comprises a polyester. In some embodiments, the polyester comprises a biodegradable polyester such as polycaprolactone (PCL). In some cases, the formulation comprises about 10-30, 10-25, 10-20, 10-15, 15-30, 15-25, 15-20, 20-30, 20-25, or 25-30 percent by weight PCL. For instance, the formulation comprises about 15-25, or 15, 16, 17, 18, 19, 20, 21, 22, 23, 24, or 25 percent by weight PCL. In some cases, the formulation comprises PCL having a molecular weight of 50,000 g/mol. In some embodiments, the polyester comprises a polyglycolide or poly(glycolic acid) (PGA). In some cases, the formulation comprises about 0.5-20, 0.5-18, 0.5-16, 0.5-14, 0.5-12, 0.5-10, 0.5-8, 0.5-6, 0.5-4, 0.5-2, 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent by weight PGA. For instance, the formulation comprises about 1, 2, 3, 4, 5, 6, 7, 8, 9, 10, 11, 12, 13, 14, or 15 percent by weight PGA. In some cases, the PGA has a molecular weight of about 38,000-54,000. In some embodiments, the polyester comprises a polylactide, such as poly(D, L-lactide). In some cases, the formulation comprises about 0.5-20, 0.5-18, 0.5-16, 0.5-14, 0.5-12, 0.5-10, 0.5-8, 0.5-6, 0.5-4, 0.5-2, 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent by weight polylactide. For instance, the formulation comprises about 1, 2, 3, 4, 5, 6, 7, 8, 9, 10, 11, 12, 13, 14, or 15 percent by weight polylactide.

In some embodiments, the polymer comprises a copolymer. In some cases, the copolymer comprises polyglycolide. In some cases, the copolymer comprises PCL and polyglycolide. For instance, the copolymer comprises about 80-99, 80-98, 80-97, 80-96, 80-95, 80-94, 80-93, 80-92, 80-91, 80-90, 80-89, 80-88, 80-87, 80-86, 80-85, 85-99, 85-98, 85-97, 85-96, 85-95, 85-94, 85-93, 85-92, 85-91, 85-90, 90-99, 90-98, 90-97, 90-96, 90-95, 90-94, 90-93, 90-92, 90-91, 95-99, 95-98, 95-97, or 95-96 percent molar PCL, and about 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent molar polyglycolide. In some cases, the copolymer comprises about 90-95 percent by mole PCL and about 5-10 percent by mole polyglycolide. In some cases, the copolymer comprises PDS and polyglycolide. For instance, the copolymer comprises about 80-99, 80-98, 80-97, 80-96, 80-95, 80-94, 80-93, 80-92, 80-91, 80-90, 80-89, 80-88, 80-87, 80-86, 80-85, 85-99, 85-98, 85-97, 85-96, 85-95, 85-94, 85-93, 85-92, 85-91, 85-90, 90-99, 90-98, 90-97, 90-96, 90-95, 90-94, 90-93, 90-92, 90-91, 95-99, 95-98, 95-97, or 95-96 percent molar PDS, and about 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent molar polyglycolide. In some cases, the copolymer comprises about 90-95 percent by mole PDS and ab out 5-10 percent by mole polyglycolide. In some cases, the copolymer comprises PDS-glycolide copolymer. For instance, the formulation comprises about 10-30, 10-25, 10-20, 10-15, 15-30, 15-25, 15-20, 20-30, 20-25, 25-30, or 20 percent by weight PDS-glycolide copolymer. In some cases, the copolymer comprises poly(D, L-lactide-co-glycolide) copolymer. For instance, the formulation comprises about 10-30, 10-25, 10-20, 10-15, 15-30, 15-25, 15-20, 20-30, 20-25, 25-30, or 20 percent by weight poly(D, L-lactide-co-glycolide) copolymer. In some cases, the copolymer comprises lactide (e.g., poly(D, L-lactide)) and polyglycolide. For instance, the copolymer comprises about 35-65, 35-60, 35-55, 35-50, 35-45, 35-40, 40-65, 40-60, 40-55, 40-50, 40-45, 45-65, 45-60, 45-55, 45-50, 50-65, 50-60, 50-55, 55-65, 55-60, 60-65 percent molar lactide (e.g., poly(D, L-lactide)), and about 35-65, 35-60, 35-55, 35-50, 35-45, 35-40, 40-65, 40-60, 40-55, 40-50, 40-45, 45-65, 45-60, 45-55, 45-50, 50-65, 50-60, 50-55, 55-65, 55-60, 60-65 percent molar glycolide. In some cases, the copolymer comprises poly(D, L-lactide-co-glycolide) copolymer. For instance, the formulation comprises about 10-30, 10-25, 10-20, 10-15, 15-30, 15-25, 15-20, 20-30, 20-25, 25-30, or 20 percent by weight poly(D, L-lactide-co-glycolide) copolymer. In some cases, the copolymer comprises lactide (e.g., L-lactide) and PDS. For instance, the copolymer comprises about 35-65, 35-60, 35-55, 35-50, 35-45, 35-40, 40-65, 40-60, 40-55, 40-50, 40-45, 45-65, 45-60, 45-55, 45-50, 50-65, 50-60, 50-55, 55-65, 55-60, 60-65 percent molar PDS, and about 35-65, 35-60, 35-55, 35-50, 35-45, 35-40, 40-65, 40-60, 40-55, 40-50, 40-45, 45-65, 45-60, 45-55, 45-50, 50-65, 50-60, 50-55, 55-65, 55-60, 60-65 percent molar lactide (e.g., L-lactide). In some cases, the copolymer comprises PDS-L-lactide copolymer. For instance, the formulation comprises about 10-30, 10-25, 10-20, 10-15, 15-30, 15-25, 15-20, 20-30, 20-25, 25-30, or 20 percent by weight PDS-L-lactide copolymer. In some cases, the copolymer comprises dioxanone. In some cases, the copolymer comprises dioxanone and lactide (e.g., L-lactide). For instance, the copolymer comprises about 80-99, 80-98, 80-97, 80-96, 80-95, 80-94, 80-93, 80-92, 80-91, 80-90, 80-89, 80-88, 80-87, 80-86, 80-85, 85-99, 85-98, 85-97, 85-96, 85-95, 85-94, 85-93, 85-92, 85-91, 85-90, 90-99, 90-98, 90-97, 90-96, 90-95, 90-94, 90-93, 90-92, 90-91, 95-99, 95-98, 95-97, or 95-96 percent molar dioxanone, and about 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent molar lactide (e.g., L-lactide). In some cases, the copolymer comprises about 85-95 percent by mole dioxanone and about 5-15 percent by mole lactide (e.g., L-lactide).

In some instances, the copolymer has a faster resorption rate than a single polymer. For instance, the copolymers used in example embodiments of ink formulation #2 (polycaprolactone/polyglycolide copolymer (95:5)), ink formulation #3 (polycaprolactone/polyglycolide copolymer (90:10)), and ink formulation #4 (poly(D,L-lactide-co-glycolide) copolymer (50:50)) have faster resorption rates than polycaprolactone. The resorption rates vary from slowest to fastest as: polycaprolactone, polycaprolactone/polyglycolide copolymer (95:5), polycaprolactone/glycolide copolymer (90:10), polydioxanone/L-lactide copolymer (90:10), poly(D,L-lactide-co-glycolide) copolymer (50:50).

In some embodiments, the formulation comprises two or more polymers. In some embodiments, the formulation is about 5-30, 5-25, 5-20, 5-15, 5-10, 10-30, 10-25, 10-20, 10-15, 15-30, 15-25, 15-20, 20-30, 20-25, or 25-30 percent by weight of a first polymer, and about 5-30, 5-25, 5-20, 5-15, 5-10, 10-30, 10-25, 10-20, 10-15, 15-30, 15-25, 15-20, 20-30, 20-25, or 25-30 percent by weight of a second polymer. In non-limiting examples, the formulation is about 10-30 percent by weight of a first polymer, and about 10-30 percent by weight of a second polymer, or about 10, 11, 12, 13, 14, 15, 16, 17, 18, 19, 20, 21, 22, 23, 24, 25, 26, 27, 28, 29, or 30 percent by weight of the first polymer, and about 10, 11, 12, 13, 14, 15, 16, 17, 18, 19, 20, 21, 22, 23, 24, 25, 26, 27, 28, 29, or 30 percent by weight of the second polymer. In some cases, the first and/or second polymer comprises PEG. In some cases, the first polymer comprises PCL and the second polymer comprises PEG. In some cases, the first polymer comprises PD S and the second polymer comprises PEG. In some cases, the first polymer comprises a copolymer and the second polymer comprises PEG. The copolymer may comprise PCL and polyglycolide (e.g., 95 mol % polycaprolactone, 5 mol % polyglycolide; 90 mol % polycaprolactone, 10 mol % polyglycolide). The copolymer may comprise polylactide (e.g., poly(D,L-lactide) and polyglycolide (e.g., 50 mol % poly(D,L-lactide), 50 mol % polyglycolide). The copolymer may comprise PDS-glycolide copolymer (e.g., 90 mol % PDS, 10 mol % polyglycolide). The copolymer may comprise PDS-L-lactide copolymer (e.g., 90 mol % PDS, 10 mol % L-lactide).

In some embodiments, the formulation comprises one or more particulates. The particulate may be a pore former. The particulate may be water soluble. The particulate may comprise a salt and/or sugar. Non-limiting examples of particulates include sodium chloride, calcium chloride, sucrose, trehalose (e.g., α,α trehalose dihydrate), and mannitol (e.g., D-mannitol). In some cases, the particulate comprises sucrose. In some embodiments, the formulation comprises about 1-10, 1-9, 1-8, 1-7, 1-6, 1-5, 2-10, 2-9, 2-8, 2-7, 2-6, 2-5, 3-10, 3-9, 3-8, 3-7, 3-6, 3-5, 4-10, 4-9, 4-8, 4-7, 4-6, 4-5, 5-10, 5-9, 5-8, 5-7, or 5-6 percent by weight particulate. In some cases, the formulation comprises about 1-10%, or about 1%, 2%, 3%, 4%, 5%, 6%, 7%, 8%, 9% or 10% particulate. In some embodiments, the particulate or pore former has an average size of about 1 micron to about 500 microns in diameter. For instance, about 1 micron to about 450 microns, about 1 micron to about 400 microns, about 1 micron to about 350 microns, about 1 micron to about 300 microns, about 1 micron to about 250 microns, about 1 micron to about 200 microns, about 1 micron to about 150 microns, about 50 microns to about 500 microns, about 50 microns to about 450 microns, about 50 microns to about 400 microns, about 50 microns to about 350 microns, about 50 microns to about 300 microns, about 50 microns to about 250 microns, about 50 microns to about 200 microns, about 50 microns to about 150 microns, about 100 microns to about 500 microns, about 100 microns to about 450 microns, about 100 microns to about 400 microns, about 100 microns to about 350 microns, about 100 microns to about 300 microns, about 100 microns to about 250 microns, about 100 microns to about 200 microns, about 100 microns to about 150 microns, about 150 microns to about 500 microns, about 150 microns to about 450 microns, about 150 microns to about 400 microns, about 150 microns to about 350 microns, about 150 microns to about 300 microns, about 150 microns to about 250 microns, or ab out 150 microns to ab out 200 microns in diameter. In some cases, the particular or pore former has an average size of about 50 microns to about 250 microns, about 60 microns to about 240 microns, about 70 microns to about 230 microns, about 80 microns to about 220 microns, or about 90 microns to about 210 microns in diameter. In some embodiments, the particulate of pore former has an average size of about 100 microns to about 200 microns, e.g., about 110 microns to about 190 microns, about 120 microns to about 180 microns, about 130 microns to about 170 microns, about 140 microns to about 160 microns, or about 100 microns, about 110 microns, about 120 microns, about 130 microns, about 140 microns, about 150 microns, about 160 microns, about 170 microns, about 180 microns, about 190 microns, or about 200 microns in diameter. In some embodiments, the particulate or pore former has an average size of about 150 microns in diameter. In some embodiments, upon removal of the particulate or pore former, a structure formed from the formulation has micropores that provide additional surface area to the structure for contact with a therapeutic agent as compared to a structure formed with a formulation lacking the particulate or pore former. In some embodiments, the micropores of the structure have an average pore size of about 1 micron to about 500 microns, or about 50 microns to about 250 microns, or about 150 microns in diameter.

In some embodiments, the formulation comprises one or more blowing agents. In some embodiments, the blowing agent comprises about 5-20, 5-18, 5-16, 5-14, 5-12, 5-10, 5-8, 5-6, 6-20, 6-18, 6-16, 6-14, 6-12, 6-10, 6-8, 8-20, 8-18, 8-16, 8-14, 8-12, 8-10, 10-20, 10-18, 10-16, 10-14, 10-12, 5-15, or about 5, 6, 7, 8, 9, 10, 11, 12, 13, 14, or 15 percent by weight blowing agent. In some cases, the blowing agent releases carbon dioxide base during printing to create a foamed structure that can increase porosity of the 3D printed structure. Non-limiting examples of blowing agents include baking powder (e.g., monocalcium phosphate, sodium bicarbonate, corn starch) and azodicarbonamide. In some cases, the blowing agent comprises sodium bicarbonate. In some cases, the formulation comprises ab out 5-15, or ab out 5, 6, 7, 8, 9, 10, 11, 12, 13, 14, or 15 percent by weight sodium bicarbonate. In some embodiments, the blowing agent provides for micropores in the structure having an average diameter of about 1 micron to about 500 microns, or about 50 microns to about 250 microns, or about 150 microns.

In some embodiments, a formulation comprises a ceramic material (e.g., β-TCP) and a polymer. Polymers include PEO, PPO, PDS, PEG, polyester, copolymers, or a combination thereof. In some embodiments, the formulation is about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70 percent ceramic material (e.g., β-TCP) by weight, e.g., about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% ceramic material (e.g., β-TCP) by weight. In some cases, the formulation comprises about 5-20, 5-15, 5-10, 10-20, 10-15, or 15-20 percent poloxamer 407 by weight. In some cases, the formulation comprises about 10-30, 10-25, 10-20, 10-15, 15-30, 15-25, 15-20, 20-30, 20-25, or 25-30 percent by weight PEG. In some cases, the formulation comprises about 10-30, 10-25, 10-20, 10-15, 15-30, 15-25, 15-20, 20-30, 20-25, or 25-30 percent by weight PCL. In some cases, the formulation comprises about 10-30, 10-25, 10-20, 10-15, 15-30, 15-25, 15-20, 20-30, 20-25, or 25-30 percent by weight PDS. In some embodiments, the formulation further comprises an antifoaming agent. In some embodiments, the formulation further comprises a dispersing agent. In some embodiments, the formulation further comprises a solvent. In some embodiments, the formulation further comprises a plasticizer. In some embodiments, the formulation further comprises a particulate. In some cases, the formulation further comprises a blowing agent.

In some embodiments, a formulation comprises a ceramic material (e.g., β-TCP) and a particulate. The particulate may be water soluble. Non-limiting examples of particulates include salts and sugars, e.g., sodium chloride, calcium chloride, sucrose, trehalose (e.g., α,α trehalose dihydrate), and mannitol (e.g., D-mannitol). In some embodiments, the formulation is about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70 percent ceramic material (e.g., (3-TCP) by weight, e.g., about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% ceramic material (e.g., (3-TCP) by weight. In some embodiments, the formulation comprises about 1-10, 1-9, 1-8, 1-7, 1-6, 1-5, 2-10, 2-9, 2-8, 2-7, 2-6, 2-5, 3-10, 3-9, 3-8, 3-7, 3-6, 3-5, 4-10, 4-9, 4-8, 4-7, 4-6, 4-5, 5-10, 5-9, 5-8, 5-7, or 5-6 percent by weight particulate. In some embodiments, the formulation further comprises water. In some embodiments, the formulation further comprises a polymer. In some embodiments, the formulation further comprises an antifoaming agent. In some embodiments, the formulation further comprises a dispersing agent. In some embodiments, the formulation further comprises a solvent. In some embodiments, the formulation further comprises a plasticizer. In some cases, the formulation further comprises a blowing agent.

In some embodiments, a formulation comprises a ceramic material (e.g., β-TCP) and a blowing agent. Non-limiting examples of blowing agents include baking powder (e.g., monocalcium phosphate, sodium bicarbonate, corn starch) and azodicarbonamide. The blowing agent may comprise sodium bicarbonate. In some embodiments, the formulation is about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70 percent ceramic material (e.g., β-TCP) by weight, e.g., about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% ceramic material (e.g., β-TCP) by weight. In some embodiments, the blowing agent comprises about 5-20, 5-18, 5-16, 5-15, 5-14, 5-12, 5-10, 5-8, 5-6, 6-20, 6-18, 6-16, 6-14, 6-12, 6-10, 6-8, 8-20, 8-18, 8-16, 8-14, 8-12, 8-10, 10-20, 10-18, 10-16, 10-14, 10-12, 5-15, or about 5, 6, 7, 8, 9, 10, 11, 12, 13, 14, or 15 percent by weight blowing agent. In some cases, the formulation comprises about 5-15, or about 5, 6, 7, 8, 9, 10, 11, 12, 13, 14, or 15 percent by weight sodium bicarbonate. In some embodiments, the formulation further comprises water. In some embodiments, the formulation further comprises a polymer. In some embodiments, the formulation further comprises an antifoaming agent. In some embodiments, the formulation further comprises a dispersing agent. In some embodiments, the formulation further comprises a solvent. In some embodiments, the formulation further comprises a plasticizer. In some embodiments, the formulation further comprises a particulate.

In another aspect, a formulation comprises a ceramic material and one or more polymers. In some embodiments, the formulation comprises about 30% to about 70% a ceramic material (e.g., β-TCP). For instance, the formulation comprises about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 60-70, 60-65, 65-70, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 percent by weight a ceramic material (e.g., β-TCP). In some embodiments, the formulation comprises a first polymer, e.g., about 10-30, 10-25, 10-20, 10-15, 15-30, 15-25, 15-20, 20-30, 20-25, or 25-30 percent by weight first polymer. The first polymer may be polycaprolactone (PCL). The first polymer may be polydioxanone (PDS). In some embodiments, the formulation comprises a second polymer, e.g., about 10-30, 10-25, 10-20, 10-15, 15-30, 15-25, 15-20, 20-30, 20-25, or 25-30 percent by weight second polymer. The second polymer may be polyethylene glycol (PEG). In a non-limiting embodiment, the formulation comprises about 30-70% by weight ceramic, about 10-30% by weight a first polymer, and about 10-30% by weight a second polymer. For example, the formulation may comprise about 30-70% by weight β-TCP, about 10-30% by weight PCL, and about 10-30% by weight PEG. As another example, the formulation may comprise about 30-70% by weight β-TCP, about 10-30% by weight PDS, and about 10-30% by weight PEG.

In some further embodiments, the formulation comprises a particulate. The particulate may be water soluble. In some cases, the particulate comprises sucrose. In some embodiments, the formulation comprises about 1-10, 1-9, 1-8, 1-7, 1-6, 1-5, 2-10, 2-9, 2-8, 2-7, 2-6, 2-5, 3-10, 3-9, 3-8, 3-7, 3-6, 3-5, 4-10, 4-9, 4-8, 4-7, 4-6, 4-5, 5-10, 5-9, 5-8, 5-7, or 5-6 percent by weight particulate. For example, the formulation may comprise about 30-70% by weight β-TCP, about 10-30% by weight PCL, about 10-30% by weight PEG, and about 1-10% by weight particulate.

In some further embodiments, the formulation comprises a blowing agent. In some cases, the blowing agent comprises sodium bicarbonate. In some embodiments, the formulation comprises about 5-20, 5-18, 5-16, 5-14, 5-12, 5-10, 5-8, 5-6, 6-20, 6-18, 6-16, 6-14, 6-12, 6-10, 6-8, 8-20, 8-18, 8-16, 8-14, 8-12, 8-10, 10-20, 10-18, 10-16, 10-14, 10-12, 5-15, or about 5, 6, 7, 8, 9, 10, 11, 12, 13, 14, or 15 percent by weight blowing agent. For example, the formulation may comprise ab out 30-70% by weight β-TCP, ab out 10-30% by weight PCL, ab out 10-30% by weight PEG, and about 5-20% by weight blowing agent.

In some further embodiments, the polymer of the formulation is a copolymer, such as a PCL and polyglycolide copolymer. For instance, the copolymer comprises about 80-99, 80-98, 80-97, 80-96, 80-95, 80-94, 80-93, 80-92, 80-91, 80-90, 80-89, 80-88, 80-87, 80-86, 80-85, 85-99, 85-98, 85-97, 85-96, 85-95, 85-94, 85-93, 85-92, 85-91, 85-90, 90-99, 90-98, 90-97, 90-96, 90-95, 90-94, 90-93, 90-92, 90-91, 95-99, 95-98, 95-97, or 95-96 percent molar PCL, and about 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent molar polyglycolide. In some cases, the copolymer comprises about 90-95 percent by mole PCL and about 5-10 percent by mole polyglycolide. In an example, the formulation may comprise about 30-70% by weight β-TCP, about 10-30% by weight PCL and polyglycolide copolymer (e.g., 95 mol % polycaprolactone, 5 mol % polyglycolide), and about 10-30% by weight PEG. In an example, the formulation may comprise about 30-70% by weight β-TCP, about 10-30% by weight PCL and polyglycolide copolymer (e.g., 90 mol % polycaprolactone, 10 mol % polyglycolide), and about 10-30% by weight PEG.

In some further embodiments, the polymer of the formulation is a copolymer, such as a PDS and polyglycolide copolymer. For instance, the copolymer comprises about 80-99, 80-98, 80-97, 80-96, 80-95, 80-94, 80-93, 80-92, 80-91, 80-90, 80-89, 80-88, 80-87, 80-86, 80-85, 85-99, 85-98, 85-97, 85-96, 85-95, 85-94, 85-93, 85-92, 85-91, 85-90, 90-99, 90-98, 90-97, 90-96, 90-95, 90-94, 90-93, 90-92, 90-91, 95-99, 95-98, 95-97, or 95-96 percent molar PDS, and about 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent molar polyglycolide. In some cases, the copolymer comprises about 90-95 percent by mole PDS and about 5-10 percent by mole polyglycolide. In an example, the formulation may comprise about 30-70% by weight β-TCP, about 10-30% by weight PDS and polyglycolide copolymer (e.g., 90 mol % PDS, 10 mol % polyglycolide), and about 10-30% by weight PEG.

In some further embodiments, the polymer of the formulation is a copolymer, such as a PDS and lactide copolymer. For instance, the copolymer comprises about 80-99, 80-98, 80-97, 80-96, 80-95, 80-94, 80-93, 80-92, 80-91, 80-90, 80-89, 80-88, 80-87, 80-86, 80-85, 85-99, 85-98, 85-97, 85-96, 85-95, 85-94, 85-93, 85-92, 85-91, 85-90, 90-99, 90-98, 90-97, 90-96, 90-95, 90-94, 90-93, 90-92, 90-91, 95-99, 95-98, 95-97, or 95-96 percent molar PDS, and about 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, orb-10 percent molar lactide. In some cases, the copolymer comprises ab out 90-95 percent by mole PDS and about 5-10 percent by mole lactide. In an example, the formulation may comprise about 30-70% by weight β-TCP, about 10-30% by weight PDS and lactide copolymer (e.g., 90 mol % PDS, 10 mol % lactide), and about 10-30% by weight PEG.

In another aspect, a formulation comprises a ceramic material and one or more polymers. In some embodiments, the formulation comprises about 30% to about 70% a ceramic material (e.g., β-TCP). For instance, the formulation comprises about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 60-70, 60-65, 65-70, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 percent by weight a ceramic material (e.g., β-TCP). In some embodiments, the formulation comprises a first polymer, e.g., about 10-30, 10-25, 10-20, 10-15, 15-30, 15-25, 15-20, 20-30, 20-25, or 25-30 percent by weight first polymer. The first polymer may be a copolymer. In some embodiments, the copolymer comprises a lactide (e.g., poly(D, L-lactide)) and polyglycolide. In some embodiments, the copolymer comprises poly(D, L-lactide-co-glycolide) copolymer. In some embodiments, the copolymer comprises about 50 mol % poly(D, L-lactide) and ab out 50 mol % polyglycolide. In some embodiments, the formulation comprises a second polymer, e.g., about 10-30, 10-25, 10-20, 10-15, 15-30, 15-25, 15-20, 20-30, 20-25, or 25-30 percent by weight second polymer. The second polymer may be polyethylene glycol (PEG). In a non-limiting embodiment, the formulation comprises about 30-70% by weight ceramic, about 10-30% by weight a first polymer, and about 10-30% by weight a second polymer. For example, the formulation may comprise about 30-70% by weight β-TCP, about 10-30% by weight copolymer, and about 10-30% by weight PEG.

In some further embodiments, a formulation comprises a ceramic material and one or more polymers. In some embodiments, the formulation comprises about 30% to about 70% a ceramic material (e.g., β-TCP). For instance, the formulation comprises about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 60-70, 60-65, 65-70, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 percent by weight a ceramic material (e.g., β-TCP). In some embodiments, the formulation comprises a first polymer, e.g., about 10-30, 10-25, 10-20, 10-15, 15-30, 15-25, 15-20, 20-30, 20-25, or 25-30 percent by weight first polymer. The first polymer may be a copolymer, such as a dioxanone and lactide (e.g., L-lactide) copolymer. For instance, the copolymer comprises about 80-99, 80-98, 80-97, 80-96, 80-95, 80-94, 80-93, 80-92, 80-91, 80-90, 80-89, 80-88, 80-87, 80-86, 80-85, 85-99, 85-98, 85-97, 85-96, 85-95, 85-94, 85-93, 85-92, 85-91, 85-90, 90-99, 90-98, 90-97, 90-96, 90-95, 90-94, 90-93, 90-92, 90-91, 95-99, 95-98, 95-97, or 95-96 percent molar dioxanone, and about 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, orb-10 percent molar lactide. In some cases, the copolymer comprises about 90-95 percent by mole dioxanone and about 5-10 percent by mole lactide. In some embodiments, the formulation comprises a second polymer, e.g., about 10-30, 10-25, 10-20, 10-15, 15-30, 15-25, 15-20, 20-30, 20-25, or 25-30 percent by weight second polymer. The second polymer may be polyethylene glycol (PEG). In a non-limiting embodiment, the formulation comprises about 30-70% by weight ceramic, about 10-30% by weight a first polymer, and about 10-30% by weight a second polymer. For example, the formulation may comprise about 30-70% by weight β-TCP, about 10-30% by weight copolymer, and about 10-30% by weight PEG In an example, the formulation may comprise about 30-70% by weight β-TCP, about 10-30% by weight PDS and lactide copolymer (e.g., 90 mol % dioxanone, 10 mol % L-lactide), and about 10-30% by weight PEG.

In one aspect, the formulation has a low viscosity that may be useful during manufacture for extruding through a small diameter nozzle. The nozzle may have a diameter of about 240 μm to about 500 μm or about 280 to about 450 μm, e.g., about 240, 260, 280,300, 320, 340, 360, 380, 400, 420, 440, 460, 480, or 500 μm. In a nonlimiting example embodiment, the formulation is melt-mixed in a dual asymmetric centrifugal mixer to create a homogenous liquid ink.

In one aspect, the formulation has a higher viscosity that may be useful during manufacture by forming the formulation into a filament. The filament may then be used for fused filament fabrication.

In one aspect, the formulation is in the form of a filament. For instance, as further described in Example 2, ink formulations were prepared into filaments for use in 3D printing structures on a fused filament fabrication (FFF) 3D printer. In some embodiments, the filament formulation has a diameter of about 1 to about 3 mm, or about 1 to about 2.75 mm, about 1 to about 2.5 mm, about 1 to about 2.25 mm, about 1 to about 2 mm, about 1 to about 1.75 mm, about 1 to about 1.5 mm, about 1.25 to about 3 mm, about 1.25 to about 2.75 mm, about 1.25 to about 2.5 mm, about 1.25 to about 2.25 mm, about 1.25 to about 2 mm, about 1.25 to about 1.75 mm, about 1.25 to about 1.5 mm, about 1.5 to about 3 mm, about 1.5 to about 2.75 mm, about 1.5 to about 2.5 mm, about 1.5 to about 2.25 mm, about 1.5 to about 2 mm, about 1.5 to about 1.75 mm, about 1.75 to about 3 mm, about 1.75 to about 2.75 mm, about 1.75 to about 2.5 mm, about 1.75 to about 2.25 mm, about 1.75 to about 2 mm, about 2 to about 3 mm, about 2 to about 2.75 mm, about 2 to about 2.5 mm, or about 2 to about 2.25 mm. As a non-limiting example, the filament formulation has a diameter of about 1.5 mm to about 2 mm, or about 1.5 mm, about 1.75 mm, or about 2 mm.

In one aspect, the formulation is in the form of a pellet. For instance, as further described in example 2, ink formulations were prepared into pellets for use in 3D structures. In some embodiments, the pellets can be made into filaments. In some embodiments, pellets can be made into powders. In some embodiments, pellets have a length of about 1 to about 6 mm, or about 1 to about 5.5 mm, about 1 to about 5 mm, about 1 to about 4.5 mm, about 1 to about 4 mm, about 1 to about 3.5 mm, about 1 to about 3 mm, about 1 to about 2.5 mm, about 1 to about 2 mm, about 1 to about 1.5 mm, about 1.5 to about 6 mm, about 1.5 to about 5.5 mm, about 1.5 to about 5 mm, ab out 1.5 to ab out 4.5 mm, ab out 1.5 to about 4 mm, ab out 1.5 to ab out 3.5 mm, ab out 1.5 to about 3 mm, about 1.5 to about 2.5 mm, about 2 to about 6 mm, about 2 to about 5.5 mm, about 2 to about 5 mm, about 2 to about 4.5 mm, about 2 to about 4 mm, about 2 to about 3.5 mm, about 2 to about 3 mm, about 2 to about 2.5 mm, about 2.5 to about 6 mm, about 2.5 to about 5.5 mm, about 2.5 to about 5 mm, about 2.5 to about 4.5 mm, about 2.5 to about 4 mm, about 2.5 to about 3.5 mm, about 2.5 to about 3 mm, about 3 to about 6 mm, about 3 to about 5.5 mm, about 3 to about 5 mm, about 3 to about 4.5 mm, about 3 to about 4 mm, about 3 to about 3.5 mm, about 3.5 to about 6 mm, about 3.5 to about 5.5 mm, about 3.5 to about 5 mm, about 3.5 to about 4.5 mm, about 3.5 to about 4 mm, about 4 to about 6 mm, about 4 to about 5.5 mm, about 4 to about 5 mm, about 4 to about 4.5 mm, about 4.5 to about 6 mm, about 4.5 to about 5.5 mm, about 4.5 to about 5 mm, about 5 to about 6 mm, about 5 to about 5.5 mm, or about 5.5 to about 6 mm. As a non-limiting example embodiment, the pellets have a length of about 2.5 mm to about 4.5 mm, or about 2.5 mm, about 3 mm, about 3.5 mm, about 4 mm, or about 4.5 mm. In some embodiments, a pellet encompasses a variety of different shapes including spears, rods, granules, blocks, particles, and particles of any suitable shape.

In one aspect, the formulation is in the form of a powder. In some embodiments, the powder could be produced from a pellet. In a nonlimiting example embodiment, components of the formulation are melt-mixed into homogenous ink, and cryomilled to form powder. In a nonlimiting example embodiment, components of the formulation are dissolved in a solvent-based slurry and spray dried to form a powder. In some embodiments, powders are used in selective laser sintering.

### 3D Printed Structures

In another aspect, provided herein are 3D printed structures. The structures may be prepared using a formulation and/or method of manufacture described herein.

In some embodiments a three-dimensional structure has micropores. The micropores may be formed after removal of a particulate or pore former. The micropores may be formed by a use of a blowing agent during formulation. In some embodiments, the micropores provide additional surface area to the structure for contact with a therapeutic agent as compared to a structure lacking micropores. In a non-limiting example, the therapeutic agent comprises a targeting moiety that is non-covalently bound to a ceramic material of the structure. In some embodiments, the micropores have an average diameter of about 1 micron to ab out 500 microns. For instance, about 1 micron to about 450 microns, about 1 micron to about 400 microns, about 1 micron to about 350 microns, about 1 micron to about 300 microns, about 1 micron to about 250 microns, about 1 micron to about 200 microns, about 1 micron to about 150 microns, about 50 microns to about 500 microns, about 50 microns to about 450 microns, about 50 microns to about 400 microns, about 50 microns to about 350 microns, about 50 microns to about 300 microns, about 50 microns to about 250 microns, about 50 microns to about 200 microns, about 50 microns to about 150 microns, about 100 microns to about 500 microns, about 100 microns to about 450 microns, about 100 microns to about 400 microns, about 100 microns to about 350 microns, about 100 microns to about 300 microns, about 100 microns to about 250 microns, about 100 microns to about 200 microns, about 100 microns to about 150 microns, about 150 microns to about 500 microns, about 150 microns to about 450 microns, about 150 microns to about 400 microns, about 150 microns to about 350 microns, about 150 microns to about 300 microns, about 150 microns to about 250 microns, or about 150 microns to about 200 microns in diameter. In some cases, the micropores have an average diameter of about 50 microns to about 250 microns, about 60 microns to about 240 microns, about 70 microns to about 230 microns, about 80 microns to about 220 microns, or about 90 microns to about 210 microns. In some embodiments, the micropores an average diameter of about 100 microns to about 200 microns, e.g., about 110 microns to about 190 microns, about 120 microns to about 180 microns, about 130 microns to about 170 microns, about 140 microns to about 160 microns, or about 100 microns, about 110 microns, about 120 microns, about 130 microns, about 140 microns, ab out 150 microns, about 160 microns, ab out 170 microns, about 180 microns, about 190 microns, or about 200 microns. In some embodiments, the micropores have an average diameter of about 150 microns.

In some embodiments a three-dimensional structure has a density of ab out 1 g/cm³ to about 3 g/cm³. In some embodiments a three-dimensional structure has a density of about 1 g/cm³ to about 2 g/cm³. (e.g., about 1, 1.1, 1.15, 1.2, 1.25, 1.3, 1.35, 1.4, 1.45, 1.5, 1.5, 1.55, 1.6, 1.65, 1.7, 1.75, 1.8, 1.85, 1.9, 1.95, or 2 g/cm³ or any value therebetween).

In some embodiments a three-dimensional structure has an open porosity of about 15% to about 50%, about 15% to about 45%, about 15% to about 40%, about 20% to about 50%, about 20% to about 45%, about 25% to about 40%, about 25% to about 50%, about 25% to about 45%, or about 25% to about 40%. In some embodiments, the open porosity is about 25% to about 40%, e.g., about 25%, 30%, 35%, or 40%, or any value therebetween).

In some embodiments a three-dimensional structure has a strut diameter of about 300 nm to about 600 nm, about 325 nm to about 600 nm, about 350 nm to about 600 nm, about 375 nm to about 600 nm, about 400 nm to about 600 nm, about 425 nm to about 600 nm, about 450 nm to about 600 nm, about 475 nm to about 600 nm, about 500 nm to about 600 nm, about 525 nm to about 600 nm, about 550 nm to about 600 nm, about 300 nm to about 575 nm, about 325 nm to about 575 nm, about 350 nm to about 575 nm, about 375 nm to about 575 nm, about 400 nm to about 575 nm, about 425 nm to about 575 nm, about 450 nm to about 575 nm, about 475 nm to about 575 nm, about 500 nm to about 575 nm, about 525 nm to about 575 nm, about 550 nm to about 575 nm, about 300 nm to about 550 nm, about 325 nm to about 550 nm, about 350 nm to about 550 nm, about 375 nm to about 550 nm, about 400 nm to about 550 nm, about 425 nm to about 550 nm, about 450 nm to about 550 nm, about 475 nm to about 550 nm, about 500 nm to about 550 nm, about 525 nm to about 550 nm, about 300 nm to about 525 nm, about 325 nm to about 525 nm, about 350 nm to about 525 nm, about 375 nm to about 525 nm, about 400 nm to about 525 nm, about 425 nm to about 525 nm, about 450 nm to about 525 nm, about 475 nm to about 525 nm, about 500 nm to about 525 nm, about 300 nm to about 500 nm, about 325 nm to about 500 nm, about 350 nm to about 500 nm, about 375 nm to about 500 nm, about 400 nm to about 500 nm, about 425 nm to about 500 nm, about 450 nm to about 500 nm, about 475 nm to about 500 nm, about 300 nm to about 475 nm, about 325 nm to about 475 nm, about 350 nm to about 475 nm, about 375 nm to about 475 nm, about 400 nm to about 475 nm, about 425 nm to about 475 nm, about 450 nm to about 475 nm, ab out 300 nm to about 450 nm, about 325 nm to about 450 nm, about 350 nm to about 450 nm, about 375 nm to about 450 nm, about 400 nm to about 450 nm, about 425 nm to about 450 nm, about 300 nm to about 400 nm, about 325 nm to about 400 nm, about 350 nm to about 400 nm, or about 375 nm to about 400 nm.

In some embodiments, the structure comprises a ceramic material such as a calcium phosphate. In some embodiments, the structure comprises about 50-100, 50-95, 50-90, 50-85, 50-80, 50-75, 50-70, 50-65, 50-60, 50-55, 55-100, 55-95, 55-90, 55-85, 55-80, 55-75, 55-70, 55-65, 55-60, 60-100, 60-95, 60-90, 60-85, 60-80, 60-75, 60-70, 60-65, 65-100, 65-95, 65-90, 65-85, 65-80, 65-75, 65-70, 70-100, 70-95, 70-90, 70-85, 70-80, 70-75, 75-100, 75-95, 75-90, 75-85, 75-80, 80-100, 80-95, 80-90, 80-85, 85-100, 85-95, 85-90, 90-100, 90-95, 95-100, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent ceramic material. In some cases, the ceramic material is calcium phosphate, such as beta-tricalcium phosphate ((3-TCP).

In a non-limiting example, a structure has about 50-90% ceramic material such as β-TCP. In some cases, the structure has about 50, 55, 60, 65, 70, 75, 80, 85, or 90% ceramic material such as β-TCP. In some embodiments, the structure has about 10-50% polymer such as polycaprolactone (PCL) or polydioxanone (PDS). In some cases, the structure has about 10, 15, 20, 25, 30, 35, 40, 45, or 50% polymer such as PCL or PDS. Example structures include those having: about 85-90% ceramic (e.g., β-TCP) and about 10-15% polymer (e.g., PCL or PDS) by weight, about 80-85% ceramic (e.g., β-TCP) and about 15-20% polymer (e.g., PCL or PDS) by weight, about 75-80% ceramic (e.g., β-TCP) and about 20-25% polymer (e.g., PCL or PDS) by weight, about 70-75% ceramic (e.g., β-TCP) and about 25-30% polymer (e.g., PCL or PDS) by weight, about 65-70% ceramic (e.g., β-TCP) and about 30-35% polymer (e.g., PCL or PDS) by weight, about 60-65% ceramic (e.g., β-TCP) and about 35-40% polymer (e.g., PCL or PDS) by weight, about 55-60% ceramic (e.g., β-TCP) and about 40-45% polymer (e.g., PCL or PDS) by weight, about 50-55% ceramic (e.g., β-TCP) and about 45-50% polymer (e.g., PCL or PDS) by weight, about 90% ceramic (e.g., β-TCP) and about 10% polymer (e.g., PCL or PDS) by weight, about 89% ceramic (e.g., β-TCP) and about 11% polymer (e.g., PCL or PDS) by weight, about 88% ceramic (e.g., β-TCP) and about 12% polymer (e.g., PCL or PDS) by weight, about 87% ceramic (e.g., β-TCP) and about 13% polymer (e.g., PCL or PDS) by weight, about 86% ceramic (e.g., β-TCP) and about 14% polymer (e.g., PCL or PDS) by weight, about 85% ceramic (e.g., (3-TCP) and about 15% polymer (e.g., PCL or PDS) by weight, about 84% ceramic (e.g., β-TCP) and ab out 16% polymer (e.g., PCL or PDS) by weight, ab out 83% ceramic (e.g., β-TCP) and about 17% polymer (e.g., PCL or PDS) by weight, about 82% ceramic (e.g., β-TCP) and about 18% polymer (e.g., PCL or PDS) by weight, about 81% ceramic (e.g., β-TCP) and about 19% polymer (e.g., PCL or PDS) by weight, about 80% ceramic (e.g., β-TCP) and about 20% polymer (e.g., PCL or PDS) by weight, about 79% ceramic (e.g., β-TCP) and about 21% polymer (e.g., PCL or PDS) by weight, about 78% ceramic (e.g., β-TCP) and about 22% polymer (e.g., PCL or PDS) by weight, about 77% ceramic (e.g., β-TCP) and about 23% polymer (e.g., PCL or PDS) by weight, about 76% ceramic (e.g., β-TCP) and about 24% polymer (e.g., PCL or PDS) by weight, about 75% ceramic (e.g., β-TCP) and about 25% polymer (e.g., PCL or PDS) by weight, about 74% ceramic (e.g., β-TCP) and about 26% polymer (e.g., PCL or PDS) by weight, about 73% ceramic (e.g., β-TCP) and about 27% polymer (e.g., PCL or PDS) by weight, about 72% ceramic (e.g., (3-TCP) and about 28% polymer (e.g., PCL or PDS) by weight, about 71% ceramic (e.g., β-TCP) and about 29% polymer (e.g., PCL or PDS) by weight, ab out 70% ceramic (e.g., β-TCP) and about 30% polymer (e.g., PCL or PDS) by weight, about 69% ceramic (e.g., β-TCP) and about 31% polymer (e.g., PCL or PDS) by weight, about 68% ceramic (e.g., β-TCP) and about 32% polymer (e.g., PCL or PDS) by weight, about 67% ceramic (e.g., β-TCP) and about 33% polymer (e.g., PCL or PDS) by weight, about 66% ceramic (e.g., β-TCP) and about 34% polymer (e.g., PCL or PDS) by weight, about 65% ceramic (e.g., β-TCP) and about 35% polymer (e.g., PCL or PDS) by weight, about 64% ceramic (e.g., β-TCP) and about 36% polymer (e.g., PCL or PDS) by weight, about 63% ceramic (e.g., β-TCP) and about 37% polymer (e.g., PCL or PDS) by weight, about 62% ceramic (e.g., β-TCP) and about 38% polymer (e.g., PCL or PDS) by weight, about 61% ceramic (e.g., β-TCP) and about 39% polymer (e.g., PCL or PDS) by weight, about 60% ceramic (e.g., β-TCP) and about 40% polymer (e.g., PCL or PDS) by weight, about 59% ceramic (e.g., (3-TCP) and about 41% polymer (e.g., PCL or PDS) by weight, about 58% ceramic (e.g., β-TCP) and about 42% polymer (e.g., PCL or PDS) by weight, ab out 57% ceramic (e.g., β-TCP) and about 43% polymer (e.g., PCL or PDS) by weight, about 56% ceramic (e.g., β-TCP) and about 44% polymer (e.g., PCL or PDS) by weight, about 55% ceramic (e.g., β-TCP) and about 45% polymer (e.g., PCL or PDS) by weight, about 54% ceramic (e.g., β-TCP) and about 46% polymer (e.g., PCL or PDS) by weight, about 53% ceramic (e.g., β-TCP) and about 47% polymer (e.g., PCL or PDS) by weight, about 52% ceramic (e.g., β-TCP) and about 48% polymer (e.g., PCL or PDS) by weight, about 51% ceramic (e.g., β-TCP) and about 49% polymer (e.g., PCL or PDS) by weight, and about 50% ceramic (e.g., β-TCP) and about 50% polymer (e.g., PCL or PDS) by weight.

The structure may be manufactured using 3D printing from an ink comprising about 30-70% by weight β-TCP powder, about 10-30% by weight first polymer, and about 10-30% by weight second polymer. In some cases, the first polymer comprises PCL. In some cases, the first polymer comprises PDS. In some cases, the second polymer comprises PEG. In some cases, the ink further comprises about 1-10% by weight particulate (e.g., sucrose). In some cases, the ink further comprises about 5-20% blowing agent (e.g., sodium bicarbonate).

In some embodiments the three-dimensional structure has a density of about 1 g/cm³ to about 2 g/cm³ or about 1.25 g/cm³ to about 1.75 g/cm³. In some embodiments the three-dimensional structure has an open porosity of about 20% to about 40%, about 25% to about 35%, e.g., about 25%, 26%, 27%, 28%, 29%, 30%, 31%, 32%, 33%, 34%, or 35%. In some embodiments a three-dimensional structure has a strut diameter of about 400 μm to about 500 μm, about 400 μm to about 450 μm, or about 425 μm to about 450 μm.

In some embodiments the three-dimensional structure has a density of about 1 g/cm³ to about 2 g/cm³ or about 1 g/cm³ to about 1.5 g/cm³. In some embodiments the three-dimensional structure has an open porosity of about 30% to about 50%, about 35% to about 45%, e.g., about 35%, 36%, 37%, 38%, 39%, 40%, 41%, 42%, 43%, 44%, or 45%. In some embodiments a three-dimensional structure has a strut diameter of about 325 μm to about 425 μm, about 350 μm to about 400 μm, or about 360 μm to about 390 μm.

In some embodiments the three-dimensional structure has a density of about 1 g/cm³ to about 2 g/cm³ or about 1 g/cm³ to about 1.5 g/cm³. In some embodiments the three-dimensional structure has an open porosity of about 30% to about 50%, about 35% to about 45%, e.g., about 35%, 36%, 37%, 38%, 39%, 40%, 41%, 42%, 43%, 44%, or 45%. In some embodiments a three-dimensional structure has a strut diameter of about 350 μm to about 450 μm, about 350 μm to about 400 μm, or about 380 μm to about 405 μm.

In a non-limiting example, a structure has about 50-90% ceramic material such as β-TCP. In some cases, the structure has about 50, 55, 60, 65, 70, 75, 80, 85, or 90% ceramic material such as β-TCP. In some embodiments, the structure has about 10-50% copolymer such as polycaprolactone/polyglycolide copolymer (PCL/PGA, e.g., 90:10, 95:5), poly(D,L-lactide-co-glycolide) copolymer (PLGA, e.g., 50:50), PDS-glycolide copolymer (PDS/PGA, e.g., 90:10), PDS-L-lactide copolymer (PDS/PLA, e.g., 90:10), or Dioxanone/L-lactide copolymer (e.g., 90:10). In some cases, the structure has about 10, 15, 20, 25, 30, 35, 40, 45, or 50% polymer such as PCL/PGA, PDS/PGA, PDS/PLA, PLGA, or Dioxanone/L-lactide. Example structures include those having: about 85-90% ceramic (e.g., β-TCP) and about 10-15% polymer (e.g., PCL/PGA, PDS/PGA, PDS/PLA, PLGA, or Dioxanone/L-lactide) by weight, about 80-85% ceramic (e.g., β-TCP) and about 15-20% polymer (e.g., PCL/PGA, PDS/PGA, PDS/PLA, PLGA, or Dioxanone/L-lactide) by weight, about 75-80% ceramic (e.g., β-TCP) and about 20-25% polymer (e.g., PCL/PGA, PDS/PGA, PDS/PLA, PLGA, or Dioxanone/L-lactide) by weight, about 70-75% ceramic (e.g., β-TCP) and about 25-30% polymer (e.g., PCL/PGA, PDS/PGA, PDS/PLA, PLGA, or Dioxanone/L-lactide) by weight, about 65-70% ceramic (e.g., β-TCP) and about 30-35% polymer (e.g., PCL/PGA, PDS/PGA, PDS/PLA, PLGA, or Dioxanone/L-lactide) by weight, about 60-65% ceramic (e.g., β-TCP) and ab out 35-40% polymer (e.g., PCL/PGA, PD S/PGA, PD S/PLA, PLGA, or Dioxanone/L-lactide) by weight, about 55-60% ceramic (e.g., β-TCP) and about 40-45% polymer (e.g., PCL/PGA, PDS/PGA, PDS/PLA, PLGA, or Dioxanone/L-lactide) by weight, about 50-55% ceramic (e.g., β-TCP) and about 45-50% polymer (e.g., PCL/PGA, PDS/PGA, PDS/PLA, PLGA, or Dioxanone/L-lactide) by weight, about 90% ceramic (e.g., β-TCP) and about 10% polymer (e.g., PCL/PGA, PDS/PGA, PDS/PLA, PLGA, or Dioxanone/L-lactide) by weight, about 89% ceramic (e.g., β-TCP) and about 11% polymer (e.g., PCL/PGA, PDS/PGA, PDS/PLA, PLGA, or Dioxanone/L-lactide) by weight, about 88% ceramic (e.g., β-TCP) and about 12% polymer (e.g., PCL/PGA, PDS/PGA, PDS/PLA, PLGA, or Dioxanone/L-lactide) by weight, about 87% ceramic (e.g., β-TCP) and about 13% polymer (e.g., PCL/PGA, PDS/PGA, PDS/PLA, PLGA, or Dioxanone/L-lactide) by weight, about 86% ceramic (e.g., β-TCP) and about 14% polymer (e.g., PCL/PGA, PDS/PGA, PDS/PLA, PLGA, or Dioxanone/L-lactide) by weight, about 85% ceramic (e.g., β-TCP) and about 15% polymer (e.g., PCL/PGA, PDS/PGA, PDS/PLA, PLGA, or Dioxanone/L-lactide) by weight, about 84% ceramic (e.g., β-TCP) and about 16% polymer (e.g., PCL/PGA, PDS/PGA, PDS/PLA, PLGA, or Dioxanone/L-lactide) by weight, about 83% ceramic (e.g., β-TCP) and about 17% polymer (e.g., PCL/PGA, PDS/PGA, PDS/PLA, PLGA, or Dioxanone/L-lactide) by weight, about 82% ceramic (e.g., β-TCP) and about 18% polymer (e.g., PCL/PGA, PDS/PGA, PDS/PLA, PLGA, or Dioxanone/L-lactide) by weight, about 81% ceramic (e.g., β-TCP) and about 19% polymer (e.g., PCL/PGA, PDS/PGA, PDS/PLA, PLGA, or Dioxanone/L-lactide) by weight, about 80% ceramic (e.g., β-TCP) and about 20% polymer (e.g., PCL/PGA, PDS/PGA, PDS/PLA, PLGA, or Dioxanone/L-lactide) by weight, about 79% ceramic (e.g., β-TCP) and about 21% polymer (e.g., PCL/PGA, PDS/PGA, PDS/PLA, PLGA, or Dioxanone/L-lactide) by weight, about 78% ceramic (e.g., β-TCP) and about 22% polymer (e.g., PCL/PGA, PDS/PGA, PDS/PLA, PLGA, or Dioxanone/L-lactide) by weight, about 77% ceramic (e.g., β-TCP) and about 23% polymer (e.g., PCL/PGA, PDS/PGA, PDS/PLA, PLGA, or Dioxanone/L-lactide) by weight, about 76% ceramic (e.g., β-TCP) and about 24% polymer (e.g., PCL/PGA, PDS/PGA, PDS/PLA, PLGA, or Dioxanone/L-lactide) by weight, about 75% ceramic (e.g., β-TCP) and about 25% polymer (e.g., PCL/PGA, PDS/PGA, PDS/PLA, PLGA, or Dioxanone/L-lactide) by weight, about 74% ceramic (e.g., β-TCP) and about 26% polymer (e.g., PCL/PGA, PDS/PGA, PDS/PLA, PLGA, or Dioxanone/L-lactide) by weight, about 73% ceramic (e.g., β-TCP) and about 27% polymer (e.g., PCL/PGA, PDS/PGA, PDS/PLA, PLGA, or Dioxanone/L-lactide) by weight, about 72% ceramic (e.g., β-TCP) and about 28% polymer (e.g., PCL/PGA, PDS/PGA, PDS/PLA, PLGA, or Dioxanone/L-lactide) by weight, about 71% ceramic (e.g., β-TCP) and about 29% polymer (e.g., PCL/PGA, PDS/PGA, PDS/PLA, PLGA, or Dioxanone/L-lactide) by weight, about 70% ceramic (e.g., β-TCP) and about 30% polymer (e.g., PCL/PGA, PDS/PGA, PDS/PLA, PLGA, or Dioxanone/L-lactide) by weight, about 69% ceramic (e.g., β-TCP) and about 31% polymer (e.g., PCL/PGA, PDS/PGA, PDS/PLA, PLGA, or Dioxanone/L-lactide) by weight, about 68% ceramic (e.g., β-TCP) and about 32% polymer (e.g., PCL/PGA, PDS/PGA, PDS/PLA, PLGA, or Dioxanone/L-lactide) by weight, about 67% ceramic (e.g., β-TCP) and about 33% polymer (e.g., PCL/PGA, PDS/PGA, PDS/PLA, PLGA, or Dioxanone/L-lactide) by weight, about 66% ceramic (e.g., β-TCP) and about 34% polymer (e.g., PCL/PGA, PDS/PGA, PDS/PLA, PLGA, or Dioxanone/L-lactide) by weight, about 65% ceramic (e.g., β-TCP) and about 35% polymer (e.g., PCL/PGA, PDS/PGA, PDS/PLA, PLGA, or Dioxanone/L-lactide) by weight, about 64% ceramic (e.g., β-TCP) and about 36% polymer (e.g., PCL/PGA, PDS/PGA, PDS/PLA, PLGA, or Dioxanone/L-lactide) by weight, about 63% ceramic (e.g., β-TCP) and about 37% polymer (e.g., PCL/PGA, PDS/PGA, PDS/PLA, PLGA, or Dioxanone/L-lactide) by weight, about 62% ceramic (e.g., β-TCP) and about 38% polymer (e.g., PCL/PGA, PDS/PGA, PDS/PLA, PLGA, or Dioxanone/L-lactide) by weight, about 61% ceramic (e.g., β-TCP) and about 39% polymer (e.g., PCL/PGA, PDS/PGA, PDS/PLA, PLGA, or Dioxanone/L-lactide) by weight, about 60% ceramic (e.g., β-TCP) and about 40% polymer (e.g., PCL/PGA, PDS/PGA, PDS/PLA, PLGA, or Dioxanone/L-lactide) by weight, about 59% ceramic (e.g., β-TCP) and about 41% polymer (e.g., PCL/PGA, PDS/PGA, PDS/PLA, PLGA, or Dioxanone/L-lactide) by weight, about 58% ceramic (e.g., β-TCP) and about 42% polymer (e.g., PCL/PGA, PDS/PGA, PDS/PLA, PLGA, or Dioxanone/L-lactide) by weight, about 57% ceramic (e.g., β-TCP) and about 43% polymer (e.g., PCL/PGA, PDS/PGA, PDS/PLA, PLGA, or Dioxanone/L-lactide) by weight, about 56% ceramic (e.g., β-TCP) and about 44% polymer (e.g., PCL/PGA, PDS/PGA, PDS/PLA, PLGA, or Dioxanone/L-lactide) by weight, about 55% ceramic (e.g., β-TCP) and about 45% polymer (e.g., PCL/PGA, PDS/PGA, PDS/PLA, PLGA, or Dioxanone/L-lactide) by weight, about 54% ceramic (e.g., β-TCP) and about 46% polymer (e.g., PCL/PGA, PDS/PGA, PDS/PLA, PLGA, or Dioxanone/L-lactide) by weight, about 53% ceramic (e.g., β-TCP) and about 47% polymer (e.g., PCL/PGA, PDS/PGA, PDS/PLA, PLGA, or Dioxanone/L-lactide) by weight, about 52% ceramic (e.g., β-TCP) and about 48% polymer (e.g., PCL/PGA, PDS/PGA, PDS/PLA, PLGA, or Dioxanone/L-lactide) by weight, about 51% ceramic (e.g., β-TCP) and about 49% polymer (e.g., PCL/PGA, PDS/PGA, PDS/PLA, PLGA, or Dioxanone/L-lactide) by weight, and about 50% ceramic (e.g., β-TCP) and about 50% polymer (e.g., PCL/PGA, PDS/PGA, PDS/PLA, PLGA, or Dioxanone/L-lactide) by weight.

The structure may be manufactured using 3D printing from an ink comprising about 30-70% by weight β-TCP powder, about 10-30% by weight first polymer, and about 10-30% by weight second polymer. In some cases, the first polymer comprises PCL/PGA, PDS/PGA, PDS/PLA, PLGA, or Dioxanone/L-lactide. In some cases, the second polymer comprises PEG.

In some embodiments the three-dimensional structure has a density of about 1 g/cm³ to about 2 g/cm³ or about 1.25 g/cm³ to about 1.75 g/cm³. In some embodiments the three-dimensional structure has an open porosity of about 15% to about 35%, about 20% to about 30%, e.g., about 20%, 21%, 22%, 23%, 24%, 25%, 26%, 27%, 28%, 29%, or 30%. In some embodiments a three-dimensional structure has a strut diameter of ab out 350 μm to about 450 μm, about 375 μm to about 425 μm, or about 385 μm to about 415 μm.

In some embodiments the three-dimensional structure has a density of about 1 g/cm³ to about 2 g/cm³ or about 1.5 g/cm³ to about 2 g/cm³. In some embodiments the three-dimensional structure has an open porosity of about 15% to about 35%, about 20% to about 30%, e.g., about 20%, 21%, 22%, 23%, 24%, 25%, 26%, 27%, 28%, 29%, or 30%. In some embodiments a three-dimensional structure has a strut diameter of about 500 μm to about 600 μm, about 550 μm to about 600 μm, or about 555 μm to about 585 μm.

In some embodiments the three-dimensional structure has a density of about 1 g/cm³ to about 2 g/cm³ or about 1.25 g/cm³ to about 1.75 g/cm³. In some embodiments the three-dimensional structure has an open porosity of about 20% to about 40%, about 25% to about 35%, e.g., about 25%, 26%, 27%, 28%, 29%, 30%, 31%, 32%, 33%, 34%, or 35%. In some embodiments a three-dimensional structure has a strut diameter of ab out 400 μm to about 500 μm, about 450 μm to about 500 μm, or about 450 μm to about 475 μm.

In some embodiments, the compositions of ink formulations herein are varied to optimize specific surface area. The surface area may be optimized for combination with a certain therapeutic agent. For example, the structure has a surface area of about 0.2-2 m²/g for combination with a BMP protein (e.g., tBMP-2). In some embodiments, the surface area of a structure herein is about 0.2-2, 0.2-1.8, 0.2-1.6, 0.2-1.4, 0.2-1.2, 0.2-1, 0.2-0.8, 0.2-0.6, 0.2-0.4, 0.4-2, 0.4-1.8, 0.4-1.6, 0.4-1.4, 0.4-1.2, 0.4-1, 0.4-0.8, 0.4-0.6, 0.6-2, 0.6-1.8, 0.6-1.6, 0.6-1.4, 0.6-1.2, 0.6-1, 0.6-0.8, 0.8-2, 0.8-1.8, 0.8-1.6, 0.8-1.4, 0.8-1.2, 0.8-1, 1-2, 1-1.8, 1-1.6, 1-1.4, 1-1.2, 1.2-2, 1.2-1.8, 1.2-1.6, 1.2-1.4, 1.4-2, 1.4-1.8, 1.4-1.6, 1.6-2, 1.6-1.8, or 1.8-2 m²/g. In some embodiments, the surface area is calculated by Brunauer-Emmett-Teller (BET) by gas physisorption.

In some embodiments, the compositions of ink formulations herein are varied to optimize resorption rate of one or more materials of the scaffold. For instance, the polymers are selected based on resorption rate. The resorption rates vary from slowest to fastest as: polycaprolactone, polycaprolactone/polyglycolide copolymer (95:5), polycaprolactone/glycolide copolymer (90:10), polydioxanone/L-lactide copolymer (90:10), poly(D,L-lactide-co-glycolide) copolymer (50:50).

### Methods of Manufacture

In another aspect, provided are methods of manufacturing a structure using 3D printing techniques.

In some embodiments, the method comprises syringe-based melt extrusion bioprinting Example inks for this method may be low in viscosity for extrusion of the ink through a narrow nozzle. Non-limiting example methods of manufacturing using this method are described in Example 2, for instance, with regard to printing ink formulations #1, #2, #3, #4.

In one aspect, the method is an extrusion based method comprising a 3D printing method that extrudes the material out of a nozzle.

In some embodiments, the extrusion based method encompasses bioprinting (syringe-based pneumatic printing) or Fused Granular Fabrication (FGF) where pellets of feedstock are fed from a hopper into a mini-screw extrusion head which melts and pushes the material out of a fine nozzle. In some embodiments, the inks are formed into small (e.g., 2-5 mm) pellets or granules.

In some embodiments, the method comprises fused filament fabrication (FFF). Example inks for this method may be formed into filaments for printing on FFF 3D printers. Non-limiting example methods of manufacturing using this method are described in Example 2, for instance, with regard to printing ink formulations #5 and #6.

In some embodiments, the method comprises pelletized fused deposition modeling. Fused deposition modeling (FDM) is an additive manufacturing process. Three dimensional objects are formed through extrusion and deposition of individual layers of thermoplastic materials. FDM involves the melt extrusion of filament materials through a heated nozzle and deposition as thin solid layers on a platform. A thermoplastic polymer material is fed into a temperature-controlled FDM extrusion head and it is heated to a semi-liquid state. Afterward, the FDM extrusion head extrudes and deposits the material in ultra-thin layers onto a base with precision. The material solidifies, laminating to the preceding layer. In this way, parts are fabricated in layers, where each layer is built by extruding a small bead of material, called a road, in a particular pattern, such that the layer is covered with the adjacent roads. After each layer is completed, extrusion head height is increased and sub sequent layers are built to construct the part. Usually, FDM is used to fabricate solid models. In order to fabricate porous structures, raster fill gaps have a positive value which is applied to impart a channel within a build layer. Arranged in a regular manner, the channels are interconnected even in three dimensions. Layer by layer fabrication allows design of a pore morphology which varies across a scaffold structure.

In some embodiments, the method comprises selective laser sintering (SLS). Selective laser sintering (SLS) is a process wherein a dispenser deposits layers of powdered material into a target area. There is a laser control mechanism that typically includes a computer with the article design stored on it. The laser control mechanism modulates and moves a laser beam to selectively irradiate the powder layer within defined boundaries of the design, melting the powder on which the laser beam falls. This is done to selectively sinter sequential powder layers. The method produces a completed article comprised of a plurality of layers sintered together.

In some embodiments, after 3D printing, the resulting subject is soaked in water to dissolve certain components of the ink, e.g., PEG, particulate (e.g., pore forming agent, sucrose), blowing agent (e.g., sodium bicarbonate), or a combination thereof. The structure may then be dried, sterilized, treated with a therapeutic as described elsewhere herein, or a combination thereof.

Any of the 3D-printed structures described herein can be coated with a tetherable protein (for example, tBMP2). Following completion of the structures using any of the methods discussed herein, the structures can be washed in an acidic sodium acetate buffer. This can be one, two, or more washes. The washing can then be followed by a two-hour incubation of the structures in sodium acetate buffer that contains a 1 mg/mL concentration of tBMP2 protein. The tetherable tBMP2 binds to the β-TCP surface of the implantable structures in a monolayer.

In further embodiments, the ink formulations discussed herein can include a light-sensitive resin that is mixed with the ceramic powder for digital light processing (DLP), an additive manufacturing technique that is faster than robocasting or melt extrusion. Components in a photosensitive, ceramic-filled resin for DLP 3D printing of bone implants typically include ceramic powder (e.g., β-TCP, hydroxyapatite, bioglass, typically <10 μm particle size), one or more crosslinking acrylates or methacrylates (e.g., polyethylene glycol diacrylate, polycaprolactone methacrylate), a plasticizer to reduce resin viscosity (e.g., water), a dispersant to promote breakdown of powder agglomerates (e.g., Darvan® 821-A), photoinitiator to initiate the photocrosslinking reaction (e.g., Lithium phenyl-2,4,6-trimethylbenzoylphosphinate), and a photoabsorber to retain high x-y resolution (e.g., tartrazine). Once resin formulations are prepared by asymmetric centrifugal mixing of the components, the ink is exposed layer by layer to a DLP image, causing the lighted pixels to selectively solidify when the resin encounters the light Once the implantable structure has been built up layer by layer, it can be thermally processed to burn out the included polymer and densify the ceramic (e.g., a polyethylene glycol diacrylate-containing resin), or left as-is, resulting in a flexible ceramic/polymer composite implant (e.g., a polycaprolactone methacrylate-containing resin).

### Devices

In another aspect, provided are devices and kits comprising a 3D printed structure described herein and a therapeutic agent. In some embodiments, a device comprises the therapeutic agent connected to, dispersed within, or otherwise combined with the 3D printed structure. As used herein, a therapeutic agent is inclusive of a plurality of therapeutic agents, such as 2, 3, 4, or 5 therapeutic agents.

Therapeutic Agents

In some embodiments, the therapeutic agent comprises a mammalian growth factor or a functional portion thereof. Mammalian growth factors can be osteoinductive molecules that are capable of initiating and enhancing the bone repair process. A functional portion of the mammalian growth factor is a region that has a therapeutic effect. For instance, a functional portion of a mammalian growth factor is osteoinductive. As another example, a functional portion of a mammalian growth factor is capable of initiating and/or enhancing bone repair. A functional portion of a mammalian growth factor may have osteogenic activity.

Non-limiting examples of mammalian growth factors are described herein. In some instances, the mammalian growth factor comprises: epidermal growth factor (EGF), platelet derived growth factor (PDGF), insulin like growth factor (IGF-1), fibroblast growth factor (FGF), fibroblast growth factor 2 (FGF2), fibroblast growth factor 18 (FGF18), transforming growth factor alpha (TGF-α), transforming growth factor beta (TGF-β), transforming growth factor beta 1 (TGF-β1), transforming growth factor beta 3 (TGF-β3), osteogenic protein 1 (OP-1), osteogenic protein 2 (OP-2), osteogenic protein 3 (OP-3), bone morphogenetic protein 2 (BMP-2), bone morphogenetic protein 3 (BMP-3), bone morphogenetic protein 4 (BMP-4), bone morphogenetic protein 5 (BMP-5), bone morphogenetic protein 6 (BMP-6), bone morphogenetic protein 7 (BMP-7), bone morphogenetic protein (BMP-9), bone morphogenetic protein 10 (BMP-10), bone morphogenetic protein 11 (BMP-11), bone morphogenetic protein 12 (BMP-12), bone morphogenetic protein 13 (BMP-13), bone morphogenetic protein 15 (BMP-15), dentin phosphoprotein (DPP), vegetal related growth factor (Vgr), growth differentiation factor 1 (GDF-1), growth differentiation factor 3 (GDF-3), growth differentiation factor 5 (GDF-5), growth differentiation factor 6 (GDF-6), growth differentiation factor 7 (GDF-7), growth differentiation factor 8 (GDF8), growth differentiation factor 11 (GDF11), growth differentiation factor 15 (GDF15), vascular endothelial growth factor (VEGF), hyaluronic acid binding protein (HABP), and collagen binding protein (CBP), fibroblast growth factor 18 (FGF-18), keratinocyte growth factor (KGF), tumor necrosis factor alpha (TNFα), tumor necrosis factor (TNF)-related apoptosis inducing ligand (TRAIL), wnt family member 1 (WNT1), wnt family member 2 (WNT2), wnt family member 2B (WNT2B), wnt family m emb er 3 (WNT3), wnt family member 3 A (WNT3 A), wnt family member 4 (WNT4), wnt family member 5A (WNT5A), wnt family member 5B (WNT5B), wnt family member 6 (WNT6), wnt family member 7A (WNT7A), wnt family member 7B (WNT7B), wnt family member 8A (WNT8A), wnt family member 8B (WNT8B), wnt family member 9A (WNT9A), wnt family member 9B (WNT9B), wnt family member 10A (WNT10A), wnt family member 10B (WNT10B), wnt family member 11 (WNT11), or wnt family member 16 (WNT16), or a mature peptide or functional portion thereof.

In some embodiments, the mammalian growth factor is a human growth factor. Non-limiting examples of human growth factors and mature peptides and/or functional portions thereof are provided in Table 1. In some embodiments, the mammalian growth factor comprises a sequence that is at least 70% identical (e.g., at least 75% identical, at least 80% identical, at least 85% identical, at least 90% identical, at least 95% identical, or at least 99% identical) to any of the sequences in Table 1 or any secreted human growth factor, and has osteogenic activity. In some embodiments, the amino acids in a mammalian growth factor that are conserved between different species are likely important for osteogenic activity and may not be mutated, while amino acids in a mammalian growth factor that are not conserved between different species are not likely important for osteogenic activity and may be mutated.

In some embodiments, the mammalian growth factor comprises BMP-2. In some embodiments, the mammalian growth factor is a mature peptide of BMP-2 (e.g., does not comprise a signal sequence). In some embodiments, the mammalian growth factor comprises a functional portion of BMP-2. In some embodiments, the functional portion of BMP-2 comprises a sequence at least about 70%, 75%, 80%, 85%, 90%, 95%, or 100% identical to: QAKHKQRKRLKSSCKRHPLYVDFSDVGWNDWIVAPPGYHAFYCHGECPFPLADHLNS TNHAIVQTLVNSVNSKIPKACCVPTELSAISMLYLDENEKVVLKNYQDMVVEGCGCR (SEQ ID NO: 454). In some embodiments, the mammalian growth factor comprises a sequence at least about 70%, 75%, 80%, 85%, 90%, 95%, or 100% identical to SEQ ID NO: 454. In some embodiments, the mammalian growth factor comprises a sequence at least about 90% identical to SEQ ID NO: 454. In some embodiments, the mammalian growth factor comprises SEQ ID NO: 454.

In some embodiments, the mammalian growth factor is a non-human mammalian growth factor. The non-human mammalian growth factor may be homologous to a human growth factor, such as one or more of the human growth factors of Table 1. In some embodiments, a non-human mammalian growth factor is homologous to a human growth factor if the non-human mammalian growth factor is at least ab out 80% identical to the human mammalian growth factor as determined using the NCBI Blast alignment algorithm as of the date of this filing. In some cases, the coverage is at least about 90%. In some embodiments, a non-human mammalian growth factor is homologous to a human growth factor if the non-human mammalian growth factor is at least about 80% positive as compared to the human mammalian growth factor as determined using the NCBI Blast alignment algorithm as of the date of this filing. In some cases, the coverage is at least about 90%. In some embodiments, a non-human mammalian growth factor is homologous to a human growth factor if the non-human mammalian growth factor aligned with the human growth factor using the NCBI Blast as of the date of this filing has an E value of less than about 1E-40, at least about 1E-50, 1E-60, 1E-70, or 1E-10, with a query cover of at least about 90%.

TABLE 1
Therapeutic Growth Factors

| | | SEQ ID |
|---|---|---|
| Name | Protein Sequence | NO: |
| EGF | NSDSECPLSHDGYCLHDGVCMYIEALDKYACNCVVGYIGERCQYRDLK | 442 |
| | WWELR | |
| PDGF | EEAEIPREVIERLARSQIHSIRDLQRLLEIDSVGSEDSLDTSLRAHGVHATK | 443 |
| | HVPEKRPLPIRRKR | |
| IGF-1 | GPETLCGAELVDALQFVCGDRGFYFNKPTGYGSSSRRAPQTGIVDECCFR | 444 |
| | SCDLRRLEMYCAPLKPAKSA | |
| FGF | FNLPPGNYKKPKLLYCSNGGHFLRILPDGTVDGTRDRSDQHIQLQLSAES | 445 |
| | VGEVYIKSTETGQYLAMDTDGLLYGSQTPNEECLFLERLEENHYNTYISK | |
| | KHAEKNWFVGLKKNGSCKRGPRTHYGQKAILFLPLPVSSD | |
| FGF2 | PALPEDGGSGAFPPGHFKDPKRLYCKNGGFFLRIHPDGRVDGVREKSDPH | 446 |
| | IKLQLQAEERGVVSIKGVCANRYLAMKEDGRLLASKCVTDECFFFERLES | |
| | NNYNTYRSRKYTSWYVALKRTGQYKLGSKTGPGQKAILFLPMSAKS | |
| FGF18 | EENVDFRIHVENQTRARDDVSRKQLRLYQLYSRTSGKHIQVLGRRISARG | 447 |
| | EDGDKYAQLLVETDTFGSQVRIKGKETEFYLCMNRKGKLVGKPDGTSKE | |
| | CVFIEKVLENNYTALMSAKYSGWYVGFTKKGRPRKGPKTRENQQDVHF | |
| | MKRYPKGQPELQKPFKYTTVTKRSRRIRPTHPA | |
| TGF-α | ENSTSPLSADPPVAAAVVSHFNDCPDSHTQFCFHGTCRFLVQEDKPACVC | 448 |
| | HSGYVGARCEHADLLAVVAASQKKQAITALVVVSIVALAVLIITCVLIHC | |
| | CQVRKHCEWCRALICRHEKPSALLKGRTACCHSETVV | |
| TGF-α | VVSHFNDCPDSHTQFCFHGTCRFLVQEDKPACVCHSGYVGARCEHADLL | 449 |
| | A | |
| TGF-ß1 | ALDTNYCFSSTEKNCCVRQLYIDFRKDLGWKWIHEPKGYHANFCLGPCP | 450 |
| | YIWSLDTQYSKVLALYNQHNPGASAAPCCVPQALEPLPIVYYVGRKPKV | |
| | EQLSNMIVRSCKCS | |
| TGF-ß3 | ALDTNYCFRNLEENCCVRPLYIDFRQDLGWKWVHEPKGYYANFCSGPCP | 451 |
| | YLRSADTTHSTVLGLYNTLNPEASASPCCVPQDLEPLTILYYVGRTPKVE | |
| | QLSNMVVKSCKCS | |
| OP-2 | AVRPLRRRQPKKSNELPQANRLPGIFDDVHGSHGRQVCRRHELYVSFQD | 452 |
| (BMP-8) | LGWLDWVIAPQGYSAYYCEGECSFPLDSCMNATNHAILQSLVHLMMPD | |
| | AVPKACCAPTKLSATSVLYYDSSNNVILRKHRNMVVKACGCH | |
| BMP8A | AVRPLRRRQPKKSNELPQANRLPGIFDDVRGSHGRQVCRRHELYVSFQD | 453 |
| | LGWLDWVIAPQGYSAYYCEGECSFPLDSCMNATNHAILQSLVHLMKPN | |
| | AVPKACCAPTKLSATSVLYYDSSNNVILRKHRNMVVKACGCH | |
| BMP-2 | QAKHKQRKRLKSSCKRHPLYVDFSDVGWNDWIVAPPGYHAFYCHGECP | 454 |
| | FPLADHLNSTNHAIVQTLVNSVNSKIPKACCVPTELSAISMLYLDENEKV | |
| | VLKNYQDMVVEGCGCR | |
| BMP-3 | QWIEPRNCARRYLKVDFADIGWSEWIISPKSFDAYYCSGACQFPMPKSLK | 455 |
| | PSNHATIQSIVRAVGVVPGIPEPCCVPEKMSSLSILFFDENKNVVLKVYPN | |
| | MTVESCACR | |
| BMP-4 | SPKHHSQRARKKNKNCRRHSLYVDFSDVGWNDWIVAPPGYQAFYCHGD | 456 |
| | CPFPLADHLNSTNHAIVQTLVNSVNSSIPKACCVPTELSAISMLYLDEYDK | |
| | VVLKNYQEMVVEGCGCR | |
| BMP-5 | AANKRKNQNRNKSSSHQDSSRMSSVGDYNTSEQKQACKKHELYVSFRD | 457 |
| | LGWQDWIIAPEGYAAFYCDGECSFPLNAHMNATNHAIVQTLVHLMFPD | |
| | HVPKPCCAPTKLNAISVLYFDDSSNVILKKYRNMVVRSCGCH | |
| BMP-6/ | SASSRRRQQSRNRSTQSQDVARVSSASDYNSSELKTACRKHELYVSFQDL | 458 |
| VGR | GWQDWIIAPKGYAANYCDGECSFPLNAHMNATNHAIVQTLVHLMNPEY | |
| | VPKPCCAPTKLNAISVLYFDDNSNVILKKYRNMVVRACGCH | |
| BMP-7/ | STGSKQRSQNRSKTPKNQEALRMANVAENSSSDQRQACKKHELYVSFRD | 459 |
| OP-1 | LGWQDWIIAPEGYAAYYCEGECAFPLNSYMNATNHAIVQTLVHFINPET | |
| | VPKPCCAPTQLNAISVLYFDDSSNVILKKYRNMVVRACGCH | |
| BMP-9 | SAGAGSHCQKTSLRVNFEDIGWDSWIIAPKEYEAYECKGGCFFPLADDVT | 460 |
| | PTKHAIVQTLVHLKFPTKVGKACCVPTKLSPISVLYKDDMGVPTLKYHY | |
| | EGMSVAECGCR | |
| BMP-10 | NAKGNYCKRTPLYIDFKEIGWDSWIIAPPGYEAYECRGVCNYPLAEHLTP | 461 |
| | TKHAIIQALVHLKNSQKASKACCVPTKLEPISILYLDKGVVTYKFKYEGM | |
| | AVSECGCR | |
| BMP-11/ | NLGLDCDEHSSESRCCRYPLTVDFEAFGWDWIIAPKRYKANYCSGQCEY | 462 |
| GDF-11 | MFMQKYPHTHLVQQANPRGSAGPCCTPTKMSPINMLYFNDKQQIIYGKI | |
| | PGMVVDRCGCS | |
| BMP-12 | TALAGTRTAQGSGGGAGRGHGRRGRSRCSRKPLHVDFKELGWDDWIIA | 463 |
| | PLDYEAYHCEGLCDFPLRSHLEPTNHAIIQTLLNSMAPDAAPASCCVPAR | |
| | LSPISILYIDAANNVVYKQYEDMVVEACGCR | |
| BMP-13/ | TAFASRHGKRHGKKSRLRCSKKPLHVNFKELGWDDWIIAPLEYEAYHCE | 464 |
| GDF-6 | GVCDFPLRSHLEPTNHAIIQTLMNSMDPGSTPPSCCVPTKLTPISILYIDAG | |
| | NNVVYKQYEDMVVESCGCR | |
| BMP-15 | QADGISAEVTASSSKHSGPENNQCSLHPFQISFRQLGWDHWIIAPPFYTPN | 465 |
| | YCKGTCLRVLRDGLNSPNHAIIQNLINQLVDQSVPRPSCVPYKYVPISVL | |
| | MIEANGSILYKEYEGMIAESCTCR | |
| DPP isoform | IPVPQSKPLERHVEKSMNLHLLARSNVSVQDELNASGTIKESGVLVHEGD | 466 |
| 1 | RGRQENTQDGHKGEGNGSKWAEVGGKSFSTYSTLANEEGNIEGWNGDT | |
| | GKAETYGHDGIHGKEENITANGIQGQVSIIDNAGATNRSNINGNTDKNTQ | |
| | NGDVGDAGHNEDVAVVQEDGPQVAGSNNSTDNEDEIIENSCRNEGNTSE | |
| | ITPQINSKRNGTKEAEVTPGTGEDAGLDNSDGSPSGNGADEDEDEGSGDD | |
| | EDEEAGNGKDSSNNSKGQEGQDHGKEDDHDSSIGQNSDSKEYYDPEGKE | |
| | DPHNEVDGDKTSKSEENSAGIPEDNGSQRIEDTQKLNHRESKRVENRITK | |
| | ESETHAVGKSQDKGIEIKGPSSGNRNITKEVGKGNEGKEDKGQHGMILG | |
| | KGNVKTQGEVVNIEGPGQKSEPGNKVGHSNTGSDSNSDGYDSYDFDDKS | |
| | MQG | |
| DPP isoform | IPVPQSKPLERHVEKSMNLHLLARSNVSVQDELNASGTIKESGVLVHEGD | 467 |
| 2 | RGRQENTQDGHKGEGNGSKWAEVGGKSFSTYSTLANEEGNIEGWNGDT | |
| | GKAETYGHDGIHGKEENITANGIQGQVSIIDNAGATNRSNTNGNTDKNTQ | |
| | NGDVGDAGHNEDVAVVQEDGPQVAGSNNSTDNEDEIIENSCRNEGNTSE | |
| | ITPQINSKRNGTKEAEVTPGTGEDAGLDNSDGSPSGNGADEDEDEGSGDD | |
| | EDEEAGNGKDSSNNSKGQEGQDHGKEDDHDSSIGQNSDSKEYYDPEGKE | |
| | DPHNEVDGDKTSKSEENSAGIPEDNGSQRIEDTQKLNHRESKRVENRITK | |
| | ESETHAVGKSQDKGIEIKGPSSGNRNITKEVGKGNEGKEDKGQHGMILG | |
| | KGNVKTQGEVVNIEGPGQKSEPGNKVGHSNTGSDSNSDGYDSYDFDDKS | |
| | MQGDDPNSSDESNGNDDANSESDNNSSSRGDASYNSDESKDNGNGSDS | |
| | KGAEDDDSDSTSDTNNSDSNGNGNNGNDDNDKSDSGKGKSDSSDSDSS | |
| | DSSNSSDSSDSSDSDSSDSNSSSDSDSSDSDSSDSSDSDSSDSSNSSDSSDSS | |
| | DSSDSSDSSDSSDSKSDSSKSESDSSDSDSKSDSSDSNSSDSSDNSDSSDSS | |
| | NSSNSSDSSDSSDSSDSSSSSDSSNSSDSSDSSDSSNSSESSDSSDSSDSDSS | |
| | DSSDSSNSNSSDSDSSNSSDSSDSSNSSDSSDSSDSSNSSDSSDSSDSSNSSD | |
| | SSDSSDSSDSSDSSNSSDSNDSSNSSDSSDSSNSSDSSNSSDSSDSSDSSDSD | |
| | SSNSSDSSNSSDSSDSSNSSDSSDSSDSSDGSDSDSSNRSDSSNSSDSSDSSD | |
| | SSNSSDSSDSSDSNESSNSSDSSDSSNSSDSDSSDSSNSSDSSDSSNSSDSSE | |
| | SSNSSDNSNSSDSSNSSDSSDSSDSSNSSDSSNSSDSSNSSDSSDSNSSDSSD | |
| | SSNSSDSSDSSDSSDSSDSSDSSNSSDSSDSSDSSDSSNSSDSSNSSDSSNSS | |
| | DSSDSSDSSDSSDSSDSSDSSDSSNSSDSSDSSDSSDSSDSSDSSDSSDSSES | |
| | SDSSDSSNSSDSSDSSDSSDSSDSSDSSDSSDSSDSSNSSDSSDSSDSSDSSD | |
| | SSNSSDSSDSSESSDSSDSSDSSDSSDSSDSSDSSDSSDSSNSSDSSDSSDSS | |
| | DSSDSSDSSDSSDSSDSSDSSDSSDSSDSSDSSDSSDSSDSNESSDSSDSSDS | |
| | SDSSNSSDSSDSSDSSDSTSDSNDESDSQSKSGNGNNNGSDSDSDSEGSDS | |
| | NHSTSDD | |
| DPP isoform | DDPNSSDESNGNDDANSESDNNSSSRGDASYNSDESKDNGNGSDSKGAE | 468 |
| 3 | DDDSDSTSDTNNSDSNGNGNNGNDDNDKSDSGKGKSDSSDSDSSDSSNS | |
| | SDSSDSSDSDSSDSNSSSDSDSSDSDSSDSSDSDSSDSSNSSDSSDSSDSSDS | |
| | SDSSDSSDSKSDSSKSESDSSDSDSKSDSSDSNSSDSSDNSDSSDSSNSSNS | |
| | SDSSDSSDSSDSSSSSDSSNSSDSSDSSDSSNSSESSDSSDSSDSDSSDSSDSS | |
| | NSNSSDSDSSNSSDSSDSSNSSDSSDSSDSSNSSDSSDSSDSSNSSDSSDSSD | |
| | SSDSSDSSNSSDSNDSSNSSDSSDSSNSSDSSNSSDSSDSSDSSDSDSSNSSD | |
| | SSNSSDSSDSSNSSDSSDSSDSSDGSDSDSSNRSDSSNSSDSSDSSDSSNSSD | |
| | SSDSSDSNESSNSSDSSDSSNSSDSDSSDSSNSSDSSDSSNSSDSSESSNSSD | |
| | NSNSSDSSNSSDSSDSSDSSNSSDSSNSSDSSNSSDSSDSNSSDSSDSSNSSD | |
| | SSDSSDSSDSSDSSDSSNSSDSSDSSDSSDSSNSSDSSNSSDSSNSSDSSDSS | |
| | DSSDSSDSSDSSDSSDSSNSSDSSDSSDSSDSSDSSDSSDSSDSSESSDSSDS | |
| | SNSSDSSDSSDSSDSSDSSDSSDSSDSSDSSNSSDSSDSSDSSDSSDSSNSSD | |
| | SSDSSESSDSSDSSDSSDSSDSSDSSDSSDSSDSSNSSDSSDSSDSSDSSDSS | |
| | DSSDSSDSSDSSDSSDSSDSSDSSDSSDSSDSSDSNESSDSSDSSDSSDSSNS | |
| | SDSSDSSDSSDSTSDSNDESDSQSKSGNGNNNGSDSDSDSEGSDSNHSTSD | |
| | D | |
| GDF-1 | DAEPVLGGGPGGACRARRLYVSFREVGWHRWVIAPRGFLANYCQGQCA | 469 |
| | LPVALSGSGGPPALNHAVLRALMHAAAPGAADLPCCVPARLSPISVLFFD | |
| | NSDNVVLRQYEDMVVDECGCR | |
| GDF-3 | AAIPVPKLSCKNLCHRHQLFINFRDLGWHKWIIAPKGFMANYCHGECPFS | 470 |
| | LTISLNSSNYAFMQALMHAVDPEIPQAVCIPTKLSPISMLYQDNNDNVILR | |
| | HYEDMVVDECGCG | |
| GDF-5 | APLATRQGKRPSKNLKARCSRKALHVNFKDMGWDDWIIAPLEYEAFHC | 471 |
| | EGLCEFPLRSHLEPTNHAVIQTLMNSMDPESTPPTCCVPTRLSPISILFIDSA | |
| | NNVVYKQYEDMVVESCGCR | |
| GDF8 | DFGLDCDEHSTESRCCRYPLTVDFEAFGWDWIIAPKRYKANYCSGECEF | 472 |
| | VFLQKYPHTHLVHQANPRGSAGPCCTPTKMSPINMLYFNGKEQIIYGKIP | |
| | AMVVDRCGCS | |
| GDF15 | ARARNGDHCPLGPGRCCRLHTVRASLEDLGWADWVLSPREVQVTMCIG | 473 |
| | ACPSQFRAANMHAQIKTSLHRLKPDTVPAPCCVPASYNPMVLIQKTDTG | |
| | VSLQTYDDLLAKDCHCI | |
| VEGF | APMAEGGGQNHHEVVKFMDVYQRSYCHPIETLVDIFQEYPDEIEYIFKPS | 474 |
| | CVPLMRCGGCCNDEGLECVPTEESNITMQIMRIKPHQGQHIGEMSFLQHN | |
| | KCECRPKKDRARQEKKSVRGKGKGQKRKRKKSRYKSWSVYVGARCCL | |
| | MPWSLPGPHPCGPCSERRKHLFVQDPQTCKCSCKNTDSRCKARQLELNE | |
| | RTCRCDKPRR | |
| HABP | FSLMSLLESLDPDWTPDQYDYSYEDYNQEENTSSTLTHAENPDWYYTED | 475 |
| Isoform 1 | QADPCQPNPCEHGGDCLVHGSTFTCSCLAPFSGNKCQKVQNTCKDNPCG | |
| | RGQCLITQSPPYYRCVCKHPYTGPSCSQVVPVCRPNPCQNGATCSRHKRR | |
| | SKFTCACPDQFKGKFCEIGSDDCYVGDGYSYRGKMNRTVNQHACLYWN | |
| | SHLLLQENYNMFMEDAETHGIGEHNFCRNPDADEKPWCFIKVINDKVK | |
| | WEYCDVSACSAQDVAYPEESPTEPSTKLPGFDSCGKTEIAERKIKR | |
| HABP | IYGGFKSTAGKHPWQASLQSSLPLTISMPQGHFCGGALIHPCWVLTAAHC | 477 |
| Isoform 2 | TDIKTRHLKVVLGDQDLKKEEFHEQSFRVEKIFKYSHYNERDEIPHNDIAL | |
| | LKLKPVDGHCALESKYVKTVCLPDGSFPSGSECHISGWGVTETGKGSRQ | |
| | LLDAKVKLIANTLCNSRQLYDHMIDDSMICAGNLQKPGQDTCQGDSGGP | |
| | LTCEKDGTYYVYGIVSWGLECGKRPGVYTQVTKFLNWIKATIKSESGF | |
| CBP | AEVKKPAAAAAPGTAEKLSPKAATLAERSAGLAFSLYQAMAKDQAVEN | 479 |
| | ILVSPVVVASSLGLVSLGGKATTASQAKAVLSAEQLRDEEVHAGLGELLR | |
| | SLSNSTARNVTWKLGSRLYGPSSVSFADDFVRSSKQHYNCEHSKINFRDK | |
| | RSALQSINEWAAQTTDGKLPEVTKDVERTDGALLVNAMFFKPHWDEKF | |
| | HHKMVDNRGFMVTRSYTVGVMMMHRTGLYNYYDDEKEKLQIVEMPL | |
| | AHKLSSLIILMPHHVEPLERLEKLLTKEQLKIWMGKMQKKAVAISLPKGV | |
| | VEVTHDLQKHLAGLGLTEAIDKNKADLSRMSGKKDLYLASVFHATAFEL | |
| | DTDGNPFDQDIYGREELRSPKLFYADHPFIFLVRDTQSGSLLFIGRLVRPK | |
| | GDKMRDEL | |
| KGF | CNDMTPEQMATNVNCSSPERHTRSYDYMEGGDIRVRRLFCRTQWYLRID | 480 |
| | KRGKVKGTQEMKNNYNIMEIRTVAVGIVAIKGVESEFYLAMNKEGKLY | |
| | AKKECNEDCNFKELILENHYNTYASAKWTHNGGEMFVALNQKGIPVRG | |
| | KKTKKEQKTAHFLPMAIT | |
| TNFα | GPQREEFPRDLSLISPLAQAVRSSSRTPSDKPVAHVVANPQAEGQLQWLN | 481 |
| | RRANALLANGVELRDNQLVVPSEGLYLIYSQVLFKGQGCPSTHVLLTHTI | |
| | SRIAVSYQTKVNLLSAIKSPCQRETPEGAEAKPWYEPIYLGGVFQLEKGD | |
| | RLSAEINRPDYLDFAESGQVYFGIIAL | |
| TRAIL | TNELKQMQDKYSKSGIACFLKEDDSYWDPNDEESMNSPCWQVKWQLR | 482 |
| | QLVRKMILRTSEETISTVQEKQQNISPLVRERGPQRVAAHITGTRGRSNTL | |
| | SSPNSKNEKALGRKINSWESSRSGHSFLSNLHLRNGELVIHEKGFYYIYSQ | |
| | TYFRFQEEIKENTKNDKQMVQYIYKYTSYPDPILLMKSARNSCWSKDAE | |
| | YGLYSIYQGGIFELKENDRIFVSVTNEHLIDMDHEASFFGAFLVG | |
| WNT1 | ANSSGRWWGIVNVASSTNLLTDSKSLQLVLEPSLQLLSRKQRRLIRQNPG | 483 |
| | ILHSVSGGLQSAVRECKWQFRNRRWNCPTAPGPHLFGKIVNRGCRETAFI | |
| | FAITSAGVTHSVARSCSEGSIESCTCDYRRRGPGGPDWHWGGCSDNIDFG | |
| | RLFGREFVDSGEKGRDLRFLMNLHNNEAGRTTVFSEMRQECKCHGMSG | |
| | SCTVRTCWMRLPTLRAVGDVLRDRFDGASRVLYGNRGSNRASRAELLR | |
| | LEPEDPAHKPPSPHDLVYFEKSPNFCTYSGRLGTAGTAGRACNSSSPALD | |
| | GCELLCCGRGHRTRTQRVTERCNCTFHWCCHVSCRNCTHTRVLHECL | |
| WNT2 | SWWYMRATGGSSRVMCDNVPGLVSSQRQLCHRHPDVMRAISQGVAEW | 484 |
| | TAECQHQFRQHRWNCNTLDRDHSLFGRVLLRSSRESAFVYAISSAGVVF | |
| | AITRACSQGEVKSCSCDPKKMGSAKDSKGIFDWGGCSDNIDYGIKFARAF | |
| | VDAKERKGKDARALMNLHNNRAGRKAVKRFLKQECKCHGVSGSCTLR | |
| | TCWLAMADFRKTGDYLWRKYNGAIQVVMNQDGTGFTVANERFKKPTK | |
| | NDLVYFENSPDYCIRDREAGSLGTAGRVCNLTSRGMDSCEVMCCGRGY | |
| | DTSHVTRMTKCGCKFHWCCAVRCQDCLEALDVHTCKAPKNADWTTAT | |
| WNT2B | SWWYIGALGARVICDNIPGLVSRQRQLCQRYPDIMRSVGEGAREWIREC | 485 |
| | QHQFRHHRWNCTTLDRDHTVFGRVMLRSSREAAFVYAISSAGVVHAITR | |
| | ACSQGELSVCSCDPYTRGRHHDQRGDFDWGGCSDNIHYGVRFAKAFVD | |
| | AKEKRLKDARALMNLHNNRCGRTAVRRFLKLECKCHGVSGSCTLRTCW | |
| | RALSDFRRTGDYLRRRYDGAVQVMATQDGANFTAARQGYRRATRTDL | |
| | VYFDNSPDYCVLDKAAGSLGTAGRVCSKTSKGTDGCEIMCCGRGYDTT | |
| | RVTRVTQCECKFHWCCAVRCKECRNTVDVHTCKAPKKAEWLDQT | |
| WNT3 | GYPIWWSLALGQQYTSLGSQPLLCGSIPGLVPKQLRFCRNYIEIMPSVAEG | 486 |
| | VKLGIQECQHQFRGRRWNCTTIDDSLAIFGPVLDKATRESAFVHAIASAG | |
| | VAFAVTRSCAEGTSTICGCDSHHKGPPGEGWKWGGCSEDADFGVLVSRE | |
| | FADARENRPDARSAMNKHNNEAGRTTILDHMHLKCKCHGLSGSCEVKT | |
| | CWWAQPDFRAIGDFLKDKYDSASEMVVEKHRESRGWVETLRAKYSLFK | |
| | PPTERDLVYYENSPNFCEPNPETGSFGTRDRTCNVTSHGIDGCDLLCCGR | |
| | GHNTRTEKRKEKCHCIFHWCCYVSCQECIRIYDVHTCK | |
| WNT3A | SYPIWWSLAVGPQYSSLGSQPILCASIPGLVPKQLRFCRNYVEIMPSVAEG | 487 |
| | IKIGIQECQHQFRGRRWNCTTVHDSLAIFGPVLDKATRESAFVHAIASAG | |
| | VAFAVTRSCAEGTAAICGCSSRHQGSPGKGWKWGGCSEDIEFGGMVSRE | |
| | FADARENRPDARSAMNRHNNEAGRQAIASHMHLKCKCHGLSGSCEVKT | |
| | CWWSQPDFRAIGDFLKDKYDSASEMVVEKHRESRGWVETLRPRYTYFK | |
| | VPTERDLVYYEASPNFCEPNPETGSFGTRDRTCNVSSHGIDGCDLLCCGR | |
| | GHNARAERRREKCRCVFHWCCYVSCQECTRVYDVHTCK | |
| WNT4 | SNWLYLAKLSSVGSISEEETCEKLKGLIQRQVQMCKRNLEVMDSVRRGA | 488 |
| | QLAIEECQYQFRNRRWNCSTLDSLPVFGKVVTQGTREAAFVYAISSAGV | |
| | AFAVTRACSSGELEKCGCDRTVHGVSPQGFQWSGCSDNIAYGVAFSQSF | |
| | VDVRERSKGASSSRALMNLHNNEAGRKAILTHMRVECKCHGVSGSCEV | |
| | KTCWRAVPPFRQVGHALKEKFDGATEVEPRRVGSSRALVPRNAQFKPHT | |
| | DEDLVYLEPSPDFCEQDMRSGVLGTRGRTCNKTSKAIDGCELLCCGRGF | |
| | HTAQVELAERCSCKFHWCCFVKCRQCQRLVELHTCR | |
| WNT5A | IIGAQPLCSQLAGLSQGQKKLCHLYQDHMQYIGEGAKTGIKECQYQFRH | 489 |
| | RRWNCSTVDNTSVFGRVMQIGSRETAFTYAVSAAGVVNAMSRACREGE | |
| | LSTCGCSRAARPKDLPRDWLWGGCGDNIDYGYRFAKEFVDARERERIHA | |
| | KGSYESARILMNLHNNEAGRRTVYNLADVACKCHGVSGSCSLKTCWLQ | |
| | LADFRKVGDALKEKYDSAAAMRLNSRGKLVQVNSRFNSPTTQDLVYIDP | |
| | SPDYCVRNESTGSLGTQGRLCNKTSEGMDGCELMCCGRGYDQFKTVQT | |
| | ERCHCKFHWCCYVKCKKCTEIVDQFVCK | |
| WNT5B | QLLTDANSWWSLALNPVQRPEMFIIGAQPVCSQLPGLSPGQRKLCQLYQ | 490 |
| | EHMAYIGEGAKTGIKECQHQFRQRRWNCSTADNASVFGRVMQIGSRETA | |
| | FTHAVSAAGVVNAISRACREGELSTCGCSRTARPKDLPRDWLWGGCGD | |
| | NVEYGYRFAKEFVDAREREKNFAKGSEEQGRVLMNLQNNEAGRRAVY | |
| | KMADVACKCHGVSGSCSLKTCWLQLAEFRKVGDRLKEKYDSAAAMRV | |
| | TRKGRLELVNSRFTQPTPEDLVYVDPSPDYCLRNESTGSLGTQGRLCNKT | |
| | SEGMDGCELMCCGRGYNQFKSVQVERCHCKFHWCCFVRCKKCTEIVDQ | |
| | YICK | |
| WNT6 | LWWAVGSPLVMDPTSICRKARRLAGRQAELCQAEPEVVAELARGARLG | 491 |
| | VRECQFQFRFRRWNCSSHSKAFGRILQQDIRETAFVFAITAAGASHAVTQ | |
| | ACSMGELLQCGCQAPRGRAPPRPSGLPGTPGPPGPAGSPEGSAAWEWGG | |
| | CGDDVDFGDEKSRLFMDARHKRGRGDIRALVQLHNNEAGRLAVRSHTR | |
| | TECKCHGLSGSCALRTCWQKLPPFREVGARLLERFHGASRVMGTNDGK | |
| | ALLPAVRTLKPPGRADLLYAADSPDFCAPNRRTGSPGTRGRACNSSAPDL | |
| | SGCDLLCCGRGHRQESVQLEENCLCRFHWCCVVQCHRCRVRKELSLCL | |
| WNT7A | LGASIICNKIPGLAPRQRAICQSRPDAIIVIGEGSQMGLDECQFQFRNGRW | 492 |
| | NCSALGERTVFGKELKVGSREAAFTYAIIAAGVAHAITAACTQGNLSDCG | |
| | CDKEKQGQYHRDEGWKWGGCSADIRYGIGFAKVFVDAREIKQNARTLM | |
| | NLHNNEAGRKILEENMKLECKCHGVSGSCTTKTCWTTLPQFRELGYVLK | |
| | DKYNEAVHVEPVRASRNKRPTFLKIKKPLSYRKPMDTDLVYIEKSPNYCE | |
| | EDPVTGSVGTQGRACNKTAPQASGCDLMCCGRGYNTHQYARVWQCNC | |
| | KFHWCCYVKCNTCSERTEMYTCK | |
| WNT7B | ALSSVVALGANIICNKIPGLAPRQRAICQSRPDAIIVIGEGAQMGINECQYQ | 493 |
| | FRFGRWNCSALGEKTVFGQELRVGSREAAFTYAITAAGVAHAVTAACSQ | |
| | GNLSNCGCDREKQGYYNQAEGWKWGGCSADVRYGIDFSRRFVDAREIK | |
| | KNARRLMNLHNNEAGRKVLEDRMQLECKCHGVSGSCTTKTCWTTLPKF | |
| | REVGHLLKEKYNAAVQVEVVRASRLRQPTFLRIKQLRSYQKPMETDLVY | |
| | IEKSPNYCEEDAATGSVGTQGRLCNRTSPGADGCDTMCCGRGYNTHQYT | |
| | KVWQCNCKFHWCCFVKCNTCSERTEVFTCK | |
| WNT8A | VNNFLITGPKAYLTYTTSVALGAQSGIEECKFQFAWERWNCPENALQLST | 494 |
| | HNRLRSATRETSFIHAISSAGVMYIITKNCSMGDFENCGCDGSNNGKTGG | |
| | HGWIWGGCSDNVEFGERISKLFVDSLEKGKDARALMNLHNNRAGRLAV | |
| | RATMKRTCKCHGISGSCSIQTCWLQLAEFREMGDYLKAKYDQALKIEMD | |
| | KRQLRAGNSAEGHWVPAEAFLPSAEAELIFLEESPDYCTCNSSLGIYGTE | |
| | GRECLQNSHNTSRWERRSCGRLCTECGLQVEERKTEVISSCNCKFQWCC | |
| | TVKCDQCRHVVSKYYCARSPGSAQSLGKGSA | |
| WNT8B | WSVNNFLMTGPKAYLIYSSSVAAGAQSGIEECKYQFAWDRWNCPERAL | 495 |
| | QLSSHGGLRSANRETAFVHAISSAGVMYTLTRNCSLGDFDNCGCDDSRN | |
| | GQLGGQGWLWGGCSDNVGFGEAISKQFVDALETGQDARAAMNLHNNE | |
| | AGRKAVKGTMKRTCKCHGVSGSCTTQTCWLQLPEFREVGAHLKEKYHA | |
| | ALKVDLLQGAGNSAAGRGAIADTFRSISTRELVHLEDSPDYCLENKTLGL | |
| | LGTEGRECLRRGRALGRWERRSCRRLCGDCGLAVEERRAETVSSCNCKF | |
| | HWCCAVRCEQCRRRVTKYFCSRAERPRGGAAHKPGRKP | |
| WNT9A | YFGLTGSEPLTILPLTLEPEAAAQAHYKACDRLKLERKQRRMCRRDPGV | 496 |
| | AETLVEAVSMSALECQFQFRFERWNCTLEGRYRASLLKRGFKETAFLYAI | |
| | SSAGLTHALAKACSAGRMERCTCDEAPDLENREAWQWGGCGDNLKYSS | |
| | KFVKEFLGRRSSKDLRARVDFHNNLVGVKVIKAGVETTCKCHGVSGSCT | |
| | VRTCWRQLAPFHEVGKHLKHKYETALKVGSTTNEAAGEAGAISPPRGRA | |
| | SGAGGSDPLPRTPELVHLDDSPSFCLAGRFSPGTAGRRCHREKNCESICCG | |
| | RGHNTQSRVVTRPCQCQVRWCCYVECRQCTQREEVYTCKG | |
| WNT9B | SYFGLTGREVLTPFPGLGTAAAPAQGGAHLKQCDLLKLSRRQKQLCRRE | 497 |
| | PGLAETLRDAAHLGLLECQFQFRHERWNCSLEGRMGLLKRGFKETAFLY | |
| | AVSSAALTHTLARACSAGRMERCTCDDSPGLESRQAWQWGVCGDNLK | |
| | YSTKFLSNFLGSKRGNKDLRARADAHNTHVGIKAVKSGLRTTCKCHGVS | |
| | GSCAVRTCWKQLSPFRETGQVLKLRYDSAVKVSSATNEALGRLELWAP | |
| | ARQGSLTKGLAPRSGDLVYMEDSPSFCRPSKYSPGTAGRVCSREASCSSL | |
| | CCGRGYDTQSRLVAFSCHCQVQWCCYVECQQCVQEELVYTCKH | |
| WNT10A | MPRSAPNDILDLRLPPEPVLNANTVCLTLPGLSRRQMEVCVRHPDVAASA | 498 |
| | IQGIQIAIHECQHQFRDQRWNCSSLETRNKIPYESPIFSRGFRESAFAYAIA | |
| | AAGVVHAVSNACALGKLKACGCDASRRGDEEAFRRKLHRLQLDALQRG | |
| | KGLSHGVPEHPALPTASPGLQDSWEWGGCSPDMGFGERFSKDFLDSREP | |
| | HRDIHARMRLHNNRVGRQAVMENMRRKCKCHGTSGSCQLKTCWQVTP | |
| | EFRTVGALLRSRFHRATLIRPHNRNGGQLEPGPAGAPSPAPGAPGPRRRA | |
| | SPADLVYFEKSPDFCEREPRLDSAGTVGRLCNKSSAGSDGCGSMCCGRG | |
| | HNILRQTRSERCHCRFHWCCFVVCEECRITEWVSVCK | |
| WNT10B | NEILGLKLPGEPPLTANTVCLTLSGLSKRQLGLCLRNPDVTASALQGLHIA | 499 |
| | VHECQHQLRDQRWNCSALEGGGRLPHHSAILKRGFRESAFSFSMLAAGV | |
| | MHAVATACSLGKLVSCGCGWKGSGEQDRLRAKLLQLQALSRGKSFPHS | |
| | LPSPGPGSSPSPGPQDTWEWGGCNHDMDFGEKFSRDFLDSREAPRDIQAR | |
| | MRIHNNRVGRQVVTENLKRKCKCHGTSGSCQFKTCWRAAPEFRAVGAA | |
| | LRERLGRAIFIDTHNRNSGAFQPRLRPRRLSGELVYFEKSPDFCERDPTMG | |
| | SPGTRGRACNKTSRLLDGCGSLCCGRGHNVLRQTRVERCHCRFHWCCY | |
| | VLCDECKVTEWVNVCK | |
| WNT11 | IKWLALSKTPSALALNQTQHCKQLEGLVSAQVQLCRSNLELMHTVVHA | 500 |
| | AREVMKACRRAFADMRWNCSSIELAPNYLLDLERGTRESAFVYALSAA | |
| | AISHAIARACTSGDLPGCSCGPVPGEPPGPGNRWGGCADNLSYGLLMGA | |
| | KFSDAPMKVKKTGSQANKLMRLHNSEVGRQALRASLEMKCKCHGVSGS | |
| | CSIRTCWKGLQELQDVAADLKTRYLSATKVVHRPMGTRKHLVPKDLDIR | |
| | PVKDSELVYLQSSPDFCMKNEKVGSHGTQDRQCNKTSNGSDSCDLMCC | |
| | GRGYNPYTDRVVERCHCKYHWCCYVTCRRCERTVERYVCK | |
| WNT16 | NWMWLGIASFGVPEKLGCANLPLNSRQKELCKRKPYLLPSIREGARLGIQ | 501 |
| | ECGSQFRHERWNCMITAAATTAPMGASPLFGYELSSGTKETAFIYAVMA | |
| | AGLVHSVTRSCSAGNMTECSCDTTLQNGGSASEGWHWGGCSDDVQYG | |
| | MWFSRKFLDFPIGNTTGKENKVLLAMNLHNNEAGRQAVAKLMSVDCRC | |
| | HGVSGSCAVKTCWKTMSSFEKIGHLLKDKYENSIQISDKTKRKMRRREK | |
| | DQRKIPIHKDDLLYVNKSPNYCVEDKKLGIPGTQGRECNRTSEGADGCNL | |
| | LCCGRGYNTHVVRHVERCECKFIWCCYVRCRRCESMTDVHTCK | |

Targeting Moieties

In some embodiments, the device or kit comprises a targeting moiety that tethers the therapeutic agent to the structure. In some embodiments, the targeting moiety is connected to the therapeutic agent, and the moiety non-covalently binds to the structure. As a non-limiting example, the targeting moiety is covalently connected to the therapeutic agent via a peptide bond. For instance, targeting moiety comprises a targeting peptide, and the targeting peptide is linked to the therapeutic agent via a peptide bond.

In some embodiments, the targeting moiety has an affinity for the structure, or a component of the structure, e.g., to a ceramic material of the structure such as calcium phosphate. In some embodiments, the dissociation constant (KD) for binding between the targeting moiety and the structure or component thereof is: (i) at least about 1 fM, at least about 10 fM, at least about 100 fM, or at least about 1 pM; and (ii) less than about 100 uM, less than about 90 uM, less than about 80 uM, less than about 70 uM, less than about 60 uM, less than about 50 uM, less than about 40 uM, less than about 30 uM, less than about 20 uM, less than about 10 uM, less than about 5 uM, less than about 1 uM, or less than about 100 pM. For example, the targeting moiety may bind to beta-tricalcium phosphate with an affinity of about 100 fM to about 100 uM, about 1 pM to about 100 μM, about 10 pM to about 100 μM, about 100 pM to about 100 μM, or about 1 μM to about 100 μM.

In some embodiments, the targeting moiety comprises one or more targeting peptides that each bind to the structure. In some embodiments, the targeting peptide binds to the ceramic material of the structure. For example, the targeting peptide binds to calcium phosphate (e.g., tricalcium phosphate, beta tricalcium phosphate, alpha tricalcium phosphate), hydroxyapatite, fluorapatite, bone (e.g., demineralized bone), glasses (bioglasses) such as silicates, vanadates, and related ceramic minerals, or chelated divalent metal ions, or a combination thereof. In some embodiments, the targeting peptide comprises two or more targeting peptides. In some embodiments, two or more targeting peptides is no more than about 50, 45, 40, 35, 30, 25, 20, 15, or 10 targeting peptides. In some embodiments, two or more targeting peptides is about 2, 3, 4, 5, 6, 7, 8, 9, 10, 11, 12, 13, 14, 15, 20, 25, or 30 targeting peptides. In some embodiments, two or more targeting peptides is about 2 to about 10 targeting peptides. In some embodiments, two or more targeting peptides is about 5 targeting peptides.

In some embodiments, the targeting peptide comprises a sequence at least about 70%, 75%, 80%, 85%, 90%, 95%, or 100% identical to SEQ ID NO: 1. In some embodiments, the targeting peptide comprises a sequence at least about 70%, 75%, 80%, 85%, 90%, 95%, or 100% identical to SEQ ID NO: 2. In some embodiments, the targeting peptide comprises a sequence at least about 70%, 75%, 80%, 85%, 90%, 95%, or 100% identical to SEQ ID NO: 3. In some embodiments, the targeting peptide comprises a sequence at least about 70%, 75%, 80%, 85%, 90%, 95%, or 100% identical to SEQ ID NO: 4. In some embodiments, the targeting peptide comprises a sequence at least about 70%, 75%, 80%, 85%, 90%, 95%, or 100% identical to SEQ ID NO: 5. In some embodiments, the targeting peptide comprises a sequence at least about 70%, 75%, 80%, 85%, 90%, 95%, or 100% identical to SEQ ID NO: 6. In some embodiments, the targeting peptide comprises a sequence at least ab out 70%, 75%, 80%, 85%, 90%, 95%, or 100% identical to SEQ ID NO: 7. In some embodiments, the targeting peptide comprises a sequence at least about 70%, 75%, 80%, 85%, 90%, 95%, or 100% identical to SEQ ID NO: 8. In some embodiments, the targeting peptide comprises a sequence at least about 70%, 75%, 80%, 85%, 90%, 95%, or 100% identical to SEQ ID NO: 9. In some embodiments, the targeting peptide comprises a sequence at least about 70%, 75%, 80%, 85%, 90%, 95%, or 100% identical to SEQ ID NO: 10. In some embodiments, the targeting peptide comprises a sequence at least about 70%, 75%, 80%, 85%, 90%, 95%, or 100% identical to SEQ ID NO: 11. In some embodiments, the targeting peptide comprises a sequence at least about 70%, 75%, 80%, 85%, 90%, 95%, or 100% identical to SEQ ID NO: 12. In some embodiments, the targeting peptide comprises a sequence at least about 70%, 75%, 80%, 85%, 90%, 95%, or 100% identical to SEQ ID NO: 13. In some embodiments, the targeting peptide comprises a sequence at least about 70%, 75%, 80%, 85%, 90%, 95%, or 100% identical to SEQ ID NO: 14. In some embodiments, the targeting peptide comprises a sequence at least about 70%, 75%, 80%, 85%, 90%, 95%, or 100% identical to SEQ ID NO: 15. In some embodiments, the targeting peptide comprises a sequence at least about 70%, 75%, 80%, 85%, 90%, 95%, or 100% identical to SEQ ID NO: 16. In some embodiments, the targeting peptide comprises a sequence at least about 70%, 75%, 80%, 85%, 90%, 95%, or 100% identical to SEQ ID NO: 17. In some embodiments, the targeting peptide comprises a sequence at least about 70%, 75%, 80%, 85%, 90%, 95%, or 100% identical to SEQ ID NO: 18. In some embodiments, the targeting peptide comprises a sequence at least about 70%, 75%, 80%, 85%, 90%, 95%, or 100% identical to SEQ ID NO: 19. In some embodiments, the targeting peptide comprises a sequence at least about 70%, 75%, 80%, 85%, 90%, 95%, or 100% identical to SEQ ID NO: 20. In some embodiments, the targeting peptide comprises a sequence at least about 70%, 75%, 80%, 85%, 90%, 95%, or 100% identical to SEQ ID NO: 21. In some embodiments, the targeting peptide comprises a sequence at least about 70%, 75%, 80%, 85%, 90%, 95%, or 100% identical to SEQ ID NO: 22. In some embodiments, the targeting peptide comprises a sequence at least about 70%, 75%, 80%, 85%, 90%, 95%, or 100% identical to SEQ ID NO: 23. In some embodiments, the targeting peptide comprises a sequence at least about 70%, 75%, 80%, 85%, 90%, 95%, or 100% identical to SEQ ID NO: 24. In some embodiments, the targeting peptide comprises a sequence at least about 70%, 75%, 80%, 85%, 90%, 95%, or 100% identical to SEQ ID NO: 25. In some embodiments, the targeting peptide comprises a sequence at least about 70%, 75%, 80%, 85%, 90%, 95%, or 100% identical to SEQ ID NO: 26. In some embodiments, the targeting peptide comprises a sequence at least about 70%, 75%, 80%, 85%, 90%, 95%, or 100% identical to SEQ ID NO: 27. In some embodiments, the targeting peptide comprises a sequence at least about 70%, 75%, 80%, 85%, 90%, 95%, or 100% identical to SEQ ID NO: 28. In some embodiments, the targeting peptide comprises a sequence at least about 70%, 75%, 80%, 85%, 90%, 95%, or 100% identical to SEQ ID NO: 29. In some embodiments, the targeting peptide comprises a sequence at least about 70%, 75%, 80%, 85%, 90%, 95%, or 100% identical to SEQ ID NO: 30. In some embodiments, the targeting peptide comprises a sequence at least about 70%, 75%, 80%, 85%, 90%, 95%, or 100% identical to SEQ ID NO: 31. In some embodiments, the targeting peptide comprises a sequence at least about 70%, 75%, 80%, 85%, 90%, 95%, or 100% identical to SEQ ID NO: 32. In some embodiments, the targeting peptide comprises a sequence at least about 70%, 75%, 80%, 85%, 90%, 95%, or 100% identical to SEQ ID NO: 33. In some embodiments, the targeting peptide comprises a sequence at least about 70%, 75%, 80%, 85%, 90%, 95%, or 100% identical to SEQ ID NO: 34. In some embodiments, the targeting peptide comprises a sequence at least about 70%, 75%, 80%, 85%, 90%, 95%, or 100% identical to SEQ ID NO: 35. In some embodiments, the targeting peptide comprises a sequence at least about 70%, 75%, 80%, 85%, 90%, 95%, or 100% identical to SEQ ID NO: 36. In some embodiments, the targeting peptide comprises a sequence at least about 70%, 75%, 80%, 85%, 90%, 95%, or 100% identical to SEQ ID NO: 37. In some embodiments, the targeting peptide comprises a sequence at least about 70%, 75%, 80%, 85%, 90%, 95%, or 100% identical to SEQ ID NO: 38. In some embodiments, the targeting peptide comprises a sequence at least about 70%, 75%, 80%, 85%, 90%, 95%, or 100% identical to SEQ ID NO: 39. In some embodiments, the targeting peptide comprises a sequence at least about 70%, 75%, 80%, 85%, 90%, 95%, or 100% identical to SEQ ID NO: 40. In some embodiments, the targeting peptide comprises a sequence at least about 70%, 75%, 80%, 85%, 90%, 95%, or 100% identical to SEQ ID NO: 41. In some embodiments, the targeting peptide comprises a sequence at least about 70%, 75%, 80%, 85%, 90%, 95%, or 100% identical to SEQ ID NO: 42. In some embodiments, the targeting peptide comprises a sequence at least about 70%, 75%, 80%, 85%, 90%, 95%, or 100% identical to SEQ ID NO: 43. In some embodiments, the targeting peptide comprises a sequence at least about 70%, 75%, 80%, 85%, 90%, 95%, or 100% identical to SEQ ID NO: 44. In some embodiments, the targeting peptide comprises a sequence at least about 70%, 75%, 80%, 85%, 90%, 95%, or 100% identical to SEQ ID NO: 45. In some embodiments, the targeting peptide comprises a sequence at least about 70%, 75%, 80%, 85%, 90%, 95%, or 100% identical to SEQ ID NO: 46. In some embodiments, the targeting peptide comprises a sequence at least about 70%, 75%, 80%, 85%, 90%, 95%, or 100% identical to SEQ ID NO: 47. In some embodiments, the targeting peptide comprises a sequence at least about 70%, 75%, 80%, 85%, 90%, 95%, or 100% identical to SEQ ID NO: 48. In some embodiments, the targeting peptide comprises a sequence at least about 70%, 75%, 80%, 85%, 90%, 95%, or 100% identical to SEQ ID NO: 49. In some embodiments, the targeting peptide comprises a sequence at least about 70%, 75%, 80%, 85%, 90%, 95%, or 100% identical to SEQ ID NO: 50. In some embodiments, the targeting peptide comprises a sequence at least about 70%, 75%, 80%, 85%, 90%, 95%, or 100% identical to SEQ ID NO: 51. In some embodiments, the targeting peptide comprises a sequence at least about 70%, 75%, 80%, 85%, 90%, 95%, or 100% identical to SEQ ID NO: 52. In some embodiments, the targeting peptide comprises a sequence at least about 70%, 75%, 80%, 85%, 90%, 95%, or 100% identical to SEQ ID NO: 53. In some embodiments, the targeting peptide comprises a sequence at least about 70%, 75%, 80%, 85%, 90%, 95%, or 100% identical to SEQ ID NO: 54. In some embodiments, the targeting peptide comprises a sequence at least about 70%, 75%, 80%, 85%, 90%, 95%, or 100% identical to SEQ ID NO: 55. In some embodiments, the targeting peptide comprises a sequence at least about 70%, 75%, 80%, 85%, 90%, 95%, or 100% identical to SEQ ID NO: 56. In some embodiments, the targeting peptide comprises a sequence at least about 70%, 75%, 80%, 85%, 90%, 95%, or 100% identical to SEQ ID NO: 57. In some embodiments, the targeting peptide comprises a sequence at least about 70%, 75%, 80%, 85%, 90%, 95%, or 100% identical to SEQ ID NO: 58. In some embodiments, the targeting peptide comprises a sequence at least about 70%, 75%, 80%, 85%, 90%, 95%, or 100% identical to SEQ ID NO: 59. In some embodiments, the targeting peptide comprises a sequence at least about 70%, 75%, 80%, 85%, 90%, 95%, or 100% identical to SEQ ID NO: 60. In some embodiments, the targeting peptide comprises a sequence at least about 70%, 75%, 80%, 85%, 90%, 95%, or 100% identical to SEQ ID NO: 61.

TABLE 2
Targeting Peptides

| | SEQ |
|---|---|
| | ID |
| Sequence | NO: |
| LLADTTHHRPWT | 1 |
| VIGESTHHRPWS | 2 |
| LIADSTHHSPWT | 3 |
| ILAESTHHKPWT | 4 |
| ILAETTHHRPWS | 5 |
| IIGESSHHKPFT | 6 |
| GLGDTTHHRPWG | 7 |
| VLGDTTHHKPWT | 8 |
| IVADSTHHRPWT | 9 |
| STADTSHHRPS | 10 |
| TSGGESTHHRPS | 11 |
| TSGGESSHHKPS | 12 |
| TGSGDSSHHRPS | 13 |
| GSSGESTHHKPST | 14 |
| VGADSTHHRPVT | 15 |
| GAADTTHHRPVT | 16 |
| AGADTTHHRPVT | 17 |
| GGADTTHHRPAT | 18 |
| GGADTTHHRPGT | 19 |
| LLADTTHHRPWTVIGESTHHRPWS | 20 |
| LLADTTHHRPWTVIGESTHHRPWSIIGESSHHKPFT | 21 |
| LLADTTHHRPWTVIGESTHHRPWSIIGESSHHKPFTGLGDTTHHRPWGILAESTHHKPWT | 22 |
| LLADTTHHRPWTILAESTHHKPWT | 23 |
| LLADTTHHRPWTILAESTHHKPWTLLADTTHHRPWTILAESTHHKPWTLLADTTHHRPW | 24 |
| T | |
| LLADTTHHRPWTGLGDTTHHRPWG | 25 |
| LLADTTHHRPWTGLGDTTHHRPWGLLADTTHHRPWT | 26 |
| LLADTTHHRPWTGLGDTTHHRPWGLLADTTHHRPWTGLGDTTHHRPWGLLADTTHHRP | 27 |
| WT | |
| LLADTTHHRPWTGLGDTTHHRPWGLLADTTHHRPWTGLGDTTHHRPWGLLADTTHHRP | 28 |
| WTGLGDTTHHRPWGLLADTTHHRPWT | |
| STADTSHHRPSTSGGESTHHRPSTSGGESSHHKPSTGSGDSSHHRPSGSSGESTHHKPST | 29 |
| VGADSTHHRPVTGAADTTHHRPVTAGADTTHHRPVTGGADTTHHRPATGGADTTHHRP | 30 |
| GT | |
| STADTSHHRPSLLADTTHHRPWTTSGGESTHHRPSVGADSTHHRPVTTSGGESSHHKPSG | 31 |
| AADTTHHRPVTTGSGDSSHHRPSGSSGESTHHKPSTGGADTTHHRPAT | |
| AAADTTHHRPWT | 32 |
| AAADTTHHRPWTAAADTTHHRPWTAAADTTHHRPWTAAADTTHHRPWTAAADTTHH | 33 |
| RPWT | |
| LLADAAHHRPWTLLADAAHHRPWTLLADAAHHRPWTLLADAAHHRPWTLLADAAHH | 34 |
| RPWT | |
| LLADTTAARPWTLLADTTAARPWTLLADTTAARPWTLLADTTAARPWTLLADTTAARP | 35 |
| WT | |
| LLADTTHHRPWTLLADTTHHRPWT | 36 |
| LLADTTHHRPWTLLADTTHHRPWTLLADTTHHRPWT | 37 |
| LLADTTHHRPWTLLADTTHHRPWTLLADTTHHRPWTLLADTTHHRPWTLLADTTHHRP | 38 |
| WT | |
| STSGSTVIGESTHHRPWSLIADSTHHSPWTILAESTHHKPWTILAETTHHRPWSIIGESSHH | 39 |
| KPFTGLGDTTHHRPWGVLGDTTHHKPWTIVADSTHHRPWTGQVLPTTTPSSPSTTSGS | |
| LLADTTHHRPWTVIGESTHHRPWSIIGESSHHKPFTGLGDTTHHRPWG | 40 |
| VIGESTHHRPWSIIGESSHHKPFTGLGDTTHHRPWGILAESTHHKPWT | 41 |
| (X1)(X2), wherein X1 comprises SEQ ID NO: 1 and X2 comprises one or more of SEQ ID NOS: | 42 |
| 1-41. | |
| (X1)(X2), wherein X1 comprises SEQ ID NO: 2 and X2 comprises one or more of SEQ ID NOS: | 43 |
| 1-41. | |
| (X1)(X2), wherein X1 comprises SEQ ID NO: 4 and X2 comprises one or more of SEQ ID NOS: | 44 |
| 1-41. | |
| (X1)(X2), wherein X1 comprises SEQ ID NO: 6 and X2 comprises one or more of SEQ ID NOS: | 45 |
| 1-41. | |
| (X1)(X2), wherein X1 comprises SEQ ID NO: 7 and X2 comprises one or more of SEQ ID NOS: | 46 |
| 1-41. | |
| (X1)(X2), wherein X1 comprises SEQ ID NO: 1 and X2 comprises one or more of SEQ ID NOS: | 47 |
| 2, 4, 6, or 7. | |
| (X1)(X2), wherein X1 comprises SEQ ID NO: 2 and X2 comprises one or more of SEQ ID NOS: | 48 |
| 1,4, 6, or 7. | |
| (X1)(X2), wherein X1 comprises SEQ ID NO: 4 and X2 comprises one or more of SEQ ID NOS: | 49 |
| 1, 2, 6, or 7. | |
| (X1)(X2), wherein X1 comprises SEQ ID NO: 6 and X2 comprises one or more of SEQ ID NOS: | 50 |
| 1, 4, 2, or 7. | |
| (X1)(X2), wherein X1 comprises SEQ ID NO: 7 and X2 comprises one or more of SEQ ID NOS: | 51 |
| 1, 4, 6, or 2. | |
| (X1)(X2), wherein X1 comprises SEQ ID NO: 1 and X2 comprises two or more of SEQ ID NOS: | 52 |
| 2, 4, 6, or 7. | |
| (X1)(X2), wherein X1 comprises SEQ ID NO: 2 and X2 comprises two or more of SEQ ID NOS: | 53 |
| 1, 4, 6, or 7. | |
| (X1)(X2), wherein X1 comprises SEQ ID NO: 4 and X2 comprises two or more of SEQ ID NOS: | 54 |
| 1, 2, 6, or 7. | |
| (X1)(X2), wherein X1 comprises SEQ ID NO: 6 and X2 comprises two or more of SEQ ID NOS: | 55 |
| 1, 4, 2, or 7. | |
| (X1)(X2), wherein X1 comprises SEQ ID NO: 7 and X2 comprises two or more of SEQ ID NOS: | 56 |
| 1, 4, 6, or 2. | |
| (X1)(X2), wherein X1 comprises SEQ ID NO: 1 and X2 comprises three or more of SEQ ID | 57 |
| NOS: 2, 4, 6, or 7. | |
| (X1)(X2), wherein X1 comprises SEQ ID NO: 2 and X2 comprises three or more of SEQ ID | 58 |
| NOS: 1, 4, 6, or 7. | |
| (X1)(X2), wherein X1 comprises SEQ ID NO: 4 and X2 comprises three or more of SEQ ID | 59 |
| NOS: 1, 2, 6, or 7. | |
| (X1)(X2), wherein X1 comprises SEQ ID NO: 6 and X2 comprises three or more of SEQ ID | 60 |
| NOS: 1, 4, 2, or 7. | |
| (X1)(X2), wherein X1 comprises SEQ ID NO: 7 and X2 comprises three or more of SEQ ID | 61 |
| NOS: 1, 4, 6, or 2. | |

In some embodiments, a targeting peptide comprises one or more sequences of Table 2. In some embodiments, the targeting peptide comprises a sequence at least about 70%, 75%, 80%, 85%, 90%, 95%, or 100% identical to a sequence of Table 2.

TABLE 3
Additional Targeting Peptides

| | SEQ ID |
|---|---|
| Sequence | NO |
| ACAPLMFSQC | 62 |
| ACHASLKHRC | 63 |
| ACLSTKTNIC | 64 |
| ACTTPSKHQC | 65 |
| AHFSPNLLLGG | 66 |
| AHSLKSITNHGL | 67 |
| AKQTVPV | 68 |
| AKTLMPSPFPRT | 69 |
| AMSQTMTAAIEK | 70 |
| ANPPLSL | 71 |
| ANPYHRH | 72 |
| APLSLSL | 73 |
| APYHPTIPASVHGGGK | 74 |
| ASAVGSLSIRWX | 75 |
| ASGPTNV | 76 |
| ASHNPKL | 77 |
| ASWVDSRQPSAA | 78 |
| ATFSPPL | 79 |
| ATWSHHLSSAGL | 80 |
| ATWSHHLSSAGLGGGS | 81 |
| CAHLSPHKC | 82 |
| CDIPWRNEC | 83 |
| CDPLRQHSC | 84 |
| CDSLGHWLC | 85 |
| CDYTTRHSC | 86 |
| CHGTLNPEC | 87 |
| CHHNLSWEC | 88 |
| CHIWTLASC | 89 |
| CHNTFSPRC | 90 |
| CIPLHASLC | 91 |
| CITTTSLSC | 92 |
| CKLTTCKDC | 93 |
| CKNHTTFWC | 94 |
| CLKLLSRSC | 95 |
| CLLKAHPSC | 96 |
| CLNQLKQAC | 97 |
| CLSTKTNIC | 98 |
| CMNFPSPHC | 99 |
| CNYPTLKSC | 100 |
| CPQLTVGQHRT | 101 |
| CPQSPTYTC | 102 |
| CPSSAIHTC | 103 |
| CPTSTARIC | 104 |
| CQASSFPSC | 105 |
| CQPYFWYRC | 106 |
| CQTLTPSIC | 107 |
| CSKLGHLWC | 108 |
| CSKTPERIX | 109 |
| CSNNNRMTC | 110 |
| CSPILSLSC | 111 |
| CSPTNFTRC | 112 |
| CSRPAMNVC | 113 |
| CSTKAYPNC | 114 |
| CSTSSCGSC | 115 |
| CSYWGHRDC | 116 |
| CTAHDANAC | 117 |
| CTANSEKTC | 118 |
| CTHPKASMC | 119 |
| CTKTINGKC | 120 |
| CTNMQSPLC | 121 |
| CTPFTKLPC | 122 |
| CTPTTDSIC | 123 |
| CTQQNGHPC | 124 |
| CTTPSKHQC | 125 |
| CTYNVAKPC | 126 |
| DKLHRLA | 127 |
| DLNYFTLSSKRE | 128 |
| DLPPTLHTTGSP | 129 |
| DMRQQRS | 130 |
| DQYWGLR | 131 |
| DSSNPIFWRPSS | 132 |
| DSSNPIFWRPSSGGGS | 133 |
| EFLGVPASLVNP | 134 |
| EPNHTRF | 135 |
| EPRRAVAAL | 136 |
| EPRRAVAEL | 137 |
| EPRREVAEL | 138 |
| EPRREVCEL | 139 |
| ESDLTHALHWLG | 140 |
| ESLKSIS | 141 |
| ETRTQLL | 142 |
| ETVCASS | 143 |
| ETYARPL | 144 |
| ETYQQPL | 145 |
| EVHSTDRYRSIP | 146 |
| FGLQPTGDIARR | 147 |
| FSMDDPERVRSP | 148 |
| FSPLHTSTYRPS | 149 |
| FTLPTIR | 150 |
| FVNLLGQ | 151 |
| GDFNSGHHTTTR | 152 |
| GGGAAAA | 153 |
| GIHVPWMPPVAF | 154 |
| GIHVPWMPPVAFGGGS | 155 |
| GPSNNLPWSNTP | 156 |
| GSAGLKYPLYKS | 157 |
| GSCPPKK | 158 |
| GSLFKAL | 159 |
| GTQTPQP | 160 |
| GTSRLFS | 161 |
| GVHKHFYSRWLG | 162 |
| HAPLTRSPAPNL | 163 |
| HAPVQPN | 164 |
| HGSLTTLXRYEP | 165 |
| HHFHLPKLRPPV | 166 |
| HHQRSPA | 167 |
| HHTWDTRIWQAF | 168 |
| HMLAQTF | 169 |
| HNVTTRTQRLMP | 170 |
| HPTTPIHMPNF | 171 |
| HQFISPEPFLIS | 172 |
| HQFPXSNLVWKP | 173 |
| HQWDHKY | 174 |
| HRDPXSXPSAXRP | 175 |
| HRLGHMS | 176 |
| HSACHASLKHRC | 177 |
| HSACKLTTCKDG | 178 |
| HSACLSTKTNIC | 179 |
| HSMPHMGTYLLT | 180 |
| HSTGPTR | 181 |
| HTLLSTT | 182 |
| HYPTVNF | 183 |
| IAHVPETRLAQM | 184 |
| IFSMGTALARPL | 185 |
| IGYPVLP | 186 |
| INFQFLKPSTTR | 187 |
| INKHPQQVSTLL | 188 |
| IQHQAKT | 189 |
| ISPSHSQAQADL | 191 |
| KAFDKHG | 192 |
| KATITGM | 193 |
| KEIPPIPLLAPS | 194 |
| KEIPPIPLLAPSGGGS | 195 |
| KIPKACCVPTELSAISMLYL | 196 |
| KIPKASSVPTELSAIATLYL | 197 |
| AAAAEPRRAVAAL | |
| KIPKASSVPTELSAISTLYL | 198 |
| KIPKASSVPTELSAISTLYL | 199 |
| AAAAEPRRAVAAL | |
| KIPKASSVPTELSAISTLYL | 200 |
| AAAAEPRRAVAEL | |
| KIPKASSVPTELSAISTLYL | 201 |
| AAAAEPRREVAEL | |
| KIPKASSVPTELSAISTLYL | 202 |
| AAAAXPRRXVAXL | |
| KIPKASSVPTELSAISTLYL | 203 |
| XPRRXVAXL | |
| KLHASLA | 204 |
| KLSAWSF | 205 |
| KLTWQELYQLKYKGI | 206 |
| KLTWQELYQLKYKGIGGG | 207 |
| AAAAEPRREVAEL | |
| KMNHMPN | 208 |
| KPMQFVH | 209 |
| KTSSWAN | 210 |
| LASTTHV | 211 |
| LDYPIPQTVLHH | 212 |
| LFAAVPSTQFFR | 213 |
| LGFDPTSTRFYT | 214 |
| LGPGKAF | 215 |
| LKPFSGA | 216 |
| LLADTTHHRPWP | 217 |
| LLADTTHHRPWT | 218 |
| LLADTTHHRPWTGGGS | 219 |
| LLPLKFK | 220 |
| LPFQPPI | 221 |
| LPLTPLP | 222 |
| LPRDLHATPQQI | 223 |
| LPSIHNL | 224 |
| LPWAPNLPDSTA | 225 |
| LPWTEPSFWRTP | 226 |
| LPWTEPSFWRTPGGGS | 227 |
| LQKSPSL | 228 |
| LQPSQPQRFAPT | 229 |
| LRAFPSLPHTVT | 230 |
| LSAPMEY | 231 |
| LSKNPLL | 232 |
| LSLRASAATDFQ | 233 |
| LSPPMQLQPTYS | 234 |
| LTPTMFNMHGVL | 235 |
| LTQTLQY | 236 |
| MHNVSDSNDSAI | 237 |
| MKVHERS | 238 |
| MPQTLVLPRSLL | 239 |
| MQFTPAPSPSDH | 240 |
| MTSQTLR | 241 |
| MYPLPAP | 242 |
| NERQMEL | 243 |
| NFAMNLR | 244 |
| NITQLGS | 245 |
| NKPLSTL | 246 |
| NNVSQKWQQRLI | 247 |
| NNVSQKWQQRLIGGGS | 248 |
| NPDHPDIPQDVHGGGK | 249 |
| NPMIMNQ | 250 |
| NPQMQRS | 251 |
| NPRSQAT | 252 |
| NPYAPTIPQSVAGGGK | 253 |
| NPYHPTIPQSVH | 254 |
| NPYHPTIPQSVHGGGK | 255 |
| NSMIAHNKTRMH | 256 |
| NSMIAHNKTRMHGGGS | 257 |
| NSSMLGMLPSSF | 258 |
| NTSSSQGTQRLG | 259 |
| NTTTDIPSPSQF | 260 |
| NYPTLKS | 261 |
| NYSHLRVKLPTP | 262 |
| NYSHLRVKLPTPGGGS | 263 |
| PAKQKAH | 264 |
| PDIPLSR | 265 |
| PGQWPSSLTLYK | 266 |
| PHNPGKL | 267 |
| PIDAFFD | 268 |
| PLTQPSH | 269 |
| PPKDSRG | 270 |
| PPNMARA | 271 |
| PSMKHWR | 272 |
| PTNKPHT | 273 |
| PTTMTRW | 274 |
| PTTWGHL | 275 |
| PXGPXGPXGPXGPXGPXA | 276 |
| PXGPXGPXGPXGPXGPXG | |
| QHNFRGASSSAP | 277 |
| QIPQMRILHPYG | 278 |
| QIQKPPRTPPSL | 279 |
| QLTQTMWKDTTL | 280 |
| QNLPPERYSEAT | 281 |
| QNPRQIY | 282 |
| QNYLLPK | 283 |
| QPGLWPS | 284 |
| QRSWTLDSALSM | 285 |
| QRSWTLDSALSMGGGS | 286 |
| QSLSFAGPPAWQ | 287 |
| QSSYNPI | 288 |
| QTHARHQ | 289 |
| QTHSSLW | 290 |
| QTTMTPLWPSFS | 291 |
| RCMSEVISFNCP | 292 |
| RHTLPLH | 293 |
| RPHTITN | 294 |
| RSPYYNKWSSKF | 295 |
| RTPLQPLEDFRP | 296 |
| SAGHIHEAHRPL | 297 |
| SAISDHRAHRSH | 298 |
| SAKGRAD | 299 |
| SAKKVFS | 300 |
| SASGTPS | 301 |
| SEPTYWRPNMSG | 302 |
| SFAPDIKYPVPS | 303 |
| SFQSMSLMTLVV | 304 |
| SFWHHHSPRSPL | 305 |
| SGHQLLLNKMPN | 306 |
| SGHQLLLNKMPNGGGS | 307 |
| SIFAHQTPTHKN | 308 |
| SIPKMIPTESLL | 309 |
| SIPSHSIHSAKA | 310 |
| SIRTSMNPPNLL | 311 |
| SKTSSTS | 312 |
| SLLTPWL | 313 |
| SLPHYIDNPFRQ | 314 |
| SLSKANILHLYG | 315 |
| SLVTADASFTPS | 316 |
| SMAAKSS | 317 |
| SMVYGNRLPSAL | 318 |
| SMYDTHS | 319 |
| SPEMKPR | 320 |
| SPNFSWLPLGTT | 321 |
| SPNLPWSKLSAY | 322 |
| SPNNPRE | 323 |
| SPNNTRE | 324 |
| SPSLMARSSPYW | 325 |
| SQHSTQD | 326 |
| SQTLPYSNAPSP | 327 |
| SRTGAHH | 328 |
| SSHHHRH | 329 |
| SSPPRVY | 330 |
| SSSMAKM | 331 |
| SSTLKTFFGFPD | 332 |
| SSTLKTFFGFPDGGGS | 333 |
| SSTQAHPFAPQL | 334 |
| SSTQVQHTLLQT | 335 |
| SSVPGRP | 336 |
| SSYEYHA | 337 |
| STLASMR | 338 |
| STPNSYSLPQAR | 339 |
| STQAHPW | 340 |
| STSAKHW | 341 |
| STVVMQPPPRPA | 342 |
| SVFLPTRHSPDL | 343 |
| SVQTRPLFHSHF | 344 |
| SVSVGMKPSPRP | 345 |
| SVSVGMNAESXA | 346 |
| SVSVGMNAESYG | 347 |
| SVSVGTEAESXA | 348 |
| SWPLYSRDSGLG | 349 |
| SYIDSMVPSTQT | 350 |
| SYKTTDSDTSPL | 351 |
| SYSQMDPPRSLPGGGS | 352 |
| TAAASNLRAVPP | 353 |
| TAPLSHPPRPGA | 354 |
| TDHPPKA | 355 |
| TGLAKTA | 356 |
| TGLLPNSSGAGI | 357 |
| TGPPSRQPAPLH | 358 |
| TGPTSLS | 359 |
| THPVVFEDERLF | 360 |
| TIHSKPA | 361 |
| TKDWLPS | 362 |
| TLAFQTA | 363 |
| TLAPTFR | 364 |
| TLDKYTRLLSRY | 365 |
| TLGLPML | 366 |
| TLLRTQV | 367 |
| TLMTTPP | 368 |
| TLPSPLALLTVH | 369 |
| TLQRMGQ | 370 |
| TLSNGHRYLELL | 371 |
| TMGFTAPRFPHY | 372 |
| TMRNPITSLISV | 373 |
| TMRNPITSLISVGGGS | 374 |
| TMTNMAK | 375 |
| TPLSYLKGLVTV | 376 |
| TPLTSPSLVRPQ | 377 |
| TPSPKLLQVFQA | 378 |
| TPSTGLGMSPAV | 379 |
| TPVYSLKLGPWP | 380 |
| TQTWPQSSSHGL | 381 |
| TRFYDSL | 382 |
| TRLVPSRYYHHP | 383 |
| TSPIPQMRTVPP | 384 |
| TTKNFNK | 385 |
| TTLSPRT | 386 |
| TTNSSMTMQLQR | 387 |
| TTTLPVQPTLRN | 388 |
| TTTWTTTARWPL | 389 |
| TTYNSPP | 390 |
| TVAQMPPHWQLT | 391 |
| TVLGTFP | 392 |
| TWNSNSTQYGNR | 393 |
| TWTLPAMHPRPA | 394 |
| VHLTHGQ | 395 |
| VHPRPSL | 396 |
| VHTSLLQKHPLP | 397 |
| VLPNIYMTLSA | 398 |
| VMDFASPAHVLP | 399 |
| VNQEYWFFPRRP | 400 |
| VPPISXTFLFXSTXS | 401 |
| VPPLHPALSRXN | 402 |
| VSPFLSPTPLLF | 403 |
| VSRLGTPSMHPS | 404 |
| VVKSNGE | 405 |
| VYSSPLSQLPR | 406 |
| WLPPRTQ | 407 |
| WPANKLSTKSMY | 408 |
| WPFNHFPWWNVP | 409 |
| WPTYLNPSSLKA | 410 |
| WSAHIVPYSHKP | 411 |
| WWPNSLNWVPRP | 412 |
| WYPNHLA | 413 |
| XITXGAY | 414 |
| XPRRAVAAL | 415 |
| XPRRAVAXL | 416 |
| XPRRXVAXL | 417 |
| XXFPLXG | 418 |
| YATQHNWRLKHE | 419 |
| YCPMRLCTDC | 420 |
| YELQMPLTLPLN | 421 |
| YEPAAAE | 422 |
| YGKGFSPYFHVT | 423 |
| YPHYSLPGSSTL | 424 |
| YPIMSHTCCHGV | 425 |
| YPKALRN | 426 |
| YPSLLKMQPQFS | 427 |
| YQPRPFVTTSPM | 428 |
| YSAPLARSNVVM | 429 |
| YTRLSHNPYTLS | 430 |
| YTTHVLPFAPSS | 431 |
| YTWQTIREQYEM | 432 |

In some embodiments, a targeting peptide comprises one or more sequences of Table 3. In some embodiments, the targeting peptide comprises a sequence at least about 70%, 75%, 80%, 85%, 90%, 95%, or 100% identical to a sequence of Table 3.

Additional targeting peptides useful in the present disclosure include any one of SEQ ID NO: 1 to SEQ ID NO: 558 of U.S. Pat. No. 7,572,766. In some embodiments, the targeting peptide comprises a sequence at least about 70%, 75%, 80%, 85%, 90%, 95%, or 100% identical to any one of SEQ ID NO: 1 to SEQ ID NO: 558 of U.S. Pat. No. 7,572,766.

In some embodiments, the device or kit comprises a chimeric polypeptide comprising the targeting peptide and a targeting moiety. In some cases, the chimeric polypeptide comprises a sequence at least about 70%, 75%, 80%, 85%, 90%, 95%, or 100% identical to SEQ ID NO: 433 (ASGAGGSEGGGSEGGTSGATGAGTSTSGGGASTGGGTGQAKHKQRKRLKSSCKRHPL YVDFSDVGWNDWIVAPPGYHAFYCHGECPFPLADHLNSTNHAIVQTLVNSVNSKIPKA CCVPTELSAISMLYLDENEKVVLKNYQDMVVEGCGCR). In some cases, the chimeric polypeptide comprises a sequence at least about 70%, 75%, 80%, 85%, 90%, 95%, or 100% identical to SEQ ID NO: 434 (VIPIGSLLADTTHEIRPWTVIGESTHHRPWSIIGESSHHKPFTGLGDTTHHRPWGILAESTH HKPWTASGAGGSEGGGSEGGTSGATGAGTSTSGGGASTGGGTGQAKHKQRKRLK SSC KRHPLYVDFSDVGWNDWIVAPPGYHAFYCHGECPFPLADHLNSTNHAIVQTLVNSVNS KIPKACCVPTELSAISMLYLDENEKVVLKNYQDMVVEGCGCR). In some cases, the chimeric polypeptide comprises a sequence at least about 70%, 75%, 80%, 85%, 90%, 95%, or 100% identical to SEQ ID NO: 435 (LLADTTHHRPWTVIGESTHHRPWSIIGESSHHKPFTGLGDTTHHRPWGILAESTHHKPW TASGAGGSEGGGSEGGTSGATGAGTSTSGGGASTGGGTGQAKHKQRKRLKSSCKRHPL YVDFSDVGWNDWIVAPPGYHAFYCHGECPFPLADHLNSTNHAIVQTLVNSVNSKIPKA CCVPTELSAISMLYLDENEKVVLKNYQDMVVEGCGCR). In some cases, the chimeric polypeptide comprises a sequence at least about 70%, 75%, 80%, 85%, 90%, 95%, or 100% identical to SEQ ID NO: 436 (VIGESTHHRPWSIIGESSHHKPFTGLGDTTHHRPWGILAESTHHKPWTASGAGGSEGGG SEGGTSGATGAGTSTSGGGASTGGGTGQAKHKQRKRLKSSCKRHPLYVDFSDVGWND WIVAPPGYHAFYCHGECPFPLADHLNSTNHAIVQTLVNSVNSKIPKACCVPTELSAISML YLDENEKVVLKNYQDMVVEGCGCR). In some cases, the chimeric polypeptide comprises a sequence at least about 70%, 75%, 80%, 85%, 90%, 95%, or 100% identical to SEQ ID NO: 437 (IIGESSHHKPFTGLGDTTHHRPWGILAESTHHKPWTASGAGGSEGGGSEGGTSGATGA GTSTSGGGASTGGGTGQAKHKQRKRLKSSCKRHPLYVDFSDVGWNDWIVAPPGYHAF YCHGECPFPLADHLNSTNHAIVQTLVNSVNSKIPKACCVPTELSAISMLYLDENEKVVL KNYQDMVVEGCGCR). In some cases, the chimeric polypeptide comprises a sequence at least about 70%, 75%, 80%, 85%, 90%, 95%, or 100% identical to SEQ ID NO: 438 (GLGDTTHHRPWGILAESTHHKPWTASGAGGSEGGGSEGGTSGATGAGTSTSGGGAST GGGTGQAKHKQRKRLKSSCKRHPLYVDFSDVGWNDWIVAPPGYHAFYCHGECPFPLA DHLNSTNHAIVQTLVNSVNSKIPKACCVPTELSAISMLYLDENEKVVLKNYQDMVVEG CGCR). In some cases, the chimeric polypeptide comprises a sequence at least about 70%, 75%, 80%, 85%, 90%, 95%, or 100% identical to SEQ ID NO: 439 (ILAESTHHKPWTASGAGGSEGGGSEGGTSGATGAGTSTSGGGASTGGGTGQAKHKQR KRLKSSCKRHPLYVDFSDVGWNDWIVAPPGYHAFYCHGECPFPLADHLNSTNHAIVQT LVNSVNSKIPKACCVPTELSAISMLYLDENEKVVLKNYQDMVVEGCGCR). In some cases, the chimeric polypeptide comprises a sequence at least about 70%, 75%, 80%, 85%, 90%, 95%, or 100% identical to SEQ ID NO: 440 ((X)QAKHKQRKRLKSSCKRHPLYVDFSDVGWNDWIVAPPGYHAFYCHGECPFPLADHL NSTNHAIVQTLVNSVNSKIPKACCVPIELSAISMLYLDENEKVVLKNYQDMVVEGCGC R), wherein X comprises a targeting peptide and optionally a linker. For example, the targeting peptide comprises one or more of SEQ ID NOS: 1-41. In some cases, the chimeric polypeptide comprises a sequence at least about 70%, 75%, 80%, 85%, 90%, 95%, or 100% identical to SEQ ID NO: 441 ((X)ASGAGGSEGGGSEGGTSGATGAGTSTSGGGASTGGGTGQAKHKQRKRLKSSCKRH PLYVDFSDVGWNDWIVAPPGYHAFYCHGECPFPLADHLNSTNHAIVQTLVNSVNSKIP KACCVPTELSAISMLYLDENEKVVLKNYQDMVVEGCGCR), wherein X comprises a targeting peptide and optionally a linker. For example, the targeting peptide comprises one or more of SEQ ID NOS: 1-41.

In some embodiments, a therapeutic agent is not connected to a structure using a targeting moiety. For example, the therapeutic agent may interact with the structure via non-covalent b onds. The therapeutic agent may be connected to a structure by hydrogen bonding, ionic bonding hydrophobic interactions, or van der Waals forces. The therapeutic agent may also be connected to a structure using covalent bonds. Examples of methods for connecting using covalent bonds includes chemical linkers and spacers that are used for modifying active groups within proteins such as amines, thiols and carbohydrates.

In some embodiments, provided is a device comprising a structure that is seeded with cells. Non-limiting examples of cells include osteocytes and other bone cells, chondrocytes, and meniscal cells. In some instances, the cells can be added to the completed implantable structures.

Device Manufacture

Further provided herein are methods of manufacturing a device comprising a structure and a therapeutic agent. Some methods comprise: (a) providing a first solution of a therapeutic agent (e.g., a chimeric polypeptide comprising the therapeutic agent and a targeting moiety), (b) providing a 3D structure, and (c) combining (a) and (b). In some embodiments, the therapeutic agent is present in the first solution at a concentration of about 0.25-1.5, 0.25-1.25, 0.25-1, 0.25-0.75, 0.25-0.5, 0.5-1.5, 0.5-1.25, 0.5-1, 0.5-0.75, 0.75-1.5, 0.75-1.25, 0.75-1, 1-1.5, 1-1.25, 1.25-1.5, 0.25, 0.5, 1, 1.25, or 1.5 mg/mL. In some methods, step (c) comprises incubating the first solution and structure for about 10-240, 10-180, 10-120, 20-240, 20-180, 20-120, 30-240, 30-180, 30-120, 40-240, 40-180, 40-120, 50-240, 50-180, 50-120, 60-240, 60-180, 60-120, 70-240, 70-180, 70-120, 80-240, 80-180, 80-120, 90-240, 90-180, 90-120, 100-240, 100-180, 100-120, 110-240, 110-180, 110-120, 10, 20, 30, 40, 50, 60, 70, 80, 90, 100, 110, 120, 130, 140, 150, 160, 170, 180, 190, 200, 210, 220, 230, or 240 minutes. In some methods, step (c) comprises incubating the first solution and structure with movement, such as rotation and/or shaker (e.g., using a plate shaker). In some cases, the first solution comprises a buffer. For example, the buffer comprises sodium acetate and acetic acid. In some cases, the first solution has a pH from about 4 to about 5, or about 4, 4.1, 4.15, 4.2, 4.25, 4.3, 4.35, 4.4, 4.45, 4.5, 4.45, 4.6, 4.65, 4.7, 4.75, 4.8, 4.85, 4.9, 4.95, or 5. In some cases, the first solution comprises a salt. For example, the first solution comprises sodium chloride, such as about 50-150, 50-140, 50-130, 50-120, 50-110, 50-100, 50-90, 50-80, 50-70, 50-60, 60-150, 60-140, 60-130, 60-120, 60-110, 60-100, 60-90, 60-80, 60-70, 70-150, 70-140, 70-130, 70-120, 70-110, 70-100, 70-90, 70-80, 80-150, 80-140, 80-130, 80-120, 80-110, 80-100, 80-90, 90-150, 90-140, 90-130, 90-120, 90-110, 90-100, 100-150, 100-140, 100-130, 100-120, 100-110, 110-150, 110-140, 110-130, 110-120, 120-150, 120-140, 120-130, 130-150, 130-140, or 140-150. In some embodiments, the first solution comprises 10 mM sodium acetate, 7 mM acetic acid, 100 mM NaCl, pH=4.75. In some embodiments, the method further comprises (d) washing the 3D structure of step (c) with a second solution, such as phosphate buffered saline (PBS). In some embodiments, the method further comprises drying the 3D structure of step (c) or step (d).

In some embodiments, the mass of the therapeutic agent (e.g., a therapeutic agent alone or a therapeutic agent connected to a targeting moiety) per cubic centimeter of the structure in a device is between about 0.05 and 50 (mg/cc), e.g., about 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, or 50 mg/cc or any number therebetween. For example, the therapeutic agent is about 1, 1.1, 1.2, 1.3, 1.4, 1.5, 1.6, 1.7, 1.8, 1.9, or 2 mg per cubic centimeter device. One method of measuring the amount of therapeutic peptide bound to the structure includes: (1) measuring the mass of therapeutic peptide input in the first solution, (2) measuring the mass of the therapeutic agent remaining in the first solution after combination with and removal from the structure, (3) measuring the mass of the therapeutic agent in the second solution if a wash step is included, (4) summing (2) and (3); and subtracting the sum of (4) from (1).

### Methods of Treatment

In another aspect, provided are methods of treating a subject with a structure herein. In some methods, the subject is treated with a device comprising a therapeutic peptide and the structure. In some instances, the subject has a bone fracture or a bone defect. In some instances, the subject requires a vertebral fusion of the spine. In some instances, the subject has a cartilage tear or cartilage defect. In some instances, the subject has cartilage loss.

In some embodiments, the subject is suffering from a defect in bone, cartilage, soft tissue, tendon, fascia, ligament, organ, osteotendinous tissue, dermal, or osteochondral, or a combination of one or more of the aforementioned defects. In some embodiments, a defect is a lack of bone, cartilage, soft tissue, tendon, fascia, ligament, organ, osteotendinous tissue, dermal, or osteochondral, or a combination of one or more of the aforementioned defects. In some embodiments, a defect in the subject arises from trauma. In some embodiments, a defect in the subject arises due to a congenital condition. In some embodiments, a defect in the subject arises due to an acquired condition. In some embodiments, a defect refers to the absence, loss, and/or break in a tissue and/or organ of the body. In some embodiments, a “bone defect” refers to the absence or loss (e.g., partial loss) of bone at an anatomical location in a subject where it would otherwise be present in a control healthy subject. A bone defect may be the result of an infection (e.g., osteomyelitis), a tumor, a trauma, or an adverse event of a treatment. A bone defect may also affect the muscles, soft tissue, tendons, or joints surrounding the bone defect and cause injury. In some embodiments, a bone defect includes damage to a soft tissue. In some embodiments, a “cartilage defect” refers to the absence or loss (e.g., partial loss) of cartilage at an anatomical location in a subject where it would otherwise be present in a control healthy subject. A cartilage defect may be the result of disease, osteochondritis, osteonecrosis, or trauma. For example, a cartilage defect may affect the knee joint.

Non-limiting examples of conditions suitable for treatment with a structure or device described herein include osteoarthritis, disc degeneration, congenital defect, spinal stenosis, spondylolisthesis, spondylosis, bone fracture, scoliosis, kyphosis, spinal fusion (PLF, and interbody fusions), trauma repair of bone, dental repair, craniomaxillofacial repair, ankle fusion, kyphoplasty, balloon osteoplasty, scaphoid facture repair, tendeno-osseous repair, osteoporosis, avascular necrosis, congenital skeletal malformations, costal reconstruction, subchondral bone repair, cartilage repair (e.g., at low doses), or trauma, or a combination thereof. BMP2 is also involved in hair follicle development, therefore the methods may comprise treatment to hair follicles. The trauma may be to the bone, cartilage, soft tissue, tendon, fascia, ligament, organ, osteotendinous tissue, or dermal tissue, or osteochondral tissue. In some embodiments, the method is to treat an osteochondral injury.

The methods of treatment may comprise spinal fusion. In some embodiments, spinal fusion is a surgical technique to join two or more vertebrae. In some embodiments, the spinal fusion comprises PLF. In some embodiments, the spinal fusion comprises interbody fusions.

Provided herein are methods of promoting bone or cartilage formation in a subject in need thereof that include administering to the subject a therapeutically effective amount of any of the structures or devices described herein. Some embodiments of these methods can further include first selecting a subject in need of bone or cartilage formation. In some embodiments, the structure or device is administered to the subject proximal to the desired site of bone or cartilage formation in the subject.

Also provided herein are methods of replacing and/or repairing bone or cartilage in a subject in need thereof that include administering to the subject a therapeutically effective amount of any of the structure or devices described herein. Some embodiments of these methods can further include first selecting a subject in need of bone replacement, bone repair, cartilage replacement, or cartilage repair. In some embodiments, the structure or device is administered to the subject proximal to the desired site of bone or cartilage replacement or repair in the subject.

Also provided herein are methods of treating a bone fracture or bone loss in a subject in need thereof, the method comprising administering to the subject a therapeutically effective amount of any of the structure or devices described herein. Some embodiments of these methods can further include first selecting a subject having a bone fracture or bone loss. In some embodiments, the structure or device is administered to the subject proximal to the bone fracture or the site of bone loss in the subject.

Also provided herein are methods of repairing soft tissue in a subject in need thereof that include administering to the subject a therapeutically effective amount of any of the structure or devices described herein. Some embodiments of these methods can further include first selecting a subject having a bone fracture or bone loss. In some embodiments, the composition is administered to the subject proximal to the bone fracture or the site of bone loss in the subject.

Also provided herein are methods of localized delivery of a therapeutic to a subject in need thereof that include administering to the subject a therapeutically effective amount of any of the structure or devices described herein. Some embodiments of these methods can further include first selecting a subject having a bone fracture or bone loss. In some embodiments, the structure or device is administered to the subject proximal to the bone fracture or the site of bone loss in the subject.

Methods of determining the efficacy of treatment of a bone fracture or bone loss in a subject are known in the art and include, e.g., imaging techniques (e.g., magnetic resonance imaging, X-ray, or computed tomography).

Methods of detecting bone or cartilage formation, or replacement or repair of bone or cartilage in a subject are also known in the art and include, e.g., imaging techniques (e.g., magnetic resonance imaging, X-ray, or computed tomography).

Suitable animal models for treatment of a bone fraction or bone loss, bone or cartilage formation, or bone or cartilage replacement or repair are known in the art. Non-limiting examples of such animal models are described in the Examples and in, e.g., Drosse et al., Tissue Engineering Part C 14(1):79-88, 2008; Histing et al., Bone 49:591-599, 2011; and Poser et al., Hindawi Publishing Corporation, BioMed Research International; Article ID 348635, 2014.

As used herein, a method of treatment comprises administering to the subject a structure or device herein. In some embodiments, administration comprises implanting a polypeptide or composition herein.

In some embodiments, a polypeptide and/or composition herein comprising BMP-2 is administered to the subject. In some embodiments, the BMP2 comprises a sequence at least about 70%, 75%, 80%, 85%, 90%, 95%, or 100% identical to SEQ ID NO: 454. In some embodiments, the BMP-2 is administered to induce formation of bone in the subject. In some embodiments, the BMP-2 is administered to induce formation of cartilage. In some embodiments, the BMP-2 is administered in a spinal fusion.

The terminology used herein is for the purpose of describing particular cases only and is not intended to be limiting. The singular forms “a”, “an” and “the” are intended to include the plural forms as well, unless the context clearly indicates otherwise. To the extent that the terms “including”, “includes”, “having”, “has”, “with”, or variants thereof are used in either the detailed description and/or the claims, such terms are intended to be inclusive in a manner similar to the term “comprising.”

In some embodiments, the term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value.

The term “subject” as used herein refers to any mammal. A subject therefore refers to, for example, mice, rats, dogs, cats, horses, cows, pigs, guinea pigs, rats, humans, monkeys, and the like. When the subject is a human, the subject may be referred to herein as a patient. In some embodiments, the subject or “subject in need of treatment” may be a canine (e.g., a dog), feline (e.g., a cat), equine (e.g., a horse), ovine, bovine, porcine, caprine, primate, e.g., a simian (e.g., a monkey (e.g., marmoset, baboon), or an ape (e.g., a gorilla, chimpanzee, orangutan, or gibbon), a human, or a rodent (e.g., a mouse, a guinea pig, a hamster, or a rat). In some embodiments, the subject or “subject in need of treatment” may be a non-human mammal, especially mammals that are conventionally used as models for demonstrating therapeutic efficacy in humans (e.g., murine, lapine, porcine, canine, or primate animals) may be employed.

In some embodiments, the term “therapeutically effective amount” refers to an amount of a polypeptide or composition effective to “treat” a disease, condition or disorder in a subject. In some cases, therapeutically effective amount of the polypeptide or composition reduces the severity of symptoms of the disease, condition or disorder. In some instances, the disease, condition or disorder comprises a defect in an organ or tissue.

In some embodiments, “affinity” refers to the strength of the sum total of non-covalent interactions between a β-TCP binding sequence (or a chimeric polypeptide or polypeptide comprising a β-TCP binding sequence) and its binding partner (e.g., β-TCP). Affinity can be measured by common methods known in the art, including those described herein. Affinity can be determined, for example, using surface plasmon resonance (SPR) technology (e.g., BIACORE®) or biolayer interferometry (e.g., FORTEBIO®).

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U. S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Each of the embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

A number of embodiments of the disclosure have been described. Nevertheless, it will be understood that various modifications may be made without departing from the spirit and scope of the disclosure. Additionally, while specific formulations for the inks are described, variations of the specific quantities of each ink ingredient are possible. Accordingly, other embodiments are within the scope of the following claims.

### Example Embodiments

1. A device comprising: a therapeutic agent and a printed three-dimensional structure, the printed three-dimensional structure comprising about 50% to about 90% by weight ceramic and about 10% to about 50% by weight polymer.
2. The device of embodiment 1, wherein the therapeutic agent is non-covalently bound to the printed three-dimensional structure.
3. The device of embodiment 1 or embodiment 2, wherein the ceramic comprises calcium phosphate, hydroxyapatite, fluorapatite, bone, silicate, or vanadate, or a combination thereof.
4. The device of any one of embodiments 1-3, wherein the ceramic comprises beta-tricalcium phosphate (β-TCP).
5. The device of any one of embodiments 1-4, wherein the structure comprises about 70% to about 80% by weight ceramic.
6. The device of embodiment 5, wherein the structure comprises about 75% by weight ceramic.
7. The device of any one of embodiments 1-6, wherein the polymer comprises a polyester.
8. The device of any one of embodiments 1-7, wherein the polymer comprises polycaprolactone (PCL), polyglycolide, lactide, or polydioxanone (PDS), or a combination thereof.
9. The device of any one of embodiments 1-8, wherein the polymer comprises polycaprolactone (PCL).
10. The device of any one of embodiments 1-9, wherein the polymer comprises polyglycolide.
11. The device of any one of embodiments 1-10, wherein the polymer comprises polydioxanone (PDS).
12. The device of any one of embodiments 1-11, wherein the polymer comprises a lactide.
13. The device of embodiment 12, wherein the lactide comprises L-lactide.
14. The device of embodiment 12 or embodiment 13, wherein the lactide comprises poly(D,L-lactide).
15. The device of any one of embodiments 1-14, wherein the polymer comprises a copolymer.
16. The device of embodiment 15, wherein the copolymer comprises polycaprolactone (PCL) and polyglycolide.
17. The device of embodiment 16, wherein the copolymer comprises ab out 90-95 percent by mole PCL and about 5-10 percent by mole polyglycolide.
18. The device of any one of embodiments 15-17, wherein the copolymer comprises poly(D,L-lactide-co-glycolide) copolymer.
19. The device of embodiment 18, wherein the copolymer comprises ab out 40-60 percent by mole poly(D,L-lactide) and about 40-60 percent by mole polyglycolide.
20. The device of any one of embodiments 15-19, wherein the copolymer comprises PDS-glycolide copolymer.
21. The device of embodiment 20, wherein the copolymer comprises ab out 90-95 percent by mole PDS and about 5-10 percent by mole glycolide.
22. The device of any one of embodiments 15-21, wherein the copolymer comprises PDS-L-lactide copolymer.
23. The device of embodiment 20, wherein the copolymer comprises about 90-95 percent by mole PDS and about 5-10 percent by mole L-lactide.
24. The device of any one of embodiments 1-23, wherein the structure comprises about 15% to about 25% by weight polymer.
25. The device of embodiment 24, wherein the structure comprises about 25% by weight polymer.
26. The device of any one of embodiments 1-25, wherein the printed three-dimensional structure is formed from an ink comprising about 30% to about 70% by weight the ceramic, about 10% to about 30% by weight the polymer, and optionally one or more additional agents.
27. The device of embodiment 26, wherein the ink comprises about 50% to about 70% by weight the ceramic.
28. The device of embodiment 27, wherein the ink comprises about 60% by weight the ceramic.
29. The device of any one of embodiments 26-28, wherein the ink comprises about 15% to about 25% by weight the ceramic.
30. The device of embodiment 29, wherein the ink comprises about 20% by weight the ceramic.
31. The device of any one of embodiments 26-30, wherein the ink comprises the one or more additional agents.
32. The device of any one of embodiments 26-31, wherein the ink comprises about 1% to about 30% by weight the one or more additional agents.
33. The device of any one of embodiments 26-32, wherein the one or more additional agents comprises an additional polymer, particulate, or blowing agent, or a combination thereof 34. The device of embodiment 33, wherein the one or more additional agents comprises the additional polymer.
35. The device of embodiment 33 or embodiment 34, wherein the additional polymer comprises a first polyethylene glycol and optionally a second polyethylene glycol wherein if present the second polyethylene glycol has a different molecular weight than the first polyethyleneglycol.
36. The device of any one of embodiments 33-35, wherein the additional polymer is present in the ink at about 10% to about 30% by weight.
37. The device of any one of embodiments 33-36, wherein the one or more additional agents comprises the particulate.
38. The device of any one of embodiments 33-37, wherein the particulate comprises sodium chloride, calcium chloride, sucrose, trehalose (e.g., α,α trehalose dihydrate), or mannitol (e.g., D-mannitol), or a combination thereof.
39. The device of any one of embodiments 33-38, wherein the particulate is present in the ink at about 1% to about 10% by weight.
40. The device of any one of embodiments 33-39, wherein the one or more additional agents comprises the blowing agent.
41. The device of any one of embodiments 33-40, wherein the blowing agent comprises baking powder (e.g., monocalcium phosphate, sodium bicarbonate, corn starch) and/or azodicarbonamide.
42. The device of any one of embodiments 33-41, wherein the particulate is present in the ink at about 5% to about 20% by weight.
43. The device of any one of embodiments 1-42, wherein the therapeutic agent comprises a mammalian growth factor or a functional portion thereof.
44. The device of any one of embodiments 1-43, wherein the therapeutic agent comprises one or more polypeptides selected from Table 1, or a functional portion thereof.
45. The device of any one of embodiments 1-44, wherein the therapeutic agent comprises a bone morphogenetic protein (BMP).
46. The device of any one of embodiments 1-45, wherein the therapeutic agent comprises a targeting moiety, and the targeting moiety is non-covalently bound to the printed three-dimensional structure.
47. The device of embodiment 46, wherein the targeting moiety is bound to the printed three-dimensional structure with an affinity of about 1 pM to about 100 μm.
48. The device of embodiment 46 or embodiment 47, wherein the targeting moiety comprises a polypeptide at least about 70%, 75%, 80%, 85%, 90%, 95%, or 100% identical to any one of the sequences of Tables 2-3.
49. The device of embodiment 46 or embodiment 47, wherein the targeting moiety comprises about 2, 3, 4, 5, 6, 7, 8, 9, or 10 sequences selected from a sequence from Tables 2-3.
50. The device of any one of embodiments 1-49, wherein the therapeutic agent comprises a chimeric polypeptide comprising a sequence at least about 70%, 75%, 80%, 85%, 90%, 95%, or 100% identical to any one of SEQ ID NOS: 433-441.
51. A method of treating a condition in a subject in need thereof, the method comprising administering to the subject the device of any one of embodiments 1-50.
52. The method of embodiment 51, wherein the condition comprises a bone defect, cartilage defect, soft tissue defect, tendon defect, fascia defect, ligament defect, organ defect, osteotendinous tissue defect, dermal defect, osteochondral defect, osteoporosis, avascular necrosis, or congenital skeletal malformation, or a combination thereof.
53. The method of embodiment 51 or embodiment 52, wherein the method comprises spinal fusion.
54. The method of embodiment 53, wherein the spinal fusion comprises posterior lumbar fusion (PLF) and/or interbody fusion.
55. The method of embodiment 51 or embodiment 52, wherein the method comprises bone repair, dental repair, craniomaxillofacial repair, ankle fusion, kyphoplasty, osteoplasty, scaphoid fracture repair, tendeno-osseous repair, costal reconstruction, subchondral bone repair, cartilage repair, or surgical implantation of the three-dimensional structure or device, or a combination thereof.
56. A formulation comprising about 30% to about 70% by weight ceramic, about 10% to about 30% by weight polymer, and optionally one or more additional agents.
57. The formulation of embodiment 56, wherein the ceramic comprises calcium phosphate, hydroxyapatite, fluorapatite, bone, silicate, or vanadate, or a combination thereof
58. The formulation of embodiment 56 or embodiment 57, wherein the ceramic comprises beta-tricalcium phosphate (β-TCP).
59. The formulation any one of embodiments 56-58, wherein the ink comprises about 50% to about 70% by weight the ceramic.
60. The formulation of embodiment 59, wherein the ink comprises about 60% by weight the ceramic.
61. The formulation of any one of embodiments 56-60, wherein the polymer comprises a polyester.
62. The formulation of any one of embodiments 56-61, wherein the polymer comprises polycaprolactone (PCL), polyglycolide, lactide, or polydioxanone (PDS), or a combination thereof.
63. The formulation of any one of embodiments 56-62, wherein the polymer comprises polycaprolactone (PCL).
64. The formulation of any one of embodiments 56-63, wherein the polymer comprises polyglycolide.
65. The formulation of any one of embodiments 56-64, wherein the polymer comprises polydioxanone (PDS).
66. The formulation of any one of embodiments 56-65, wherein the polymer comprises a lactide.
67. The formulation of any one of embodiments 56-66, wherein the lactide comprises L-lactide.
68. The formulation of any one of embodiments 56-67, wherein the lactide comprises poly(D,L-lactide).
69. The formulation of any one of embodiments 56-68, wherein the polymer comprises a copolymer.
70. The formulation of embodiment 69, wherein the copolymer comprises polycaprolactone (PCL) and polyglycolide.
71. The formulation of embodiment 70, wherein the copolymer comprises about 90-95 percent by mole PCL and about 5-10 percent by mole polyglycolide.
72. The formulation of any one of embodiments 69-71, wherein the copolymer comprises poly(D,L-lactide-co-glycolide) copolymer.
73. The formulation of any one of embodiments 69-72, wherein the copolymer comprises about 40-60 percent by mole poly(D,L-lactide) and about 40-60 percent by mole polyglycolide.
74. The formulation of any one of embodiments 69-73, wherein the copolymer comprises PDS-glycolide copolymer.
75. The formulation of embodiment 74, wherein the copolymer comprises about 90-95 percent by mole PDS and about 5-10 percent by mole glycolide.
76. The formulation of any one of embodiments 69-75, wherein the copolymer comprises PDS-L-lactide copolymer.
77. The formulation of embodiment 76, wherein the copolymer comprises about 90-95 percent by mole PDS and about 5-10 percent by mole L-lactide.
78. The formulation of any one of embodiments 56-77, wherein the ink comprises the one or more additional agents.
79. The formulation of any one of embodiments 56-78, wherein the ink comprises about 1% to about 30% by weight the one or more additional agents.
80. The formulation of any one of embodiments 56-79, wherein the one or more additional agents comprises an additional polymer, particulate, or blowing agent, or a combination thereof.
81. The formulation of embodiment 80, wherein the one or more additional agents comprises the additional polymer.
82. The formulation of embodiment 80 or embodiment 81, wherein the additional polymer comprises a first polyethylene glycol and optionally a second polyethylene glycol wherein if present the second polyethylene glycol has a different molecular weight than the first polyethylene glycol.
83. The formulation of any one of embodiments 80-82, wherein the additional polymer is present in the ink at about 10% to about 30% by weight.
84. The formulation of any one of embodiments 80-83, wherein the one or more additional agents comprises the particulate.
85. The formulation of any one of embodiments 80-84, wherein the particulate comprises sodium chloride, calcium chloride, sucrose, trehalose (e.g., α,α trehalose dihydrate), or mannitol (e.g., D-mannitol), or a combination thereof.
86. The formulation of any one of embodiments 80-85, wherein the particulate is present in the ink at about 1% to about 10% by weight.
87. The formulation of any one of embodiments 80-86, wherein the one or more additional agents comprises the blowing agent.
88. The formulation of any one of embodiments 80-87, wherein the blowing agent comprises baking powder (e.g., monocalcium phosphate, sodium bicarbonate, corn starch) and/or azodicarbonamide.
89. The formulation of any one of embodiments 80-88, wherein the particulate is present in the ink at about 5% to about 20% by weight.
90. A method of preparing a three-dimensional structure, the method comprising using the formation of any one of embodiments 56-89 in a three-dimensional printing method.
91. A three-dimensional structure prepared using the ink formulation of any one of embodiments 56-89.
92. The structure of embodiment 91, comprising about 50% to about 90% by weight ceramic.
93. The structure of embodiment 91, comprising about 50% to about 90% by weight tricalcium phosphate.
94. The structure of any one of embodiments 91-93, comprising about 10% to about 50% by weight polymer.
95. The structure of embodiment 94, wherein the polymer comprises a polyester.
96. The structure of embodiment 94 or embodiment 95, wherein the polymer comprises polycaprolactone (PCL), polyglycolide, lactide, or polydioxanone (PDS), or a combination thereof.
97. The structure of any one of embodiments 94-96, wherein the polymer comprises polycaprolactone (PCL).
98. The structure of any one of embodiments 94-97, wherein the polymer comprises polyglycolide.
99. The structure of any one of embodiments 94-98, wherein the polymer comprises polydioxanone (PDS).
100. The structure of any one of embodiments 94-99, wherein the polymer comprises a lactide.
101. The structure of embodiment 100, wherein the lactide comprises L-lactide.
102. The structure of embodiment 100 or embodiment 101, wherein the lactide comprises poly(D,L-lactide).
103. The structure of any one of embodiments 94-102, wherein the polymer comprises a copolymer.
104. The structure of embodiment 103, wherein the copolymer comprises polycaprolactone (PCL) and polyglycolide.
105. The structure of embodiment 104, wherein the copolymer comprises about 90-95 percent by mole PCL and about 5-10 percent by mole polyglycolide.
106. The structure of any one of embodiments 103-105, wherein the copolymer comprises poly(D,L-lactide-co-glycolide) copolymer.
107. The structure of any one of embodiments 103-106, wherein the copolymer comprises about 40-60 percent by mole poly(D,L-lactide) and about 40-60 percent by mole polyglycolide.
108. The structure of any one of embodiments 103-107, wherein the copolymer comprises PDS-glycolide copolymer.
109. The structure of embodiment 108, wherein the copolymer comprises about 90-95 percent by mole PDS and about 5-10 percent by mole glycolide.
110. The structure of any one of embodiments 103-109, wherein the copolymer comprises PDS-L-lactide copolymer.
111. The structure of embodiment 110, wherein the copolymer comprises about 90-95 percent by mole PDS and about 5-10 percent by mole L-lactide.
112. The structure of any one of embodiments 94-111, wherein the structure comprises about 15% to about 25% by weight polymer.
113. The structure of embodiment 112, wherein the structure comprises about 25% by weight polymer.
114. A three-dimensional structure comprising about 50% to about 90% by weight ceramic, and about 10% to about 30% polymer.
115. The structure of embodiment 114, wherein the ceramic comprises calcium phosphate, hydroxyapatite, fluorapatite, bone, silicate, or vanadate, or a combination thereof.
116. The structure of embodiment 114 or embodiment 115, wherein the ceramic comprises beta-tricalcium phosphate (β-TCP).
117. The structure of any one of embodiments 114-116, comprising about 65% to about 85% by weight ceramic.
118. The structure of embodiment 117, comprising about 70% to about 80% by weight ceramic.
119. The structure of embodiment 118, comprising about 75% by weight ceramic.
120. The structure of any one of embodiments 114-119, wherein the polymer comprises a polyester.
121. The structure of any one of embodiments 114-120, wherein the polymer comprises polycaprolactone (PCL), polyglycolide, lactide, or polydioxanone (PDS), or a combination thereof.
122. The structure of any one of embodiments 114-121, wherein the polymer comprises polycaprolactone (PCL).
123. The structure of any one of embodiments 114-122, wherein the polymer comprises polyglycolide.
124. The structure of any one of embodiments 114-123, wherein the polymer comprises polydioxanone (PDS).
125. The structure of any one of embodiments 114-124, wherein the polymer comprises a lactide.
126. The structure of embodiment 125, wherein the lactide comprises L-lactide.
127. The structure of embodiment 125 or embodiment 126, wherein the lactide comprises poly(D,L-lactide).
128. The structure of any one of embodiments 114-127, wherein the polymer comprises a copolymer.
129. The structure of embodiment 128, wherein the copolymer comprises polycaprolactone (PCL) and polyglycolide.
130. The structure of embodiment 129, wherein the copolymer comprises about 90-95 percent by mole PCL and about 5-10 percent by mole polyglycolide.
131. The structure of any one of embodiments 128-130, wherein the copolymer comprises poly(D,L-lactide-co-glycolide) copolymer.
132. The structure of embodiment 131, wherein the copolymer comprises about 40-60 percent by mole poly(D,L-lactide) and about 40-60 percent by mole polyglycolide.
133. The structure of any one of embodiments 128-132, wherein the copolymer comprises PDS-glycolide copolymer.
134. The structure of embodiment 133, wherein the copolymer comprises about 90-95 percent by mole PDS and about 5-10 percent by mole glycolide.
135. The structure of any one of embodiments 128-134, wherein the copolymer comprises PDS-L-lactide copolymer.
136. The structure of embodiment 135, wherein the copolymer comprises about 90-95 percent by mole PDS and about 5-10 percent by mole L-lactide.
137. The structure of any one of embodiments 114-136, wherein the structure comprises about 15% to about 25% by weight polymer.
138. The structure of embodiment 137, wherein the structure comprises about 25% by weight polymer.
139. The structure of any one of embodiments 91-138, prepared by a three-dimensional printing method.
140. A method of manufacturing the three-dimensional structure of any one of embodiments 91-139, the method comprising depositing an ink formulation in a three-dimensional form.
141. The method of embodiment 140, wherein the ink formulation comprises the ink formulation of any one of embodiments 56-89.
142. A method of treating a condition in a subject in need thereof, the method comprising delivering to an organ or tissue of the subject the structure of any one of embodiments 91-139.
143. A method of treating a condition in a subject in need thereof, the method comprising delivering to an organ or tissue of the subject the structure manufactured by the method of any one of embodiments 90, 140 or 141.
144. The method of embodiment 142 or embodiment 143, wherein the condition comprises a bone defect, cartilage defect, soft tissue defect, tendon defect, fascia defect, ligament defect, organ defect, osteotendinous tissue defect, dermal defect, osteochondral defect, osteoporosis, avascular necrosis, or congenital skeletal malformation, or a combination thereof.
145. The method of any one of embodiments 142-144, wherein the method comprises spinal fusion.
146. The method of embodiment 145, wherein the spinal fusion comprises posterior lumbar fusion (PLF) and/or interbody fusion.
147. The method of embodiment 142 or embodiment 143, wherein the method comprises bone repair, dental repair, craniomaxillofacial repair, ankle fusion, kyphoplasty, osteoplasty, scaphoid fracture repair, tendeno-osseous repair, costal reconstruction, subchondral bone repair, cartilage repair, or surgical implantation of the three-dimensional structure or device, or a combination thereof.
148. The method of any one of embodiments 142-147, further comprising treating the subject with a therapeutic agent.
149. A method of delivering a therapeutic agent to a subject in need thereof, the method comprising delivering to an organ or tissue of the subject a device comprising a therapeutic agent and the structure of any one of embodiments 91-139.
150. The method of embodiment 148 or embodiment 149, wherein the therapeutic agent comprises a mammalian growth factor or a functional portion thereof
151. The method of any one of embodiments 148-150, wherein the therapeutic agent comprises one or more polypeptides selected from Table 1, or a functional portion thereof.
152. The method of any one of embodiments 148-151, wherein the therapeutic agent comprises a bone morphogenetic protein (BMP).
153. The method of any one of embodiments 148-152, wherein the therapeutic agent comprises a targeting moiety that non-covalently binds to the structure.
154. The method of embodiment 153, wherein the targeting moiety binds to the printed three-dimensional structure with an affinity of about 1 pM to about 100 μm.
155. The method of embodiment 153 or embodiment 154, wherein the targeting moiety comprises a polypeptide at least about 70%, 75%, 80%, 85%, 90%, 95%, or 100% identical to any one of the sequences of Tables 5-6.
156. The method of embodiment 153 or embodiment 154, wherein the targeting moiety comprises about 2, 3, 4, 5, 6, 7, 8, 9, or 10 sequences selected from the sequences of Tables 5-6.
157. The method of any one of embodiments 148-156, wherein the therapeutic agent comprises or is part of a chimeric polypeptide comprising a sequence at least about 70%, 75%, 80%, 85%, 90%, 95%, or 100% identical to any one of SEQ ID NOS: 433-441.
158. The device of any one of embodiments 1-50, the method of any one of embodiments 51-55, 90, 140-157, or the structure of any one of embodiments 91-139, wherein the structure has a density of about 1 g/cm³ to about 2 g/cm³.
159. The device of any one of embodiments 1-50, the method of any one of embodiments 51-55, 90, 140-157, or the structure of any one of embodiments 91-139, wherein the structure has an open porosity of about 15% to about 50%.
160. The device of any one of embodiments 1-50, the method of any one of embodiments 51-55, 90, 140-157, or the structure of any one of embodiments 91-139, wherein the structure has a strut diameter of about 300 μm to about 600 μm.
161. A filament comprising the formulation of any one of embodiments 56-89.
162. The filament of embodiment 161, having a diameter of about 1.5 mm to about 2 mm.
163. A pellet comprising the formulation of any one of embodiments 56-89.
164. The pellet of embodiment 163, having a length of about 3 mm to about 4 mm.

### EXAMPLES

### Example 1: Ink Formulations and 3D Printed Scaffolds

Ink Formulation and Scaffold #1:

This 3D printing ink material is a flexible, polymer-ceramic composite material containing β-TCP that may be tethered to a therapeutic agent, such as tBMP2. This formulation contains two sacrificial pore formers (water soluble polyethylene glycol and water soluble sucrose) to expose more β-TCP surface area for tBMP2 binding. This ink is a low viscosity formulation that was extruded through a 400 μm diameter nozzle on an Allevi 3 pneumatic bioprinter. After 3D printing was complete, the resulting scaffold was soaked in water to dissolve the sacrificial polyethylene glycol and sucrose pore formers. The resulting scaffold #1 contains 75% by weight β-TCP powder, and 25% by weight polycaprolactone.

This ink is also converted into a filament form and utilized to prepare a 3D printed scaffold using a fused filament fabrication (FFF or FDM) 3D printer. After 3D printing is complete, the resulting scaffold is soaked in water to dissolve the sacrificial polyethylene glycol and sucrose pore formers. The resulting scaffold contains 75% by weight β-TCP powder, and 25% by weight polycaprolactone.

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 4
Example ink formulation #1

| | Wt % | Component | |
|---|---|---|---|
| Component | in ink | characteristics | Purpose |
| β-TCP Powder | 57 | Spray dried powder, | Spherical β-TCP Powder |
| | | 10-38 micron | for good extrudability |
| | | particle size | during 3D printing, binding |
| | | | sites for targeting moiety |
| Poly- | 19 | 50,000 MW, | Flexible polymer binder for |
| caprolactone | | fine powder, | β-TCP powder; not water |
| | | Tmelt = 60 C. | soluble |
| Polyethylene | 19 | 1500 MW, flake, | Water soluble polymer with |
| Glycol | | Tmelt = 60 C. | low melt viscosity for good |
| | | | extrusion |
| Sucrose | 5 | Fine powder | Water soluble particulate, |
| | | | sacrificial pore former |

Ink Formulation #2

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. This formulation employs a 95 mol % caprolactone 5 mol % glycolide copolymer for faster bioresorption characteristics compared to polycaprolactone. This ink is a low viscosity formulation that was extruded through a 320 μm diameter nozzle on an Allevi 3 pneumatic bioprinter. After 3D printing is complete, the resulting scaffold was soaked in water to dissolve the sacrificial polyethylene glycol pore former. The resulting scaffold #2 contains 75% by weight β-TCP powder, and 25% by weight caprolactone/glycolide copolymer (95:5).

This ink is also converted into a filament form and utilized to prepare a 3D printed scaffold using a fused filament fabrication (FFF or FDM) 3D printer. After 3D printing is complete, the resulting scaffold is soaked in water to dissolve the sacrificial polyethylene glycol pore former. The resulting scaffold contains 75% by weight β-TCP powder, and 25% by weight caprolactone/glycolide copolymer (95:5).

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 5
Example ink formulation #2

| | Wt % | Component | |
|---|---|---|---|
| Component | in ink | characteristics | Purpose |
| β-TCP Powder | 60 | Spray dried powder, | Spherical β-TCP Powder |
| | | 10-38 micron | for good extrudability |
| | | particle size | during 3D printing, |
| | | | binding sites for |
| | | | therapeutic |
| Caprolactone/ | 20 | 95 mol % | Flexible polymer binder |
| Glycolide | | polycaprolactone, | for β-TCP powder; not |
| copolymer | | 5 mol % | water soluble; faster |
| | | polyglycolide, | bioresorption compared to |
| | | pellets, Tmelt = | polycaprolactone |
| | | 52-62 C. | |
| Polyethylene | 20 | 1500 MW, flake, | Water soluble polymer |
| Glycol | | Tmelt = 60 C. | with low melt viscosity |
| | | | for good extrusion |

Ink Formulation #3

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. This formulation employs a 90 mol % caprolactone 10 mol % glycolide copolymer for fast bioresorption characteristics compared to polycaprolactone. This ink is a low viscosity formulation that was extruded through a 320 μm diameter nozzle on an Allevi 3 pneumatic bioprinter. After 3D printing was complete, the resulting scaffold was soaked in water to dissolve the sacrificial polyethylene glycol pore former. The resulting scaffold #3 contains 75% by weight β-TCP powder, and 25% by weight caprolactone/glycolide copolymer (90:10).

This ink is also converted into a filament form and utilized to prepare a 3D printed scaffold using a fused filament fabrication (FFF or FDM) 3D printer. After 3D printing is complete, the resulting scaffold is soaked in water to dissolve the sacrificial polyethylene glycol pore former. The resulting scaffold contains 75% by weight β-TCP powder, and 25% by weight caprolactone/glycolide copolymer (90:10).

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 6
Example ink formulation #3

| | Wt % | Component | |
|---|---|---|---|
| Component | in ink | characteristics | Purpose |
| β-TCP Powder | 60 | Spray dried powder, | Spherical β-TCP Powder |
| | | 10-38 micron | for good extrudability |
| | | particle size | during 3D printing, |
| | | | binding sites for |
| | | | therapeutic |
| Caprolactone/ | 20 | 90 mol % | Flexible polymer binder |
| Glycolide | | polycaprolactone, | for β-TCP powder; not |
| copolymer | | 10 mol % | water soluble; fast |
| | | polyglycolide, | bioresorption compared to |
| | | chips | polycaprolactone |
| Polyethylene | 20 | 1500 MW, flake, | Water soluble polymer |
| Glycol | | Tmelt = 60 C. | with low melt viscosity |
| | | | for good extrusion |

Ink Formulation #4

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. This formulation employs a 50 mol %:50 mol % poly(D,L-lactide-co-glycolide) copolymer for fast bioresorption characteristics compared to polycaprolactone. This ink is a low viscosity formulation that was extruded through a 400 μm diameter nozzle on an Allevi 3 pneumatic bioprinter. After 3D printing was complete, the resulting scaffold was soaked in water to dissolve the sacrificial polyethylene glycol pore former. The resulting scaffold #4 contains 75% by weight β-TCP powder, and 25% by weight poly(D,L-lactide-co-glycolide) copolymer (50:50).

This ink is also converted into a filament form and utilized to prepare a 3D printed scaffold using a fused filament fabrication (FFF or FDM) 3D printer. After 3D printing is complete, the resulting scaffold is soaked in water to dissolve the sacrificial polyethylene glycol pore former. The resulting scaffold contains 75% by weight β-TCP powder, and 25% by weight poly(D,L-lactide-co-glycolide) copolymer (50:50).

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 7
Example ink formulation #4

| | Wt % | Component | |
|---|---|---|---|
| Component | in ink | characteristics | Purpose |
| β-TCP Powder | 60 | Spray dried powder, | Spherical β-TCP Powder |
| | | 10-38 micron | for good extrudability |
| | | particle size | during 3D printing, |
| | | | binding sites for |
| | | | therapeutic |
| poly(D,L- | 20 | 50 mol % poly(D,L- | Flexible polymer binder |
| lactide-co- | | lactide), 50 mol % | for β-TCP powder; not |
| glycolide) | | polyglycolide, | water soluble; fast |
| copolymer | | chunks, 38,000-54,000 | bioresorption compared |
| | | MW, Tg = 46-50 C. | to polycaprolactone |
| Polyethylene | 20 | 1500 MW, flake, | Water soluble polymer |
| Glycol | | Tmelt = 60 C. | with low melt viscosity |
| | | | for good extrusion |

Ink Formulation #5

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. This ink is a moderate viscosity formulation that was formed into a 1.75 mm filament for 3D printing on a RepRap style FFF 3D printers (e.g. Prusa i3 MK3 S 3D printer) with a 400 μm diameter nozzle. The higher molecular weight polyethylene glycol (8000 MW for FFF 3D printing vs. 1500 MW for syringe-based bioprinting) results in a higher viscosity material which aids in extrusion of 1.75 mm diameter filaments. After 3D printing was complete, the resulting scaffold was soaked in water to dissolve the sacrificial polyethylene glycol pore former. The resulting scaffold #5 contains 75% by weight β-TCP powder, and 25% by weight polycaprolactone.

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 8
Example ink formulation #5

| | Wt % | Component | |
|---|---|---|---|
| Component | in ink | characteristics | Purpose |
| β-TCP Powder | 60 | Spray dried | Spherical β-TCP Powder |
| | | powder, | for good extrudability |
| | | 10-38 micron | during 3D printing, |
| | | particle size | binding sites for |
| | | | therapeutic |
| Polycaprolactone | 20 | 50,000 MW, fine | Flexible polymer binder |
| | | powder, | for β-TCP powder; not |
| | | Tmelt = 60 C. | water soluble |
| Polyethylene | 20 | 8,000 MW, flake, | Water soluble polymer |
| Glycol | | Tmelt = 60 C. | with low melt viscosity |
| | | | for good extrusion |

Ink Formulation #6

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. This ink also contains a blowing agent (sodium bicarbonate) which thermally decomposes and releases CO2 gas during 3D printing to create a foamed structure, thus increasing the porosity of the 3D printed scaffold. This ink is a moderate viscosity formulation that was formed into a 1.75 mm filament for 3D printing on a RepRap style FFF 3D printers (e.g. Prusa i3 MK3S 3D printer) with a 400 μm diameter nozzle. The higher molecular weight polyethylene glycol (8000 MW for FFF 3D printing vs. 1500 MW for syringe-based bioprinting) results in a higher viscosity material which aids in extrusion of 1.75 mm diameter filaments. After 3D printing was complete, the resulting scaffold was soaked in water to dissolve the sacrificial polyethylene glycol pore former and sodium carbonate by-product from the sodium bicarbonate thermal decomposition. The resulting scaffold #6 contains 75% by weight β-TCP powder, and 25% by weight polycaprolactone.

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 9
Example ink formulation #6

| | Wt % | Component | |
|---|---|---|---|
| Component | in ink | characteristics | Purpose |
| β-TCP Powder | 54 | Spray dried powder, | Spherical β-TCP Powder |
| | | 10-38 micron | for good extrudability |
| | | particle size | during 3D printing, |
| | | | binding sites for |
| | | | therapeutic |
| Polycaprolactone | 18 | 50,000 MW, fine | Flexible polymer binder |
| | | powder, | for β-TCP powder; not |
| | | Tmelt = 60 C. | water soluble |
| Polyethylene | 18 | 8,000 MW, flake, | Water soluble polymer |
| Glycol | | Tmelt = 60 C. | with low melt viscosity |
| | | | for good extrusion |
| Sodium | 10 | Fine powder, | Blowing agent |
| Bicarbonate | | thermal | |
| | | decomposition | |
| | | temperature 80 C. | |

Ink Formulation #7

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. This formulation employs a 90 mol %:10 mol % poly(dioxanone-co-L-lactide) copolymer for faster bioresorption characteristics compared to polycaprolactone. This ink is a low viscosity formulation that can be extruded through a 400 μm diameter nozzle on an Allevi 3 pneumatic bioprinter. After 3D printing is complete, the resulting scaffold is soaked in water to dissolve the sacrificial polyethylene glycol pore former. The resulting scaffold contains 75% by weight β-TCP powder, and 25% by weight dioxanone/L-lactide copolymer (90:10).

This ink is also converted into a filament form and utilized to prepare a 3D printed scaffold using a fused filament fabrication (FFF or FDM) 3D printer. After 3D printing is complete, the resulting scaffold is soaked in water to dissolve the sacrificial polyethylene glycol pore former. The resulting scaffold contains 75% by weight β-TCP powder, and 25% by weight dioxanone/L-lactide copolymer (90:10).

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 10
Example ink formulation #7

| | Wt % | Component | |
|---|---|---|---|
| Component | in ink | characteristics | Purpose |
| β-TCP Powder | 60 | Spray dried powder, | Spherical β-TCP Powder |
| | | 10-38 micron | for good extrudability |
| | | particle size | during 3D printing, |
| | | | binding sites for |
| | | | therapeutic |
| Dioxanone/ | 20 | 90 mol % | Flexible polymer binder |
| L-lactide | | polydioxanone, | for β-TCP powder; not |
| copolymer | | 10 mol % poly-L- | water soluble; faster |
| | | lactide, white | bioresorption compared |
| | | chips, | to polycaprolactone |
| | | Tmelt = ~110 C. | |
| Polyethylene | 20 | 1500 MW, flake, | Water soluble polymer |
| Glycol | | Tmelt = 60 C. | with low melt viscosity |
| | | | for good extrusion |

Ink Formulation #8

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. This ink is a moderate viscosity formulation that can be formed into a 1.75 mm filament for 3D printing on a RepRap style FFF 3D printers (e.g. Prusa i3 MK3 S 3D printer) with a 400 μm diameter nozzle. The higher molecular weight blend of polyethylene glycol (8000 MW and 20000 MW for FFF 3D printing vs. 1500 MW for syringe-based bioprinting) results in a higher viscosity material which aids in extrusion of 1.75 mm diameter filaments. After 3D printing is complete, the resulting scaffold is soaked in water to dissolve the sacrificial polyethylene glycol pore former. The resulting scaffold contains 75% by weight β-TCP powder, and 25% by weight polycaprolactone.

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 11
Example ink formulation #8

| | Wt % | Component | |
|---|---|---|---|
| Component | in ink | characteristics | Purpose |
| β-TCP Powder | 60 | Spray dried | Spherical β-TCP Powder |
| | | powder, | for good extrudability |
| | | 10-38 micron | during 3D printing, |
| | | particle size | binding sites for |
| | | | therapeutic |
| Polycaprolactone | 20 | 50,000 MW, fine | Flexible polymer binder |
| | | powder, | for β-TCP powder; not |
| | | Tmelt = 60 C. | water soluble |
| Polyethylene | 10 | 8,000 MW, flake, | Water soluble polymer |
| Glycol | | Tmelt = 60 C. | with low melt viscosity |
| Polyethylene | 10 | 20,000 MW, flake, | Water soluble polymer |
| Glycol | | Tmelt = 60 C. | with moderate melt |
| | | | viscosity |

Ink Formulation #9

This 3D printing ink material is a flexible, polymer-ceramic composite material containing β-TCP that may be tethered to a therapeutic agent, such as tBMP2. This formulation contains two sacrificial pore formers (water soluble polyethylene glycol and water soluble glucose) to expose more β-TCP surface area for agent binding. This ink is a moderate viscosity formulation that can be formed into a 1.75 mm filament for 3D printing on a RepRap style FFF 3D printers (e.g. Prusa i3 MK3 S 3D printer) with a 400 μm diameter nozzle. The higher molecular weight blend of polyethylene glycol (8000 MW and 20000 MW for FFF 3D printing vs. 1500 MW for syringe-based bioprinting) results in a higher viscosity material which aids in extrusion of 1.75 mm diameter filaments. After 3D printing is complete, the resulting scaffold is soaked in water to dissolve the sacrificial polyethylene glycol and sucrose pore formers. The resulting scaffold contains 75% by weight β-TCP powder, and 25% by weight polycaprolactone.

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 12
Example ink formulation #9

| | Wt % | Component | |
|---|---|---|---|
| Component | in ink | characteristics | Purpose |
| β-TCP Powder | 60 | Spray dried powder, | Spherical β-TCP Powder |
| | | 10-38 micron | for good extrudability |
| | | particle size | during 3D printing, |
| | | | binding sites for |
| | | | therapeutic |
| Polycaprolactone | 20 | 50,000 MW, fine | Flexible polymer binder |
| | | powder, | for β-TCP powder; not |
| | | Tmelt = 60 C. | water soluble |
| Polyethylene | 10 | 8,000 MW, flake, | Water soluble polymer |
| Glycol | | Tmelt = 60 C. | with low melt viscosity |
| Polyethylene | 10 | 20,000 MW, flake, | Water soluble polymer |
| Glycol | | Tmelt = 60 C. | with moderate melt |
| | | | viscosity |
| Sucrose | 5 | Fine powder | Water soluble particulate, |
| | | | sacrificial pore former |

Ink Formulation #10

This 3D printing ink material is a flexible, polymer-ceramic composite material containing β-TCP that may be tether to a therapeutic agent, such a tBMP2. This formulation contains a sacrificial pore former (water soluble polyethylene glycol) to expose more β-TCP surface area for agent binding. This formulation employs a 95 mol % caprolactone 5 mol % glycolide copolymer for faster bioresorption characteristics compared to polycaprolactone. This ink is a moderate viscosity formulation that can be formed into a 1.75 mm filament for 3D printing on a RepRap style FFF 3D printers (e.g. Prusa i3 MK3 S 3D printer) with a 400 μm diameter nozzle. The higher molecular weight blend of polyethylene glycol (8000 MW and 20000 MW for FFF 3D printing vs. 1500 MW for syringe-based bioprinting) results in a higher viscosity material which aids in extrusion of 1.75 mm diameter filaments. After 3D printing is complete, the resulting scaffold is soaked in water to dissolve the sacrificial polyethylene glycol pore former. The resulting scaffold contains 75% by weight β-TCP powder, and 25% by weight caprolactone/glycolide copolymer (95:5).

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 13
Example ink formulation #10

| | Wt % | Component | |
|---|---|---|---|
| Component | in ink | characteristics | Purpose |
| β-TCP Powder | 60 | Spray dried powder, | Spherical β-TCP Powder |
| | | 10-38 micron | for good extrudability |
| | | particle size | during 3D printing, |
| | | | binding sites for |
| | | | therapeutic |
| Caprolactone/ | 20 | 95 mol % | Flexible polymer binder |
| Glycolide | | polycaprolactone, | for β-TCP powder; not |
| copolymer | | 5 mol % | water soluble; faster |
| (95:5) | | polyglycolide, | bioresorption compared |
| | | pellets, Tmelt = | to polycaprolactone |
| | | 52-62 C. | |
| Polyethylene | 10 | 8,000 MW, flake, | Water soluble polymer with |
| Glycol | | Tmelt = 60 C. | low melt viscosity |
| Polyethylene | 10 | 20,000 MW, flake, | Water soluble polymer with |
| Glycol | | Tmelt = 60 C. | moderate melt viscosity |

Ink Formulation #11

This 3D printing ink material is a flexible, polymer-ceramic composite material containing β-TCP that may be tether to a therapeutic agent, such as tBMP2. This formulation contains a sacrificial pore former (water soluble polyethylene glycol) to expose more β-TCP surface area for agent binding. This formulation employs a 90 mol % caprolactone 10 mol % glycolide copolymer for fast bioresorption characteristics compared to polycaprolactone. This ink is a moderate viscosity formulation that can be formed into a 1.75 mm filament for 3D printing on a RepRap style FFF 3D printers (e.g. Prusa i3 MK3 S 3D printer) with a 400 μm diameter nozzle. The higher molecular weight blend of polyethylene glycol (8000 MW and 20000 MW for FFF 3D printing vs. 1500 MW for syringe-based bioprinting) results in a higher viscosity material which aids in extrusion of 1.75 mm diameter filaments. After 3D printing is complete, the resulting scaffold is soaked in water to dissolve the sacrificial polyethylene glycol pore former. The resulting scaffold contains 75% by weight β-TCP powder, and 25% by weight caprolactone/glycolide copolymer (90:10).

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 14
Example ink formulation #11

| | Wt % | Component | |
|---|---|---|---|
| Component | in ink | characteristics | Purpose |
| β-TCP Powder | 60 | Spray dried powder, | Spherical β-TCP Powder |
| | | 10-38 micron | for good extrudability |
| | | particle size | during 3D printing, |
| | | | binding sites for |
| | | | therapeutic |
| Caprolactone/ | 20 | 95 mol % | Flexible polymer binder |
| Glycolide | | polycaprolactone, | for β-TCP powder; not |
| copolymer | | 5 mol % | water soluble; fast |
| (90:10) | | polyglycolide, chips | bioresorption compared |
| | | | to polycaprolactone |
| Polyethylene | 10 | 8,000 MW, flake, | Water soluble polymer |
| Glycol | | Tmelt = 60 C. | with low melt viscosity |
| Polyethylene | 10 | 20,000 MW, flake, | Water soluble polymer with |
| Glycol | | Tmelt = 60 C. | moderate melt viscosity |

Ink Formulation #12

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. This formulation employs a 50 mol %:50 mol % poly(D,L-lactide-co-glycolide) copolymer for fast bioresorption characteristics compared to polycaprolactone. This ink is a moderate viscosity formulation that can be formed into a 1.75 mm filament for 3D printing on a RepRap style FFF 3D printers (e.g. Prusa i3 MK3 S 3D printer) with a 400 μm diameter nozzle. The higher molecular weight blend of polyethylene glycol (8000 MW and 20000 MW for FFF 3D printing vs. 1500 MW for syringe-based bioprinting) results in a higher viscosity material which aids in extrusion of 1.75 mm diameter filaments. After 3D printing is complete, the resulting scaffold is soaked in water to dissolve the sacrificial polyethylene gly col pore former. The resulting scaffold contains 75% by weight β-TCP powder, and 25% by weight poly(D,L-lactide-co-glycolide) copolymer (50:50).

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 15
Example ink formulation #12

| | Wt % | Component | |
|---|---|---|---|
| Component | in ink | characteristics | Purpose |
| β-TCP Powder | 60 | Spray dried | Spherical β-TCP Powder |
| | | powder, | for good extrudability |
| | | 10-38 micron | during 3D printing, |
| | | particle size | binding sites for |
| | | | therapeutic. |
| poly(D,L- | 20 | 50 mol % poly(D,L- | Flexible polymer binder |
| lactide-co- | | lactide), 50 mol % | for β-TCP powder; not |
| glycolide) | | polyglycolide, | water soluble; fast |
| copolymer | | chunks, 38,000- | bioresorption compared to |
| | | 54,000 MW, | polycaprolactone |
| | | Tg = 46-50 C. | |
| Polyethylene | 10 | 8,000 MW, flake, | Water soluble polymer |
| Glycol | | Tmelt = 60 C. | with low melt viscosity |
| Polyethylene | 10 | 20,000 MW, flake, | Water soluble polymer |
| Glycol | | Tmelt = 60 C. | with moderate melt |
| | | | viscosity |

Ink Formulation #13

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. 3D printing is performed using the formulation in a syringe ink and filament form. After 3D printing is complete, the resulting scaffold is soaked in water to dissolve the sacrificial polyethylene glycol pore former. The resulting scaffold contains β-TCP and PDS.

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 16
Example ink formulation #13

| Component | Wt % in ink |
|---|---|
| β-TCP Powder | 50-80 |
| Polydioxanone (PDS) | 10-30 |
| Polyethylene Glycol | 10-30 |

Ink Formulation #14

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. 3D printing is performed using the formulation in a syringe ink and filament form. After 3D printing is complete, the resulting scaffold is soaked in water to dissolve the sacrificial polyethylene glycol pore former. The resulting scaffold contains β-TCP and PDS-glycolide copolymer (90:10).

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 17
Example ink formulation #14

| Component | Wt % in ink |
|---|---|
| β-TCP Powder | 50-80 |
| PDS-Glycolide Copolymer (90:10) | 10-30 |
| Polyethylene Glycol | 10-30 |

Ink Formulation #15

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. 3D printing is performed using the formulation in a syringe ink and filament form. After 3D printing is complete, the resulting scaffold is soaked in water to dissolve the sacrificial polyethylene glycol pore former. The resulting scaffold contains β-TCP and -L-Lactide Copolymer (90:10).

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 18
Example ink formulation #15

| Component | Wt % in ink |
|---|---|
| β-TCP Powder | 50-80 |
| PDS-L-Lactide Copolymer (90:10) | 10-30 |
| Polyethylene Glycol | 10-30 |

### Example 2: Ink Preparation and Scaffold Manufacture by 3D Printing

Ink Formulation and Scaffold #1

Method: To make 5.3 cc batch of ink, 5.6 g of β-TCP powder, 1.87 g of polycaprolactone powder, 1.87 g of polyethylene glycol flake and 0.49 g sucrose were added to a glass mixing container. The glass jar was placed in a dual asymmetric centrifugal mixer (FlackTek Speedmixer) and mixed at low intensity (300 rpm) for 2 min to homogenize the powder blend before high rpm mixing. The mixer was mixed for 5 min at high intensity (3500 rpm). During mixing, the internal friction causes the polycaprolactone and polyethylene glycol to melt, changing the ink to a viscous molten liquid. This liquid phase mixing facilitates intimate dispersion of the β-TCP powder and sucrose powder into the molten polymer blend. The blended ink was allowed to cool for 10-15 min, then mixed for 5 more minutes at 3500 rpm. The mixing/cooling process was repeated for a total of four 5 min mixes at 3500 rpm. After the fourth mix at 3500 rpm, the ink charge was poured out on a glass plate and two spatulas were used to form into a roughly 1 cm diameter×6 cm long cylinder. While ink was still semi-molten, it was cut into several ˜1-2 cm long pieces with straight razor.

3D Printing: solid polymer/β-TCP pieces were transferred to a 5 cc stainless steel syringe (for use in Allevi 3 Bioprinter). Extruder CORE printing head was heated to 135° C. and allowed to dwell for approximately 30 min to ensure melting of the ink. The ink was printed with 400 micron I.D. conical metallic Luer lock tip using 70 psi pressure and 7 mm/s nozzle velocity. The scaffold was 3D printed on painter's tape applied to a smooth glass or polymer surface, such as a glass microscope slide, larger glass plate, or 96 well plate lid.

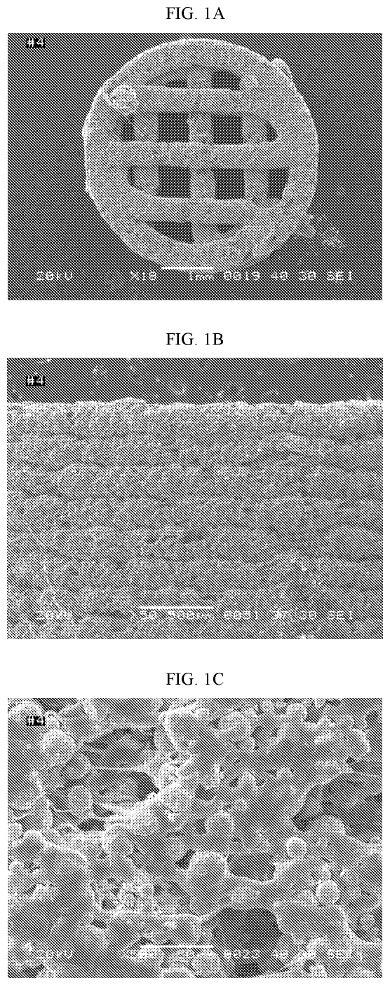

Post Processing: 3D printed structures were soaked overnight in distilled water to dissolve the polyethylene glycol and sucrose from the printed material, thus creating a porous and flexible β-TCP/polycaprolactone composite. Scaffolds were dried for at least twelve hours to ensure residual water has evaporated from the porous scaffold before binding with a therapeutic agent such as tBMP2 protein. Scaffolds were sterilized by soaking for 2-4 hours in a 70% ethanol solution and allowed to dry in biosafety cabinet for approximately 12 hours. Images of the scaffold are shown in FIGS. 1A-1C.

3D printing is also performed using a FFF 3D printer. The ink #1 is extruded into filaments and the filaments are loaded in a FFF 3D printer to generate a 3D printed scaffold. The scaffold is processed using the post processing method outlined above.

Ink Formulation and Scaffold #2

Method: To make a 5 cc batch of ink, 5.6 g of β-TCP powder, 1.87 g of 95:5 caprolactone/glycolide copolymer pellets, and 1.87 g of polyethylene glycol flake were added to a glass mixing container. The glass jar was placed in dual asymmetric centrifugal mixer (FlackTek Speedmixer) and mixed at low intensity (300 rpm) for 2 min to homogenize the powder blend before high rpm mixing. The mixture was mixed for 5 min at high intensity (3500 rpm). During mixing, the internal friction causes the 95:5 caprolactone/glycolide copolymer and polyethylene glycol to melt, changing the ink to a viscous molten liquid. This liquid phase mixing facilitates intimate dispersion of the β-TCP powder into the molten polymer blend. The blended ink was allowed to cool for 10-15 min, and then mixed for 5 more minutes at 3500 rpm. The mixing/cooling process was repeated for a total of four 5 min mixes at 3500 rpm. After the fourth mix at 3500 rpm, the ink charge was poured out on a glass plate and two spatulas were used to form into a roughly 1 cm diameter×6 cm long cylinder. While ink was still semi-molten, it was cut into several ˜1-2 cm long pieces with straight razor.

3D Printing: The solid polymer/β-TCP pieces were transferred to a 5 cc stainless steel syringe (for use in Allevi 3 Bioprinter). Extruder CORE printing head was heated to 130° C. and allowed to dwell for approximately 30 min to ensure melting of the ink. Ink was printed with 320 micron I.D. conical metallic Luer lock tip using 80 psi pressure and 6 mm/s nozzle velocity. Scaffolds were 3D printed on painter's tape applied to a smooth glass or polymer surface, such as a glass microscope slide, larger glass plate, or 96 well plate lid.

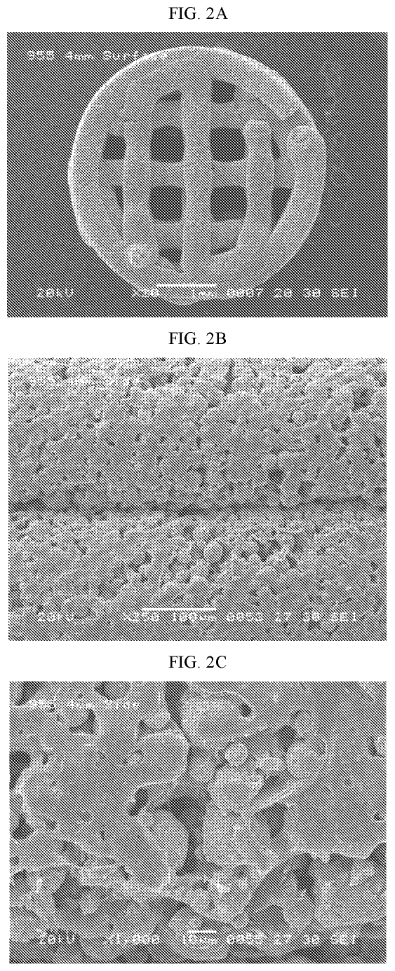

Post Processing: 3D printed structures were soaked overnight in distilled water to dissolve the polyethylene glycol from the printed material, thus creating a porous and flexible β-TCP/95:5 caprolactone/glycolide copolymer composite. The scaffolds were dried for at least twelve hours to ensure residual water evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 2A-2C.

3D printing is also performed using a FFF 3D printer. The ink #2 is extruded into filaments and the filaments are loaded in a FFF 3D printer to generate a 3D printed scaffold. The scaffold is processed using the post processing method outlined above.

Ink Formulation and Scaffold #3

Method: To make a 5 cc batch of ink, 5.6 g of β-TCP powder, 1.87 g of 90:10 caprolactone/glycolide copolymer chips, and 1.87 g of polyethylene glycol flake were added to a glass mixing container. The mixture was placed in a glass jar in dual asymmetric centrifugal mixer (FlackTek Speedmixer) and mixed at low intensity (300 rpm) for 2 min to homogenize the powder blend before high rpm mixing. It was then mixed for 5 min at high intensity (3500 rpm). During mixing, the internal friction causes the 90:10 caprolactone/glycolide copolymer and polyethylene glycol to melt, changing the ink to a viscous molten liquid. This liquid phase mixing facilitates intimate dispersion of the β-TCP powder into the molten polymer blend. The blended ink was allowed to cool for 10-15 min and then mixed for 5 more minutes at 3500 rpm. The mixing/cooling process was repeated for a total of four 5 min mixes at 3500 rpm. After the fourth mix at 3500 rpm, the ink charge was poured out on a glass plate and two spatulas were used to form into a roughly 1 cm diameter×6 cm long cylinder. While the ink was still semi-molten, it was cut into several ˜1-2 cm long pieces with straight razor.

3D Printing: the solid polymer/β-TCP pieces were transferred to a 5 cc stainless steel syringe (for use in Allevi 3 Bioprinter). Extruder CORE printing head was heated to 130° C. and allowed to dwell for approximately 30 min to ensure melting of the ink. Ink was printed with 320 micron I.D. conical metallic Luer lock tip using 45 psi pressure and 7 mm/s nozzle velocity. Scaffolds were be 3D printed on painter's tape applied to a smooth glass or polymer surface, such as a glass microscope slide, larger glass plate, or 96 well plate lid.

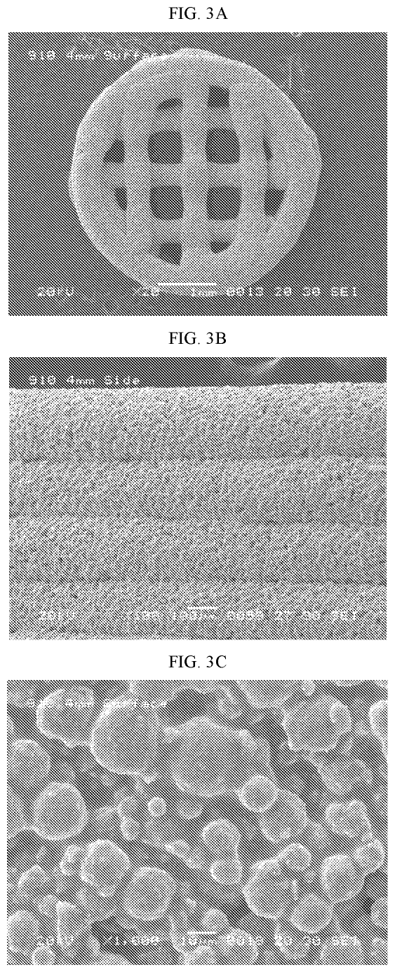

Post Processing: 3D printed structures were soaked overnight in distilled water to dissolve the polyethylene glycol from the printed material, thus creating a porous and flexible β-TCP/90:10 caprolactone/glycolide copolymer composite. Scaffolds were dried for at least twelve hours to ensure residual water has evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 3A-3C.

3D printing is also performed using a FFF 3D printer. The ink #3 is extruded into filaments and the filaments are loaded in a FFF 3D printer to generate a 3D printed scaffold. The scaffold is processed using the post processing method outlined above.

Ink Formulation and Scaffold #4

Method: To make a 2.5 cc batch of ink, 2.8 g of β-TCP powder, 0.94 g of 50:50 poly(D,L-lactide-co-glycolide) copolymer chunks, and 0.94 g of polyethylene glycol flake were added to a glass mixing container. The glass jar was placed in dual asymmetric centrifugal mixer (FlackTek Speedmixer) and mixed at low intensity (300 rpm) for 2 min to homogenize the powder blend before high rpm mixing. It was mixed for 5 min at high intensity (3500 rpm). During mixing the internal friction causes the 50:50 poly(D,L-lactide-co-glycolide) copolymer and polyethylene glycol to flow, changing the ink to a viscous molten liquid. This liquid phase mixing facilitates intimate dispersion of the β-TCP powder into the molten polymer blend. The blended ink was allowed to cool for 10-15 min and then mixed for 5 more minutes at 3500 rpm. The mixing/cooling process was repeated for a total of four 5 min mixes at 3500 rpm. After the fourth mix at 3500 rpm, the ink charge was poured out on a glass plate and two spatulas were used to form into a roughly 1 cm diameter×3 cm long cylinder. While ink was still semi-molten, it was cut into several ˜1-2 cm long pieces with straight razor.

3D Printing: The solid polymer/β-TCP pieces were transferred to a 5 cc stainless steel syringe (for use in Allevi 3 Bioprinter). Extruder CORE printing head was heated to 85° C. and allowed to dwell for approximately 30 min to ensure melting of the ink. Ink was printed with a 400 micron I.D. conical metallic Luer lock tip using 60 psi pressure and 7 mm/s nozzle velocity. Scaffolds were 3D printed on painter's tape applied to a smooth glass or polymer surface, such as a glass microscope slide, larger glass plate, or 96 well plate lid.

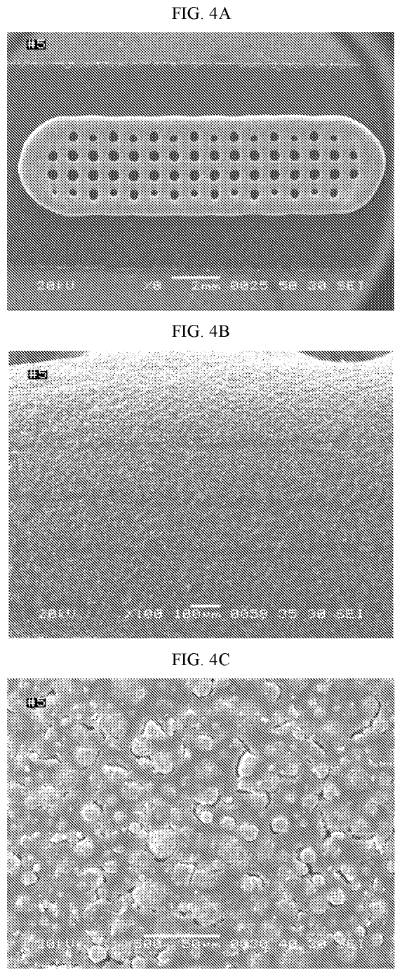

Post Processing: 3D printed structures are soaked overnight in distilled water to dissolve the polyethylene glycol from the printed material, thus creating a porous and flexible β-TCP/50:50 poly(D,L-lactide-co-glycolide) composite. Scaffolds were dried for at least twelve hours to ensure residual water has evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 4A-4C.

3D printing is also performed using a FFF 3D printer. The ink #4 is extruded into filaments and the filaments are loaded in a FFF 3D printer to generate a 3D printed scaffold. The scaffold is processed using the post processing method outlined above.

Ink Formulation and Scaffold #5

Method: To make a 5 cc batch of ink, 5.6 g of β-TCP powder, 1.87 g of polycaprolactone powder, and 1.87 g of polyethylene glycol flake were added to a glass mixing container. The glass jar was placed in dual asymmetric centrifugal mixer (FlackTek Speedmixer) and mix at low intensity (300 rpm) for 2 min to homogenize the powder blend before high rpm mixing. It was mixed for 5 min at high intensity (3500 rpm). During mixing, the internal friction causes the polycaprolactone and polyethylene glycol to melt, changing the ink to a viscous molten liquid. This liquid phase mixing facilitates intimate dispersion of the β-TCP powder into the molten polymer blend. The molten ink was poured onto a glass plate and flatten with a spatula to approximately 3 mm thick layer for cooling. After cooling, shears were used to cut into approximately 3-4 mm pellets. This was repeated for two additional 5 cc batches to create a total 15 cc of pellets for filament extrusion.

Filament Fabrication: 15 cc of 3-4 mm pellets were loaded into the hopper of a filament extruder (Filabot EX2 Filament Extruder). A 3× length extended melt filter nozzle (with filter screens removed) with 1.75 mm diameter hole size was used. A cooling fan was placed near the extrusion nozzle to speed up solidification of the extruded filament. The 1.75 mm diameter filament was extruded at extrusion temperature to 62° C. and speed at ½ on the analog dial (approximately 1 cm/second extrusion speed).

3D Printing: The 1.75 mm filament was loaded in a Prusa i3 MK3 S 3D printer. This filament material was printed using a 400 micron brass nozzle, 105° C. extruder temperature, and 15 mm/s print speed.

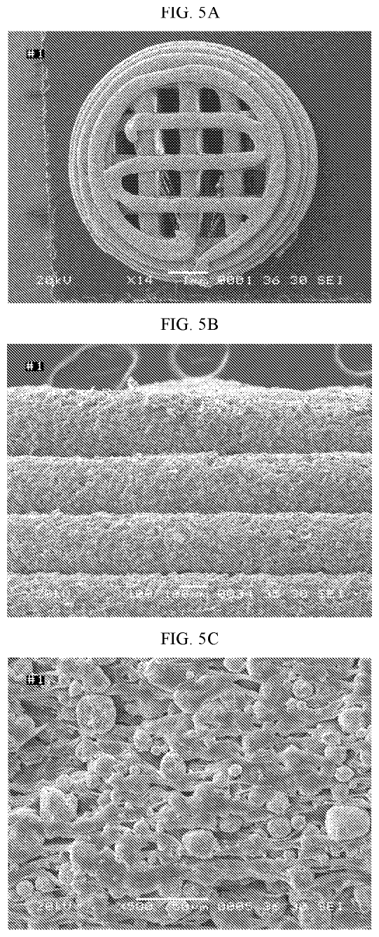

Post Processing: 3D printed structures were soaked overnight in distilled water to dissolve the polyethylene glycol from the printed material, thus creating a porous and flexible β-TCP/polycaprolactone composite. Scaffolds were dried for at least twelve hours to ensure residual water has evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 5A-5C.

Ink Formulation and Scaffold #6

Method: To make a 5.5 cc batch of ink, 5.6 g of β-TCP powder, 1.87 g of polycaprolactone powder, 1.87 g of polyethylene glycol flake, and 1.04 g of sodium bicarbonate were added to a glass mixing container. The glass jar was placed in dual asymmetric centrifugal mixer (FlackTek Speedmixer) and mixed at low intensity (300 rpm) for 2 min to homogenize the powder blend before high rpm mixing. It was mixed for 2 min at high intensity (3500 rpm). During mixing the internal friction causes the polycaprolactone and polyethylene glycol to melt, changing the ink to a viscous molten liquid. This liquid phase mixing facilitates intimate dispersion of the β-TCP powder and sodium bicarbonate powder into the molten polymer blend. The molten ink was poured onto a glass plate and flattened with a spatula to approximately 3 mm thick layer for cooling. After cooling, shears were used to cut into approximately 3-4 mm pellets. The was repeated for two additional 5.5 cc batches to create a total 16.5 cc of pellets for filament extrusion.

Filament Fabrication: 16.5 cc of 3-4 mm pellets were loaded into the hopper of a filament extruder (Filabot EX2 Filament Extruder). A 3× length extended melt filter nozzle (with filter screens removed) with 1.75 mm diameter hole size was used. A cooling fan was placed near the extrusion nozzle to speed up solidification of the extruded filament. The 1.75 mm diameter filament was extruded at extrusion temperature to 62° C. and speed at ½ on the analog dial (approximately 1 cm/second extrusion speed).

3D Printing: The 1.75 mm filament was loaded in a Prusa i3 MK3 S 3D printer. This filament material was printed using a 400 micron brass nozzle, 155° C. extruder temperature, and 15 mm/s print speed.

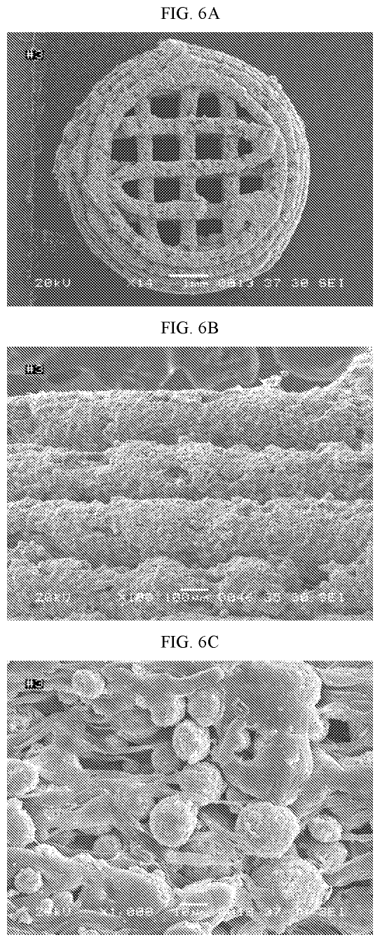

Post Processing: 3D printed structures were soaked overnight in distilled water to dissolve the polyethylene glycol and sodium carbonate (by-product of the sodium bicarbonate thermal decomposition) from the foamy printed material, thus creating a porous and flexible (3-TCP/polycaprolactone composite. Scaffolds were dried for at least twelve hours to ensure residual water has evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 6A-6C.

Ink Formulation and Scaffold #7

Method: To make a 10 cc batch of ink, 11.2 g of β-TCP powder, 3.74 g of Dioxanone/L-lactide (90:10) copolymer chips, and 3.74 g of polyethylene glycol flake were added to a glass mixing container. The glass jar was placed in a dual asymmetric centrifugal mixer (FlackTek Speedmixer) and mixed at low intensity (300 rpm) for 2 min to homogenize the powder blend before high rpm mixing. The glass jar was transferred to a hot plate and heated until it reached 185° C. (measured with IR thermometer). Next, the glass jar was immediately transferred back to the dual asymmetric centrifugal mixer and mixed for 5 min at high intensity (3500 rpm). Liquid phase mixing facilitates intimate dispersion of the β-TCP powder into the molten polymer blend. Next, the glass jar was transferred back to the hotplate and the temperature was increased to 185° C. Upon reaching the temperature, the glass jar was immediately transferred back to the dual asymmetric centrifugal mixer and mixed for 5 more minutes at 3500 rpm. The mixing/heating process was repeated for a total of four 5 min mixes at 3500 rpm. After the fourth mix at 3500 rpm, the ink charge was poured out on a glass plate and two spatulas were used to form into a roughly 1 cm diameter×6 cm long cylinder. While ink was still semi-molten, it was cut into several ˜1-2 cm long pieces with straight razor.

3D Printing: solid polymer/β-TCP pieces were transferred to a 5 cc stainless steel syringe (for use in Allevi 3 Bioprinter). Extruder CORE printing head was heated to 110° C. and allowed to dwell for approximately 30 min to ensure melting of the ink. The ink was printed with 400 micron I.D. conical metallic Luer lock tip using 15 psi pressure and 10 mm/s nozzle velocity. The scaffold was 3D printed on painter's tape applied to a smooth glass or polymer surface, such as a glass microscope slide, larger glass plate, or 96 well plate lid.

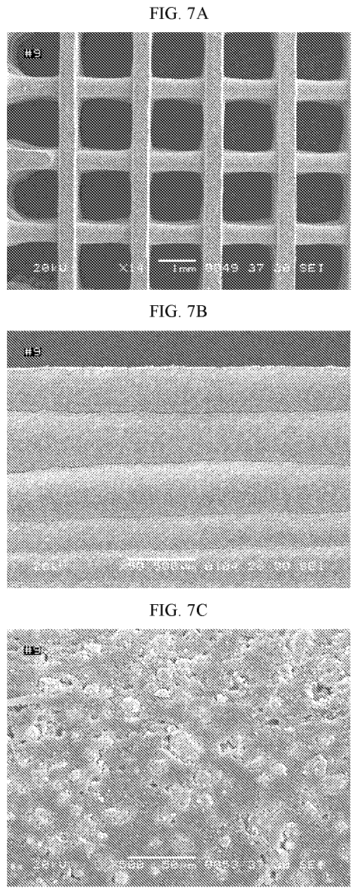

Post Processing: 3D printed structures were soaked overnight in distilled water to dissolve the polyethylene glycol from the printed material. The process created a porous and flexible β-TCP/90:10 dioxanone-L-lactide copolymer composite. Scaffolds were dried for at least twelve hours to ensure residual water has evaporated from the porous scaffold before binding with a therapeutic agent such as tBMP2 protein. Scaffolds were sterilized by soaking for 2-4 hours in a 70% ethanol solution and allowed to dry in biosafety cabinet for approximately 12 hours. Images of the scaffold are shown in FIGS. 7A-7C.

3D printing is also performed using a FFF 3D printer. The ink #7 is extruded into filaments and the filaments are loaded in a FFF 3D printer to generate a 3D printed scaffold. The scaffold is processed using the post processing method outlined above.

Ink Formulation and Scaffold #8

Method: To make a 16 cc batch of ink, 18 g of β-TCP powder, 6 g of polycaprolactone powder, 3 g of polyethylene glycol (8,000 MW) flake and 3 g of polyethylene glycol (20,000 MW) flake were added to a teflon mixing container. The teflon container was placed in dual asymmetric centrifugal mixer (FlackTek Speedmixer) and mixed at low intensity (300 rpm) for 2 min to homogenize the powder blend before high rpm mixing. Next, the teflon container was mixed for 2.5 min at high intensity (3500 rpm). During mixing, the internal friction causes the polycaprolactone and polyethylene glycol to melt, changing the ink to a viscous molten liquid. This liquid phase mixing facilitates intimate dispersion of the β-TCP powder into the molten polymer blend. The molten ink was poured onto a glass plate and flatten with a spatula to approximately 3 mm thick layer for cooling. It was cooled for 10-15 min. The mixture was returned to the teflon container and mixed for 2.5 more minutes at a high intensity (3500 rpm). The mixing/cooling process was repeated for a total of four 2.5 min mixes at 3500 rpm. After the ink was cooled after the fourth and final mix, shears were used to cut it into approximately 3-4 mm pellets.

Filament Fabrication: 16 cc of 3-4 mm pellets were loaded into the hopper of a filament extruder (Filabot EX2 Filament Extruder). A 3× length extended melt filter nozzle (with filter screens removed) with 1.75 mm diameter hole size was used. A cooling fan was placed near the extrusion nozzle to speed up solidification of the extruded filament. The 1.75 mm diameter filament was extruded at extrusion temperature to 62° C. and speed at ½ on the analog dial (approximately 1 cm/second extrusion speed).

3D Printing: The 1.75 mm filament was loaded in a Prusa i3 MK3S 3D printer. This filament material was printed using a 400 micron brass nozzle, 105° C. extruder temperature, and 15 mm/s print speed.

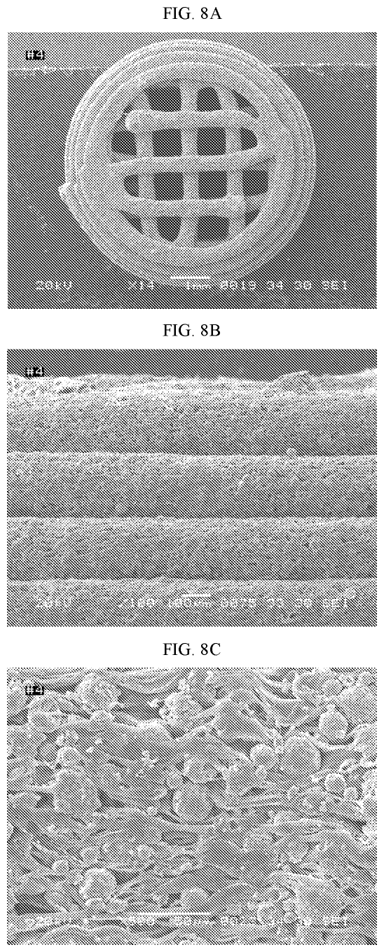

Post Processing: 3D printed structures were soaked overnight in distilled water to dissolve the polyethylene glycol from the printed material, thus creating a porous and flexible β-TCP/polycaprolactone composite. Scaffolds were dried for at least twelve hours to ensure residual water has evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 8A-8C.

Ink Formulation and Scaffold #9

Method: To make a 16.3 cc batch of ink, 17.1 g of β-TCP powder, 5.7 g of polycaprolactone powder, 2.85 g of polyethyleneglycol (8,000 MW) flake, 2.85 g of polyethylene glycol (20,000 MW) flake, and 1.5 g of sucrose were added to a teflon mixing container. The teflon container was placed in dual asymmetric centrifugal mixer (FlackTek Speedmixer) and mixed at low intensity (300 rpm) for 2 min to homogenize the powder blend before high rpm mixing. Next, the teflon container was mixed for 2 min at high intensity (3500 rpm). During mixing, the internal friction causes the polycaprolactone and polyethylene glycol to melt, changing the ink to a viscous molten liquid. This liquid phase mixing facilitates intimate dispersion of the β-TCP powder into the molten polymer blend. The molten ink was poured onto a glass plate and flatten with a spatula to approximately 3 mm thick layer for cooling. It was cooled for 10-15 min. The mixture was returned to the teflon container and mixed for 2 more minutes at a high intensity (3500 rpm). The mixing/cooling process was repeated for a total of four 2 min mixes at 3500 rpm. After the ink was cooled after the fourth and final mix, shears were used to cut it into approximately 3-4 mm pellets.

Filament Fabrication: 16.3 cc of 3-4 mm pellets were loaded into the hopper of a filament extruder (Filabot EX2 Filament Extruder). A 3× length extended melt filter nozzle (with filter screens removed) with 1.75 mm diameter hole size was used. A cooling fan was placed near the extrusion nozzle to speed up solidification of the extruded filament. The 1.75 mm diameter filament was extruded at extrusion temperature to 62° C. and speed at ½ on the analog dial (approximately 1 cm/second extrusion speed).

3D Printing: The 1.75 mm filament was loaded in a Prusa i3 MK3S 3D printer. This filament material was printed using a 400 micron brass nozzle, 140° C. extruder temperature, and 10 mm/s print speed.

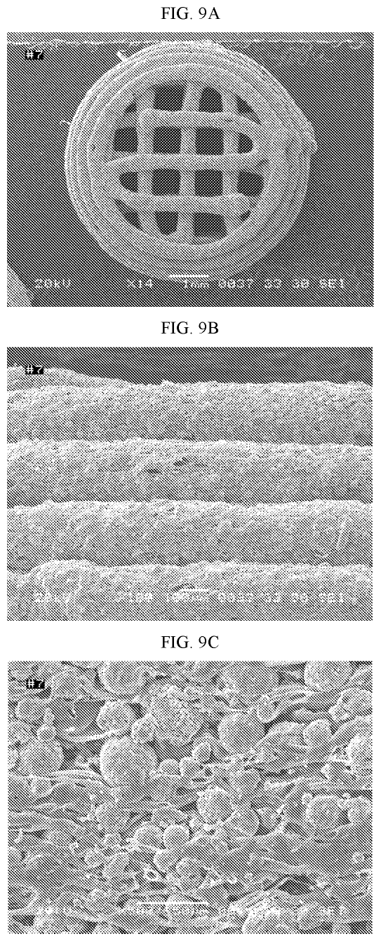

Post Processing: 3D printed structures were soaked overnight in distilled water to dissolve the polyethylene glycol and sucrose from the printed material, thus creating a porous and flexible β-TCP/polycaprolactone composite. Scaffolds were dried for at least twelve hours to ensure residual water has evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 9A-9C.

Ink Formulation and Scaffold #10

Method: To make a 16 cc batch of ink, 18 g of β-TCP powder, 6 g of caprolactone/glycolide copolymer (95:5) pellets, 3 g of polyethylene glycol (8,000 MW) flake and 3 g of polyethyleneglycol (20,000 MW) flake were added to a teflon mixing container. The teflon container was placed in dual asymmetric centrifugal mixer (FlackTek Speedmixer) and mixed at low intensity (300 rpm) for 2 min to homogenize the powder blend before high rpm mixing. Next, the teflon container was mixed for 2.5 min at high intensity (3500 rpm). During mixing, the internal friction causes the caprolactone/glycolide copolymer (95:5) and polyethylene glycol to melt, changing the ink to a viscous molten liquid. This liquid phase mixing facilitates intimate dispersion of the β-TCP powder into the molten polymer blend. The molten ink was poured onto a glass plate and flatten with a spatula to approximately 3 mm thick layer for cooling. It was cooled for 10-15 min. The mixture was returned to the teflon container and mixed for 4 more minutes at a high intensity (3500 rpm). The mixing/cooling process was repeated for a total of one 2.5 minute mix and three 4 min mixes at 3500 rpm. After the ink was cooled after the fourth and final mix, shears were used to cut it into approximately 3-4 mm pellets.

Filament Fabrication: 16 cc of 3-4 mm pellets were loaded into the hopper of a filament extruder (Filabot EX2 Filament Extruder). A 3× length extended melt filter nozzle (with filter screens removed) with 1.75 mm diameter hole size was used. A cooling fan was placed near the extrusion nozzle to speed up solidification of the extruded filament. The 1.75 mm diameter filament was extruded at extrusion temperature to 62° C. and speed at ½ on the analog dial (approximately 1 cm/second extrusion speed).

3D Printing: The 1.75 mm filament was loaded in a Prusa i3 MK3S 3D printer. This filament material was printed using a 400 micron brass nozzle, 150° C. extruder temperature, and 10 mm/s print speed.

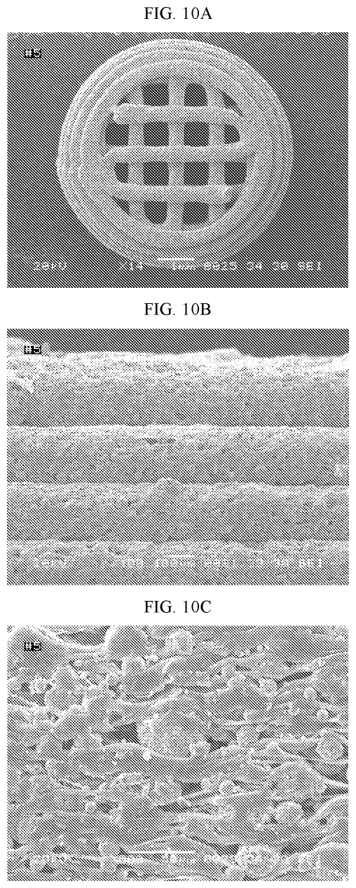

Post Processing: 3D printed structures were soaked overnight in distilled water to dissolve the polyethylene glycol from the printed material, thus creating a porous and flexible β-TCP/caprolactone/glycolide copolymer (95:5) composite. Scaffolds were dried for at least twelve hours to ensure residual water has evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 10A-10C.

Ink Formulation and Scaffold #11

Method: To make a 16 cc batch of ink, 18 g of β-TCP powder, 6 g of caprolactone/glycolide copolymer (90:10) chips, 3 g of polyethylene glycol (8,000 MW) flake and 3 g of polyethyleneglycol (20,000 MW) flake were added to a teflon mixing container. The teflon container was placed in dual asymmetric centrifugal mixer (FlackTek Speedmixer) and mixed at low intensity (300 rpm) for 2 min to homogenize the powder blend before high rpm mixing. Next, the teflon container was mixed for 2 min at high intensity (3500 rpm). During mixing, the internal friction causes the caprolactone/glycolide copolymer (90:10) and polyethylene glycol to melt, changing the ink to a viscous molten liquid. This liquid phase mixing facilitates intimate dispersion of the β-TCP powder into the molten polymer blend. The molten ink was poured onto a glass plate and flatten with a spatula to approximately 3 mm thick layer for cooling. It was cooled for 10-15 min. The mixture was returned to the teflon container and mixed for 5 more minutes at a high intensity (3500 rpm). The mixing/cooling process was repeated for a total of one 2 minute mix and three 5 min mixes at 3500 rpm. After the ink was cooled after the fourth and final mix, shears were used to cut it into approximately 3-4 mm pellets.

Filament Fabrication: 16 cc of 3-4 mm pellets were loaded into the hopper of a filament extruder (Filabot EX2 Filament Extruder). A 3× length extended melt filter nozzle (with filter screens removed) with 1.75 mm diameter hole size was used. A cooling fan was placed near the extrusion nozzle to speed up solidification of the extruded filament. The 1.75 mm diameter filament was extruded at extrusion temperature to 62° C. and speed at ½ on the analog dial (approximately 1 cm/second extrusion speed).

3D Printing: The 1.75 mm filament was loaded in a Prusa i3 MK3S 3D printer. This filament material was printed using a 400 micron brass nozzle, 150° C. extruder temperature, print bed temperature of 40° C., and 10 mm/s print speed.

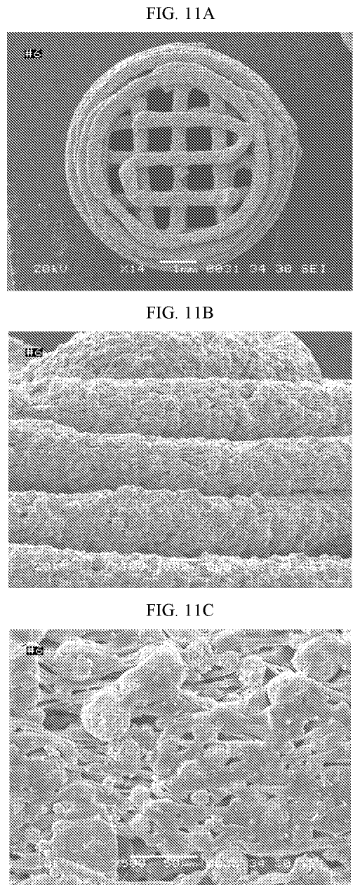

Post Processing: 3D printed structures were soaked overnight in distilled water to dissolve the polyethylene glycol from the printed material, thus creating a porous and flexible β-TCP/caprolactone/glycolide copolymer (90:10) composite. Scaffolds were dried for at least twelve hours to ensure residual water has evaporated from the porous scaffold b eforebinding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 11A-11C.

Ink Formulation and Scaffold #12

Method: To make a 8 cc batch of ink, 9 g of β-TCP powder, 3 g of Poly(D,L-lactide-co-glycolide) copolymer (50:50) chunks, 1.5 g of polyethylene glycol (8,000 MW) flake, and 1.5 g of polyethylene glycol (20,000 MW) flake were added to a teflon mixing container. The teflon container was placed in dual asymmetric centrifugal mixer (FlackTek Speedmixer) and mixed at low intensity (300 rpm) for 2 min to homogenize the powder blend before high rpm mixing. Next, the teflon container was mixed for 2 min at high intensity (3500 rpm). During mixing, the internal friction causes the Poly(D,L-lactide-co-glycolide) copolymer (50:50) and polyethylene glycol to melt, changing the ink to a viscous molten liquid. This liquid phase mixing facilitates intimate dispersion of the β-TCP powder into the molten polymer blend. The molten ink was poured onto a glass plate and flatten with a spatula to approximately 3 mm thick layer for cooling. It was cooled for 10-15 min. The mixture was returned to the teflon container and mixed for 2.5 more minutes at a high intensity (3500 rpm). The mixing/cooling process was repeated for a total of one 2 minute mix and two 2.5 min mixes at 3500 rpm. After the ink was cooled after the third and final mix, shears were used to cut it into approximately 3-4 mm pellets.

Filament Fabrication: 8 cc of 3-4 mm pellets were loaded into the hopper of a filament extruder (Filabot EX2 Filament Extruder). A 3× length extended melt filter nozzle (with filter screens removed) with 1.75 mm diameter hole size was used. A cooling fan was placed near the extrusion nozzle to speed up solidification of the extruded filament. The 1.75 mm diameter filament was extruded at extrusion temperature to 62° C. and speed at ½ on the analog dial (approximately 1 cm/second extrusion speed).

3D Printing: The 1.75 mm filament was loaded in a Prusa i3 MK3S 3D printer. This filament material was printed using a 400 micron brass nozzle, 140° C. extruder temperature, and 10 mm/s print speed.

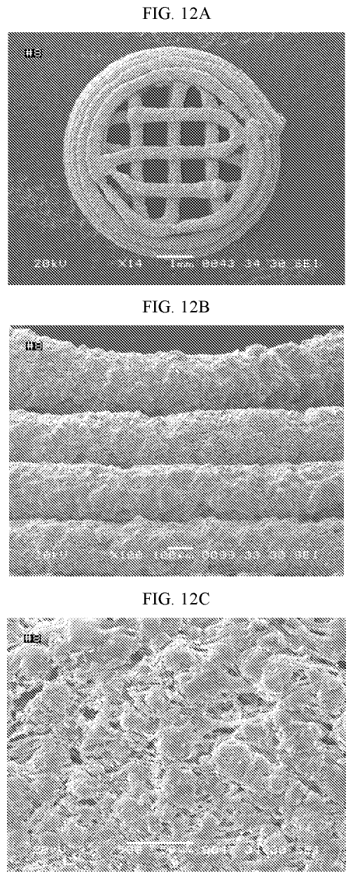

Post Processing: 3D printed structures were soaked overnight in distilled water to dissolve the polyethylene glycol from the printed material, thus creating a porous and flexible β-TCP/Poly(D,L-lactide-co-glycolide) copolymer (50:50) composite. Scaffolds were dried for at least twelve hours to ensure residual water has evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 12A-12C.

Ink Formulation and Scaffold #13

Ink #13 is printed using a syringe-based melt extrusion printing method, for example, using methods as described for printing inks #1-#4. Ink #13 is printed using fused filament fabrication 3D printing, for example, using methods as described for printing ink #5 and ink #6.

Ink Formulation and Scaffold #14 Ink #14 is printed using a syringe-based melt extrusion printing method, for example, using methods as described for printing inks #1-#4. Ink #14 is printed using fused filament fabrication 3D printing, for example, using methods as described for printing ink #5 and ink #6.

Ink Formulation and Scaffold #15 Ink #9 is printed using a syringe-based melt extrusion printing method, for example, using methods as described for printing inks #1-#4. Ink #15 is printed using fused filament fabrication 3D printing, for example, using methods as described for printing ink #5 and ink #6.

Structure Properties

Physical properties of the scaffolds #1-#6 were determined and are outlined in Table 19.

TABLE 19
Properties of Examples Scaffolds

| Ink | Density (g/cm³) | Open Porosity (%) | Strut Diameter (μm) |
|---|---|---|---|
| Ink #1 | 1.45 | 30 | 434 |
| Ink #2 | 1.56 | 23 | 397 |
| Ink #3 | 1.70 | 25 | 569 |
| Ink #4 | 1.49 | 32 | 464 |
| Ink #5 | 1.23 | 39 | 378 |
| Ink #6 | 1.22 | 39 | 392 |
| Ink #7 | 1.50 | 33 | 499 |
| Ink #8 | 1.22 | 37 | 359 |
| Ink #9 | 1.23 | 37 | 351 |
| Ink #10 | 1.28 | 37 | 374 |
| Ink #11 | 1.45 | 29 | 335 |
| Ink #12 | 1.30 | 37 | 364 |

The structures of this example are tested using Brunauer-Emmett-Teller (BET) surface area analysis by gas physisorption.

A compression test is also performed on the structures.

### Example 3: Therapeutic Agent

A chimeric polypeptide comprising the BMP therapeutic peptide connected to five b eta-tricalcium phosphate binding peptides was expressed and purified using standard expression and purification methods. The chimeric polypeptide is referred to as tBMP-2 and has the following sequence:

(SEQ ID NO: 434)
MPIGSLLADTTHHRPWTVIGESTHHRPWSIIGESSHHKPFTGLGDTTHH
RPWGILAESTHHKPWTASGAGGSEGGGSEGGTSGATGAGTSTSGGGAST
GGGTGQAKHKQRKRLKSSCKRHPLYVDFSDVGWNDWIVAPPGYHAFYCH
GECPFPLADHLNSTNHAIVQTLVNSVNSKIPKACCVPTELSAISMLYLD
ENEKVVLKNYQDMVVEGCGCR.

### Example 4: Device Manufacture

The 3D printed structures are combined with the tBMP-2 therapeutic agent to create a device. 0.75 mg/mL tBMP-2 binding solution (10 mM sodium acetate, 7 mM acetic acid, 100 mM NaCl, pH=4.75) is prepared and sterilized with 0.22 μm filter. In biosafety cabinet, tBMP-2 binding solution is added to a sterile polypropylene tube with sterile pipette such that a ratio of 15 mg of tBMP-2 per g of 3D printed scaffold is achieved. A sterile scaffold is added to binding solution with sterile tweezers, then the polypropylene tube is closed and wrapped with para film. The tube is placed on a LabLine Instruments Titer Plate Shaker, set at speed 6, and shaken for 2 hours. In a biosafety cabinet, the scaffold+tBMP-2 is removed with sterile tweezers and placed in a different sterile polypropylene tube containing sterile PBS (same volume as the tBMP-2 binding solution). The lid is closed, wrapped with parafilm, and returned to the Titer Plate Shaker to shake for 5 minutes at speed 6. The tube is opened in the biosafety cabinet, the scaffold+tBMP-2 is removed with sterile tweezers, and placed in a sterile petri dish. The scaffold+tBMP-2 is allowed to dry overnight in the biosafety cabinet, resulting in the tBMP-2 device.

The mass of tBMP-2 remaining in the binding solution and mass of tBMP-2 in the PBS wash solution is measured using A280 absorbance measurements. The sum of these masses is calculated and then subtracted from the initial mass of tBMP-2 in binding solution to arrive at the mass of tBMP-2 which remains bound to the 3D printed scaffold.

### Example 5: Animal Models

Demonstrating the utility of the embodiments described herein is done by demonstrating bone regeneration in an animal model (e.g., rats, rabbits, goats, pigs, dogs, sheep) and assessing through μCT imaging and histological analysis. Indications can include lumbar spinal fusion (a 3D printed insert for spinal fusion cage), posterolateral (PLF) spine fusion (3D printed scaffold that spans transverse processes), tibial segmental defects (a 3D printed scaffold based on patient CT data), and/or alveolar ridge augmentation (a 3D printed thin barrier membrane).

What is claimed is:

1. A three-dimensional structure comprising about 50% to about 90% by weight ceramic and about 10% to about 50% by weight polymer, wherein the structure has a plurality of micropores.

2. The structure of claim 1, wherein the micropores have an average pore size of about 1 micron to about 500 microns, or about 50 microns to about 250 microns, or about 150 microns in diameter.

3. The structure of claim 1 or claim 2, wherein the micropores provide additional surface area to the structure for contact with a therapeutic agent.

4. The structure of any one of claims 1 to 3, wherein micropores are formed after removal of one or more pore formers from the structure.

5. The structure of claim 4, wherein the pore former comprises a second polymer, a sugar, or a salt, or a combination thereof.

6. The structure of any one of claims 1 to 5, wherein micropores are formed by release of carbon dioxide from a blowing agent present during manufacture of the structure.

7. The structure of claim 6, wherein the blowing agent comprises sodium bicarbonate.

8. A three-dimensional structure comprising about 50% to about 90% by weight ceramic and about 10% to about 50% by weight polymer, wherein the structure has an open porosity of about 15% to about 50%.

9. A three-dimensional structure comprising about 50% to about 90% by weight ceramic and about 10% to about 50% by weight polymer, wherein the structure has a density of about 1 g/cm3 to about 2 g/cm3.

10. A three-dimensional structure comprising about 50% to about 90% by weight ceramic and about 10% to about 50% by weight polymer, wherein the structure has a strut diameter of about 300 micrometers to about 600 micrometers.

11. The structure of any one of claims 1 to 10, wherein the ceramic comprises calcium phosphate, hydroxyapatite, fluorapatite, bone, silicate, or vanadate, or a combination thereof.

12. The structure of any one of claims 1 to 10, wherein the polymer comprises polycaprolactone, caprolactone/glycolide copolymer, poly(D,L-lactide-co-glycolide) copolymer, or dioxanone/L-lactide copolymer, or a combination thereof.

13. The structure of any one of claims 1 to 10, wherein the ceramic comprises calcium phosphate, hydroxyapatite, fluorapatite, bone, silicate, or vanadate, or a combination thereof, and the polymer comprises polycaprolactone, caprolactone/glycolide copolymer, poly(D,L-lactide-co-glycolide) copolymer, or dioxanone/L-lactide copolymer, or a combination thereof.

14. The structure of any one of claims 1 to 10, wherein the polymer comprises polycaprolactone and the ceramic comprises calcium phosphate.

15. The structure of claim 14, comprising about 65% to about 85% by weight calcium phosphate and about 15% to about 35% by weight polycaprolactone.

16. A structure comprising about 65% to about 85% by weight calcium phosphate and about 15% to about 35% by weight polycaprolactone.

17. The structure of any one of claims 1 to 10, wherein the polymer comprises caprolactone/glycolide copolymer and the ceramic comprises calcium phosphate.

18. The structure of claim 17, comprising about 65% to about 85% by weight calcium phosphate and about 15% to about 35% by weight caprolactone/glycolide copolymer.

19. A structure comprising about 65% to about 85% by weight calcium phosphate and about 15% to about 35% by weight caprolactone/glycolide copolymer.

20. The structure of any one of claims 1 to 10, wherein the polymer comprises poly(D,L-lactide-co-glycolide) copolymer or dioxanone/L-lactide copolymer, and the ceramic comprises calcium phosphate.

21. The structure of claim 20, comprising about 65% to about 85% by weight calcium phosphate and about 15% to about 35% by weight poly(D,L-lactide-co-glycolide) copolymer or dioxanone/L-lactide copolymer.

22. A structure comprising about 65% to about 85% by weight calcium phosphate and about 15% to about 35% by weight poly(D,L-lactide-co-glycolide) copolymer or dioxanone/L-lactide copolymer.

23. The structure of any one of claims 1 to 22, formed from an ink comprises the ceramic, the polymer, and a sacrificial pore former.

24. The structure of any one of claims 1 to 23, formed from a filament using an extrusion based three dimensional printing method.

25. A device comprising the structure of any one of claims 1 to 24, and a therapeutic agent.

26. The device of claim 25, wherein the therapeutic agent comprises a bone morphogenetic protein (BMP).

27. The device of claim 25 or claim 26, wherein the therapeutic agent comprises a targeting moiety, and the targeting moiety is non-covalently bound to the three-dimensional structure.

28. The device of claim 27, wherein the targeting moiety comprises a polypeptide at least about 70%, 75%, 80%, 85%, 90%, 95%, or 100% identical to any one of the sequences of Tables 2-3.

29. The device of any one of claims 24-28, wherein the therapeutic agent comprises a chimeric polypeptide comprising a sequence at least about 70%, 75%, 80%, 85%, 90%, 95%, or 100% identical to any one of SEQ ID NOS: 433-441.
